(19) United States
(12) Patent Application Publication
(10) Pub. No.: US 2008/0305111 A1
(43) Pub. Date: Dec. 11, 2008

(54) Anti-C35 antibodies for treating cancer

(75) Inventors: Elizabeth E. Evans, Bloomfield, NY (US); Mark J. Paris, West Henrietta, NY (US); Deepak M. Sahasrabudhe, Rochester, NY (US); Maurice Zauderer, Pittsford, NY (US); Ernest S. Smith, Ontario, NY (US)

Correspondence Address: STERNE, KESSLER, GOLDSTEIN & FOX P.L.L.C., 1100 NEW YORK AVENUE, N.W., WASHINGTON, DC, 20005, US

(73) Assignee: VACCINEX, INC., Rochester, NY (US)

(21) Appl. No.: 11/812,996

(22) Filed: Jun. 22, 2007

Related U.S. Application Data
(60) Provisional application No. 60/815,562, filed on Jun. 22, 2006.

(51) Int. Cl. A61K 39/395; C12N 5/00; C07K 16/00; C07H 21/00; C12N 15/63; C12P 21/00; G01N 33/53; A61P 35/00

(52) U.S. Cl. 424/139.1; 435/375; 424/174.1; 530/387.1; 536/23.53; 435/320.1; 435/69.6; 435/325; 435/7.2

(57) ABSTRACT

The present invention is directed to methods of killing cancer cells, the methods comprising administering at least one C35 antibody and a chemotherapeutic agent. In some preferred embodiments, two C35 antibodies are administered with a chemotherapeutic agent. The present invention is further directed to C35 antibodies useful in these methods.

### CROSS-REFERENCE TO RELATED APPLICATIONS

This application claims the benefit of U.S. Provisional Application No. 60/815,562, filed Jun. 22, 2006, the entire contents of which are herein incorporated by reference.

### STATEMENT REGARDING FEDERALLY-SPONSORED RESEARCH

This invention was made with United States Government support under Award No. 70NANB4H3002, awarded by the National Institute of Standards and Technology (NIST).

The United States Government has certain rights in the invention.

### BACKGROUND OF THE INVENTION

1. Field of the Invention

The present invention is directed to methods of killing cancer cells, the methods comprising administering at least one C35 antibody and a chemotherapeutic agent. In some preferred embodiments, two C35 antibodies are administered with a chemotherapeutic agent. The present invention is further directed to the C35 antibodies useful in these methods.

2. Background Art

Cell growth is a carefully regulated process which responds to specific needs of the body. Occasionally, the intricate, and highly regulated controls dictating the rules for cellular division break down. When this occurs, the cell begins to grow and divide independently of its homeostatic regulation resulting in a condition commonly referred to as cancer. In fact, cancer is the second leading cause of death among Americans aged 2544.

Current therapies for cancer include chemotherapy and radiation therapy. Chemotherapeutic drugs kill cancer cells mainly by inducing apoptosis (Fisher, D. E., Cell 78:539-542 (1994); Fung, C. Y., and D. E. Fisher, J. Clin. Oncol. 13:801-807 (1995); Lowe, S. W., et al., Cell 74:957-967 (1993)). Radiation therapy kills cancer cells by inducing apoptosis and by other mechanisms. However, chemotherapy and radiation therapy do not kill all cells in a given tumor, and cells that survive such treatment continue to grow. Thus, these treatments are often insufficient for eradicating an entire tumor. There is therefore a need for improved therapeutic methods of treating cancer.

Immunotherapeutic strategies for cancer have also been developed that target surface membrane markers differentially expressed in tumor cells using antibodies (e.g., U.S. Pat. No. 5,770,195, “Monoclonal Antibodies to the HER2Receptor”, Filed: May 23, 1995; Issued, Jun. 23, 1998). Many antigens differentially expressed in tumors are, however, not exposed on the surface of tumor cells. As a result, such intracellular antigens are not suitable as targets for antibody-based therapeutics. Therefore, there is a need for additional targets for immunotherapeutic methods of treating cancer.

### BRIEF SUMMARY OF THE INVENTION

### Methods of Killing Cancer Cells

The present invention provides a method of killing cancer cells by administering an effective amount of a therapeutic agent, and administering an effective amount of at least one, preferably two, or more than two antibodies that bind to C35, a cancer-associated antigen which is expressed intracellularly in cancer cells, but which becomes exposed on the cell surface in cancer cells that are undergoing apoptosis.

In some embodiments, the therapeutic agent is a chemotherapeutic agent. In some embodiments, the chemotherapeutic agent is an apoptosis inducing agent. The timing of administration of the apoptosis-inducing therapy and the antibody or antibodies, is planned such that one or more of the antibodies reach the cancer cell at the time that apoptosis is being or has been induced. In some embodiments, at least one C35 antibody is conjugated to or complexed with a toxin, which insures that the cell to which the antibody binds will be killed, and/or surrounding cancer cells that are exposed to the toxin are killed. In one embodiment, the toxin is a radioisotope. In another embodiment, the toxin is a chemotherapeutic agent.

In one embodiment, the method involves administering a chemotherapeutic agent before, followed by, or simultaneous with the administration of a one or more, and preferably two antibodies or fragments or variants thereof. In some embodiments, at least one of the antibodies is conjugated to a radioactive agent.

In another embodiment, the method involves administering one or more, and preferably two antibodies or fragments or variants thereof that are not conjugated to or complexed with a toxin, and cells which bind the antibodies or fragments die. In a preferred embodiment, the one or more antibodies are administered with a chemotherapeutic agent that is not conjugated to the antibodies.

The method of the invention may be performed in vitro or in vivo, and may be used as a therapeutic in a patient, including a mammal such as a human.

Antibodies against C35 and Methods of Using C35 Antibodies

The present invention also provides antibodies that bind C35 polypeptides. The present invention encompasses antibodies (including molecules comprising, or alternatively consisting of, antibody fragments or variants thereof) that immunospecifically bind to a C35 polypeptide or polypeptide fragment or variant of a C35 polypeptide such as that of SEQ ID NO:2.

The present inventors have generated mouse and human antibodies that immunospecifically bind one or more C35 polypeptides (e.g., SEQ ID NO:2) and polynucleotides encoding VH and VL regions from these antibodies. Thus, the invention encompasses these polynucleotides, including those set forth in SEQ ID NOs:70 and 71, and those listed in Tables 2, 3 and 4 below, some of which were deposited with the American Type Culture Collection (“ATCC”) on the dates listed in Tables 2 and 3 and given the ATCC Deposit Numbers identified in Tables 2 and 3. The ATCC is located at 10801 University Boulevard, Manassas, Va. 20110-2209, USA. The ATCC deposit was made pursuant to the terms of the Budapest Treaty on the international recognition of the deposit of microorganisms for purposes of patent procedure.

The present invention also encompasses the deposited polynucleotide clones that encode VH and VL regions that immunospecifically bind one or more C35 polypeptides (e.g., SEQ ID NO:2), cells comprising the deposited polynucleotides, antibodies comprising VH and/or VL regions encoded by the deposited polynucleotides or portions thereof (e.g., VH or VL CDRs), polynucleotides encoding such antibodies, and cells comprising such polynucleotides. The present invention also encompasses cells comprising the polynucleotides of SEQ ID NO:s 70 and 71, antibodies comprising VH and/or VL regions encoded by the nucleotides of SEQ ID NO:s 70 and 71, or the VH and/or VL regions encoded by the polypeptides of SEQ ID NO:s 62 and 66, polynucleotides encoding such antibodies, and cells comprising such polynucleotides. Such antibodies may or may not have the same epitope specificity as the original antibodies comprising the VH and VL regions encoded by the polynucleotides, and may or may not have an affinity for C35 the same as or higher than the affinity of the original antibodies. In one embodiment, the antibodies of the present invention bind a C35 epitope contained within residues 105 to 115 of SEQ ID NO:2. In another embodiment, the antibodies of the present invention bind a C35 epitope contained within residues 48 to 104 of SEQ ID NO:2. In another embodiment, the antibodies of the present invention bind a C35 epitope contained within residues 48 to 104 of SEQ ID NO:2.

Further, the present invention encompasses, antibodies comprising, or alternatively consisting of, fragments or variants of these antibodies (e.g., scFvs, diabodies, triabodies, tetrabodies, minibodies, heavy chains, VH regions, VH CDRs (Complementarity Determining Regions), light chains, VL regions, or VL CDRs) having an amino acid sequence of any one of the VH, VH CDRs, VLs, VL CDRs encoded by a polynucleotide of the invention. Such antibodies may or may not have the same epitope specificity as the original antibodies comprising the VH and VL regions encoded by the deposited polynucleotides, and may or may not have an affinity for C35 the same as or higher than the affinity of the original antibodies.

The present invention also provides antibodies or fragments or variants thereof that bind one or more C35 polypeptides, and which are coupled to a detectable label, such as an enzyme, a fluorescent label, a luminescent label, or a bioluminescent label. The present invention also provides antibodies or fragments or variants thereof that bind one or more C35 polypeptides, and which are coupled to a therapeutic or a toxin, e.g., a radioactive material. In one embodiment, the antibodies of the present invention are coupled to a radioisotope.

The present invention also provides for a nucleic acid molecule(s), generally isolated, encoding an antibody (including molecules, such as scFvs, diabodies, triabodies, tetrabodies, minibodies, VH regions, or VL regions, that comprise, or alternatively consist of, an antibody fragment or variant thereof) of the invention. The present invention also provides a host cell transformed with a nucleic acid molecule encoding an antibody (including molecules, such as scFvs, diabodies, triabodies, tetrabodies, minibodies, VH regions, or VL regions, that comprise, or alternatively consist of, an antibody fragment or variant thereof) of the invention and progeny thereof. The present invention also provides a method for the production of an antibody (including a molecule comprising, or alternatively consisting of, an antibody fragment or variant thereof) of the invention. The present invention further provides a method of expressing an antibody (including a molecule comprising, or alternatively consisting of, an antibody fragment or variant thereof) of the invention from a nucleic acid molecule.

The present invention relates to methods and compositions for treating cancer comprising administering to a mammal, preferably a human, an effective amount of one or more, and preferably two antibodies or fragments or variants thereof, or related molecules, that immunospecifically bind a C35 polypeptide or a fragment or variant thereof. In preferred embodiments, the present invention relates to antibody-based methods and compositions for treating breast cancer, ovarian cancer, bladder cancer, lung cancer, prostate cancer, pancreatic cancer, colon cancer, and melanoma.

In a preferred embodiment, the present invention relates, to a combination therapy for treating cancer comprising administering to a mammal, preferably a human, an effective amount of a chemotherapeutic agent and an effective amount of one or more antibodies, or fragments or variants thereof. In some embodiments, the antibodies or fragments or variants thereof are conjugated with a toxin, e.g., a radioactive material. In some embodiments, the antibody or fragment thereof of is conjugated to an agent selected from the group consisting of a therapeutic agent, a prodrug, a peptide, a protein, an enzyme, a virus, a lipid, a biological response modifier, a pharmaceutical agent, or PEG. In a preferred embodiment, the present invention is directed to a method of killing cancer cells that express C35 comprising administering to the cells two antibodies or fragments or variants thereof that specifically bind C35 and an effective amount of a therapeutic agent. In a particular embodiment, the therapeutic agent is a chemotherapeutic agent. In a more specific embodiment, the chemotherapeutic agent is paclitaxel. In another particular embodiment, at least one, and preferably two of the C35 antibodies or fragments are selected from the group consisting of 1B3 (Mab 11), 1F2 (Mab 76), MAb 163, MAb 165, Mab 171, MAbc009, and variants or derivatives thereof. In a more specific embodiment, the two antibodies are 1B3 and 1F2 or fragments or variants thereof.

The present invention also encompasses methods and compositions for detecting, diagnosing, or prognosing cancer comprising administering to a mammal, preferably a human, an effective amount of one or more antibodies or fragments or variants thereof, or related molecules, that immunospecifically bind to C35 or a fragment or variant thereof. In preferred embodiments, the present invention relates to antibody-based methods and compositions for detecting, diagnosing, or prognosing breast cancer, ovarian cancer, bladder cancer, lung cancer, prostate cancer, pancreatic cancer, colon cancer, and melanoma.

Another embodiment of the present invention includes the use of the antibodies of the invention as a diagnostic tool to monitor the expression of C35 or in cancer. In certain embodiments, the method may also be employed as a diagnostic to confirm the efficacy of an apoptosis inducing regimen.

These and other aspects of the invention are described in further detail below.

### BRIEF DESCRIPTION OF THE FIGURES

FIG. 1 shows C35 surface staining of breast tumor cells following radiation induced apoptosis in 21MT1 breast tumor cells that express the C35 tumor antigen. FIG. 1A shows that untreated live cells (PI negative), that are not undergoing apoptosis (Annexin V negative) do not express C35 on the surface membrane as evidenced by absence of differential staining with anti-C35 antibody and the isotype control antibody. FIG. 1B shows, similarly, that irradiated tumor cells that remain viable (PI negative) and have not been induced to undergo apoptosis (Annexin V negative) also do not express C35 on the tumor cell surface membrane. FIG. 1C shows, in contrast, that irradiated tumor cells that are viable (PI negative), but undergoing apoptosis (Annexin V positive), are clearly differentially stained with anti-C35 antibodies as compared to isotype control antibody.

FIG. 2 shows C35 surface staining of breast tumor cells following mitomycin C drug induced apoptosis. FIG. 2A shows that untreated live cells (PI negative), that are not undergoing apoptosis (Annexin V negative), do not express C35 on the surface membrane as evidenced by absence of differential staining with anti-C35 antibody and the isotype control antibody. FIG. 2B shows, similarly, that mitomycin C treated tumor cells that remain viable (PI negative) and have not been induced to undergo apoptosis (Annexin V negative) also do not express C35 on the tumor cell surface membrane. FIG. 2C shows, in contrast, that mitomycin C treated tumor cells that are viable (PI negative), but undergoing apoptosis (Annexin V positive), are clearly differentially stained with anti-C35 antibodies as compared to isotype control antibody.

FIGS. 3A-3C show that anti-C35 monoclonal antibody localizes to necrotic regions of a C35+ tumor. BALB/c mice were engrafted on opposite flanks with syngeneic non-small cell lung cancer derived Line 1 tumor cells that either had or had not been transfected with human C35. C35 protein expression was confirmed by immunohistochemical staining with anti-C35 antibodies. After 14 days in vivo growth, animals received intravenous injection of 125I-labeled anti-C35 antibody. Animals were sacrificed 120 hrs after injection of radiolabeled antibodies and the concentration of anti-C35 antibodies in C35-positive and C35-negative tumors was determined by exposure of a tumor section to film. FIG. 4A shows that radiolabeled anti-C35 antibodies concentrate only in the C35-positive and not the C35-negative tumors. FIGS. 4B and 4C compare the distribution of label and an H&E stain for intact cells within the tumors, confirming that under these conditions the labeled anti-C35 antibodies concentrated specifically in the necrotic regions of the C35-positive tumor.

FIG. 4 shows that Taxol™ (paclitaxel) induces apoptosis, resulting in exposure of C35 on the surface of apoptotic tumor cells. 24 hours following treatment with 0.5 uM Taxol™, 21MT1 tumor cells were stained with annexin V-FITC, propidium iodide, and with either 100 ng anti-C35 antibody 1F2 (dark line) or isotype control (grey fill) antibody. Both antibodies were directly conjugated to Alexa-647. Histograms were gated on the cells undergoing apoptosis (annexinV positive/PI negative). Antibodies were pre-incubated with PAB buffer (FIG. 3A), 100-fold molar excess recombinant C35 protein (FIG. 3B), or 100-fold molar excess β-galactosidase protein (FIG. 3C).

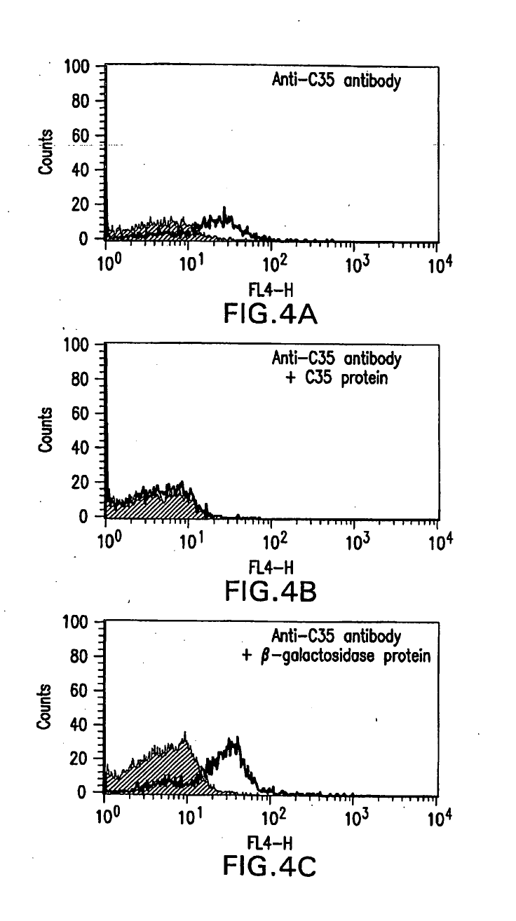

FIG. 5 shows the effect on tumor volume of combination radioimmunotherapy with ¹³¹I-labeled 1B3 anti-C35 murine monoclonal antibody and chemotherapy (fluorouracil, 150 mg/kg; leucovorin, 100 mg/kg) in Swiss nude mice grafted with Colau.C35 tumor cells. Chemotherapy was initiated on day 11 after tumor graft and 300 μCi of ¹³¹I-labeled 1B3 anti-C35 antibody was administered on day 14. Tumor growth was followed for up to 8 weeks.

FIG. 6 shows the effects on tumor volume of the combined modality treatment of chemotherapy and radioimmunotherapy. Swiss nude mice were grafted with Colau.C35 cells on day 0. Chemotherapy: Cisplatin administered at 2 mg/kg i.v. on days 15 & 18; 5FU/LV administered at 180/120 mg/kg i.v. on day 18. Radioimmunotherapy: 300 μCi (˜50 μg) of ¹³¹I-labeled murine 1B3 anti-C35 IgG was administered on day 21.

FIG. 7 shows equivalent expression in naturally-expressing and C35-transfected human breast and colon tumors. Cells were stained with Alexa-647 conjugated anti-C35 MAb 1F2 or isotype control. MFI X is the ratio of the mean fluorescence intensity of 1F2/mean fluorescence intensity of isotype control. H16N₂, derived from normal breast epithelium, and MDAMB231, a breast tumor, and Colau, a colon tumor, express low basal levels of C35. 21MT1, derived from breast carcinoma, naturally expresses high levels of C35. Colau and MDA231 were transduced with empty vector (null) or human C35 recombinant vector. All tumors were grown in vivo, tumors were excised, dissociated and stained.

FIG. 8 shows toxicity of chemotherapy, radioimmunotherapy, and combination therapy in Swiss nude mice as determined by weight loss.

FIG. 9 shows the expected peptide fragments following complete digestion of 6×His-tagged recombinant human C35 (rhC35) with Lys-C endoprotease. The full sequence of rhC35, including the amino terminal 6×His tag addition is shown. Amino acid positions are numbered relative to the amino terminal methionine (bold M) of the native human C35 sequence. The asterisks by the first and third lysine (K) residues indicate that digestion at these positions is inefficient, and some longer fragments may be generated.

FIG. 10 shows a comparison of 1B3 (Mab11) or 1F2 and anti-6× His tag staining of Western blots indicating the fragment of C35 to which each antibody binds.

FIG. 11 shows that MAb 165 is C35-specific. 141D10 recombinant vaccinia virus was co-infected into HeLa cells with UH8 recombinant vaccinia virus. The resulting secreted antibody was tested for binding to C35 or control protein A27L (vaccinia virus protein) by ELISA.

FIG. 12 shows that a 40 mg/kg total dose of murine C35 antibodies 1B3 (20 mg/kg dose) and 1F2 (20 mg/kg dose) in combination with a 30 mg/kg dose of paclitaxel (TAXOL®) is effective in reducing tumor growth in mice grafted with MDA-MB231.hC35 tumors.

FIG. 13 shows a Western demonstrating tumor specific binding of MAb163 to C35. Lane 1: recombinant human C35 protein (rC35), purified from E. coli (100 ng/lane); Lane 2: 21MT1-D human breast tumor cell lysate (100,000 cell equivalents/lane); and Lane 3: H₁₆N₂ normal immortalized human breast cell line lysate (100,000 cell equivalents/lane). The molecular weight markers are indicated in kiloDaltons, on left of the figure.

FIG. 14 shows an analysis of the C35 specificity of anti-C35 monoclonal antibodies MAb163 and Mab 11 by flow cytometry using C35-positive 21MT1-D breast cancer cell line and C35-negative H16N2 normal breast cell line. Staining with isotype control monoclonal antibody is represented by the black-filled area. Staining with anti-C35 antibodies is represented by the open line.

FIG. 15 shows immunofluorescence staining with MAb163 in human mammary cell lines. MAb 163 fluoresces at higher levels in the C35+ cells indicating that MAb 163 binds to C35.

FIG. 16 shows binding affinity of MAb 163 as measured using a 1:1 kinetic model for Biacore analysis. Using BIAevaluation software, the Ka and Kd of MAb 163 were calculated as follows: (a) ka (1/Ms)=2.84e5; (b) kd (1/s)=9.59e-4; (c) KA (1/M)=2.96e8; and (d) KD (nM)=3.38.

FIG. 17 shows the expected peptide fragments following partial digestion of 6-His-tagged recombinant human C35 (rhC35) with Lys-C endoprotease.

FIG. 18 shows the observed peptide fragments following a Lys-C digestion of recombinant human C35 by Coomassie blue staining and Anit-6-His staining. The predicted fragments are shown to the left of the blots for comparison.

FIG. 19 shows a comparison of MAb 163 staining of a Western blot to the Coomassie blue and anti-6-His blots, indicating the fragment of C35 to which MAb 163 binds. The predicted fragments are shown to the left of the blots for comparison. MAb 163 binding can be seen to fragments corresponding to predicted fragments 1-4, but not 5-11.

FIG. 20 depicts graphically the epitope specificity of MAb 163. MAb 163 recognizes an epitope within amino acid residues 48 to 87 of C35, with the amino acid positions numbered relative to the amino terminal methionine of the native human sequence (see FIG. 9). This region has the following amino acid sequence: EQYPGIEIESRLGGTGAFEEEINGQLVFSKLENGGFPYEK.

FIG. 21 shows the results of proliferation assays using anti-C35 antibodies MAb 163, Mab11 (chimeric 1B3), Mab 76 (chimeric 1F2) or Herceptin (anti-human Her2) to inhibit proliferation of the C35+/Her2+ BT474 breast tumor cell line as compared to the H16N2 C35-/Her2-normal breast cell line. Rituxan (anti-human CD20) was used as a negative control because both breast cell lines are CD20-negative. Herceptin was used as the positive control for Her2-positive cell lines.

FIG. 22 shows immunoprecipitation of nC35 from C35+ 21MT1 breast cells by Western blot, using rabbit polyclonal anti-C35. The number “15” indicates the molecular weight marker at 15 kDalton. The number “163” indicates the mAb163 monoclonal antibody. “Neg IgG” indicates an IgG negative control antibody.

FIG. 23 shows average tumor volume (cm²) in mice grafted with MDA231.rvC35 tumor cells after treatment with adriamycin (“ADM”) alone; a combination of the murine anti-C35 antibodies, 1F2 and 1B3; a combination of adriamycin, 1F2 and 1B3; adriamycin and 1B3; adriamycin and 1F2; adriamycin and an IgG isotype antibody (“iso”); and no antibody (“none”). Closed, black arrows indicate the administration of adriamycin on days 3 and 10, post-tumor graft. Thick, open arrows indicate the administration of the antibody treatments at days 3, 7, 10, 13, 17, 20, and 23, post-tumor graft. Measurements began on day 6, post-graft.

FIG. 24 shows the average change in tumor volume (%) of the mice grafted with MDA231.rvC35 tumor cells after treatment with adriamycin (“ADM”) alone; a combination of the murine anti-C35 antibodies, 1F2 and 1B3; a combination of adriamycin, 1F2 and 1B3; adriamycin and 1B3; adriamycin and 1F2; adriamycin and an IgG isotype antibody (“iso”); and no antibody (“none”). Closed, black arrows indicate the administration of adriamycin on days 3 and 10, post-tumor graft. Thick, open arrows indicate the administration of the antibody treatments at days 3, 7, 10, 13, 17, 20, and 23, post-tumor graft. Measurements began on day 6, post-graft.

### DETAILED DESCRIPTION OF THE INVENTION

### Overview

A number of studies have described alterations in the surface membrane of cells undergoing apoptosis. Prominent among these changes is the early loss of phospholipid asymmetry as reflected in the exposure of phosphatidylserine on the outer leaflet of the surface membrane. It has been reported that this alteration in surface membrane composition facilitates recognition and removal of apoptotic cells by macrophages (Fadok, V. A., et al., J. Immunol. 148:2207-2216 (1992)). A general method has been developed that allows detection of cells undergoing apoptosis by binding of the anticoagulant Annexin V to the exposed phosphatidylserine molecules (Koopman, G., et al., Blood 84:1415-1420 (1994)).

Of more general interest is the possibility that expression and exposure of other surface membrane molecules, in particular proteins, may be altered in apoptotic cells. A number of reports have described apoptosis specific proteins (Grand, R. J. A., et al., Exp. Cell Res. 218:439-451 (1995); U.S. Pat. No. 5,972,622, “Method of Detecting Apoptosis Using an Anti-Human GP46 Monoclonal Antibody”, Filed: Feb. 6, 1997; Issued, Oct. 26, 1999) that appear to be expressed intracellularly. Of more direct relevance is a report of a monoclonal antibody that detects a 38 kD protein antigen that becomes associated with the surface membrane and mitochondrial membranes of apoptotic cells but is undetectable in normal cells (U.S. Pat. No. 5,935,801, “Monoclonal Antibody that Detects Apoptotic Antigen”, Filed: Mar. 29, 1996; Issued, Aug. 10, 1999). Other antigens have been described that become differentially exposed on or near the surface of apoptotic keratinocytes (Casciola-Rosen, L. A., et al., J. Exp. Med. 179:1317-1330 (1994)), and in cells undergoing apoptosis during embryonic development (Rotello, R. J., et al., Development 120:1421-1431 (1994)). Three defined protein antigens, CD3, CD69 and CD25 have been shown to be upregulated on the surface membrane of apoptotic thymocytes (Kishimoto, H., et al., J. Exp. Med. 181:649-655 (1995)). In each instance these are surface markers of apoptosis in normal cells and tissues. Although the same markers might also be associated with tumor cells undergoing apoptosis, they do not allow apoptotic tumor cells to be distinguished from normal cells undergoing apoptosis as part of normal tissue turnover. Therefore, they would not be useful as targets for treating cancer.

The present inventors have determined that there is a subset of intracellular tumor-specific or tumor-associated antigens that become exposed on the tumor cell membrane under conditions of chemotherapy or radiation induced apoptosis and could be effective targets for concentrating antibody conjugated radioisotopes or toxins within the tumor. Methods using antibodies against such antigens would be particularly effective because they could enhance the therapeutic benefits of standard apoptosis-inducing chemotherapy and radiation therapy in treating cancer. The present invention identifies tumor-specific antigens that are associated with internal cell membranes—in particular, differentially expressed molecules such as the C35 cancer-specific antigen that express a prenylation motif—as a class of intracellular tumor antigens that become exposed on the surface membrane of tumor cells that have been induced to undergo apoptosis by radiation and/or chemotherapy.

The present invention describes a method that, in one embodiment, acts in conjunction with the induction of apoptosis (preferably large scale apoptosis) by chemotherapy or radiation therapy to enhance the eradication of tumors. It is based on the novel observation that a class of intracellular markers differentially expressed in tumor cells become exposed on the surface of apoptotic cells where they can be targeted by specific antibodies which can be administered unconjugated or conjugated to a toxic payload. The benefits of this method of treatment are several-fold. For example, with conjugated antibodies, this method permits delivery to the tumor environment of a toxic payload that can destroy other non-apoptotic tumor cells in the vicinity of the apoptotic target. Additionally, this method can prevent otherwise viable cells that have initiated the apoptotic process for example by treatment with an apoptosis-inducing chemotherapeutic agent, as evidenced by alterations in surface membrane constituents, from reversing the apoptotic progression and resuming growth (Hammill, A. K., et al., Exp. Cell Res. 251:16-21 (1999)).

The present invention targeting apoptotic cells should be distinguished from prior inventions targeting necrotic cells (U.S. Pat. No. 6,071,491, “Detection of Necrotic Malignant Tissue and Associated Therapy”, Filed: Aug. 9, 1999; Issued, Jun. 6, 2000). Necrosis results in release of intracellular contents into the extracellular tumor environment. Some of these intracellular antigens accumulate in that environment and could be targeted by specific antibodies. However, necrosis is associated with hypoxic regions of larger tumors that, because of the absence of oxygen radicals, are relatively resistant to radiation therapy and possibly radio-immunotherapy. Although there may be some increase in necrosis following treatment with chemotherapeutic agents (Desrues B., et al., Br. J. Cancer 72:1076-82, (1995)), the primary action of chemotherapeutic agents is to increase apoptosis. Therefore, necrosis is a less suitable target than apoptosis for immunotherapy of cancer and, in particular, eradication of smaller tumors and micrometastases that are responsible for tumor spread. Thus, methods that are effective at eradicating small tumors and micrometastases are especially useful for treating aggressive cancers.

The present invention should also be distinguished from the disclosure in patent application publication number US 2002/0052308 A1 (May 2, 2002), which discloses 842 cancer antigens, including an antigen (SEQ ID NO:966) with a large region identical to a portion of C35 (SEQ ID NO:2). US 2002/0052308 A1 generically discloses the administration of antibodies against the 842 cancer antigens “alone or in combination with other types of treatments (e.g., radiation therapy, chemotherapy, hormonal therapy, immunotherapy and anti-tumor agents)”, page 205, paragraph [0229]. However, the published application does not specify that to be effective against a C35 related target, C35-specific antibodies conjugated to a toxin should be administered after apoptosis has been induced in tumor cells by administration of an apoptosis inducing agent such as chemotherapy, radiation therapy, or other anti-tumor agents. Indeed, multiple studies of combination chemotherapy and radioimmunotherapy directed at antigens that, in contrast to C35, are naturally expressed on the tumor cell surface membrane have concluded that optimal results are obtained by administration of the radioimmunotherapeutic antibody prior to chemotherapy, that is, before apoptosis has been induced (DeNardo S. J., et al. Anticancer Res. 18:4011-18, (1998); Clarke K., et al., Clin. Cancer Res. 6:3621-28, (2000); Burke P.A., Cancer 94:1320-31 (2002); Stein, R. et al., Cancer 94:51-61 (2002); Odonnel R. T., et al., Prostate 50:27-37 (2002)). The discovery that apoptosis results in surface membrane exposure of a class of intracellular antigens including C35, which are prenylated and associated with internal membranes of untreated tumor cells is addressed in US 2005/0158323, incorporated herein by reference in its entirety. US 2005/0158323 also addresses that radioimmunotherapy directed at this class of target molecules is best administered such that the antibodies accumulate at the tumor site at approximately the same time that apoptosis has been induced in tumor cells by administration of an apoptosis inducing agent, or shortly thereafter. US 2002/0052308 A1 does not describe the subcellular location of the C35 related cancer antigen, nor does it describe how antibodies to this antigen should be administered for therapeutic effect. Nor does US 2002/0052308 A1 describe that two or more antibodies against C35 should be administered with a chemotherapeutic agent.

Others have observed that, with extracellularly expressed antigens such as Her2, administration of two different anti-Her2 antibodies directed to different epitopes of the protein resulted in anti-tumor activity in vivo and in vitro. Spiridon et al., Clin. Cancer Res. 8:1720-30 (2002) (incorporated by reference herein in its entirety). However, while Spiridon et al. observed an extracellularly expressed protein, Her2, C35 as described above, is an intracellular antigen that becomes expressed on the cell surface in association with apoptosis.

### I. DEFINITIONS

It is to be noted that the term “a” or “an” entity refers to one or more of that entity; for example, “a C35 antibody,” is understood to represent one or more C35 antibodies. As such, the terms “a” (or “an”), “one or more,” and “at least one” can be used interchangeably herein.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids, are included within the definition of “polypeptide,” and the term “polypeptide” may be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide may be derived from a natural biological source or produced by recombinant technology, but is not necessarily translated from a designated nucleic acid sequence. It may be generated in any manner, including by chemical synthesis.

A polypeptide of the invention may be of a size of about 3 or more, 5 or more, 10 or more, 20 or more, 25 or more, 50 or more, 75 or more, 100 or more, 200 or more, 500 or more, 1,000 or more, or 2,000 or more amino acids. Polypeptides may have a defined three-dimensional structure, although they do not necessarily have such structure. Polypeptides with a defined three-dimensional structure are referred to as folded, and polypeptides which do not possess a defined three-dimensional structure, but rather can adopt a large number of different conformations, and are referred to as unfolded. As used herein, the term glycoprotein refers to a protein coupled to at least one carbohydrate moiety that is attached to the protein via an oxygen-containing or a nitrogen-containing side chain of an amino acid residue, e.g., a serine residue or an asparagine residue.

By an “isolated” polypeptide or a fragment, variant, or derivative thereof is intended a polypeptide that is not in its natural milieu. No particular level of purification is required. For example, an isolated polypeptide can be removed from its native or natural environment. Recombinantly produced polypeptides and proteins expressed in host cells are considered isolated for purposed of the invention, as are native or recombinant polypeptides which have been separated, fractionated, or partially or substantially purified by any suitable technique.

Also included as polypeptides of the present invention are fragments, derivatives, analogs, or variants of the foregoing polypeptides, and any combination thereof. The terms “fragment,” “variant,” “derivative” and “analog” when referring to C35 antibodies or antibody polypeptides of the present invention include any polypeptides which retain at least some of the antigen-binding properties of the corresponding native antibody or polypeptide. Fragments of polypeptides of the present invention include proteolytic fragments, as well as deletion fragments, in addition to specific antibody fragments discussed elsewhere herein. Variants of C35 antibodies and antibody polypeptides of the present invention include fragments as described above, and also polypeptides with altered amino acid sequences due to amino acid substitutions, deletions, or insertions. Variants may occur naturally or be non-naturally occurring Non-naturally occurring variants may be produced using art-known mutagenesis techniques. Variant polypeptides may comprise conservative or non-conservative amino acid substitutions, deletions or additions. Variants of C35 antibodies include humanized versions of the antibodies as well as C35 antibodies that have been affinity matured or optimized. Affinity optimization can be performed by routine methods that are well-known in the art. Alternatively, a preferred method for increasing the affinity of antibodies of the invention is disclosed in US 2002 0123057 A1. Derivatives of C35 antibodies and antibody polypeptides of the present invention, are polypeptides which have been altered so as to exhibit additional features not found on the native polypeptide. Examples include fusion proteins. As used herein a “derivative” of a C35 antibody or antibody polypeptide refers to a subject polypeptide having one or more residues chemically derivatized by reaction of a functional side group. Also included as “derivatives” are those peptides which contain one or more naturally occurring amino acid derivatives of the twenty standard amino acids. For example, 4-hydroxyproline may be substituted for proline; 5-hydroxylysine may be substituted for lysine; 3-methylhistidine may be substituted for histidine; homoserine may be substituted for serine; and ornithine may be substituted for lysine.

The term “polynucleotide” is intended to encompass a singular nucleic acid as well as plural nucleic acids, and refers to an isolated nucleic acid molecule or construct, e.g., messenger RNA (mRNA) or plasmid DNA (pDNA). A polynucleotide may comprise a conventional phosphodiester bond or a non-conventional bond (e.g., an amide bond, such as found in peptide nucleic acids (PNA)). The term “nucleic acid” refers to any one or more nucleic acid segments, e.g., DNA or RNA fragments, present in a polynucleotide. By “isolated” nucleic acid or polynucleotide is intended a nucleic acid molecule, DNA or RNA, which has been removed from its native environment. For example, a recombinant polynucleotide encoding a C35 antibody contained in a vector is considered isolated for the purposes of the present invention. Further examples of an isolated polynucleotide include recombinant polynucleotides maintained in heterologous host cells or purified (partially or substantially) polynucleotides in solution. Isolated RNA molecules include in vivo or in vitro RNA transcripts of polynucleotides of the present invention. Isolated polynucleotides or nucleic acids according to the present invention further include such molecules produced synthetically. In addition, a polynucleotide or a nucleic acid may be or may include a regulatory element such as a promoter, ribosome binding site, or a transcription terminator.

As used herein, a “coding region” is a portion of nucleic acid which consists of codons translated into amino acids. Although a “stop codon” (TAG, TGA, or TAA) is not translated into an amino acid, it may be considered to be part of a coding region, but any flanking sequences, for example promoters, ribosome binding sites, transcriptional terminators, introns, and the like, are not part of a coding region. Two or more coding regions of the present invention can be present in a single polynucleotide construct, e.g., on a single vector, or in separate polynucleotide constructs, e.g., on separate (different) vectors. Furthermore, any vector may contain a single coding region, or may comprise two or more coding regions, e.g., a single vector may separately encode an immunoglobulin heavy chain variable region and an immunoglobulin light chain variable region. In addition, a vector, polynucleotide, or nucleic acid of the invention may encode heterologous coding regions, either fused or unfused to a nucleic acid encoding a C35 antibody or fragment, variant, or derivative thereof. Heterologous coding regions include without limitation specialized elements or motifs, such as a secretory signal peptide or a heterologous functional domain.

In certain embodiments, the polynucleotide or nucleic acid is DNA. In the case of DNA, a polynucleotide comprising a nucleic acid which encodes a polypeptide normally may include a promoter and/or other transcription or translation control elements operably associated with one or more coding regions. An operable association is when a coding region for a gene product, e.g., a polypeptide, is associated with one or more regulatory sequences in such a way as to place expression of the gene product under the influence or control of the regulatory sequence(s). For example, two DNA fragments (such as a polypeptide coding region and a promoter associated therewith) are “operably associated” if induction of promoter function results in the transcription of mRNA encoding the desired gene product and if the nature of the linkage between the two DNA fragments does not interfere with the ability of the expression regulatory sequences to direct the expression of the gene product or interfere with the ability of the DNA template to be transcribed. Thus, a promoter region would be operably associated with a nucleic acid encoding a polypeptide if the promoter was capable of effecting transcription of that nucleic acid. The promoter may be a cell-specific promoter that directs substantial transcription of the DNA only in predetermined cells. Other transcription control elements, besides a promoter, for example enhancers, operators, repressors, and transcription termination signals, can be operably associated with the polynucleotide to direct cell-specific transcription. Suitable promoters and other transcription control regions are disclosed herein.

A variety of transcription control regions are known to those skilled in the art. These include, without limitation, transcription control regions which function in vertebrate cells, such as, but not limited to, promoter and enhancer segments from cytomegaloviruses (the immediate early promoter, in conjunction with intron-A), simian virus 40 (the early promoter), and retroviruses (such as Rous sarcoma virus). Other transcription control regions include those derived from vertebrate genes such as actin, heat shock protein, bovine growth hormone and rabbit 13-globin, as well as other sequences capable of controlling gene expression in eukaryotic cells. Additional suitable transcription control regions include tissue-specific promoters and enhancers as well as lymphokine-inducible promoters (e.g., promoters inducible by interferons or interleukins).

Similarly, a variety of translation control elements are known to those of ordinary skill in the art. These include, but are not limited to ribosome binding sites, translation initiation and termination codons, and elements derived from picornaviruses (particularly an internal ribosome entry site, or IRES, also referred to as a CITE sequence).

In other embodiments, a polynucleotide of the present invention is RNA, for example, in the form of messenger RNA (mRNA).

Polynucleotide and nucleic acid coding regions of the present invention may be associated with additional coding regions which encode secretory or signal peptides, which direct the secretion of a polypeptide encoded by a polynucleotide of the present invention. According to the signal hypothesis, proteins secreted by mammalian cells have a signal peptide or secretory leader sequence which is cleaved from the mature protein once export of the growing protein chain across the rough endoplasmic reticulum has been initiated. Those of ordinary skill in the art are aware that polypeptides secreted by vertebrate cells generally have a signal peptide fused to the N-terminus of the polypeptide, which is cleaved from the complete or “full length” polypeptide to produce a secreted or “mature” form of the polypeptide. In certain embodiments, the native signal peptide, e.g., an immunoglobulin heavy chain or light chain signal peptide is used, or a functional derivative of that sequence that retains the ability to direct the secretion of the polypeptide that is operably associated with it. Alternatively, a heterologous mammalian signal peptide, or a functional derivative thereof, may be used. For example, the wild-type leader sequence may be substituted with the leader sequence of human tissue plasminogen activator (TPA) or mouse β-glucuronidase.

The present invention is directed to certain C35 antibodies, or antigen-binding fragments, variants, or derivatives thereof. Unless specifically referring to full-sized antibodies such as naturally-occurring antibodies, the term “C35 antibodies” (which is used interchangeably herein with the term “anti-C35 antibodies”) encompasses full-sized antibodies as well as antigen-binding fragments, variants, analogs, or derivatives of such antibodies, e.g., naturally occurring antibody or immunoglobulin molecules or engineered antibody molecules or fragments that bind antigen in a manner similar to antibody molecules.

The terms “antibody” and “immunoglobulin” are used interchangeably herein. An antibody or immunoglobulin comprises at least the variable domain of a heavy chain, and normally comprises at least the variable domains of a heavy chain and a light chain. Basic immunoglobulin structures in vertebrate systems are relatively well understood. See, e.g., Harlow et al., Antibodies: A Laboratory Manual, (Cold Spring Harbor Laboratory Press, 2nd ed. 1988).

As will be discussed in more detail below, the term “immunoglobulin” comprises various broad classes of polypeptides that can be distinguished biochemically. Those skilled in the art will appreciate that heavy chains are classified as gamma, mu, alpha, delta, or epsilon, (γ, μ, α, δ, ε) with some subclasses among them (e.g., γ1-γ4). It is the nature of this chain that determines the “class” of the antibody as IgG, IgM, IgA IgG, or IgE, respectively. The immunoglobulin subclasses (isotypes) e.g., IgG₁, IgG₂, IgG₃, IgG₄, IgA₁, etc. are well characterized and are known to confer functional specialization. Modified versions of each of these classes and isotypes are readily discernable to the skilled artisan in view of the instant disclosure and, accordingly, are within the scope of the instant invention. All immunoglobulin classes are clearly within the scope of the present invention, the following discussion will generally be directed to the IgG class of immunoglobulin molecules. With regard to IgG, a standard immunoglobulin molecule comprises two identical light chain polypeptides of molecular weight approximately 23,000 Daltons, and two identical heavy chain polypeptides of molecular weight 53,000-70,000. The four chains are typically joined by disulfide bonds in a “Y” configuration wherein the light chains bracket the heavy chains starting at the mouth of the “Y” and continuing through the variable region.

Light chains are classified as either kappa or lambda (κ, λ). Each heavy chain class may be bound with either a kappa or lambda light chain. In general, the light and heavy chains are covalently bonded to each other, and the “tail” portions of the two heavy chains are bonded to each other by covalent disulfide linkages or non-covalent linkages when the immunoglobulins are generated either by hybridomas, B cells or genetically engineered host cells. In the heavy chain, the amino acid sequences run from an N-terminus at the forked ends of the Y configuration to the C-terminus at the bottom of each chain.

Both the light and heavy chains are divided into regions of structural and functional homology. The terms “constant” and “variable” are used functionally. In this regard, it will be appreciated that the variable domains of both the light (V_{L}) and heavy (V_{H}) chain portions determine antigen recognition and specificity. Conversely, the constant domains of the light chain (C_{L}) and the heavy chain (C_{H}1, C_{H}2 or C_{H}3) confer important biological properties such as secretion, transplacental mobility, Fc receptor binding, complement binding, and the like. By convention the numbering of the constant region domains increases as they become more distal from the antigen binding site or amino-terminus of the antibody. The N-terminal portion is a variable region and at the C-terminal portion is a constant region; the C_{H}3 and C_{L} domains actually comprise the carboxy-terminus of the heavy and light chain, respectively.

As indicated above, the variable region allows the antibody to selectively recognize and specifically bind epitopes on antigens. That is, the V_{L} domain and V_{H} domain, or subset of the complementarity determining regions (CDRs), of an antibody combine to form the variable region that defines a three dimensional antigen binding site. This quaternary antibody structure forms the antigen binding site present at the end of each arm of the Y. More specifically, the antigen binding site is defined by three CDRs on each of the V_{H} and V_{L} chains. In some instances, e.g., certain immunoglobulin molecules derived from camelid species or engineered based on camelid immunoglobulins, a complete immunoglobulin molecule may consist of heavy chains only, with no light chains. See, e.g., Hamers-Casterman et al., Nature 363:446-448 (1993).

In naturally occurring antibodies, the six “complementarity determining regions” or “CDRs” present in each antigen binding domain are short, non-contiguous sequences of amino acids that are specifically positioned to form the antigen binding domain as the antibody assumes its three dimensional configuration in an aqueous environment. The remainder of the amino acids in the antigen binding domains, referred to as “framework” regions, show less inter-molecular variability. The framework regions largely adopt a β-sheet conformation and the CDRs form loops which connect, and in some cases form part of, the β-sheet structure. Thus, framework regions act to form a scaffold that provides for positioning the CDRs in correct orientation by inter-chain, non-covalent interactions. The antigen binding domain formed by the positioned CDRs defines a surface complementary to the epitope on the immunoreactive antigen. This complementary surface promotes the non-covalent binding of the antibody to its cognate epitope. The amino acids comprising the CDRs and the framework regions, respectively, can be readily identified for any given heavy or light chain variable region by one of ordinary skill in the art, since they have been precisely defined (see, “Sequences of Proteins of Immunological Interest,” Kabat, E., et al., U.S. Department of Health and Human Services, (1983); and Chothia and Lesk, J. Mol. Biol., 196:901-917 (1987), which are incorporated herein by reference in their entireties).

In the case where there are two or more definitions of a term which is used and/or accepted within the art, the definition of the term as used herein is intended to include all such meanings unless explicitly stated to the contrary. A specific example is the use of the term “complementarity determining region” (“CDR”) to describe the non-contiguous antigen combining sites found within the variable region of both heavy and light chain polypeptides. This particular region has been described by Kabat et al., U.S. Dept. of Health and Human Services, “Sequences of Proteins of Immunological Interest” (1983) and by Chothia et al., J. Mol. Biol. 196:901-917 (1987), which are incorporated herein by reference, where the definitions include overlapping or subsets of amino acid residues when compared against each other. Nevertheless, application of either definition to refer to a CDR of an antibody or variants thereof is intended to be within the scope of the term as defined and used herein. The appropriate amino acid residues which encompass the CDRs as defined by each of the above cited references are set forth below in Table 1 as a comparison. The exact residue numbers which encompass a particular CDR will vary depending on the sequence and size of the CDR. Those skilled in the art can routinely determine which residues comprise a particular CDR given the variable region amino acid sequence of the antibody.

TABLE 1
CDR DEFINITIONS¹

| | Kabat | Chothia |
|---|---|---|
| V_{H} CDR1 | 31-35 | 26-32 |
| V_{H} CDR2 | 50-65 | 52-58 |
| V_{H} CDR3 | 95-102 | 95-102 |
| V_{L} CDR1 | 24-34 | 26-32 |
| V_{L} CDR2 | 50-56 | 50-52 |
| V_{L} CDR3 | 89-97 | 91-96 |

¹Numbering of all CDR definitions in Table 1 is according to the numbering conventions set forth by Kabat et al. (see below).

Kabat et al. also defined a numbering system for variable domain sequences that is applicable to any antibody. One of ordinary skill in the art can unambiguously assign this system of “Kabat numbering” to any variable domain sequence, without reliance on any experimental data beyond the sequence itself. As used herein, “Kabat numbering” refers to the numbering system set forth by Kabat et al., U.S. Dept. of Health and Human Services, “Sequence of Proteins of Immunological Interest” (1983). Unless otherwise specified, references to the numbering of specific amino acid residue positions in a C35 antibody or antigen-binding fragment, variant, or derivative thereof of the present invention are according to the Kabat numbering system.

In camelid species, the heavy chain variable region, referred to as V_{H}H, forms the entire antigen-binding domain. The main differences between camelid V_{H}H variable regions and those derived from conventional antibodies (V_{H}) include (a) more hydrophobic amino acids in the light chain contact surface of V_{H} as compared to the corresponding region in V_{H}H, (b) a longer CDR3 in V_{H}H, and (c) the frequent occurrence of a disulfide bond between CDR1 and CDR3 in V_{H}H.

Antibodies or antigen-binding fragments, variants, or derivatives thereof of the invention include, but are not limited to, polyclonal, monoclonal, multispecific, human, humanized, primatized, or chimeric antibodies, single chain antibodies, epitope-binding fragments, e.g., Fab, Fab′ and F(ab′)₂, Fd, Fvs, single-chain Fvs (scFv), single-chain antibodies, disulfide-linked Fvs (sdFv), fragments comprising either a V_{L} or V_{H} domain, fragments produced by an Fab expression library, and anti-idiotypic (anti-Id) antibodies (including, e.g., anti-Id antibodies to C35 antibodies disclosed herein; also see, e.g., Hudson, P. J. and Couriau, C., Nature Med. 9: 129-134 (2003); U.S. Publication No. 20030148409; U.S. Pat. No. 5,837,242). ScFv molecules, for example, are known in the art and are described, e.g., in U.S. Pat. No. 5,892,019. Immunoglobulin or antibody molecules of the invention can be of any type (e.g., IgG, IgE, IgM, IgD, IgA, and IgY), class (e.g., IgG1, IgG2, IgG3, IgG4, IgA1 and IgA2) or subclass of immunoglobulin molecule.

Antibody fragments, including single-chain antibodies, may comprise the variable region(s) alone or in combination with the entirety or a portion of the following: hinge region, C_{H}1, C_{H}2, and C_{H}3 domains. Also included in the invention are antigen-binding fragments also comprising any combination of variable region(s) with a hinge region, C_{H}1, C_{H}2, and C_{H}3 domains. Antibodies or immunospecific fragments thereof for use in the diagnostic and therapeutic methods disclosed herein may be from any animal origin including birds and mammals. Preferably, the antibodies are human, murine, donkey, rabbit, goat, guinea pig, camel, llama, horse, or chicken antibodies. In another embodiment, the variable region may be condricthoid in origin (e.g., from sharks). As used herein, “human” antibodies include antibodies having the amino acid sequence of a human immunoglobulin and include antibodies isolated from human immunoglobulin libraries or from animals transgenic for one or more human immunoglobulins and that do not express endogenous immunoglobulins, as described infra and, for example in, U.S. Pat. No. 5,939,598 by Kucherlapati et al.

As used herein, the term “heavy chain portion” includes amino acid sequences derived from an immunoglobulin heavy chain. A polypeptide comprising a heavy chain portion comprises at least one of: a C_{H}1 domain, a hinge (e.g., upper, middle, and/or lower hinge region) domain, a C_{H}2 domain, a C_{H}3 domain, or a variant or fragment thereof. For example, a binding polypeptide for use in the invention may comprise a polypeptide chain comprising a C_{H}1 domain; a polypeptide chain comprising a C_{H}1 domain, at least a portion of a hinge domain, and a C_{H}2 domain; a polypeptide chain comprising a C_{H}1 domain and a C_{H}3 domain; a polypeptide chain comprising a C_{H}1 domain, at least a portion of a hinge domain, and a C_{H}3 domain, or a polypeptide chain comprising a C_{H}1 domain, at least a portion of a hinge domain, a C_{H}2 domain, and a C_{H}3 domain. In another embodiment, a polypeptide of the invention comprises a polypeptide chain comprising a C_{H}3 domain. Further, a binding polypeptide for use in the invention may lack at least a portion of a C_{H}2 domain (e.g., all or part of a C_{H}2 domain). As set forth above, it will be understood by one of ordinary skill in the art that these domains (e.g., the heavy chain portions) may be modified such that they vary in amino acid sequence from the naturally occurring immunoglobulin molecule.

In certain C35 antibodies, or antigen-binding fragments, variants, or derivatives thereof disclosed herein, the heavy chain portions of one polypeptide chain of a multimer are identical to those on a second polypeptide chain of the multimer. Alternatively, heavy chain portion-containing monomers of the invention are not identical. For example, each monomer may comprise a different target binding site, forming, for example, a bispecific antibody.

The heavy chain portions of a binding polypeptide for use in the diagnostic and treatment methods disclosed herein may be derived from different immunoglobulin molecules. For example, a heavy chain portion of a polypeptide may comprise a C_{H}1 domain derived from an IgG1 molecule and a hinge region derived from an IgG3 molecule. In another example, a heavy chain portion can comprise a hinge region derived, in part, from an IgG1 molecule and, in part, from an IgG3 molecule. In another example, a heavy chain portion can comprise a chimeric hinge derived, in part, from an IgG1 molecule and, in part, from an IgG4 molecule.

As used herein, the term “light chain portion” includes amino acid sequences derived from an immunoglobulin light chain. Preferably, the light chain portion comprises at least one of a V_{L} or C_{L} domain.

C35 antibodies, or antigen-binding fragments, variants, or derivatives thereof disclosed herein may be described or specified in terms of the epitope(s) or portion(s) of an antigen, e.g., a target polypeptide (C35) that they recognize or specifically bind. The portion of a target polypeptide which specifically interacts with the antigen binding domain of an antibody is an “epitope,” or an “antigenic determinant.” A target polypeptide may comprise a single epitope, but typically comprises at least two epitopes, and can include any number of epitopes, depending on the size, conformation, and type of antigen. Furthermore, it should be noted that an “epitope” on a target polypeptide may be or include non-polypeptide elements, e.g., an “epitope may include a carbohydrate side chain.

The minimum size of a peptide or polypeptide epitope for an antibody is thought to be about four to five amino acids. Peptide or polypeptide epitopes preferably contain at least seven, more preferably at least nine and most preferably between at least about 15 to about 30 amino acids. Since a CDR can recognize an antigenic peptide or polypeptide in its tertiary form, the amino acids comprising an epitope need not be contiguous, and in some cases, may not even be on the same peptide chain. In the present invention, peptide or polypeptide epitope recognized by C35 antibodies of the present invention contains a sequence of at least 4, at least 5, at least 6, at least 7, more preferably at least 8, at least 9, at least 10, at least 15, at least 20, at least 25, or between about 15 to about 30 contiguous or non-contiguous amino acids of C35.

By “specifically binds,” it is generally meant that an antibody binds to an epitope via its antigen binding domain, and that the binding entails some complementarity between the antigen binding domain and the epitope. According to this definition, an antibody is said to “specifically bind” to an epitope when it binds to that epitope, via its antigen binding domain more readily than it would bind to a random, unrelated epitope. The term “specificity” is used herein to qualify the relative affinity by which a certain antibody binds to a certain epitope. For example, antibody “A” may be deemed to have a higher specificity for a given epitope than antibody “B,” or antibody “A” may be said to bind to epitope “C” with a higher specificity than it has for related epitope “D.”

By “preferentially binds,” it is meant that the antibody specifically binds to an epitope more readily than it would bind to a related, similar, homologous, or analogous epitope. Thus, an antibody which “preferentially binds” to a given epitope would more likely bind to that epitope than to a related epitope, even though such an antibody may cross-react with the related epitope.

By way of non-limiting example, an antibody may be considered to bind a first epitope preferentially if it binds said first epitope with a dissociation constant (K_{D}) that is less than the antibody's K_{D} for the second epitope. In another non-limiting example, an antibody may be considered to bind a first antigen preferentially if it binds the first epitope with an affinity that is at least one order of magnitude less than the antibody's K_{D} for the second epitope. In another non-limiting example, an antibody may be considered to bind a first epitope preferentially if it binds the first epitope with an affinity that is at least two orders of magnitude less than the antibody's K_{D} for the second epitope.

In another non-limiting example, an antibody may be considered to bind a first epitope preferentially if it binds the first epitope with an off rate (k(off)) that is less than the antibody's k(off) for the second epitope. In another non-limiting example, an antibody may be considered to bind a first epitope preferentially if it binds the first epitope with an affinity that is at least one order of magnitude less than the antibody's k(off) for the second epitope. In another non-limiting example, an antibody may be considered to bind a first epitope preferentially if it binds the first epitope with an affinity that is at least two orders of magnitude less than the antibody's k(off) for the second epitope.

An antibody or antigen-binding fragment, variant, or derivative disclosed herein may be said to bind a target polypeptide disclosed herein or a fragment or variant thereof with an off rate (k(off)) of less than or equal to 5×10⁻² sec⁻¹, 10⁻² sec⁻¹, 5×10⁻³ sec⁻¹ or 10⁻³ sec⁻¹. More preferably, an antibody of the invention may be said to bind a target polypeptide disclosed herein or a fragment or variant thereof with an off rate (k(off)) less than or equal to 5×10⁻⁴ sec⁻¹, 10⁻⁴ sec⁻¹, 5×10⁻⁵ sec⁻¹, or 10⁻⁵ sec⁻¹, 5×10⁻⁶ sec⁻¹, 10⁻⁶ sec⁻¹, 5×10⁻⁷ sec⁻¹ or 10⁻⁷ sec⁻¹.

An antibody or antigen-binding fragment, variant, or derivative disclosed herein may be said to bind a target polypeptide disclosed herein or a fragment or variant thereof with an on rate (k(on)) of greater than or equal to 10³ M⁻¹ sec⁻¹, 5×10³ M⁻¹ sec⁻¹, 10⁴ M⁻¹ sec⁻¹ or 5×10⁴ M⁻¹ sec⁻¹. More preferably, an antibody of the invention may be said to bind a target polypeptide disclosed herein or a fragment or variant thereof with an on rate (k(on)) greater than or equal to 10⁵ M⁻¹ sec⁻¹, 5×10⁵ M⁻¹ sec⁻¹, 10⁶ M⁻¹ sec⁻¹ or 5×10⁶ M⁻¹ sec⁻¹ or 10⁷ M⁻¹ sec⁻¹.

An antibody is said to competitively inhibit binding of a reference antibody to a given epitope if it preferentially binds to that epitope to the extent that it blocks, to some degree, binding of the reference antibody to the epitope. Competitive inhibition may be determined by any method known in the art, for example, competition ELISA assays. An antibody may be said to competitively inhibit binding of the reference antibody to a given epitope by at least 90%, at least 80%, at least 70%, at least 60%, or at least 50%.

As used herein, the term “affinity” refers to a measure of the strength of the binding of an individual epitope with the CDR of an immunoglobulin molecule. See, e.g., Harlow et al., Antibodies: A Laboratory Manual, (Cold Spring Harbor Laboratory Press, 2nd ed. 1988) at pages 27-28. As used herein, the term “avidity” refers to the overall stability of the complex between a population of immunoglobulins and an antigen, that is, the functional combining strength of an immunoglobulin mixture with the antigen. See, e.g., Harlow at pages 29-34. Avidity is related to both the affinity of individual immunoglobulin molecules in the population with specific epitopes, and also the valencies of the immunoglobulins and the antigen. For example, the interaction between a bivalent monoclonal antibody and an antigen with a highly repeating epitope structure, such as a polymer, would be one of high avidity.

C35 antibodies or antigen-binding fragments, variants or derivatives thereof of the invention may also be described or specified in terms of their cross-reactivity. As used herein, the term “cross-reactivity” refers to the ability of an antibody, specific for one antigen, to react with a second antigen; a measure of relatedness between two different antigenic substances. Thus, an antibody is cross reactive if it binds to an epitope other than the one that induced its formation. The cross reactive epitope generally contains many of the same complementary structural features as the inducing epitope, and in some cases, may actually fit better than the original.

For example, certain antibodies have some degree of cross-reactivity, in that they bind related, but non-identical epitopes, e.g., epitopes with at least 95%, at least 90%, at least 85%, at least 80%, at least 75%, at least 70%, at least 65%, at least 60%, at least 55%, and at least 50% identity (as calculated using methods known in the art and described herein) to a reference epitope. An antibody may be said to have little or no cross-reactivity if it does not bind epitopes with less than 95%, less than 90%, less than 85%, less than 80%, less than 75%, less than 70%, less than 65%, less than 60%, less than 55%, and less than 50% identity (as calculated using methods known in the art and described herein) to a reference epitope. An antibody may be deemed “highly specific” for a certain epitope, if it does not bind any other analog, ortholog, or homolog of that epitope.

C35 antibodies or antigen-binding fragments, variants or derivatives thereof of the invention may also be described or specified in terms of their binding affinity to a polypeptide of the invention. Preferred binding affinities include those with a dissociation constant or Kd less than 5×10⁻²M, 10⁻²M, 5×10⁻³M, 10⁻³M, 5×10⁴M, 10⁴M, 5×10⁻⁵M, 10⁻⁵M, 5×10⁻⁶M, 10⁻⁶ M, 5×10⁻⁷M, 10⁻⁷M, 5×10⁻⁸M, 10⁻⁸M, 5×10⁻⁹M, 10⁻⁹M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M.

C35 antibodies or antigen-binding fragments, variants or derivatives thereof of the invention may be “multispecific,” e.g., bispecific, trispecific or of greater multispecificity, meaning that it recognizes and binds to two or more different epitopes present on one or more different antigens (e.g., proteins) at the same time. Thus, whether a C35 antibody is “monospecfic” or “multispecific,” e.g., “bispecific,” refers to the number of different epitopes with which a binding polypeptide reacts. Multispecific antibodies may be specific for different epitopes of a target polypeptide described herein or may be specific for a target polypeptide as well as for a heterologous epitope, such as a heterologous polypeptide or solid support material.

As used herein the term “valency” refers to the number of potential binding domains, e.g., antigen binding domains, present in a C35 antibody, binding polypeptide or antibody. Each binding domain specifically binds one epitope. When a C35 antibody, binding polypeptide or antibody comprises more than one binding domain, each binding domain may specifically bind the same epitope, for an antibody with two binding domains, termed “bivalent monospecific,” or to different epitopes, for an antibody with two binding domains, termed “bivalent bispecific.” An antibody may also be bispecific and bivalent for each specificity (termed “bispecific tetravalent antibodies”). In another embodiment, tetravalent minibodies or domain deleted antibodies can be made.

Bispecific bivalent antibodies, and methods of making them, are described, for instance in U.S. Pat. Nos. 5,731,168; 5,807,706; 5,821,333; and U.S. Appl. Publ. Nos. 2003/020734 and 2002/0155537, the disclosures of all of which are incorporated by reference herein. Bispecific tetravalent antibodies, and methods of making them are described, for instance, in WO 02/096948 and WO 00/44788, the disclosures of both of which are incorporated by reference herein. See generally, PCT publications WO 93/17715; WO 92/08802; WO 91/00360; WO 92/05793; Tutt et al., J. Immunol. 147:60-69 (1991); U.S. Pat. Nos. 4,474,893; 4,714,681; 4,925,648; 5,573,920; 5,601,819; Kostelny et al., J. Immunol. 148:1547-1553 (1992).

As previously indicated, the subunit structures and three dimensional configuration of the constant regions of the various immunoglobulin classes are well known. As used herein, the term “V_{H} domain” includes the amino terminal variable domain of an immunoglobulin heavy chain and the term “C_{H}1 domain” includes the first (most amino terminal) constant region domain of an immunoglobulin heavy chain. The C_{H}1 domain is adjacent to the V_{H} domain and is amino terminal to the hinge region of an immunoglobulin heavy chain molecule.

As used herein the term “C_{H}2 domain” includes the portion of a heavy chain molecule that extends, e.g., from about residue 244 to residue 360 of an antibody using conventional numbering schemes (residues 244 to 360, Kabat numbering system; and residues 231-340, EU numbering system; see Kabat E A et al. op. cit. The C_{H}2 domain is unique in that it is not closely paired with another domain. Rather, two N-linked branched carbohydrate chains are interposed between the two C_{H}2 domains of an intact native IgG molecule. It is also well documented that the C_{H}3 domain extends from the C_{H}2 domain to the C-terminal of the IgG molecule and comprises approximately 108 residues.

As used herein, the term “hinge region” includes the portion of a heavy chain molecule that joins the C_{H}1 domain to the C_{H}2 domain. This hinge region comprises approximately 25 residues and is flexible, thus allowing the two N-terminal antigen binding regions to move independently. Hinge regions can be subdivided into three distinct domains: upper, middle, and lower hinge domains (Roux et al., J. Immunol. 161:4083 (1998)).

As used herein the term “disulfide bond” includes the covalent bond formed between two sulfur atoms. The amino acid cysteine comprises a thiol group that can form a disulfide bond or bridge with a second thiol group. In most naturally occurring IgG molecules, the C_{H}1 and C_{L} regions are linked by a disulfide bond and the two heavy chains are linked by two disulfide bonds at positions corresponding to 239 and 242 using the Kabat numbering system (position 226 or 229, EU numbering system).

As used herein, the term “chimeric antibody” will be held to mean any antibody wherein the immunoreactive region or site is obtained or derived from a first species and the constant region (which may be intact, partial or modified in accordance with the instant invention) is obtained from a second species. In preferred embodiments the target binding region or site will be from a non-human source (e.g. mouse or primate) and the constant region is human.

As used herein, the term “engineered antibody” refers to an antibody in which the variable domain in either the heavy and light chain or both is altered by at least partial replacement of one or more CDRs from an antibody of known specificity and, if necessary, by partial framework region replacement and sequence changing. Although the CDRs may be derived from an antibody of the same class or even subclass as the antibody from which the framework regions are derived, it is envisaged that the CDRs will be derived from an antibody of different class and preferably from an antibody from a different species. An engineered antibody in which one or more “donor” CDRs from a non-human antibody of known specificity is grafted into a human heavy or light chain framework region is referred to herein as a “humanized antibody.” It may not be necessary to replace all of the CDRs with the complete CDRs from the donor variable region to transfer the antigen binding capacity of one variable domain to another. Rather, it may only be necessary to transfer those residues that are necessary to maintain the activity of the target binding site. Given the explanations set forth in, e.g., U.S. Pat. Nos. 5,585,089, 5,693,761, 5,693,762, and 6,180,370, it will be well within the competence of those skilled in the art, either by carrying out routine experimentation or by trial and error testing to obtain a functional engineered or humanized antibody.

As used herein the term “properly folded polypeptide” includes polypeptides (e.g., C35 antibodies) in which all of the functional domains comprising the polypeptide are distinctly active. As used herein, the term “improperly folded polypeptide” includes polypeptides in which at least one of the functional domains of the polypeptide is not active. In one embodiment, a properly folded polypeptide comprises polypeptide chains linked by at least one disulfide bond and, conversely, an improperly folded polypeptide comprises polypeptide chains not linked by at least one disulfide bond.

As used herein the term “engineered” includes manipulation of nucleic acid or polypeptide molecules by synthetic means (e.g. by recombinant techniques, in vitro peptide synthesis, by enzymatic or chemical coupling of peptides or some combination of these techniques).

As used herein, the terms “linked,” “fused” or “fusion” are used interchangeably. These terms refer to the joining together of two more elements or components, by whatever means including chemical conjugation or recombinant means. An “in-frame fusion” refers to the joining of two or more polynucleotide open reading frames (ORFs) to form a continuous longer ORF, in a manner that maintains the correct translational reading frame of the original ORFs. Thus, a recombinant fusion protein is a single protein containing two or more segments that correspond to polypeptides encoded by the original ORFs (which segments are not normally so joined in nature.) Although the reading frame is thus made continuous throughout the fused segments, the segments may be physically or spatially separated by, for example, in-frame linker sequence. For example, polynucleotides encoding the CDRs of an immunoglobulin variable region may be fused, in-frame, but be separated by a polynucleotide encoding at least one immunoglobulin framework region or additional CDR regions, as long as the “fused” CDRs are co-translated as part of a continuous polypeptide.

In the context of polypeptides, a “linear sequence” or a “sequence” is an order of amino acids in a polypeptide in an amino to carboxyl terminal direction in which residues that neighbor each other in the sequence are contiguous in the primary structure of the polypeptide.

The term “expression” as used herein refers to a process by which a gene produces a biochemical, for example, a polypeptide. The process includes any manifestation of the functional presence of the gene within the cell including, without limitation, gene knockdown as well as both transient expression and stable expression. It includes without limitation transcription of the gene into messenger RNA (mRNA), and the translation of such mRNA into polypeptide(s). If the final desired product is a biochemical, expression includes the creation of that biochemical and any precursors. Expression of a gene produces a “gene product.” As used herein, a gene product can be either a nucleic acid, e.g., a messenger RNA produced by transcription of a gene, or a polypeptide which is translated from a transcript. Gene products described herein further include nucleic acids with post transcriptional modifications, e.g., polyadenylation, or polypeptides with post translational modifications, e.g., methylation, glycosylation, the addition of lipids, association with other protein subunits, proteolytic cleavage, and the like.

As used herein, the terms “treat” or “treatment” refer to both therapeutic treatment and prophylactic or preventative measures, wherein the object is to prevent or slow down (lessen) an undesired physiological change or disorder, such as the progression of multiple sclerosis. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

By “subject” or “individual” or “animal” or “patient” or “mammal,” is meant any subject, particularly a mammalian subject, for whom diagnosis, prognosis, or therapy is desired. Mammalian subjects include humans, domestic animals, farm animals, and zoo, sports, or pet animals such as dogs, cats, guinea pigs, rabbits, rats, mice, horses, cattle, cows, and so on.

As used herein, phrases such as “a subject that would benefit from administration of a C35 antibody” and “an animal in need of treatment” includes subjects, such as mammalian subjects, that would benefit from administration of a C35 antibody used, e.g., for detection of a C35 polypeptide (e.g., for a diagnostic procedure) and/or from treatment, i.e., palliation or prevention of a disease, with a C35 antibody. As described in more detail herein, the C35 antibody can be used in unconjugated form or can be conjugated, e.g., to a drug, prodrug, or an isotope.

### II. C35 TARGET POLYPEPTIDE

C35 is an antigen differentially expressed in breast cancer and certain other tumor types including melanoma, colon carcinoma, ovarian cancer, hepatocellular carcinoma, and pancreatic cancer. The C35 protein has been shown to be prenylated and to associate with internal cell membranes but is not detectable on the surface membrane of viable tumor cells. The inventors have produced a number of antibodies, including mouse monoclonal antibodies, humanized antibodies, and human antibodies, that immunospecifically recognize C35 epitopes. The inventors have also demonstrated that induction of apoptosis in tumor cells by treatment either with a chemotherapeutic agent or irradiation results in surface membrane exposure of C35 that permits intact tumor cells to be recognized by C35-specific antibodies.

```
C35 Polynucleotide and amino acid sequences
(SEQ ID NOs: 1 and 2)
gccgcg atg agc ggg gag ccg ggg cag acg tcc gta gcg ccc cct ccc
              Met Ser Gly Glu Pro Gly Gln Thr Ser Val Ala Pro Pro Pro
                  1 5 10
gag gag gtc gag ccg ggc agt ggg gtc cgc atc gtg gtg gag tac tgt
Glu Glu Val Glu Pro Gly Ser Gly Val Arg Ile Val Val Glu Tyr Cys
  15 20 25 30
gaa ccc tgc ggc ttc gag gcg acc tac ctg gag ctg gcc agt gct gtg
Glu Pro Cys Gly Phe Glu Ala Thr Tyr Leu Glu Leu Ala Ser Ala Val
                                  35 40 45
aag gag cag tat ccg ggc atc gag atc gag tcg cgc ctc ggg ggc aca
Lys Glu Gln Tyr Pro Gly Ile Glu Ile Glu Ser Arg Leu Gly Gly Thr
                          50 55 60
ggt gcc ttt gag ata gag ata aat gga cag ctg gtg ttc tcc aag ctg
Gly Ala Phe Glu Ile Glu Ile Asn Gly Gln Leu Val Phe Ser Lys Leu
                  65 70 75
gag aat ggg ggc ttt ccc tat gag aaa gat ctc att gag gcc atc cga
Glu Asn Gly Gly Phe Pro Tyr Glu Lys Asp Leu Ile Glu Ala Ile Arg
          80 85 90
aga gcc agt aat gga gaa acc cta gaa aag atc acc aac agc cgt cct
Arg Ala Ser Asn Gly Glu Thr Leu Glu Lys Ile Thr Asn Ser Arg Pro
  95 100 105 110
ccc tgc gtc atc ctg tga
Pro Cys Val Ile Leu
                                115
```

### III. C35 ANTIBODIES

This invention relates to antibodies against C35 (referred to herein as “anti-C35 antibodies” or “C35 antibodies”), polynucleotides encoding such antibodies, methods of treating C35-associated cancers using C35 antibodies and polynucleotides, and methods of detection and diagnosis using C35 antibodies and polynucleotides. Also provided are vectors and host cells comprising C35 antibody polynucleotides, and methods of producing C35 antibodies. As described in more detail herein, the invention also relates to methods using C35 antibodies for cancer treatment, detection, and diagnosis. The description above regarding antibodies also applies to C35 antibodies described herein.

The present invention is further directed to antibody-based treatment methods which involve administering one C35 antibody or, in other embodiments, at least two C35 antibodies of the invention to a subject, preferably a mammal, and most preferably a human, for treating one or more C35 cancers. Therapeutic compounds of the invention include, but are not limited to, antibodies of the invention (including fragments, analogs and derivatives thereof as described herein) and nucleic acids encoding antibodies of the invention (including fragments, analogs and derivatives thereof as described herein). The antibodies of the invention can be used to treat, detect or diagnose C35-associated cancers, including breast, liver, ovarian, colon, pancreatic, and bladder cancers, and melanoma. C35 antibodies of the invention may be provided in pharmaceutically acceptable compositions as known in the art or as described herein.

Antibodies of the invention include, but are not limited to, polyclonal, monoclonal, multispecific, human, humanized or chimeric antibodies, single chain antibodies, scFvs, diabodies, triabodies, tetrabodies, minibodies, domain-deleted antibodies, Fab fragments, F(ab′)2 fragments, fragments produced by a Fab expression library, anti-idiotypic (anti-Id) antibodies (including, e.g., anti-Id antibodies to antibodies of the invention), and epitope-binding fragments of any of the above. The term “antibody,” as used herein, refers to immunoglobulin molecules and immunologically active portions of immunoglobulin molecules, i.e., molecules that contain an antigen binding site that immunospecifically binds an antigen. The immunoglobulin molecules of the invention can be of any type (e.g., IgG, IgE, IgM, IgD, IgA and IgY), class (e.g., IgG1, IgG2, IgG3, IgG4, IgA1 and IgA2) or subclass of immunoglobulin molecule.

Hybridoma cell lines 1F2.4.1 and 1B3.6.1, specific for C35 polypeptides, were prepared using hybridoma technology. (Kohler et al., Nature 256:495 (1975); Kohler et al., Eur. J. Immunol. 6:511 (1976); Kohler et al., Eur. J. Immunol. 6:292 (1976); Hammerling et al., in: Monoclonal Antibodies and T-Cell Hybridomas, Elsevier, N.Y., pp. 571-681 (1981)). Briefly, hybridoma cell lines were generated using standard PEG fusion to the non-secreting myeloma cell line NS-1 (P3/NS1/1-AG4-1, ATCC #TIB-18) of splenocytes from BALB/c mice immunized with syngeneic BCA34 fibroblast tumor cells transduced to over express C35. Following PEG fusion to NS-1, the hybridomas were grown in methylcellulose semi-solid media. Approximately 2 weeks later, hybridoma colonies were isolated into 96 well plates and individual supernatants were tested for reactivity with C35 by ELISA, Western blot, and immunohistochemistry. Positive hybridoma colonies were subcloned and screened for reactivity twice to ensure clonality. Antibodies were isolated from hybridoma supernatants by protein G affinity purification using standard methods. Antibodies from two hybridoma cell lines, 1F2 and 1B3, specifically bind recombinant C35 protein in ELISA and Western Blot assays. Antibodies from hybridoma cell line 1F2 also specifically stain formalin fixed, paraffin embedded C35 positive tumors and cell lines by immunohistochemistry. In addition, the present inventors developed intracellular staining flow cytometry assays for quantitative analysis using antibodies from hybridoma cell line 1F2 conjugated to Alexa-647 fluorochrome. Each of these antibodies is distinct, yet both are specific for C35 protein. It is possible to immunoprecipitate C35 protein from cell lysates with either of these antibodies and detect with the other. Competitive binding ELISA assays suggest that the monoclonal antibodies produced by hybridoma cell lines 1F2 and 1B3 bind different epitopes of the C35 protein.

C35 antibodies of the invention include antibodies which immunospecifically bind a C35 polypeptide, polypeptide fragment, or variant of SEQ ID NO:2, and/or an epitope, of the present invention (as determined by immunoassays well known in the art for assaying specific antibody-antigen binding).

As used herein the term “isolated” is meant to describe a compound of interest (e.g., a C35 antibody) that is in an environment different from that in which the compound naturally occurs. “Isolated” is meant to include compounds that are within samples that are substantially enriched for the compound of interest and/or in which the compound of interest is partially or substantially purified.

As used herein, the terms “substantially enriched” and “substantially purified” refers to a compound that is removed from its natural environment and is at least 60% free, preferably 75% free, and most preferably 90% free from other components with which it is naturally associated. As used here, an antibody having the “same specificity” as a reference antibody means the antibody binds the same epitope as the reference antibody. The determination of whether an antibody binds the same epitope as a reference antibody may be performed using the assays described herein below.

The antibodies derived from mouse hybridoma cell lines discussed herein are 1F2 and 1B3. Polynucleotides encoding the VL and VH regions of these antibodies were cloned into TOPO vectors as described in Example 6, which were deposited with the American Type Culture Collection (“ATCC”) on the date listed in Table 2, and given ATCC Deposit Numbers listed in Table 2. The ATCC is located at 10801 University Boulevard, Manassas, Va. 20110-2209, USA. The ATCC deposits were made pursuant to the terms of the Budapest Treaty on the international recognition of the deposit of microorganisms for purposes of patent procedure.

Clone 1F2G was deposited at the ATCC on Nov. 11, 2003 and given ATCC Deposit Number PTA-5639. Clone 1F2K was deposited at the ATCC on Nov. 11, 2003 and given ATCC Deposit Number PTA-5640. Clone 1B3G was deposited at the ATCC on Nov. 11, 2003 and given ATCC Deposit Number PTA-5637. Clone 1B3K was deposited at the ATCC on Nov. 11, 2003 and given ATCC Deposit Number PTA-5638.

TABLE 2
DEPOSITED POLYNUCLEOTIDE CLONES ENCODING
MOUSE ANTI-C35 VARIABLE REGIONS

| Polynucleotide Clone | ATCC Accession No. | Deposit Date |
|---|---|---|
| 1F2G | PTA-5639 | Nov. 11, 2003 |
| 1F2K | PTA-5640 | Nov. 11, 2003 |
| 1B3G | PTA-5637 | Nov. 11, 2003 |
| 1B3K | PTA-5638 | Nov. 11, 2003 |

The sequences of the mouse variable region genes and part of the vector of the deposited clones are set forth below.

Italics=Topo vector sequence (included in deposited clone)

dotted underline=EcoR1 cloning site of Topo vector

Lowercase=5′untranslated region including generacer primer

ATG=Murine signal peptide begin

bold=Frame work regions (FWR)

double underline=CDR1, CDR2, or CDR3

underline=5′ portion of mouse IgG1 or kappa constant region

1F2 murine anti-C35 Vgamma1 gene polynucleotide sequence (from clone 1F2G)

(SEQ ID NO: 3)

```
GAATTTAGCGGCCGC [symbol] GCCCTTcgactggagcacggg [symbol]
gaaaacatctctctcattagaggtga
tctttgaggaaaacagggtgttgcctaaaggATGAAAGTGTTGAGTCTGT
TGTACCTGTTGACAGCCATTCCTGGTATCCTGTCTGATGTACAGCTTCAG
GAGTCAGGACCTGGCCTCGTGAAACCTTCTCAGTCTCTGTCTCTCACCTG
CTCTGTCACTGGCTACTCCATCACCAGTGGTTATTTCTGGAACTGGATCC
                                                            CDR1
GGCAGTTTCCAGGGAACAAACTGGAATGGATGGGCTACATAAGCTACGAC
                                                                              CDR2
GGTAGCAATAACTCCAACCCATCTCTCAAAAATCGAATCTCCTTCACTCG
TGACACATCTAAGAACCAGTTTTTCCTGAAGTTTAATTCTGTGACTACTG
ACGACTCAGCTGCATATTACTGTACAAGAGGAACTACGGGGTTTGCTTAC
                                                                        CDR3
TGGGGCCAAGGGACTCTGGTCACTGTCTCTGCAGCCAAAACGACACCCCC
ATCTGTCTATCCACTGGCCCCTGGATCTGCTGCCCAAACTAACTCCAAGG
GC [symbol] GTTTAAACCTGCAGGACTAGTCCCTT
```

### SIGNAL PEPTIDE=18 AA

### FR 1=30 AA

### CDR 1=6 AA

### FR2=14AA

### CDR2=16AA

### FR 3=32 AA

### CDR 3=7 AA

### FR 4=11 AA

1F2 VH amino acid sequence (encoded by clone 1F2G)

1F2 VH amino acid sequence (encoded by clone 1F2G)

(SEQ ID NO:4)

DVQLQESGPGLVKPSQSLSLTCSVTGYSITSGYFWNWIRQFPGNKLEWMG
YISYDGSNNSNPSLKNRISFTRDTSKNQFFLKFNSVTTDDSAAYYCTRGT
TGFAYWGQGTLVTVSA
1F2 murine anti-C35 kappa V gene polynucleotide
sequence (from clone 1F2K)

(SEQ ID NO:5)

```
CGC [symbol] GCCCTTcgactggagcacgag [symbol]
aaaaattagctagggaccaaaatt
caaagacagaATGGATTTTCAGGTGCAGATTTTCAGCTTCCTGCTAATCA
GTGCCTCAGTCAGAATGTCCAGAGGACAAATTGTTCTCACCCAGTCTCCA
GCAATCATGTCTGCATCTCCAGGGGAGAAGGTCACCATATCCTGCAGTGC
CAGCTCAAGTGTAAGTTACATGAACTGGTACCAGCAGAAGCCAGGATCCT
          CDR1
CCCCCAAACCCTGGATTTATCACACATCCAACCTGGCTTCTGGAGTCCCT
                                                        CDR2
GCTCGCTTCAGTGGCAGTGGGTCTGGGACCTCTTACTCTCTCACAATCAG
CAGCATGGAGGCTGAAGATGCTGCCACTTATTACTGCCAACAGTATCATA
                                                                                          CDR3
GTTACCCACCCACGTTCGGAGGGGGGACCAAGCTGGAAATAAAACGGGCT
GATGCTGCACCAACTGTATCCATCTTCCCACCATCCAGTGAGCAAAGGGC
GAATTCGTTT
```

1F2 VH amino acid sequence (encoded by clone 1F2G)

### SIGNAL PEPTIDE=22 AA

### FR1=23 AA

### CDR 1=10 AA

### FR 2=15 AA

### CDR 2=7 AA

### FR 3=32 AA

### CDR 3=9 AA

### FR 4=10 AA

1F2-VK amino acid sequence (encoded by clone 1F2K)

(SEQ ID NO: 6)

QIVLTQSPAIMSASPGEKVTISCSASSSVSYMNWYQQKPGSSPKPWIYHT
SNLASGVPARFSGSGSGTSYSLTISSMEAEDAATYYCQQYHSYPPTFGGG
TKLEIK
1B3 murine anti-C35 Vgamma V-gene (encoded by
clone 1B3G) (NC1-A7 V139-D-J1 (VH36-60)
M13281)

(SEQ ID NO: 7)

```
CGC [symbol] GCCCTTcgactggagcacga [symbol]
gaaaatctctctcactggaggct
gatttttgaagaaaggggttgtagcctaaaagATGATGGTGTTAAGTCTT
CTGTACCTGTTGACAGCCCTTCCGGGTATCCTGTCAGAGGTGCAGCTTCA
GGAGTCAGGACCTAGCCTCGTGAAACCTTCTCAGACTCTGTCCCTCACCT
GTTCTGTCACTGGCGACTCCATCACCAGTGGTTACTGGAACTGGATCCGG
                                                        CDR1
AAATTCCCAGGAAATAAACTTGAATACGTGGGGTACATAAGCTACAGTGG
                                                                                    CDR2
TGGCACTTACTACAATCCATCTCTCAAAAGTCGAATCTCCATCACTCGAG
ACACATCCAAGAACCACTACTACCTGCAGTTGAATTCTGTGACTACTGAG
GACACAGCCACATATTACTGTGCAAGAGGTGCTTACTACGGGGGGGCCTT
                                                                                CDR3
TTTTCCTTACTTCGATGTCTGGGGCGCTGGGACCACGGTCACCGTCTCCT
CAGCCAAAACGACACCCCCATCTGTCTATCCACTGGCCCCTGGATCTGCT
GCCCAAACTAACTCCAAGGGC [symbol] GTTTAAACCTGC
```

### SIG PEP=18 AA

### FR1=30AA

### CDR 1=5 AA

### FR2=14 AA

### CDR2=16 AA

### FR 3=32 AA

### CDR 3=14 AA

### FR 4=11 AA

1B3 VH amino acid sequence (encoded by clone 1B3G)

(SEQ ID NO: 8)

EVQLQESGPSLVKPSQTLSLTCSVTGDSITSGYWNWIRKFPGNKLEYVGY
ISYSGGTYYNPSLKSRISITRDTSKNHYYLQLNSVTTEDTATYYCARGAY
YGGAFFPYFDVWGAGTTVTVSS
1B3 murine anti-C35 kappa V-gene (from clone 1B3K)

(SEQ ID NO: 9)

```

[symbol]
GCCCTTcccctggagcacga [symbol]
gaaaatcagttcctgccaggacac
agtttagatATGAGGTTCCAGGTTCAGGTTCTGGGGCTCCTTCTGCTCTG
GATATCAGGTGCCCACTGTGATGTCCAGATAACCCAGTCTCCATCTTTTC
TTGCTGCATCTCCTGGAGAAACCATTACTATTAATTGCAGGGCAAGTAAG
                                                                                            CDR1
TACATTAGCAAACATTTAGTCTGGTATCAGGAGAAACCTGGAGAAACTAA
AAAGCTTCTTATCTACTCTGGATCCACTTTGCAATCTGGACTTCCATCAA
                                                CDR2
GGTTCAGTGGCAGTGGATCTGGTACAGATTTCACTCTCACCATCAGTAGC
CTGGAGCCTGAAGATTTTGCAATGTATTACTGTCAACAGCATAATGAATA
                                                                              CDR3
CCCGCTCACGTTCGGTGCTGGGACCAAGCTGGAGCTGAAACGGGCTGATG
CTGCACCAACTGTATCCATCTTCCCACCATGCAGTGAGCAAAGGGC

[symbol]

```

### SP=20AA

### FR1=23 aa

### CDR1=11 aa

### FR2=15 aa

### CDR2=7 AA

### FR3=32 aa

### CDR 3=9AA

### FR 4=10 AA

1B3 VK amino acid sequence (encoded by clone 1B3K)

(SEQ ID NO: 10)

DVQITQSPSFLAASPGETITINCRASKYISKHLVWYQEKPGETKIKLLIY
SGSTLQSGLPSRFSGSGSGTDFTLTISSLEPEDFAMYYCQQHNEYPLTFG
AGTKLELK

The present inventors have also produced two C35 antibodies, MAb 165 and MAb 171, using the method disclosed in US 2002 0123057 A1, published 5 Sep. 2002. The heavy chain variable regions of MAb 165 and MAb 171 comprise the same CDR3 region as the 1B3 antibody heavy chain variable region described above. The remainders of MAbs 165 and 171 are of human origin. The present invention is directed to antibodies that immunospecifically bind C35 polypeptides, comprising any one of the VH or VL regions of SEQ ID NO:56, SEQ ID NO:58, or SEQ ID NO:60, or a combination of either VH region encoded by SEQ ID NO:56 or SEQ ID NO:60 and the VL region encoded by SEQ ID NO:58, and preferably the C35-specific antibodies MAb 165 or MAb 171. Both MAb 165 and MAb 171 comprise the same kappa light chain, UH8 VK L120.

The sequences of the heavy and light chain variable regions of MAb 165 and MAb 171 are set forth below.

### UNDERLINE=CDR1, CDR2, or CDR3

MAb 165 VH (141D10 VH H732) nucleotide sequence:

(SEQ ID NO: 56)

```
CAGGTGCAGCTGCAGGAGTCGGGCCCAGGACTGGTGAAGCCTCCGGAGAC
CCTGTCCCTCACCTGCAATGTCTCTGGTGGCTCTATCGGTAGATACTATT
                                                                                            CDR1
GGAACTGGATCCGACAGTCCCCAGGGAAGGGGCTGGAGTGGATTGGCCAT
ATCCATTACAGTGGGAGCACCATCTACCATCCCTCCCTCAAGAGTCGAGT
                                      CDR2
CAGCATATCGCTGGACACGTCCAAGAACCAGGTCTCCCTGAAGTITGAGT
TCTGTGACCGCTGCGGACACGGCCGTGTATTACTGTGCACGAGGTGCTTA
CTACGGGGGGGCCTTTTTTCCTTACTTCGATGTCTGGGGCCAAGGGACCA
    CDR3
CGGTCACCGTCTCCTCA
MAb 165 VH (141D10 VH H732) amino acid sequence:
```

(SEQ ID NO: 57)

QVQLQESGPGLVKPPETLSLTCNVSGGSIGRYYWNWIRQSPGKGLEWIGH
IHYSGSTIYHPSLKSRVSISLDTSKNQVSLKLSSVTAADTAVYYCARGAY
YGGAFFPYFDVWGQGTTVTVSS
MAb 171 VH (MSH3 VH H835) nucleotide sequence:

(SEQ ID NO: 60)

```
CAGGTGCAGCTGCAGGAGTCGGGAGGAGGCTTAGTTCAGCCTGGGGGGTC
CCTGAGACTCTCTTGTGCAGGCTCTGGATTCACCTTCAGTAGTTACTGGA
TGCACTGGGTCCGCGAAGCTCCAGGGAAGGGGCTGGTGTGGGTCTCACGT
  CDR1
ATTGACACTGATGGGAGTACCACAACCTACGCGGACTCCGTGAAGGGCCG
                                                                          CDR2
ATTCACCATCTCCAGAGACAACGCCAAGAACACACTGTATCTGCAAATGA
ACAGCCTGAGAGTCGAGGACACGGCCGTGTATTACTGTGCACGAGGTGCT
TACTACGGGGGGGCCTTTTTTCCTTACTTCGATGTCTGGGGCCAAGGGAC
                      CDR3
CACGGTCACCGTCTCCTCA
MAb 171 VH (141D10 VH H732) amino acid sequence:
```

(SEQ ID NO: 61)

QVQLQESGGGLVQPGGSLRLSCAGSGFTFSSYWMHWVRQAPGKGLVWVSR
IDTDGSTTTYADSVKGRFTISRDNAKNTLYLQMNSLRVEDTAVYYCARGA
YYGGAFFPYFDVWGQGTTVTVSS
UH8 VK L120 nucleotide sequence:

(SEQ ID NO: 58)

```
GACATCCAGATGACCCAGTCTCCATCCTCCCTGTCTGCATCTATGGGAGA
CAGAGTCACCATCACTTGCCGGGCGAGTCAGGGCATTAGGAATCATTTAG
                                                                                            CDR1
CCTGGTATCAGCAGAAACCAGGGAAAGCTCCTAATCTCCTGATCTCTGCT
                                                                                          CDR2
GCATCCACTTTGCAATCAGGGGTCCCAACTCGATTCAGTGGCAGTGGATC
TGGAACAGATTTCACTCTCACCATCAGCAGCCTGCAGCCTGAAGACTCTG
CAACTTATTACTGCCAACAGTATAATCGGTACCCCCTCACTTTCGGGCAA
                                                                            CDR3
GGGACCAAGCTCGAGATCAAA
UH8 VK L120 amino acid sequence:
```

(SEQ ID NO: 59)

DIQMTQSPSSLSASMGDRVTITCRASQGIRNHLAWYQQKPGKAPNLLISA
ASTLQSGVPTRFSGSGSGTDFTLTISSLQPEDSATYYCQQYNRYPLTFGQ
GTKLEIK

The present inventors have also produced a human C35 antibody, MAbc009, using the method disclosed in US 2002 0123057 A1. The present invention is directed to antibodies that immunospecifically bind C35 polypeptides, comprising the VH and VL regions encoded by the polynucleotide clones that are listed in Table 3, preferably the fully human C35-specific antibody MAbc009. Polynucleotides encoding the VL and VH regions of this antibody were cloned into TOPO vectors as described in Example 6, which were deposited with the American Type Culture Collection (“ATCC”) on the date listed in Table 3, and given ATCC Deposit Numbers listed in Table 3. The ATCC is located at 10801 University Boulevard, Manassas, Va. 20110-2209, USA. The ATCC deposit was made pursuant to the terms of the Budapest Treaty on the international recognition of the deposit of microorganisms for purposes of patent procedure.

Clone H0009 was deposited at the ATCC on Nov. 11, 2003 and given ATCC Deposit Number PTA-5641. Clone L0010 was deposited at the ATCC on Nov. 11, 2003 and given ATCC Deposit Number PTA-5542.

TABLE 3
DEPOSITED POLYNUCLEOTIDE CLONES ENCODING
HUMAN ANTI-C35 VARIABLE REGIONS

| Polynucleotide | Encoded Antibody | ATCC Accession | |
|---|---|---|---|
| Clone | Region | No. | Deposit Date |
| H0009 | VH of MAbc009 | PTA-5641 | Nov. 11, 2003 |
| L0010 | VL of MAbc009 | PTA-5642 | Nov. 11, 2003 |

The sequences of the human variable region genes and part of the vector of the deposited clones are set forth below.

DOTTED UNDERLINE=EcoR1 Cloning Site Of Topo Vector

ATG=human signal peptide begin

BOLD=FRAME WORK REGIONS

DOUBLE UNDERLINE=CDR1, CDR2, OR CDR3

UNDERLINE=Human IgG1GS or Kappa Constant Region

MAbc0009 VH NUCLEOTIDE SEQUENCE (from clone H0009)

(SEQ ID NO: 11)

```

[symbol]
GCCCTTAATTGCGGCCGCAAACCATGGGATGGAGCTGTATCATC
CTCTTCTTGGTAGCAACAGCTACAGGCGCGCACTCCGAGGTGCAGCTGGT
GGAGTCTGGGGGAGGCGTGGTCCAGCCTGGGAGGTCCCTGAGACTCTCCT
GTGCAGCGTCTGGATTCAACTTCGGTACCTATGCCATGCACTGGGTCCGC
                                                            CDR1
CAGGCTCAAGGCAAGGGGCTGGAGTGGGTGGCACTCATATGGTATGATGG
AACTAAGAAATACTATGCAGACTCCGTGAAGGGCCGATACACCATCTCCA
        CDR2
GAGACAATTCCCAGAACACGCTGTATCTGCAAATGAACACCCTGAGAGCC
GACGACACGGCTGTGTATTACTGTGCGAAATCAAAACTCCAGGGGCGCGT
                                                                              CDR3
TATAGACTACTGGGGCCAGGGAACCCTGGTCACCGTCTCCTCAGCCTCCA
CCAAGGGCCCATCGGTCTTCCCCCTGGCACCCTCCTCCAAGAGCACCTCT
GGGGGCACAGCGGCCCTGGGCTGCCTGGTCAAGGACTACTTAAGGGC

[symbol]

MAbc0009 VH AMINO ACID SEQUENCE (encoded by clone
H0009)
```

(SEQ ID NO: 12)

EVQLVESGGGVVQPGRSLRLSCAASGFNFGTYAMHWVRQAQGKGLEWVAL
IWYDGTKKYYADSVKGRYTISRDNSQNTLYLQMNTLRADDTAVYYCAKSK
LQGRVDYWGQGTLVTVSS
MAbc0009 VK NUCLEOTIDE SEQUENCE (from clone L0010)

(SEQ ID NO: 13)

```

[symbol]
GCCCTTAATTGCGGCCGCAAACATGGGATGGAGCTGTATCATCC
TCTTCTTGGTAGCAACAGCTACAGGCGTGCACTCCGACATCCAGATGACC
CAGTCTCCAGACTCCCTGGCTGTGTCTCTGGGCGAGAGGGCCACCATCAA
CTGCAAGTCCAGCCAGAGTGTTTTATACAGCTCCAACAATAAGAACTACT
              CDR1
TAGCTTGGTACCAGCAGAAACCAGGACAGCCTCCTAAGCTGCTCATTTAC
TGGGCATCTACCCGGGAATCCGGGGTCCCTGACCGATTCAGTGGCAGCGG
            CDR2
GTCTGGGACAGATTTCACTCTCACCATCAGCAGCCTGCAGGCTGAAGATG
TGGCAGTTTATTACTGTCAGCAATATTATAGTACTCCTCTGTGGACGTTC
                                                    CDR3
GGCCAAGGGACCAAGCTCGAGATCAAACGAACTGTGGCTGCACCATCTGT
CTTCATCTTCCCGCCATCTGATGAGCAGTTGAAATCTGGAACTGCCTCTG
TTGTGTGCCTGCTGAAAAGGGC [symbol]
MAbc0009 VK AMINO ACID SEQUENCE (encoded by clone
L0010)
```

(SEQ ID NO: 14)

IQMTQSPDSLAVSLGERATINCKSSQSVLYSSNNKNYLAWYQQKPGQPPK
LLIYWASTRESGVPDRFSGSGSGTDFTLTISSLQAEDVAVYYCQQYYSTP
LWTFGQGTKLEIK

The mouse C35 antibodies have heavy and light chain variable regions designated SEQ ID Nos:3-10. The mouse antibodies 1F2 and 1B3 have gamma1 isotype and kappa light chains. The antibodies MAb 165 and MAb 171 that have the same heavy chain variable region CDR3 as 1B3 mouse antibody have heavy and light chain variable regions designated SEQ ID NOs:56-60. The antibodies MAb 165 and MAb 171 have kappa light chains. The human antibody MAbc009 has heavy and light chain variable regions designated SEQ ID Nos:11-14. The human antibody MAbc009 has gamma1 isotype and kappa light chains.

The present inventors have also produced another human C35 antibody, MAb163, using the methods disclosed in US 2002 0123057 A1. The present invention is directed to antibodies that immunospecifically bind C35 polypeptides, comprising the VH and VL regions encoded by the polynucleotide clones that are listed in Table 4, preferably the fully human C35-specific antibody MAb 163.

TABLE 4
POLYNUCLEOTIDE CLONES ENCODING
HUMAN ANTI-C35 VARIABLE REGIONS

| Polynucleotide Clone | Encoded Antibody Region |
|---|---|
| H730 | VH of MAb163 |
| L74 | VL of MAb163 |

The sequences of the human variable region genes and part of the vector of the clones are set forth below with the CDRs underlined.

Amino acid sequence of VH of MAb163 (from clone
H730)

(SEQ ID NO: 62)

EVQLVESGGGLVKPGGSLRLSCEVSGITFSNAWMSWVRQAPGKGLEWVGR
IKSKTDGGTTDYAAPVKGRFTISRDDSKNTLYLQMNSLKTEDTAVYYCSI
GYYYDSSFKYGMDVWGQGTTVTVSS
Amino acid sequence of VH CDR 1 ofMAb163 (from
clone 11730)
(SEQ ID NO: 63)
GITFSNAWMS
Amino acid sequence of VH CDR 2 of MAb163 (from
clone H730)
(SEQ ID NO: 64)
RIKSKTDGGTTDYAAPVKG
Amino acid sequence of VH CDR 3 of MAb163 (from
clone 11730)
(SEQ ID NO: 65)
GYYYDSSFKYGMDV
Amino acid sequence of VL of MAb163 (from clone
L74)
(SEQ ID NO: 66)
DIQMTQSPATLSASVGDRVTITCRASQSISRWLAWYQQKPGQAPKVLIYK
ASTLQSGVPSRFSGSGSGTEFSLTINSLQPDDFATYYGQQYYSYLRTFGQ
GTKLEIK
Amino acid sequence of VL CDR 1 of MAb163 (from
clone L74)
(SEQ ID NO: 67)
RASQSISRWLA
Amino acid sequence of VL CDR 2 of MAb163 (from
clone L74)
(SEQ ID NO: 68)
KASTLQS
Amino acid sequence of VL CDR 3 of MAb163 (from
clone L74)
(SEQ ID NO: 69)
QQYYSYLRT
Nucleotide sequence of VH of MAb163 (from clone
H730)

(SEQ ID NO: 70)

```
GAGGTGCAGCTGGTGGAGTCTGGGGGAGGCTTGGTAAAGCCGGGGGGGTC
CCTTAGACTCTCCTGTGAAGTCTCTGGAATCACTTTCAGTAATGCCTGGA
                                                            CDR1
TGAGCTGGGTCCGCCAGGCTCCAGGGAAGGGGCTGGAGTGGGTTGGCCGT
ATTAAAAGCAAAACTGATGGTGGGACAACAGACTACGCTGCACCCGTGAA
                      CDR2
AGGCAGATTCACCATCTCAAGAGATGATTCAAAAAACACGCTGTATCTGC
AAATGAACAGCCTGAAAACCGAGGACACAGCCGTGTATTATTGTAGCATA
GGGTATTACTATGATAGTAGTTTCAAATACGGTATGGACGTCTGGGGCCA
        CDR3
AGGGACCACGGTCACCGTCTCCTCA
Nucleotide sequence of VH CDR 1 of MAb163 (from
clone H730)
(SEQ ID NO: 72)
GGAATCACTTTCAGTAATGCCTGGATGAGC
Nucleotide sequence of VH CDR 2 of MAb163 (from
clone H730)
(SEQ ID NO: 73)
CGTATTAAAAGCAAAACTGATGGTGGGACAACAGACTACGCTGCACCCGT
GAAAGGC
Nucleotide sequence of VH CDR 3 of MAb163 (from
clone H730)
(SEQ ID NO: 74)
GGGTATTACTATGATAGTAGTTTCAAATACGGTATGGACGTC
Nucleotide sequence of VL of MAb163 (from clone
L74)
```

(SEQ ID NO: 71)

```
GACATCCAGATGACCCAGTCTCCTGCCACCCTGTCTGGATCTGTAGGAGA
CAGAGTCACCATCACTTGCCGGGCCAGTCAGAGTATTAGTCGGTGGTTGG
                                                  CDR1
CCTGGTATCAGCAGAAGCCAGGACAAGCCCCTAAAGTCTTGATCTATAAG
GCGTCTACTTTACAAAGTGGGGTCCCATCAAGGTTCAGCGGCAGTGGGTC
                CDR2
TGGGACAGAATTCAGTCTCACCATCAACAGCCTGCAGCCTGATGATTTTG
CAACTTATTATTGCCAACAGTATTATAGTTATCTTCGGACGTTCGGCCAA
                                          CDR3
GGGACCAAGCTCGAGATCAAA
Nucleotide sequence of VL CDR 1 of MAb163 (from
clone L74)
```

(SEQ ID NO: 75)

CGGGCCAGTCAGAGTATTAGTCGGTGGTTGGCC
Nucleotide sequence of VL CDR 2 of MAb163 (from
clone L74)

(SEQ ID NO: 76)

AAGGCGTCTACTTTACAAAGT
Nucleotide sequence of VL CDR 3 of MAb163 (from
clone L74)

(SEQ ID NO: 77)

CAACAGTATTATAGTTATCTTCGGACG

The present invention encompasses antibodies (including molecules comprising, or alternatively consisting of, antibody fragments or variants thereof) that immunospecifically bind to a C35 polypeptide or a fragment, variant, or fusion protein thereof. A C35 polypeptide includes, but is not limited to, the C35 polypeptide of SEQ ID NO:2. C35 polypeptides may be produced through recombinant expression of nucleic acids encoding the polypeptide of SEQ ID NO:2. (See WO 01/74859 and U.S. Appl. No. 2004/0063907 for epitope-containing fragments of C35.)

Preferably, analogs of exemplified antibodies differ from exemplified antibodies by conservative amino acid substitutions. For purposes of classifying amino acids substitutions as conservative or nonconservative, amino acids may be grouped as follows: Group I (hydrophobic sidechains): met, ala, val, leu, ile; Group II (neutral hydrophilic side chains): cys, ser, thr; Group III (acidic side chains): asp, glu; Group IV (basic side chains): asn, gln, his, lys, arg; Group V (residues influencing chain orientation): gly, pro; and Group VI (aromatic side chains): trp, tyr, phe. Conservative substitutions involve substitutions between amino acids in the same class. Non-conservative substitutions constitute exchanging a member of one of these classes for a member of another.

In one embodiment of the present invention, antibodies that immunospecifically bind to a C35 polypeptide or a fragment or variant thereof, comprise a polypeptide having the amino acid sequence of any of SEQ ID NOs:62-69, or the VH region encoded by the polynucleotide referred to in Table 4 and/or SEQ ID NO:70 or the VL region encoded by the polynucleotide referred to in Table 4 and/or SEQ ID NO:71. In preferred embodiments, antibodies of the present invention comprise the amino acid sequence of a VH region encoded by clone H730 and a VL region encoded by clone L74, referred to in Table 4.

In some preferred embodiments, antibodies of the present invention comprise the amino acid sequence of a VH region encoded by clone H730 and a VL region encoded by clone L74. Molecules comprising, or alternatively consisting of, antibody fragments or variants of the VH and/or VL regions encoded by at least one of the polynucleotides referred to in Tables 2, 3, or 4 that immunospecifically bind to a C35 polypeptide are also encompassed by the invention, as are nucleic acid molecules encoding these VH and VL regions, molecules, fragments and/or variants.

The present invention also provides antibodies that immunospecifically bind to a polypeptide, or polypeptide fragment or variant of a C35 polypeptide, wherein said antibodies comprise, or alternatively consist of, a polypeptide having an amino acid sequence of any one, two, or three of the VH CDRs contained in VH regions encoded by SEQ ID NOs:62-64 or SEQ ID NO:70 or referred to in Table 4. In particular, the invention provides antibodies that immunospecifically bind a C35 polypeptide, comprising, or alternatively consisting of, a polypeptide having the amino acid sequence of a VH CDR1 contained in a VH region encoded by SEQ ID NO:70 or referred to in Table 4. In another embodiment, antibodies that immunospecifically bind a C35 polypeptide, comprise, or alternatively consist of, a polypeptide having the amino acid sequence of a VH CDR2 contained in a VH region encoded by SEQ ID NO:76 or referred to in Table 4. In a preferred embodiment, antibodies that immunospecifically bind a C35 polypeptide, comprise, or alternatively consist of a polypeptide having the amino acid sequence of a VH CDR3 contained in a VH region encoded by SEQ ID NO:70 or referred to in Table 4. Molecules comprising, or alternatively consisting of, these antibodies, or antibody fragments or variants thereof, that immunospecifically bind to C35 polypeptide or a C35 polypeptide fragment or variant thereof are also encompassed by the invention, as are nucleic acid molecules encoding these antibodies, molecules, fragments and/or variants.

The present invention also provides antibodies that immunospecifically bind to a polypeptide, or polypeptide fragment or variant of a C35 polypeptide, wherein said antibodies comprise, or alternatively consist of, a polypeptide having an amino acid sequence of any one, two, or three of the VL CDRs contained in a VL region encoded by SEQ ID NO:71 or referred to in Table 4. In particular, the invention provides antibodies that immunospecifically bind a C35 polypeptide, comprising, or alternatively consisting of, a polypeptide having the amino acid sequence of a VL CDR1 contained in a VL region encoded by SEQ ID NO:71 or referred to in Table 4. In another embodiment, antibodies that immunospecifically bind a C35 polypeptide, comprise, or alternatively consist of, a polypeptide having the amino acid sequence of a VL CDR2 contained in a VL region encoded by SEQ ID NO:71 or referred to in Table 4. In a preferred embodiment, antibodies that immunospecifically bind a C35 polypeptide, comprise, or alternatively consist of a polypeptide having the amino acid sequence of a VL CDR3 contained in a VL region encoded by SEQ ID NO:71 or referred to in Table 4. Molecules comprising, or alternatively consisting of, these antibodies, or antibody fragments or variants thereof, that immunospecifically bind to C35 polypeptide or a C35 polypeptide fragment or variant thereof are also encompassed by the invention, as are nucleic acid molecules encoding these antibodies, molecules, fragments and/or variants.

The present invention also provides antibodies (including molecules comprising, or alternatively consisting of, antibody fragments or variants) that immunospecifically bind to a C35 polypeptide or polypeptide fragment or variant of a C35 polypeptide, wherein said antibodies comprise, or alternatively consist of, one, two, three, or more VH CDRs and one, two, or three VL CDRs encoded by one or more polypeptides of SEQ ID NOs:62-69. In particular, the invention provides for antibodies that immunospecifically bind to a polypeptide or polypeptide fragment or variant of a C35 polypeptide, wherein said antibodies comprise, or alternatively consist of, a VH CDR1 and a VL CDR1, a VH CDR1 and a VL CDR2, a VH CDR1 and a VL CDR3, a VH CDR2 and a VL CDR1, VH CDR2 and VL CDR2, a VH CDR2 and a VL CDR3, a VH CDR3 and a VH CDR1, a VH CDR3 and a VL CDR2, a VH CDR3 and a VL CDR3, or any combination thereof, of the VH CDRs and VL CDRs of SEQ ID NOs:62-69 or contained in a VH region or VL region encoded by one or more polynucleotides of SEQ ID NOs:56, 58, or 60 or referred to in Tables 2, 3, or 4. The one, two, three, or more VH CDRs and one, two, three, or more VL CDRs may be from clones H0009 and L0010, clones H0009 and 1F2K, clones H0009 and 1B3K, clone H009 and SEQ ID NO:58, clones 1F2G and 1F2K, clones 1F2G and 1B3K, clones 1F2G and L0010, clone 1F2G and SEQ ID NO:58, clones 1B3G and 1B3K, clones 1B3G and 1F2K, clones 1B3G and L0010, clone 1B3G and SEQ ID NO:58, SEQ ID NO:56 and SEQ ID NO:58, SEQ ID NO:56 and clone L0010, SEQ ID NO:56 and clone 1F2K, SEQ ID NO:56 and clone 1B3K, SEQ ID NO:60 and SEQ ID NO:58, SEQ ID NO:60 and clone L0010, SEQ ID NO:60 and clone 1F2K, SEQ ID NO:60 and clone 1B3K, clone H730 and clone L74, SEQ ID NO:70 and clone L74, or clone H730 and SEQ ID NO:71. Molecules comprising, or alternatively consisting of, fragments or variants of these antibodies, that immunospecifically bind to C35 polypeptide are also encompassed by the invention, as are nucleic acid molecules encoding these antibodies, molecules, fragments or variants.

Most preferably the antibodies are human, chimeric (e.g., human mouse chimeric), or humanized antibodies or antigen-binding antibody fragments of the present invention, including, but not limited to, Fab, Fab′ and F(ab′)2, Fd, single-chain Fvs (scFv), diabodies, triabodies, tetrabodies, minibodies, single-chain antibodies, disulfide-linked Fvs (sdFv), and intrabodies, and fragments comprising either a VL or VH region. Antigen-binding antibody fragments, including single-chain antibodies, may comprise the variable region(s) alone or in combination with the entirety or a portion of the following: hinge region, CHI, CH2, and CH3 domains. Also included in the invention are antigen-binding fragments also comprising any combination of variable region(s) with a hinge region, CH1, CH2, and CH3 domains. Preferred C35 antibodies in the therapeutic methods, of the invention are those containing a deletion of the CH2 domain.

Antibodies of the present invention may be described or specified in terms of the epitope(s) or portion(s) of a polypeptide of the present invention which they recognize or specifically bind. The epitope(s) or polypeptide portion(s) may be specified as described herein, e.g., by N-terminal and C-terminal positions, or by size in contiguous amino acid residues. Antibodies which specifically bind any epitope or polypeptide of the present invention may also be excluded. Therefore, the present invention includes antibodies that specifically bind polypeptides of the present invention, and allows for the exclusion of the same.

Antibodies of the present invention may also be described or specified in terms of their binding affinity to a polypeptide of the invention. Preferred binding affinities include those with a dissociation constant or Kd less than 5×10(−7) M, 10(−7) M, 5×10(−8) M, 10(−8) M, 5×10(−9) M, 10(−9) M, 5×10(−10) M, 10(−10) M, 5×10(−11) M, 10(−11) M, 5×10(−12) M, 10(−12) M, 5×10(−13) M, 10(−13) M, 5×10(−14) M, 10(−14) M, 5×10(−15) M, or 10(−15) M.

Antibodies of the invention have an affinity for C35 the same as or similar to the affinity of the antibodies 1F2, 1B3, MAb 163, MAb 165, MAb 171, or MAbc009. Preferably, the antibodies of the invention have an affinity for C35 that is higher than the affinity of the antibodies 1F2, 1B3, MAb 163, MAb 165, MAb 171, or MAbc009. In a preferred embodiment, the antibodies of the invention have an affinity for C35 that is the same as, similar to, or higher than the affinity of MAb 163.

The invention also provides antibodies that competitively inhibit binding of an antibody to a C35 epitope as determined by any method known in the art for determining competitive binding, for example, the immunoassays and antibody binding assays described herein. In preferred embodiments, the antibody competitively inhibits binding to the epitope by at least 95%, at least 90%, at least 85%, at least 80%, at least 75%, at least 70%, at least 60%, or at least 50%.

Antibodies of the present invention may also be described or specified in terms of their cross-reactivity. Antibodies that do not bind any other analog, ortholog, or homolog of a polypeptide of the present invention are included. Antibodies that bind polypeptides with at least 99% 95%, at least 90%, at least 85%, at least 80%, at least 75%, at least 70%, at least 65%, at least 60%, at least 55%, and at least 50% identity (as calculated using methods known in the art and described herein) to a polypeptide of the present invention are also included in the present invention. In specific embodiments, antibodies of the present invention cross-react with murine, rat and/or rabbit homologs of human proteins and the corresponding epitopes thereof. Antibodies that do not bind polypeptides with less than 95%, less than 90%, less than 85%, less than 80%, less than 75%, less than 70%, less than 65%, less than 60%, less than 55%, and less than 50% identity (as calculated using methods known in the art and described herein) to a polypeptide of the present invention are also included in the present invention. In a specific embodiment, the above-described cross-reactivity is with respect to any single specific antigenic or immunogenic polypeptide, or combination(s) of 2, 3, 4, 5, or more of the specific antigenic and/or immunogenic polypeptides disclosed herein. Further included in the present invention are antibodies which bind polypeptides encoded by polynucleotides which hybridize to a polynucleotide of the present invention under stringent hybridization conditions (as described herein).

Antibodies of the present invention may be described or specified in terms of the epitope(s) or portion(s) of a polypeptide of the present invention which they recognize or specifically bind. The epitope(s) or polypeptide portion(s) may be specified as described herein, e.g., by N-terminal and C-terminal positions, by size in contiguous amino acid residues, or listed in the Tables and Figures. Antibodies which specifically bind any epitope or polypeptide of the present invention may also be excluded. Therefore, the present invention includes antibodies that specifically bind polypeptides of the present invention, and allows for the exclusion of the same.

In a specific embodiment, antibodies of the present invention bind to an epitope contained within the fragment represented by residues 105 to 115 of the native C35 sequence. In another embodiment, antibodies of the present invention bind to an epitope contained within the fragment represented by residues 53-104 of the native C35 sequence. In some embodiments, the antibodies of the present invention bind the same epitope as MAb 163.

Antibodies of the present invention may also be described or specified in terms of their cross-reactivity, or lack thereof. Antibodies that do not bind any other analog, ortholog, or homolog of a polypeptide of the present invention are included. Antibodies that bind polypeptides with at least 95%, at least 90%, at least 85%, at least 80%, at least 75%, at least 70%, at least 65%, at least 60%, at least 55%, and at least 50% identity (as calculated using methods known in the art and described herein) to a polypeptide of the present invention are also included in the present invention. In specific embodiments, antibodies of the present invention cross-react with murine, monkey, rat and/or rabbit homologs of human proteins and the corresponding epitopes thereof. Antibodies that do not bind polypeptides with less than 95%, less than 90%, less than 85%, less than 80%, less than 75%, less than 70%, less than 65%, less than 60%, less than 55%, and less than 50% identity (as calculated using methods known in the art and described herein) to a polypeptide of the present invention are also included in the present invention. In a specific embodiment, the above-described cross-reactivity is with respect to any single specific antigenic or immunogenic polypeptide, or combination(s) of 2, 3, 4, 5, or more of the specific antigenic and/or immunogenic polypeptides disclosed herein. Further included in the present invention are antibodies which bind polypeptides encoded by polynucleotides which hybridize to a polynucleotide of the present invention under stringent hybridization conditions (as described herein).

The present invention also provides antibodies that comprise, or alternatively consist of, variants (including derivatives) of the antibody molecules (e.g., the VH regions and/or VL regions) described herein, which antibodies immunospecifically bind to a C35 polypeptide or fragment or variant thereof. Standard techniques known to those of skill in the art can be used to introduce mutations in the nucleotide sequence encoding a molecule of the invention, including, for example, site-directed mutagenesis and PCR-mediated mutagenesis which result in amino acid substitutions. Preferably, the variants (including derivatives) encode less than 50 amino acid substitutions, less than 40 amino acid substitutions, less than 30 amino acid substitutions, less than 25 amino acid substitutions, less than 20 amino acid substitutions, less than 15 amino acid substitutions, less than 10 amino acid substitutions, less than 5 amino acid substitutions, less than 4 amino acid substitutions, less than 3 amino acid substitutions, or less than 2 amino acid substitutions relative to the reference VH region, VHCDR1, VHCD2, VHCDR3, VL region, VLCDR1, VLCDR2, or VLCDR3. A “conservative amino acid substitution” is one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity (e.g., the ability to bind a C35 polypeptide).

For example, it is possible to introduce mutations only in framework regions or only in CDR regions of an antibody molecule. Introduced mutations may be silent or neutral missense mutations, i.e., have no, or little, effect on an antibody's ability to bind antigen. These types of mutations may be useful to optimize codon usage, or improve a hybridoma's antibody production. Alternatively, non-neutral missense mutations may alter an antibody's ability to bind antigen. The location of most silent and neutral missense mutations is likely to be in the framework regions, while the location of most non-neutral missense mutations is likely to be in CDR, though this is not an absolute requirement. One of skill in the art would be able to design and test mutant molecules with desired properties such as no alteration in antigen binding activity or alteration in binding activity (e.g., affinity maturation or optimization or other improvements in antigen binding activity or change in antibody specificity). Following mutagenesis, the encoded protein may routinely be expressed and the functional and/or biological activity of the encoded protein, (e.g., ability to immunospecifically bind a C35 polypeptide) can be determined using techniques described herein or by routinely modifying techniques known in the art.

In a specific embodiment, an antibody of the invention (including a molecule comprising, or alternatively consisting of, an antibody fragment or variant thereof), that immunospecifically binds C35 polypeptides or fragments or variants thereof, comprises, or alternatively consists of, an amino acid sequence encoded by a nucleotide sequence that hybridizes to a nucleotide sequence that is complementary to that encoding one of the VH or VL regions encoded by one or more of the nucleic acids of SEQ ID NOs:56, 58, 60, 70 or 71 or referred to in Tables 2, 3, or 4 under stringent conditions, e.g., hybridization to filter-bound DNA in 6× sodium chloride/sodium citrate (SSC) at about 45° C. followed by one or more washes in 0.2×SSC/0.1% SDS at about 50-65° C., under highly stringent conditions, e.g., hybridization to filter-bound nucleic acid in 6×SSC at about 45° C. followed by one or more washes in 0.1×SSC/0.2% SDS at about 68° C., or under other stringent hybridization conditions which are known to those of skill in the art (see, for example, Ausubel, F. M. et al., eds., 1989, CURRENT PROTOCOLS IN MOLECULAR BIOLOGY, VOL. I, Green Publishing Associates, Inc. and John Wiley & Sons, Inc., New York at pages 6.3.1-6.3.6 and 2.10.3). Nucleic acid molecules encoding these antibodies are also encompassed by the invention.

It is well known within the art that polypeptides, or fragments or variants thereof, with similar amino acid sequences often have similar structure and many of the same biological activities. Thus, in one embodiment, an antibody (including a molecule comprising, or alternatively consisting of, an antibody fragment or variant thereof), that immunospecifically binds to a C35 polypeptide or fragments or variants of a C35 polypeptide, comprises, or alternatively consists of, a VH region having an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical, to the amino acid sequence of a VH region encoded by a nucleic acid of SEQ ID NO:56, 60, or 70 or referred to in Tables 2, 3, or 4.

In another embodiment, an antibody (including a molecule comprising, or alternatively consisting of, an antibody fragment or variant thereof), that immunospecifically binds to a C35 polypeptide or fragments or variants of a C35 polypeptide, comprises, or alternatively consists of, a VL region having an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical, to the amino acid sequence of a VL region encoded by a nucleic acid of SEQ ID NO:58 or 71 or referred to in Tables 2, 3, or 4.

The invention also encompasses antibodies (including molecules comprising, or alternatively consisting of, antibody fragments or variants thereof) that have one or more of the same biological characteristics as one or more of the antibodies described herein. By “biological characteristics” is meant, the in vitro or in vivo activities or properties of the antibodies, such as, for example, the ability to bind to C35 polypeptide (e.g., C35 polypeptide expressed on a cell surface during apoptosis); the ability to substantially inhibit or abolish C35 polypeptide mediated biological activity; the ability to kill C35-associated cancer cells (e.g., treat or diagnose C35-associated cancer), or detect C35. Optionally, the antibodies of the invention will bind to the same epitope as at least one of the antibodies specifically referred to herein. Such epitope binding can be routinely determined using assays known in the art and described herein below.

The rules described below for producing humanized antibodies derived from mouse VH and VL regions encoded by the nucleic acids referred to in Table 2 may also be used to produce antibody variants comprising the human VH and/or VL regions encoded by SEQ ID NOs: 56, 58, or 60 or by the nucleic acids referred to in Table 3.

Humanized immunoglobulins and human antibody variants of the invention have variable framework regions substantially from a human immunoglobulin (termed an acceptor immunoglobulin), and CDRs substantially from the mouse C35 VH and VL regions encoded by the clones in Table 2 or from the human C35 VH and VL regions encoded by the clones in Tables 3 and 4 and SEQ ID NOs:70 and 71 (referred to as the donor immunoglobulin). The constant region(s), if present, are also substantially from a human immunoglobulin. The humanized antibodies and human antibody variants exhibit a specific binding affinity for C35 of at least 10(2), 10(3), 10(4), 10(5), 10(6), 10(7), 10(8), 10(9), or 10(10) M(−1). Usually the upper limit of binding affinity of the humanized antibodies and human antibody variants for human C35 is within a factor of 3, 4, 5 or 10 of that of the mouse antibodies 1F2 or 1B3 or the human antibody MAbc009, or of antibodies MAb 163, MAb 165, or MAb 171. Often the lower limit of binding affinity is also within a factor of 3, 4, 5 or 10 of that of the mouse antibodies in 1F2 or 1B3 or human antibody MAbc009, or of antibodies MAb 163, MAb 165, or MAb 171. Preferred humanized immunoglobulins and human antibody variants compete with the mouse antibodies 1F2 or 1B3 or human antibody MAbc009, or antibodies MAb 163, MAb 165, or MAb 171 for binding to C35 and prevent C35 from binding to the respective mouse or human antibody.

The heavy and light chain variable regions of possible human acceptor antibodies are described by Kabat, Sequences of Proteins of Immunological Interest (National Institutes of Health, Bethesda, Md., 1987 and 1991). The human acceptor antibody is chosen such that its variable regions exhibit a high degree of sequence identity with those of the mouse C35 antibody. The heavy and light chain variable framework regions can be derived from the same or different human antibody sequences. The human antibody sequences can be the sequences of naturally occurring human antibodies or can be consensus sequences of several human antibodies.

The design of humanized immunoglobulins can be carried out as follows. When an amino acid falls under the following category, the framework amino acid of a human immunoglobulin to be used (acceptor immunoglobulin) is replaced by a framework amino acid from a CDR-providing non-human immunoglobulin (donor immunoglobulin):

(a) the amino acid in the human framework region of the acceptor immunoglobulin is unusual for human immunoglobulins at that position, whereas the corresponding amino acid in the donor immunoglobulin is typical for human immunoglobulins in that position;

(b) the position of the amino acid is immediately adjacent to one of the CDRs; or

(c) the amino acid is capable of interacting with the CDRs (see, Queen et al., WO 92/11018., and Co et al., Proc. Natl. Acad. Sci. USA 88, 2869 (1991), respectively, both of which are incorporated herein by reference). For a detailed description of the production of humanized immunoglobulins see, Queen et al. and Co et al.

Usually the CDR regions in humanized antibodies and human antibody variants are substantially identical, and more usually, identical to the corresponding CDR regions in the mouse or human antibody from which they were derived. It is possible to make one or more amino acid substitutions of CDR residues without appreciably affecting the binding affinity of the resulting humanized immunoglobulin or human antibody variant and, occasionally, substitutions of or within CDR regions can enhance binding affinity. See, e.g., Iwahashi et al., Mol. Immunol. 36: 1079-1091 (1999); Glaser et al., J. Immunol. 149(8): 2607-2614 (1992); and Tamura et al., J. Immunol. 164: 1432-1441 (2000).

Other than for the specific amino acid substitutions discussed above, the framework regions of humanized immunoglobulins and human antibody variants are usually substantially identical, and more usually, identical to the framework regions of the human antibodies from which they were derived (acceptor immunoglobulin). Of course, many of the amino acids in the framework region make little or no direct contribution to the specificity or affinity of an antibody.

Thus, many individual conservative substitutions of framework residues can be tolerated without appreciable change of the specificity or affinity of the resulting humanized immunoglobulin or human antibody variants.

Phage-display technology offers powerful techniques for selecting analogs that have substantial sequence identity to a parent sequence, while retaining binding affinity and specificity (see, e.g., Dower et al., WO 91/17271; McCafferty et al., WO 92/01047; and Huse, WO 92/06204 (each of which is incorporated by reference in its entirety for all purposes).

The VH and VL genes in the nucleic acid clones in Tables 2, 3, or 4 or SEQ ID NOs:56, 58, 60, 70 or 71 can be employed to select fully human antibodies specific for C35 according to the method taught by US 2002 0123057A1, “In vitro methods of producing and identifying immunoglobulin molecules in eukaryotic cells,” published 5 Sep. 2002. Briefly, the mouse (or human) VH linked to a human CH is employed to select fully human immunoglobulin light chains from a library of such light chains that when paired with the mouse (or human) VH confers specificity for C35. The selected fully human immunoglobulin light chains are then employed to select fully human immunoglobulin heavy chains from a library of such heavy chains that when paired with the fully human light chain confer specificity for C35. Similarly, the mouse (or human) VL linked to a human CL may be employed to select fully human immunoglobulin heavy chains from a library of such heavy chains that when paired with the mouse (or human) VL confers specificity for C35. The selected fully human immunoglobulin heavy chains are then employed to select fully human immunoglobulin light chains from a library of such light chains that when paired with the fully human heavy chain confer specificity for C35. Frequently, the fully human antibody selected in this fashion has epitope specificity that is identical or closely related to that of the original mouse (or human) C35-specific antibody.

The method of US 2002 0123057 A1 may also be used with a library of heavy or light chains of which all members have one or more non-human (e.g., mouse) CDRs. In one example, each member of the library comprises a CDR3 region derived from an isolated murine monoclonal antibody specific for C35, e.g., 1F2 or 1B3.

All fully human antibodies or antibodies having one or more non-human (e.g., mouse) CDRs (including molecules comprising, or alternatively consisting of, antibody fragments or variants thereof) selected through use of the method of US 2002 0123057 A1 starting with immunoglobulin heavy or light chain variable regions encoded by the nucleic acids of SEQ ID NOs:56, 58, 60, 70 or 71 or referred to in Tables 2, 3, or 4 are encompassed in the present invention.

The variable segments of humanized antibodies or human antibody variants produced as described supra are typically linked to at least a portion of an immunoglobulin constant region (Fc), typically that of a human immunoglobulin. Human constant region DNA sequences can be isolated in accordance with well-known procedures from a variety of human cells, such as immortalized B-cells (see Kabat et al., supra, and WO 87/02671). The antibody may contain both light chain and heavy chain constant regions. The heavy chain constant region may include CH1, hinge, CH2, CH3, and, sometimes, CH4 regions. For therapeutic purposes, the CH2 domain may be deleted or omitted.

The humanized antibody or human antibody variants include antibodies having all types of constant regions, including IgM, IgG, IgD, IgA and IgE, and any isotype, including IgG1, IgG2, IgG3 and IgG4. When it is desired that the humanized antibody or human antibody variants exhibit cytotoxic activity, the constant domain is usually a complement-fixing constant domain and the class is typically IgG1. When such cytotoxic activity is not desirable, the constant domain can be of the IgG2 class. The humanized antibody or human antibody variants may comprise sequences from more than one class or isotype.

Chimeric antibodies are also encompassed in the present invention. Such antibodies may comprise the VH region and/or VL region encoded by the nucleic acids of SEQ ID NOs:56, 58, 60, 70 or 71 or in Tables 2, 3, or 4 fused to the CH region and/or CL region of a another species, such as human or mouse or horse, etc. In preferred embodiments, a chimeric antibody comprises the VH and/or VL region encoded by the a murine anti-C35 antibody fused to human C regions. The human CH2 domain may be deleted when antibodies are used in therapeutic purposes. Chimeric antibodies encompass antibody fragments, as described above.

The variable segments of chimeric antibodies produced as described supra are typically linked to at least a portion of an immunoglobulin constant region (Fc), typically that of a human immunoglobulin. Human constant region DNA sequences can be isolated in accordance with well-known procedures from a variety of human cells, such as immortalized B-cells (see Kabat et al., supra, and WO 87/02671). The antibody may contain both light chain and heavy chain constant regions. The heavy chain constant region may include CH1, hinge, CH2, CH3, and, sometimes, CH4 regions. For therapeutic purposes, the CH2 domain may be deleted or omitted.

Chimeric antibodies include antibodies having all types of constant regions, including IgM, IgG, IgD, IgA and IgE, and any isotype, including IgG1, IgG2, IgG3 and IgG4. When it is desired that the chimeric antibody exhibit cytotoxic activity, the constant domain is usually a complement-fixing constant domain and the class is typically IgG1. When such cytotoxic activity is not desirable, the constant domain can be of the IgG2 class. The chimeric antibody may comprise sequences from more than one class or isotype.

A variety of methods are available for producing such immunoglobulins. Because of the degeneracy of the genetic code, a variety of nucleic acid sequences encode each immunoglobulin amino acid sequence. The desired nucleic acid sequences can be produced by de novo solid-phase DNA synthesis or by PCR mutagenesis of an earlier prepared variant of the desired polynucleotide. All nucleic acids encoding the antibodies described in this application are expressly included in the invention.

Once expressed, the whole antibodies, their dimers, individual light and heavy chains, or other immunoglobulin forms of the present invention can be purified according to standard procedures in the art, including ammonium sulfate precipitation, affinity columns, column chromatography, gel electrophoresis and the like (see, generally, Scopes, R., Protein Purification, Springer-Verlag, N.Y. (1982), which is incorporated herein by reference). Substantially pure immunoglobulins of at least about 90 to 95% homogeneity are preferred, and 98 to 99% or more homogeneity most preferred, for pharmaceutical uses. Once purified, partially or to homogeneity as desired, the polypeptides may then be used therapeutically (including extracorporeally), in developing and performing assay procedures, immunofluorescent stainings, and the like. (See, generally, Immunological Methods, Vols. I and II, Lefkovits and Pemis, eds., Academic Press, New York, N.Y. (1979 and 1981), or detect C35 or diagnose a C35-associated cancer.

The present invention also provides for fusion proteins comprising, or alternatively consisting of, an antibody (including molecules comprising, or alternatively consisting of, antibody fragments or variants thereof), that immunospecifically binds to C35 polypeptide, and a heterologous polypeptide. Preferably, the heterologous polypeptide to which the antibody is fused is useful for function or is useful to target the C35 polypeptide expressing cells, including but not limited to breast, ovarian, bladder, colon, and pancreatic cancer cells, and melanoma cells. In an alternative preferred embodiment, the heterologous polypeptide to which the antibody is fused is useful for T cell, macrophage, and/or monocyte cell function or is useful to target the antibody to a T cell, macrophage, or monocyte. In one embodiment, a fusion protein of the invention comprises, or alternatively consists of, a polypeptide having the amino acid sequence of any one or more of the VH regions of an antibody of the invention or the amino acid sequence of any one or more of the VL regions of an antibody of the invention or fragments or variants thereof, and a heterologous polypeptide sequence. In another embodiment, a fusion protein of the present invention comprises, or alternatively consists of, a polypeptide having the amino acid sequence of any one, two, three, or more of the VH CDRs of an antibody of the invention, or the amino acid sequence of any one, two, three, or more of the VL CDRs of an antibody of the invention, or fragments or variants thereof, and a heterologous polypeptide sequence. In a preferred embodiment, the fusion protein comprises, or alternatively consists of, a polypeptide having the amino acid sequence of a VH CDR3 of an antibody of the invention, or fragment or variant thereof, and a heterologous polypeptide sequence, which fusion protein immunospecifically binds to C35 polypeptide. In another embodiment, a fusion protein comprises, or alternatively consists of a polypeptide having the amino acid sequence of at least one VH region of an antibody of the invention and the amino acid sequence of at least one VL region of an antibody of the invention or fragments or variants thereof, and a heterologous polypeptide sequence. Preferably, the VH and VL regions of the fusion protein correspond to a single antibody (or scFv or Fab fragment) of the invention. In yet another embodiment, a fusion protein of the invention comprises, or alternatively consists of a polypeptide having the amino acid sequence of any one, two, three or more of the VH CDRs of an antibody of the invention and the amino acid sequence of any one, two, three or more of the VL CDRs of an antibody of the invention, or fragments or variants thereof, and a heterologous polypeptide sequence. Preferably, two, three, four, five, six, or more of the VHCDR(s) or VLCDR(s) correspond to a single antibody (or scFv or Fab fragment) of the invention. Nucleic acid molecules encoding these fusion proteins are also encompassed by the invention.

As discussed in more detail below, the antibodies of the present invention may be used either alone, in combination with each other, or in combination with other compositions. The antibodies may further be recombinantly fused to a heterologous polypeptide at the N- or C-terminus or chemically conjugated (including covalent and non-covalent conjugations) to polypeptides or other compositions. For example, antibodies of the present invention may be recombinantly fused or conjugated to molecules useful as labels in detection assays and effector molecules such as heterologous polypeptides, drugs, radionuclides, or toxins. See, e.g., PCT publications WO 92/08495; WO 91/14438; WO 89/12624; U.S. Pat. No. 5,314,995; and EP 396,387, which are herein incorporated by reference in their entireties.

By way of another non-limiting example, antibodies of the invention may be administered to individuals as a form of passive immunization. Alternatively, antibodies of the present invention may be used for epitope mapping to identify the epitope(s) bound by the antibody. Epitopes identified in this way may, in turn, for example, be used as vaccine candidates, i.e., to immunize an individual to elicit antibodies against the naturally occurring forms of C35 for therapeutic methods.

Antibodies of the present invention may act as agonists or antagonists of the C35 polypeptides.

Antibodies of the present invention may be used, for example, but not limited to, to purify, detect, and target the polypeptides of the present invention, including both in vitro and in vivo diagnostic and therapeutic methods. For example, the antibodies have use in immunoassays for qualitatively and quantitatively measuring levels of the polypeptides of the present invention in biological samples. See, e.g., Harlow et al., Antibodies: A Laboratory Manual, (Cold Spring Harbor Laboratory Press, 2nd ed. 1988) (incorporated by reference herein in its entirety).

The antibodies of the invention include derivatives that are modified, i.e., by the covalent attachment of any type of molecule to the antibody such that covalent attachment does not prevent the antibody from generating an anti-idiotypic response or binding C35. For example, but not by way of limitation, the antibody derivatives include antibodies that have been modified, e.g., by glycosylation, acetylation, pegylation, phosphylation, phosphorylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

Antibodies of the invention can be composed of amino acids joined to each other by peptide bonds or modified peptide bonds, i.e., peptide isosteres, and may contain amino acids other than the 20 gene-encoded amino acids. The C35 antibodies may be modified by natural processes, such as posttranslational processing, or by chemical modification techniques which are well known in the art. Such modifications are well described in basic texts and in more detailed monographs, as well as in a voluminous research literature. Modifications can occur anywhere in the C35.antibody, including the peptide backbone, the amino acid side-chains and the amino or carboxyl termini. It will be appreciated that the same type of modification may be present in the same or varying degrees at several sites in a given C35 antibody. Also, a given C35 antibody may contain many types of modifications. C35 antibodies may be branched, for example, as a result of ubiquitination, and they may be cyclic, with or without branching. Cyclic, branched, and branched cyclic C35 antibodies may result from posttranslation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination. (See, for instance, PROTEINS—STRUCTURE AND MOLECULAR PROPERTIES, 2nd Ed., T. E. Creighton, W. H. Freeman and Company, New York (1993); POSTTRANSLATIONAL COVALENT MODIFICATION OF PROTEINS, B. C. Johnson, Ed., Academic Press, New York, pgs. 1-12 (1983); Seifter et al., Meth Enzymol 182:626-646 (1990); Rattan et al., Ann NY Acad Sci 663:48-62 (1992).)

A further embodiment of the invention relates to a polypeptide which comprises the amino acid sequence of a C35 antibody sequence having an amino acid sequence which contains at least one amino acid substitution, but not more than 50 amino acid substitutions, even more preferably, not more than 40 amino acid substitutions, still more preferably, not more than 30 amino acid substitutions, and still even more preferably, not more than 20 amino acid substitutions. Of course, in order of ever-increasing preference, it is highly preferable for a polypeptide to have an amino acid sequence which comprises a C35 antibody sequence, which contains at least one, but not more than 10, 9, 8, 7, 6, 5, 4, 3, 2 or 1 amino acid substitutions. In specific embodiments, the number of additions, substitutions, and/or deletions in the C35 antibody sequence is 1-5, 5-10, 5-25, 5-50, 10-50 or 50-150. For substitutions, conservative amino acid substitutions are preferable. The substitutions may be within the framework regions or the CDRs or both.

The description in this section applies to C35 antibodies and to other antibodies useful in the method of the invention. Such antibodies may be conjugated to or complexed with a toxin, as described herein, or may be unconjugated or uncomplexed.

### IV. POLYNUCLEOTIDES ENCODING C35 ANTIBODIES

The present invention also provides for nucleic acid molecules encoding C35 antibodies, or antigen-binding fragments, variants, or derivatives thereof of the invention.

In one embodiment, the present invention provides an isolated polynucleotide comprising, consisting essentially of, or consisting of a nucleic acid encoding an immunoglobulin heavy chain variable region (VH), where at least one of the CDRs of the heavy chain variable region or at least two of the CDRs of the heavy chain variable region are at least 80%, 85%, 90%, 95%, 99% or 100% identical to reference heavy chain CDR1, CDR2, or CDR3 amino acid sequences from monoclonal C35 antibodies disclosed herein. Alternatively, the CDR1, CDR2, and CDR3 regions of the VH are at least 80%, 85%, 90%, 95%, 99% or 100% identical to reference heavy chain CDR1, CDR2, and CDR3 amino acid sequences from monoclonal C35 antibodies disclosed herein. Thus, for example, according to this embodiment a heavy chain variable region of the invention has CDR1, CDR2, or CDR3 polypeptide sequences related to the polypeptide sequences of SEQ ID NOs:62-65.

In certain embodiments, an antibody or antigen-binding fragment comprising the VH encoded by the polynucleotide specifically or preferentially binds to C35.

In another embodiment, the present invention provides an isolated polynucleotide comprising, consisting essentially of, or consisting of a nucleic acid encoding an immunoglobulin heavy chain variable region (VH) in which the CDR1, CDR2, and CDR3 regions have polypeptide sequences which are identical to the CDR1, CDR2, and CDR3 groups shown in SEQ ID NOs:62-65. In certain embodiments, an antibody or antigen-binding fragment comprising the VH encoded by the polynucleotide specifically or preferentially binds to C35.

In a further embodiment, the present invention includes an isolated polynucleotide comprising, consisting essentially of, or consisting of a nucleic acid encoding a VH at least 80%, 85%, 90%, 95%, 99%, or 100% identical to the reference VH polypeptide sequence in SEQ ID NO:62. In certain embodiments, an antibody or antigen-binding fragment comprising the VH encoded by the polynucleotide specifically or preferentially binds to C35.

In additional embodiments, the present invention includes an isolated polynucleotide which encodes a heavy chain variable region (V_{H}), where the polynucleotide comprises a V_{H} nucleic acid sequence selected from the group consisting of SEQ ID NO:70. In certain embodiments, an antibody or antigen-binding fragment comprising the VH encoded by the polynucleotide specifically or preferentially binds to C35.

In a further embodiment, the present invention includes an isolated polynucleotide comprising, consisting essentially of, or consisting of a VH-encoding nucleic acid at least 80%, 85%, 90%, 95%, 99%, or 100% identical to SEQ ID NO:70. In certain embodiments, the polynucleotide encodes a VH polypeptide which specifically or preferentially binds to C35.

In another embodiment, the present invention provides an isolated polynucleotide comprising, consisting essentially of, or consisting of a nucleic acid encoding an immunoglobulin light chain variable region (VL), where at least one of the CDRs of the light chain variable region or at least two of the CDRs of the light chain variable region are at least 80%, 85%, 90%, 95% or 100% identical to reference light chain CDR1, CDR2, or CDR3 amino acid sequences from monoclonal C35 antibodies disclosed herein. Alternatively, the CDR1, CDR2, and CDR3 regions of the VL are at least 80%, 85%, 90%, 95% or 100% identical to reference light chain CDR1, CDR2, and CDR3 amino acid sequences from monoclonal C35 antibodies disclosed herein. Thus, for example, according to this embodiment a light chain variable region of the invention has CDR1, CDR2, or CDR3 polypeptide sequences related to the polypeptide sequences in SEQ ID NOs:66-69.

In certain embodiments, the present invention provides an isolated polynucleotide comprising a nucleic acid sequence encoding at least one complementarity determining region (CDR) or a variant thereof of the MAb 163 monoclonal antibody, wherein said polynucleotide encodes a polypeptide that specifically binds to C35. In other embodiments, the present invention provides an isolated polynucleotide comprising a nucleic acid sequence encoding at least two, three, four, five, or six complementarity determining region (CDR) or a variant thereof of the MAb 163 monoclonal antibody, wherein said polynucleotide encodes a polypeptide that specifically binds to C35. In a preferred embodiment, the polynucleotide comprises at least one CDR of the MAb 163 monoclonal antibody, wherein said CDR is the heavy chain CDR3.

In certain embodiments, an antibody or antigen-binding fragment comprising the VL encoded by the polynucleotide specifically or preferentially binds to C35.

In another embodiment, the present invention provides an isolated polynucleotide comprising, consisting essentially of, or consisting of a nucleic acid encoding an immunoglobulin light chain variable region (VL) in which the CDR1, CDR2, and CDR3 regions have polypeptide sequences which are identical to CDR1, CDR2, and CDR3 shown in SEQ ID NOs:66-69. In certain embodiments, an antibody or antigen-binding fragment comprising the VL encoded by the polynucleotide specifically or preferentially binds to C35.

In a further embodiment, the present invention includes an isolated polynucleotide comprising, consisting essentially of, or consisting of a nucleic acid encoding a VL at least 80%, 85%, 90%, 95% or 100% identical to a reference VL polypeptide sequence selected from the group consisting of SEQ ID NO:71. In certain embodiments, an antibody or antigen-binding fragment comprising the VL encoded by the polynucleotide specifically or preferentially binds to C35.

In another aspect, the present invention includes an isolated polynucleotide comprising, consisting essentially of, or consisting of a nucleic acid sequence encoding a VL having a polypeptide sequence consisting of SEQ ID NO:66. In certain embodiments, an antibody or antigen-binding fragment comprising the VL encoded by the polynucleotide specifically or preferentially binds to C35.

In a further embodiment, the present invention includes an isolated polynucleotide comprising, consisting essentially of, or consisting of a nucleic acid encoding a VL at least 80%, 85%, 90%, 95% or 100% identical to a reference VL polypeptide sequence consisting of SEQ ID NO:66. In certain embodiments, an antibody or antigen-binding fragment comprising the VL encoded by the polynucleotide specifically or preferentially binds to C35.

In another aspect, the present invention includes an isolated polynucleotide comprising, consisting essentially of, or consisting of a nucleic acid sequence encoding a VL of the invention, for example, SEQ ID NO:71. In certain embodiments, an antibody or antigen-binding fragment comprising the VL encoded by the polynucleotide specifically or preferentially binds to C35.

In additional embodiments, the present invention includes an isolated polynucleotide which encodes a light chain variable region (V_{L}), where the polynucleotide comprises a V_{L} nucleic acid sequence consisting of SEQ ID NO:71. In certain embodiments, an antibody or antigen-binding fragment comprising the VL encoded by the polynucleotide specifically or preferentially binds to C35.

In a further embodiment, the present invention includes an isolated polynucleotide comprising, consisting essentially of, or consisting of a nucleic acid encoding a VL at least 80%, 85%, 90%, 95% or 100% identical to a VL polynucleotide consisting of SEQ ID NO:71. In certain embodiments, the polynucleotide encodes a VL polypeptide which specifically or preferentially binds to C35.

In certain embodiments, an antibody or antigen-binding fragment thereof comprising, consisting essentially of, or consisting of a VH or VL encoded by one or more of the polynucleotides described above specifically or preferentially binds to the same epitope as a monoclonal antibody selected from the group consisting of 1F2, 1B3, MAbc009, MAb 163, MAb 165, or MAb 171, or will competitively inhibit such a monoclonal antibody from binding to C35.

In certain embodiments, an antibody or antigen-binding fragment thereof comprising, consisting essentially of, or consisting of a VH or VL encoded by one or more of the polynucleotides described above specifically or preferentially binds to a C35 polypeptide or fragment thereof, or a C35 variant polypeptide, with an affinity characterized by a dissociation constant (K_{D}) no greater than 5×10⁻² M, 10⁻² M, 5×10⁻³ M, 10⁻³ M, 5×10⁻⁴ M, 10⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M.

Any of the polynucleotides described above may further include additional nucleic acids, encoding, e.g., a signal peptide to direct secretion of the encoded polypeptide, antibody constant regions as described herein, or other heterologous polypeptides as described herein.

Also, as described in more detail elsewhere herein, the present invention includes compositions comprising the polynucleotides comprising one or more of the polynucleotides described above. In one embodiment, the invention includes compositions comprising a first polynucleotide and second polynucleotide wherein said first polynucleotide encodes a VH polypeptide as described herein and wherein said second polynucleotide encodes a VL polypeptide as described herein. Specifically a composition which comprises, consists essentially of, or consists of a VH polynucleotide, and a VL polynucleotide, wherein said VH polynucleotide and said VL polynucleotide are SEQ ID NO:70 and SEQ ID NO:71, respectively.

The present invention also includes fragments of the polynucleotides of the invention, as described elsewhere. Additionally polynucleotides which encode fusion polynucleotides, Fab fragments, and other derivatives, as described herein, are also contemplated by the invention.

The polynucleotides may be produced or manufactured by any method known in the art.

For example, if the nucleotide sequence of the antibody is known, a polynucleotide encoding the antibody may be assembled from chemically synthesized oligonucleotides (e.g., as described in Kutmeier et al., BioTechniques 17:242 (1994)), which, briefly, involves the synthesis of overlapping oligonucleotides containing portions of the sequence encoding the antibody, annealing and ligating of those oligonucleotides, and then amplification of the ligated oligonucleotides by PCR.

Alternatively, a polynucleotide encoding a C35 antibody, or antigen-binding fragment, variant, or derivative thereof may be generated from nucleic acid from a suitable source. If a clone containing a nucleic acid encoding a particular antibody is not available, but the sequence of the antibody molecule is known, a nucleic acid encoding the antibody may be chemically synthesized or obtained from a suitable source (e.g., an antibody cDNA library, or a cDNA library generated from, or nucleic acid, preferably poly A+RNA, isolated from, any tissue or cells expressing the antibody or other C35 antibody, such as hybridoma cells selected to express an antibody) by PCR amplification using synthetic primers hybridizable to the 3′ and 5′ ends of the sequence or by cloning using an oligonucleotide probe specific for the particular gene sequence to identify, e.g., a cDNA clone from a cDNA library that encodes the antibody or other C35 antibody. Amplified nucleic acids generated by PCR may then be cloned into replicable cloning vectors using any method well known in the art.

Once the nucleotide sequence and corresponding amino acid sequence of the C35 antibody, or antigen-binding fragment, variant, or derivative thereof is determined, its nucleotide sequence may be manipulated using methods well known in the art for the manipulation of nucleotide sequences, e.g., recombinant DNA techniques, site directed mutagenesis, PCR, etc. (see, for example, the techniques described in Sambrook et al., Molecular Cloning, A Laboratory Manual, 2d Ed., Cold Spring Harbor Laboratory, Cold Spring Harbor, N.Y. (1990) and Ausubel et al., eds., Current Protocols in Molecular Biology, John Wiley & Sons, NY (1998), which are both incorporated by reference herein in their entireties), to generate antibodies having a different amino acid sequence, for example to create amino acid substitutions, deletions, and/or insertions.

A polynucleotide encoding a C35 antibody, or antigen-binding fragment, variant, or derivative thereof can be composed of any polyribonucleotide or polydeoxyribonucleotide, which may be unmodified RNA or DNA or modified RNA or DNA. For example, a polynucleotide encoding a C35 antibody, or antigen-binding fragment, variant, or derivative thereof can be composed of single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, a polynucleotide encoding a C35 antibody, or antigen-binding fragment, variant, or derivative thereof can be composed of triple-stranded regions comprising RNA or DNA or both RNA and DNA. A polynucleotide encoding a C35 antibody, or antigen-binding fragment, variant, or derivative thereof may also contain one or more modified bases or DNA or RNA backbones modified for stability or for other reasons. “Modified” bases include, for example, tritylated bases and unusual bases such as inosine. A variety of modifications can be made to DNA and RNA; thus, “polynucleotide” embraces chemically, enzymatically, or metabolically modified forms.

An isolated polynucleotide encoding a non-natural variant of a polypeptide derived from an immunoglobulin (e.g., an immunoglobulin heavy chain portion or light chain portion) can be created by introducing one or more nucleotide substitutions, additions or deletions into the nucleotide sequence of the immunoglobulin such that one or more amino acid substitutions, additions or deletions are introduced into the encoded protein. Mutations may be introduced by standard techniques, such as site-directed mutagenesis and PCR-mediated mutagenesis. Preferably, conservative amino acid substitutions are made at one or more non-essential amino acid residues.

### V. C35 ANTIBODY POLYPEPTIDES

The present invention is further directed to isolated polypeptides which make up C35 antibodies, and polynucleotides encoding such polypeptides. C35 antibodies of the present invention comprise polypeptides, e.g., amino acid sequences encoding C35-specific antigen binding regions derived from immunoglobulin molecules. A polypeptide or amino acid sequence “derived from” a designated protein refers to the origin of the polypeptide. In certain cases, the polypeptide or amino acid sequence which is derived from a particular starting polypeptide or amino acid sequence has an amino acid sequence that is essentially identical to that of the starting sequence, or a portion thereof, wherein the portion consists of at least 10-20 amino acids, at least 20-30 amino acids, at least 30-50 amino acids, or which is otherwise identifiable to one of ordinary skill in the art as having its origin in the starting sequence.

In one embodiment, the present invention provides an isolated polypeptide comprising, consisting essentially of, or consisting of an immunoglobulin heavy chain variable region (VH), where at least one of CDRs of the heavy chain variable region or at least two of the CDRs of the heavy chain variable region are at least 80%, 85%, 90% 95%, 99%, or 100% identical to reference heavy chain CDR1, CDR2 or CDR3 amino acid sequences from monoclonal C35 antibodies disclosed herein. Alternatively, the CDR1, CDR2 and CDR3 regions of the VH are at least 80%, 85%, 90%, 95%, 99% or 100% identical to reference heavy chain CDR1, CDR2 and CDR3 amino acid sequences from monoclonal C35 antibodies disclosed herein. Thus, according to this embodiment a heavy chain variable region of the invention has CDR1, CDR2, and CDR3 polypeptide sequences related to those in SEQ ID NOs:62-65. In certain embodiments, an antibody or antigen-binding fragment comprising the VH encoded by the polynucleotide specifically or preferentially binds to C35.

In another embodiment, the present invention provides an isolated polypeptide comprising, consisting essentially of, or consisting of an immunoglobulin heavy chain variable region (VH) in which the CDR1, CDR2, and CDR3 regions have polypeptide sequences which are identical to the CDR1, CDR2, and CDR3 shown SEQ ID NOs:62-65. In certain embodiments, an antibody or antigen-binding fragment comprising the VH encoded by the polynucleotide specifically or preferentially binds to C35.

In a further embodiment, the present invention includes an isolated polypeptide comprising, consisting essentially of, or consisting of a VH polypeptide at least 80%, 85%, 90%, 95%, 99% or 100% identical to a reference VH polypeptide sequence consisting of SEQ ID NO:62. In certain embodiments, an antibody or antigen-binding fragment comprising the VH polypeptide specifically or preferentially binds to C35.

In another aspect, the present invention includes an isolated polypeptide comprising, consisting essentially of, or consisting of a VH polypeptide consisting of SEQ ID NO:62. In certain embodiments, an antibody or antigen-binding fragment comprising the VH polypeptide specifically or preferentially binds to C35.

In certain embodiments, an antibody or antigen-binding fragment thereof comprising, consisting essentially of, or consisting of a one or more of the VH polypeptides described above specifically or preferentially binds to the same epitope as a monoclonal antibody selected from the group consisting of 1F2, 1B3, MAbc009, MAb 163, MAb 165, or MAb 171, or will competitively inhibit such a monoclonal antibody from binding to C35.

In certain embodiments, the present invention provides for an isolated antibody or antigen binding fragment thereof comprising at least one, two, three, four, five or six CDRs of the MAb 163 monoclonal antibody, wherein said antibody or fragment specifically binds C35. In a preferred embodiment, the antibody or antigen binding fragment thereof comprises at least three CDRS of the MAb 163 monoclonal antibody. In another embodiment, the antibody or fragment comprises one CDR of MAb 163. In a specific embodiment, the one CDR is heavy chain CDR3.

In certain embodiments, an antibody or antigen-binding fragment thereof comprising, consisting essentially of, or consisting of one or more of the VH polypeptides described above specifically or preferentially binds to a C35 polypeptide or fragment thereof, or a C35 variant polypeptide, with an affinity characterized by a dissociation constant (K_{D}) no greater than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M.

In another embodiment, the present invention provides an isolated polypeptide comprising, consisting essentially of, or consisting, of an immunoglobulin light chain variable region (VL), where at least one of the CDRs of the light chain variable region or at least two of the CDRs of the light chain variable region are at least 80%, 85%, 90%, 95%, 99% or 100% identical to reference heavy chain CDR1, CDR2, or CDR3 amino acid sequences from monoclonal C35 antibodies disclosed herein. Alternatively, the CDR1, CDR2 and CDR3 regions of the VL are at least 80%, 85%, 90%, 95%, 99% or 100% identical to reference light chain CDR1, CDR2, and CDR3 amino acid sequences from monoclonal C35 antibodies disclosed herein. Thus, according to this embodiment a light chain variable region of the invention has CDR1, CDR2, and CDR3 polypeptide sequences related to the polypeptides shown in SEQ ID NOs:66-69. In certain embodiments, an antibody or antigen-binding fragment comprising the VL polypeptide specifically or preferentially binds to C35.

In another embodiment, the present invention provides an isolated polypeptide comprising, consisting essentially of, or consisting of an immunoglobulin light chain variable region (VL) in which the CDR1, CDR2, and CDR3 regions have polypeptide sequences which are identical to the CDR1, CDR2, and CDR3 shown SEQ ID NO:66. In certain embodiments, an antibody or antigen-binding fragment comprising the VL polypeptide specifically or preferentially binds to C35.

In a further embodiment, the present invention includes an isolated polypeptide comprising, consisting essentially of, or consisting of a VL polypeptide at least 80%, 85%, 90% 95%, 99% or 100% identical to a reference VL polypeptide sequence consisting of SEQ ID NO:66. In certain embodiments, an antibody or antigen-binding fragment comprising the VL polypeptide specifically or preferentially binds to C35.

In another aspect, the present invention includes an isolated polypeptide comprising, consisting essentially of, or consisting of a VL polypeptide consisting of SEQ ID NO:66. In certain embodiments, an antibody or antigen-binding fragment comprising the VL polypeptide specifically or preferentially binds to C35.

In certain embodiments, an antibody or antigen-binding fragment thereof comprising, consisting essentially of, one or more of the VL polypeptides described above specifically or preferentially binds to the same epitope as a monoclonal antibody selected from the group consisting of 1F2, 1B3, MAbc009, MAb 163, MAb 165, or MAb 171, or will competitively inhibit such a monoclonal antibody from binding to C35.

In certain embodiments, an antibody or antigen-binding fragment thereof comprising, consisting essentially of, or consisting of a one or more of the VL polypeptides described above specifically or preferentially binds to a C35 polypeptide or fragment thereof, or a C35 variant polypeptide, with an affinity characterized by a dissociation constant (K_{D}) no greater than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10¹⁰ M, 10⁻¹⁰ M, 5×10⁻¹¹M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M.

In other embodiments, an antibody or antigen-binding fragment thereof comprises, consists essentially of or consists of a VH polypeptide, and a VL polypeptide selected from the group consisting of SEQ ID NO:62 and SEQ ID NO:66 or a combination of the two.

Any of the polypeptides described above may further include additional polypeptides, e.g., a signal peptide to direct secretion of the encoded polypeptide, antibody constant regions as described herein, or other heterologous polypeptides as described herein. Additionally, polypeptides of the invention include polypeptide fragments as described elsewhere. Additionally polypeptides of the invention include fusion polypeptide, Fab fragments, and other derivatives, as described herein.

Also, as described in more detail elsewhere herein, the present invention includes compositions comprising the polypeptides described above.

It will also be understood by one of ordinary skill in the art that C35 antibody polypeptides as disclosed herein may be modified such that they vary in amino acid sequence from the naturally occurring binding polypeptide from which they were derived. For example, a polypeptide or amino acid sequence derived from a designated protein may be similar, e.g., have a certain percent identity to the starting sequence, e.g., it may be 60%, 70%, 75%, 80%, 85%, 90%, 95%, or 99% identical to the starting sequence.

Furthermore, nucleotide or amino acid substitutions, deletions, or insertions leading to conservative substitutions or changes at “non-essential” amino acid regions may be made. For example, a polypeptide or amino acid sequence derived from a designated protein may be identical to the starting sequence except for one or more individual amino acid substitutions, insertions, or deletions, e.g., one, two, three, four, five, six, seven, eight, nine, ten, fifteen, twenty or more individual amino acid substitutions, insertions, or deletions. In certain embodiments, a polypeptide or amino acid sequence derived from a designated protein has one to five, one to ten, one to fifteen, or one to twenty individual amino acid substitutions, insertions, or deletions relative to the starting sequence.

Certain C35 antibody polypeptides of the present invention comprise, consist essentially of, or consist of an amino acid sequence derived from a human amino acid sequence. However, certain C35 antibody polypeptides comprise one or more contiguous amino acids derived from another mammalian species. For example, a C35 antibody of the present invention may include a primate heavy chain portion, hinge portion, or antigen binding region. In another example, one or more murine-derived amino acids may be present in a non-murine antibody polypeptide, e.g., in an antigen binding site of a C35 antibody. In certain therapeutic applications, C35-specific antibodies, or antigen-binding fragments, variants, or analogs thereof are designed so as to not be immunogenic in the animal to which the antibody is administered.

In certain embodiments, a C35 antibody polypeptide comprises an amino acid sequence or one or more moieties not normally associated with an antibody. Exemplary modifications are described in more detail below. For example, a single-chain fv antibody fragment of the invention may comprise a flexible linker sequence, or may be modified to add a functional moiety (e.g., PEG, a drug, a toxin, or a label).

A C35 antibody polypeptide of the invention may comprise, consist essentially of, or consist of a fusion protein. Fusion proteins are chimeric molecules which comprise, for example, an immunoglobulin antigen-binding domain with at least one target binding site, and at least one heterologous portion, i.e., a portion with which it is not naturally linked in nature. The amino acid sequences may normally exist in separate proteins that are brought together in the fusion polypeptide or they may normally exist in the same protein but are placed in a new arrangement in the fusion polypeptide. Fusion proteins may be created, for example, by chemical synthesis, or by creating and translating a polynucleotide in which the peptide regions are encoded in the desired relationship.

The term “heterologous” as applied to a polynucleotide or a polypeptide, means that the polynucleotide or polypeptide is derived from a distinct entity from that of the rest of the entity to which it is being compared. For instance, as used herein, a “heterologous polypeptide” to be fused to a C35 antibody, or an antigen-binding fragment, variant, or analog thereof is derived from a non-immunoglobulin polypeptide of the same species, or an immunoglobulin or non-immunoglobulin polypeptide of a different species.

A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a nonessential amino acid residue in an immunoglobulin polypeptide is preferably replaced with another amino acid residue from the same side chain family. In another embodiment, a string of amino acids can be replaced with a structurally similar string that differs in order and/or composition of side chain family members.

Alternatively, in another embodiment, mutations may be introduced randomly along all or part of the immunoglobulin coding sequence, such as by saturation mutagenesis, and the resultant mutants can be incorporated into C35 antibodies for use in the diagnostic and treatment methods disclosed herein and screened for their ability to bind to the desired antigen, e.g., C35.

### VI. FUSION PROTEINS AND ANTIBODY CONJUGATES

As discussed in more detail elsewhere herein, C35 antibodies, or antigen-binding fragments, variants, or derivatives thereof of the invention may further be recombinantly fused to a heterologous polypeptide at the N- or C-terminus or chemically conjugated (including covalent and non-covalent conjugations) to polypeptides or other compositions. For example, C35-specific antibodies may be recombinantly fused or conjugated to molecules useful as labels in detection assays and effector molecules such as heterologous polypeptides, drugs, radionuclides, or toxins.

See, e.g., PCT publications WO 92/08495; WO 91/14438; WO 89/12624; U.S. Pat. No. 5,314,995; and EP 396,387.

C35 antibodies, or antigen-binding fragments, variants, or derivatives thereof of the invention include derivatives that are modified, i.e., by the covalent attachment of any type of molecule to the antibody such that covalent attachment does not prevent the antibody binding C35. For example, but not by way of limitation, the antibody derivatives include antibodies, that have been modified, e.g., by glycosylation, acetylation, pegylation, phosphylation, phosphorylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

C35 antibodies, or antigen-binding fragments, variants, or derivatives thereof of the invention can be composed of amino acids joined to each other by peptide bonds or modified peptide bonds, i.e., peptide isosteres, and may contain amino acids other than the 20 gene-encoded amino acids. C35-specfic antibodies may be modified by natural processes, such as posttranslational processing, or by chemical modification techniques which are well known in the art. Such modifications are well described in basic texts and in more detailed monographs, as well as in a voluminous research literature. Modifications can occur anywhere in the C35-specific antibody, including the peptide backbone, the amino acid side-chains and the amino or carboxyl termini, or on moieties such as carbohydrates. It will be appreciated that the same type of modification may be present in the same or varying degrees at several sites in a given C35-specific antibody. Also, a given C35-specific antibody may contain many types of modifications. C35-specific antibodies may be branched, for example, as a result of ubiquitination, and they may be cyclic, with or without branching. Cyclic, branched, and branched cyclic C35-specific antibodies may result from posttranslation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination. (See, for instance, Proteins—Structure And Molecular Properties, T. E. Creighton, W. H. Freeman and Company, New York 2nd Ed., (1993); Posttranslational Covalent Modification Of Proteins, B. C. Johnson, Ed., Academic Press, New York, pgs. 1-12 (1983); Seifter et al., Meth Enzymol 182:626-646 (1990); Rattan et al., Ann NY Acad Sci 663:48-62 (1992)).

The present invention also provides for fusion proteins comprising a C35 antibody, or antigen-binding fragment, variant, or derivative thereof, and a heterologous polypeptide. The heterologous polypeptide to which the antibody is fused may be useful for function or is useful to target the C35 polypeptide expressing cells. In one embodiment, a fusion protein of the invention comprises, consists essentially of, or consists of, a polypeptide having the amino acid sequence of any one or more of the VH regions of an antibody of the invention or the amino acid sequence of any one or more of the VL regions of an antibody of the invention or fragments or variants thereof, and a heterologous polypeptide sequence. In another embodiment, a fusion protein for use in the diagnostic and treatment methods disclosed herein comprises, consists essentially of, or consists of a polypeptide having the amino acid sequence of any one, two, three of the VH CDRs of a C35-specific antibody, or fragments, variants, or derivatives thereof, or the amino acid sequence of any one, two, three of the VL CDRs of a C35-specific antibody, or fragments, variants, or derivatives thereof, and a heterologous polypeptide sequence. In one embodiment, the fusion protein comprises a polypeptide having the amino acid sequence of a V_{H} CDR3 of a C35-specific antibody of the present invention, or fragment, derivative, or variant thereof, and a heterologous polypeptide sequence, which fusion protein specifically binds to at least one epitope of C35. In another embodiment, a fusion protein comprises a polypeptide having the amino acid sequence of at least one V_{H} region of a C35-specific antibody of the invention and the amino acid sequence of at least one V_{L} region of a C35-specific antibody of the invention or fragments, derivatives or variants thereof, and a heterologous polypeptide sequence. Preferably, the V_{H} and V_{L} regions of the fusion protein correspond to a single source antibody (or scFv or Fab fragment) which specifically binds at least one epitope of C35. In yet another embodiment, a fusion protein for use in the diagnostic and treatment methods disclosed herein comprises a polypeptide having the amino acid sequence of any one, two, three or more of the V_{H} CDRs of a C35-specific antibody and the amino acid sequence of any one, two, three or more of the V_{L} CDRs of a C35-specific antibody, or fragments or variants thereof, and a heterologous polypeptide sequence. Preferably, two, three, four, five, six, or more of the V_{H}CDR(s) or V_{L}CDR(s) correspond to single source antibody (or scFv or Fab fragment) of the invention. Nucleic acid molecules encoding these fusion proteins are also encompassed by the invention.

Exemplary fusion proteins reported in the literature include fusions of the T cell receptor (Gascoigne et al., Proc. Natl. Acad. Sci. USA 84:2936-2940 (1987)); CD4 (Capon et al., Nature 337:525-531 (1989); Traunecker et al., Nature 339:68-70 (1989); Zettmeissl et al., DNA Cell Biol. USA 9:347-353 (1990); and Byrn et al., Nature 344:667-670 (1990)); L-selectin (homing receptor) (Watson et al., J. Cell. Biol. 110:2221-2229 (1990); and Watson et al., Nature 349:164-167 (1991)); CD44 (Aruffo et al., Cell 61:1303-1313 (1990)); CD28 and B7 (Linsley et al., J. Exp. Med. 173:721-730 (1991)); CTLA-4 (Lisley et al., J. Exp. Med. 174:561-569 (1991)); CD22 (Stamenkovic et al., Cell 66:1133-1144 (1991)); TNF receptor (Ashkenazi et al., Proc. Natl. Acad. Sci. USA 88:10535-10539 (1991); Lesslauer et al., Eur. J. Immunol. 27:2883-2886 (1991); and Peppel et al., J. Exp. Med. 174:1483-1489 (1991)); and IgE receptor a (Ridgway and Gorman, J. Cell. Biol. Vol. 115, Abstract No. 1448 (1991)).

As discussed elsewhere herein, C35 antibodies, or antigen-binding fragments, variants, or derivatives thereof of the invention may be fused to heterologous polypeptides to increase the in vivo half life of the polypeptides or for use in immunoassays using methods known in the art. For example, in one embodiment, PEG can be conjugated to the C35 antibodies of the invention to increase their half-life in vivo. Leong, S. R., et al., Cytokine 16:106 (2001); Adv. in Drug Deliv. Rev. 54:531 (2002); or Weir et al., Biochem. Soc. Transactions 30:512 (2002).

Moreover, C35 antibodies, or antigen-binding fragments, variants, or derivatives thereof of the invention can be fused to marker sequences, such as a peptide to facilitate their purification or detection. In preferred embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., Proc. Natl. Acad. Sci. USA 86:821-824 (1989), for instance, hexa-histidine provides for convenient purification of the fusion protein. Other peptide tags useful for purification include, but are not limited to, the “HA” tag, which corresponds to an epitope derived from the influenza hemagglutinin protein (Wilson et al., Cell 37:767 (1984)) and the “flag” tag.

Fusion proteins can be prepared using methods that are well known in the art (see for example U.S. Pat. Nos. 5,116,964 and 5,225,538). The precise site at which the fusion is made may be selected empirically to optimize the secretion or binding characteristics of the fusion protein. DNA encoding the fusion protein is then transfected into a host cell for expression.

C35 antibodies of the present invention may be used in non-conjugated form or may be conjugated to at least one of a variety of molecules, e.g., to improve the therapeutic properties of the molecule, to facilitate target detection, or for imaging or therapy of the patient. C35 antibodies, or antigen-binding fragments, variants, or derivatives thereof of the invention can be labeled or conjugated either before or after purification, when purification is performed.

In particular, C35 antibodies, or antigen-binding fragments, variants, or derivatives thereof of the invention may be conjugated to therapeutic agents, prodrugs, peptides, proteins, enzymes, viruses, lipids, biological response modifiers, pharmaceutical agents, or PEG.

Those skilled in the art will appreciate that conjugates may also be assembled using a variety of techniques depending on the selected agent to be conjugated. For example, conjugates with biotin are prepared e.g. by reacting a binding polypeptide with an activated ester of biotin such as the biotin N-hydroxysuccinimide ester. Similarly, conjugates with a fluorescent marker may be prepared in the presence of a coupling agent, e.g. those listed herein, or by reaction with an isothiocyanate, preferably fluorescein-isothiocyanate. Conjugates of the C35 antibodies, or antigen-binding fragments, variants, or derivatives thereof of the invention are prepared in an analogous manner.

The present invention further encompasses C35 antibodies, or antigen-binding fragments, variants, or derivatives thereof of the invention conjugated to a diagnostic or therapeutic agent. The C35 antibodies can be used diagnostically to, for example, monitor the development or progression of a disease as part of a clinical testing procedure to, e.g., determine the efficacy of a given treatment and/or prevention regimen. Detection can be facilitated by coupling the C35 antibody, or antigen-binding fragment, variant, or derivative thereof to a detectable substance. Examples of detectable substances include various enzymes, prosthetic groups, fluorescent materials, luminescent materials, bioluminescent materials, radioactive materials, positron emitting metals using various positron emission tomographies, and nonradioactive paramagnetic metal ions. See, for example, U.S. Pat. No. 4,741,900 for metal ions which can be conjugated to antibodies for use as diagnostics according to the present invention. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin; and examples of suitable radioactive material include ¹²⁵I, ¹³¹I, ¹¹¹In or ⁹⁹Tc.

A C35 antibody, or antigen-binding fragment, variant, or derivative thereof also can be detectably labeled by coupling it to a chemiluminescent compound. The presence of the chemiluminescent-tagged C35 antibody is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester.

One of the ways in which a C35 antibody, or antigen-binding fragment, variant, or derivative thereof can be detectably labeled is by linking the same to an enzyme and using the linked product in an enzyme immunoassay (EIA) (Voller, A., “The Enzyme Linked Immunosorbent Assay (ELISA)” Microbiological Associates Quarterly Publication, Walkersville, Md., Diagnostic Horizons 2:1-7 (1978)); Voller et al., J. Clin. Pathol. 31:507-520 (1978); Butler, J. E., Meth. Enrymol. 73:482-523 (1981); Maggio, E. (ed.), Enzyme Immunoassay, CRC Press, Boca Raton, Fla., (1980); Ishikawa, E. et al., (eds.), Enzyme Immunoassay, Kgaku Shoin, Tokyo (1981). The enzyme, which is bound to the C35 antibody will react with an appropriate substrate, preferably a chromogenic substrate, in such a manner as to produce a chemical moiety which can be detected, for example, by spectrophotometric, fluorimetric or by visual means. Enzymes which can be used to detectably label the antibody include, but are not limited to, malate dehydrogenase, staphylococcal nuclease, delta-5-steroid isomerase, yeast alcohol dehydrogenase, alpha-glycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase. Additionally, the detection can be accomplished by calorimetric methods which employ a chromogenic substrate for the enzyme. Detection may also be accomplished by visual comparison of the extent of enzymatic reaction of a substrate in comparison with similarly prepared standards.

Detection may also be accomplished using any of a variety of other immunoassays. For example, by radioactively labeling the C35 antibody, or antigen-binding fragment, variant, or derivative thereof, it is possible to detect the antibody through the use of a radioimmunoassay (RIA) (see, for example, Weintraub, B., Principles of Radioimmunoassays, Seventh Training Course on Radioligand Assay Techniques, The Endocrine Society, (March, 1986)), which is incorporated by reference herein). The radioactive isotope can be detected by means including, but not limited to, a gamma counter, a scintillation counter, or autoradiography.

A C35 antibody, or antigen-binding fragment, variant, or derivative thereof can also be detectably labeled using fluorescence emitting metals such as ¹⁵²Eu, or others of the lanthanide series. These metals can be attached to the antibody using such metal chelating groups as diethylenetriaminepentacetic acid (DTPA) or ethylenediaminetetraacetic acid (EDTA).

Techniques for conjugating various moieties to a C35 antibody, or antigen-binding fragment, variant, or derivative thereof are well known, see, e.g., Amon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. (1985); Hellstrom et al., “Antibodies For Drug Delivery”, in Controlled Drug Delivery (2nd Ed.), Robinson et al. (eds.), Marcel Dekker, Inc., pp. 623-53 (1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), Academic Press pp. 303-16 (1985), and Thorpe et al., “The Preparation And Cytotoxic Properties Of Antibody-Toxin Conjugates”, Immunol. Rev. 62:119-58 (1982). Each of these references is herein incorporated in its entirety.

### VII. EXPRESSION OF ANTIBODY POLYPEPTIDES

As is well known, RNA may be isolated from the original hybridoma cells or from other transformed cells by standard techniques, such as guanidinium isothiocyanate extraction and precipitation followed by centrifugation or chromatography. Where desirable, mRNA may be isolated from total RNA by standard techniques such as chromatography on oligo dT cellulose. Suitable techniques are familiar in the art.

In one embodiment, cDNAs that encode the light and the heavy chains of the antibody may be made, either simultaneously or separately, using reverse transcriptase and DNA polymerase in accordance with well known methods. PCR may be initiated by consensus constant region primers or by more specific primers based on the published heavy and light chain DNA and amino acid sequences. As discussed above, PCR also may be used to isolate DNA clones encoding the antibody light and heavy chains. In this case the libraries may be screened by consensus primers or larger homologous probes, such as mouse constant region probes.

DNA, typically plasmid DNA, may be isolated from the cells using techniques known in the art, restriction mapped and sequenced in accordance with standard, well known techniques set forth in detail, e.g., in the foregoing references relating to recombinant DNA techniques. Of course, the DNA may be synthetic according to the present invention at any point during the isolation process or subsequent analysis.

Following manipulation of the isolated genetic material to provide C35 antibodies, or antigen-binding fragments, variants, or derivatives thereof of the invention, the polynucleotides encoding the C35 antibodies are typically inserted in an expression vector for introduction into host cells that may be used to produce the desired quantity of C35 antibody.

Recombinant expression of an antibody, or fragment, derivative or analog thereof, e.g., a heavy or light chain of an antibody which binds to a target molecule described herein, e.g., C35, requires construction of an expression vector containing a polynucleotide that encodes the antibody. Once a polynucleotide encoding an antibody molecule or a heavy or light chain of an antibody, or portion thereof (preferably containing the heavy or light chain variable domain), of the invention has been obtained, the vector for the production of the antibody molecule may be produced by recombinant DNA technology using techniques well known in the art. Thus, methods for preparing a protein by expressing a polynucleotide containing an antibody encoding nucleotide sequence are described herein. Methods which are well known to those skilled in the art can be used to construct expression vectors containing antibody coding sequences and appropriate transcriptional and translational control signals. These methods include, for example, in vitro recombinant DNA techniques, synthetic techniques, and in vivo genetic recombination. The invention, thus, provides replicable vectors comprising a nucleotide sequence encoding an antibody molecule of the invention, or a heavy or light chain thereof, or a heavy or light chain variable domain, operably linked to a promoter. Such vectors may include the nucleotide sequence encoding the constant region of the antibody molecule (see, e.g., PCT Publication WO 86/05807; PCT Publication WO 89/01036; and U.S. Pat. No. 5,122,464) and the variable domain of the antibody may be cloned into such a vector for expression of the entire heavy or light chain.

The term “vector” or “expression vector” is used herein to mean vectors used in accordance with the present invention as a vehicle for introducing into and expressing a desired gene in a host cell. As known to those skilled in the art, such vectors may easily be selected from the group consisting of plasmids, phages, viruses and retroviruses. In general, vectors compatible with the instant invention will comprise a selection marker, appropriate restriction sites to facilitate cloning of the desired gene and the ability to enter and/or replicate in eukaryotic or prokaryotic cells.

For the purposes of this invention, numerous expression vector systems may be employed. For example, one class of vector utilizes DNA elements which are derived from animal viruses such as bovine papilloma virus, polyoma virus, adenovirus, vaccinia virus, baculovirus, retroviruses (RSV, MMTV or MOMLV) or SV40 virus. Others involve the use of polycistronic systems with internal ribosome binding sites. Additionally, cells which have integrated the DNA into their chromosomes may be selected by introducing one or more markers which allow selection of transfected host cells. The marker may provide for prototrophy to an auxotrophic host, biocide resistance (e.g., antibiotics) or resistance to heavy metals such as copper. The selectable marker gene can either be directly linked to the DNA sequences to be expressed, or introduced into the same cell by cotransformation. Additional elements may also be needed for optimal synthesis of mRNA. These elements may include signal sequences, splice signals, as well as transcriptional promoters, enhancers, and termination signals.

In particularly preferred embodiments the cloned variable region genes are inserted into an expression vector along with the heavy and light chain constant region genes (preferably human) synthesized as discussed above. Of course, any expression vector which is capable of eliciting expression in eukaryotic cells may be used in the present invention. Examples of suitable vectors include, but are not limited to plasmids pcDNA3, pHCMV/Zeo, pCR3.1, pEF1/His, pIND/GS, pRc/HCMV2, pSV40/Zeo2, pTRACER-HCMV, pUB6/V5-His, pVAX1, and pZeoSV2 (available from Invitrogen, San Diego, Calif.), and plasmid pCI (available from Promega, Madison, Wis.). In general, screening large numbers of transformed cells for those which express suitably high levels if immunoglobulin heavy and light chains is routine experimentation which can be carried out, for example, by robotic systems.

More generally, once the vector or DNA sequence encoding a monomeric subunit of the C35 antibody has been prepared, the expression vector may be introduced into an appropriate host cell. Introduction of the plasmid into the host cell can be accomplished by various techniques well known to those of skill in the art. These include, but are not limited to, transfection (including electrophoresis and electroporation), protoplast fusion, calcium phosphate precipitation, cell fusion with enveloped DNA, microinjection, and infection with intact virus. See, Ridgway, A. A. G. “Mammalian Expression Vectors” Vectors, Rodriguez and Denhardt, Eds., Butterworths, Boston, Mass., Chapter 24.2, pp. 470-472 (1988). Typically, plasmid introduction into the host is via electroporation. The host cells harboring the expression construct are grown under conditions appropriate to the production of the light chains and heavy chains, and assayed for heavy and/or light chain protein synthesis. Exemplary assay techniques include enzyme-linked immunosorbent assay (ELISA), radioimmunoassay (RIA), or fluorescence-activated cell sorter analysis (FACS), immunohistochemistry and the like.

The expression vector is transferred to a host cell by conventional techniques and the transfected cells are then cultured by conventional techniques to produce an antibody for use in the methods described herein. Thus, the invention includes host cells containing a polynucleotide encoding an antibody or fragment thereof of the invention, or a heavy or light chain thereof, operably linked to a heterologous promoter. In preferred embodiments for the expression of double-chained antibodies, vectors encoding both the heavy and light chains may be co-expressed in the host cell for expression of the entire immunoglobulin molecule, as detailed below.

As used herein, “host cells” refers to cells which harbor vectors constructed using recombinant DNA techniques and encoding at least one heterologous gene. In descriptions of processes for isolation of antibodies from recombinant hosts, the terms “cell” and “cell culture” are used interchangeably to denote the source of antibody unless it is clearly specified otherwise. In other words, recovery of polypeptide from the “cells” may mean either from spun down whole cells, or from the cell culture containing both the medium and the suspended cells.

A variety of host-expression vector systems may be utilized to express antibody molecules for use in the methods described herein. Such host-expression systems represent vehicles by which the coding sequences of interest may be produced and subsequently purified, but also represent cells which may, when transformed or transfected with the appropriate nucleotide coding sequences, express an antibody molecule of the invention in situ. These include but are not limited to microorganisms such as bacteria (e.g., E. coli, B. subtilis) transformed with recombinant bacteriophage DNA, plasmid DNA or cosmid DNA expression vectors containing antibody coding sequences; yeast (e.g., Saccharomyces, Pichia) transformed with recombinant yeast expression vectors containing antibody coding sequences; insect cell systems infected with recombinant virus expression vectors (e.g., baculovirus) containing antibody coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BLK, 293, 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Preferably, bacterial cells such as Escherichia coli, and more preferably, eukaryotic cells, especially for the expression of whole recombinant antibody molecule, are used for the expression of a recombinant antibody molecule. For example, mammalian cells such as Chinese hamster ovary cells (CHO), in conjunction with a vector such as the major intermediate early gene promoter element from human cytomegalovirus is an effective expression system for antibodies (Foecking et al., Gene 45:101 (1986); Cockett et al., Bio/Technology 8:2 (1990)).

The host cell line used for protein expression is often of mammalian origin; those skilled in the art are credited with ability to preferentially determine particular host cell lines which are best suited for the desired gene product to be expressed therein. Exemplary host cell lines include, but are not limited to, CHO (Chinese Hamster Ovary), DG44 and DUXB11 (Chinese Hamster Ovary lines, DHFR minus), HELA (human cervical carcinoma), CV1 (monkey kidney line), COS (a derivative of CV1 with SV40 T antigen), VERY, BHK (baby hamster kidney), MDCK, 293, W138, R1610 (Chinese hamster fibroblast) BALBC/3T3 (mouse fibroblast), HAK (hamster kidney line), SP2/O (mouse myeloma), P3×63-Ag3.653 (mouse myeloma), BFA-1c1BPT (bovine endothelial cells), RAJI (human lymphocyte) and 293 (human kidney). Host cell lines are typically available from commercial services, the American Tissue Culture Collection or from published literature.

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used.

For long-term, high-yield production of recombinant proteins, stable expression is preferred. For example, cell lines which stably express the antibody molecule may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which stably express the antibody molecule.

A number of selection systems may be used, including but not limited to the herpes simplex virus thymidine kinase (Wigler et al., Cell 11:223 (1977)), hypoxanthine-guanine phosphoribosyltransferase (Szybalska & Szybalski, Proc. Natl. Acad. Sci. USA 48:202 (1992)), and adenine phosphoribosyltransferase (Lowy et al., Cell 22:817 1980) genes can be employed in tk-, hgprt- or aprt-cells, respectively. Also, antimetabolite resistance can be used as the basis of selection for the following genes: dhfr, which confers resistance to methotrexate (Wigler et al., Natl. Acad. Sci. USA 77:357 (1980); O'Hare et al., Proc. Natl. Acad. Sci. USA 78:1527 (1981)); gpt, which confers resistance to mycophenolic acid (Mulligan & Berg, Proc. Natl. Acad. Sci. USA 78:2072 (1981)); neo, which confers resistance to the aminoglycoside GA418 Clinical Pharmacy 12:488-505; Wu and Wu, Biotherapy 3:87-95 (1991); Tolstoshev, Ann. Rev. Pharmacol. Toxicol. 32:573-596 (1993); Mulligan, Science 260:926-932 (1993); and Morgan and Anderson, Ann. Rev. Biochem. 62:191-217 (1993), TIB TECH 11(5):155-215 (May, 1993); and hygro, which confers resistance to hygromycin (Santerre et al., Gene 30:147 (1984). Methods commonly known in the art of recombinant DNA technology which can be used are described in Ausubel et al. (eds.), Current Protocols in Molecular Biology, John Wiley & Sons, NY (1993); Kriegler, Gene Transfer and Expression, A Laboratory Manual, Stockton Press, NY (1990); and in Chapters 12 and 13, Dracopoli et al. (eds), Current Prolocols in Human Genetics, John Wiley & Sons, NY (1994); Colberre-Garapin et al., J. Mol. Biol. 150:1 (1981), which are incorporated by reference herein in their entireties.

The expression levels of an antibody molecule can be increased by vector amplification (for a review, see Bebbington and Hentschel, The use of vectors based on gene amplification for the expression of cloned genes in mammalian cells in DNA cloning, Academic Press, New York, Vol. 3. (1987)). When a marker in the vector system expressing antibody is amplifiable, increase in the level of inhibitor present in culture of host cell will increase the number of copies of the marker gene. Since the amplified region is associated with the antibody gene, production of the antibody will also increase (Crouse et al., Mol. Cell. Biol. 3:257 (1983)).

In vitro production allows scale-up to give large amounts of the desired polypeptides. Techniques for mammalian cell cultivation under tissue culture conditions are known in the art and include homogeneous suspension culture, e.g. in an airlift reactor or in a continuous stirrer reactor, or immobilized or entrapped cell culture, e.g. in hollow fibers, microcapsules, on agarose microbeads or ceramic cartridges. If necessary and/or desired, the solutions of polypeptides can be purified by the customary chromatography methods, for example gel filtration, ion-exchange chromatography, chromatography over DEAE-cellulose or (immuno-)affinity chromatography, e.g., after preferential biosynthesis of a synthetic hinge region polypeptide or prior to or subsequent to the HIC chromatography step described herein.

Genes encoding C35 antibodies, or antigen-binding fragments, variants, or derivatives thereof of the invention can also be expressed non-mammalian cells such as bacteria or yeast or plant cells. Bacteria which readily take up nucleic acids include members of the enterobacteriaceae, such as strains of Escherichia coli or Salmonella; Bacillaceae, such as Bacillus subtilis; Pneumococcus; Streptococcus, and Haemophilus influenzae. It will further be appreciated that, when expressed in bacteria, the heterologous polypeptides typically become part of inclusion bodies. The heterologous polypeptides must be isolated, purified and then assembled into functional molecules. Where tetravalent forms of antibodies are desired, the subunits will then self-assemble into tetravalent antibodies (WO02/096948A2).

In bacterial systems, a number of expression vectors may be advantageously selected depending upon the use intended for the antibody molecule being expressed. For example, when a large quantity of such a protein is to be produced, for the generation of pharmaceutical compositions of an antibody molecule, vectors which direct the expression of high levels of fusion protein products that are readily purified may be desirable. Such vectors include, but are not limited, to the E. coli expression vector pUR278 (Ruther et al., EMBO J. 2:1791 (1983)), in which the antibody coding sequence may be ligated individually into the vector in frame with the lacZ coding region so that a fusion protein is produced; pIN vectors (Inouye & Inouye, Nucleic Acids Res. 13:3101-3109 (1985); Van Heeke & Schuster, J. Biol. Chem. 24:5503-5509 (1989)); and the like. pGEX vectors may also be used to express foreign polypeptides as fusion proteins with glutathione S-transferase (GST). In general, such fusion proteins are soluble and can easily be purified from lysed cells by adsorption and binding to a matrix glutathione-agarose beads followed by elution in the presence of free glutathione. The pGEX vectors are designed to include thrombin or factor Xa protease cleavage sites so that the cloned target gene product can be released from the GST moiety.

In addition to prokaryotes, eukaryotic microbes may also be used. Saccharomyces cerevisiae, or common baker's yeast, is the most commonly used among eukaryotic microorganisms although a number of other strains are commonly available, e.g., Pichia pastoris.

For expression in Saccharomyces, the plasmid YRp7, for example, (Stinchcomb et al., Nature 282:39 (1979); Kingsman et al., Gene 7:141 (1979); Tschemper et al., Gene 10:157 (1980)) is commonly used. This plasmid already contains the TRP1 gene which provides a selection marker for a mutant strain of yeast lacking the ability to grow in tryptophan, for example ATCC No. 44076 or PEP4-1 (Jones, Genetics 85:12 (1977)). The presence of the trpl lesion as a characteristic of the yeast host cell genome then provides an effective environment for detecting transformation by growth in the absence of tryptophan.

In an insect system, Autographa californica nuclear polyhedrosis virus (AcNPV) is typically used as a vector to express foreign genes. The virus grows in Spodoptera frugiperda cells. The antibody coding sequence may be cloned individually into non-essential regions (for example the polyhedrin gene) of the virus and placed under control of an AcNPV promoter (for example the polyhedrin promoter).

Once an antibody molecule of the invention has been recombinantly expressed, it may be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. Alternatively, a preferred method for increasing the affinity of antibodies of the invention is disclosed in US 2002/0123057 A1.

### VIII. TREATMENT METHODS USING THERAPEUTIC C35 ANTIBODIES

The present invention is directed to using C35 antibodies to treat hyperproliferative diseases, for example, to treat cancer. In some embodiments, one C35 antibody may be administered. In other embodiments, two or more, and preferably two C35 antibodies are administered. Further, the antibody or antibodies may be administered with a therapeutic agent. In a particular embodiment, the therapeutic agent is a chemotherapeutic agent. In a more particular embodiment, the chemotherapeutic agent is paclitaxel. In another particular embodiment, the chemotherapeutic agent is adriamycin.

In embodiments where at least two C35 antibodies are administered, the antibodies can each bind to different epitopes within C35. For example, one antibody can bind to an epitope located within residues 105-115 of C35 (SEQ ID NO:2) while the other can bind an epitope located within resides 48-104 of C35 (SEQ ID NO:2). In a particular embodiments, the C35 antibodies that bind eptiopes within these regions of C35 are 1B3 and 1F2. In another embodiment, at least one of the at least two C35 antibodies binds to an epitope within residues 48-87 of C35 (SEQ ID NO:2). In a particular embodiment, at least one of the antibodies that binds to an epitope within residues 48-87 of C35 (SEQ ID NO:2) is MAb163.

The present invention also includes administering two C35 antibodies that bind the same epitope. For example, two different C35 antibodies that bind to an epitope located within residues 105-115 of C35 (SEQ ID NO:2) can be administered. Similarly, two different C35 antibodies that bind to an epitope located within residues 48-104 of C35 (SEQ ID NO:2) can be administered.

In some embodiments, the C35 antibody or antibodies for use in the methods of the present invention can be selected based on their ability to bind to a C35 polypeptide or fragment thereof, or a C35 variant polypeptide, with an affinity characterized by a dissociation constant (K_{D}) which is less than the K_{D} of a reference monoclonal antibody. The present invention includes all C35 antibodies disclosed herein as reference monoclonal antibodies for the purposes of these embodiments. In a particular embodiment, monoclonal antibodies 1B3 and 1F2 as disclosed herein are the reference antibodies.

In another embodiment, the reference monoclonal antibody is MAb 163. Accordingly, in some embodiments, the C35 antibody or antibodies bind to a C35 polypeptide or fragment thereof, or a C35 variant polypeptide, with an affinity characterized by a dissociation constant (K_{D}) which is less than the K_{D} of MAb 163 (see Example 16, herein below).

In some embodiments, at least one C35 antibody or fragment used in the methods of the present invention specifically binds to a C35 polypeptide or fragment thereof, or a C35 variant polypeptide with an affinity characterized by a dissociation constant (K_{D}) no greater than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M.

In some embodiments, the present invention includes administering one C35 antibody with a chemotherapeutic agent. Any C35 antibody disclosed herein may be used in this method. In some embodiments, the C35 antibody is administered before, after, or concurrently with the administration of the chemotherapeutic agent. In a preferred embodiment, MAb 163 is administered with a chemotherapeutic agent. In one embodiment, the chemotherapeutic agent is paclitaxel.

In some preferred embodiments, the present invention includes administering at least two C35 antibodies with a chemotherapeutic agent. Any combination of C35 antibodies may be administered and all combinations are included in the present invention. For example, any of the following combinations could be used: 1F2 with 1B3, 1F2 with MAbc009, 1F2 with MAb 163, 1F2 with MAb 165, 1F2 with MAb 171, 1B3 with MAbc009, 1B3 with MAb 163, 11B3 with MAb 165, 1B3 with MAb 171, MAbc009 with MAb 163, MAbc009 with MAb 165, MAbc009 with MAb 171, MAb 163 with MAb 165, MAb 163 with MAb 171, or MAb 165 with MAb 171. Also encompassed in the present invention are administration of variants (e.g. humanized versions, affinity optimized versions) or derivatives of any of these antibodies in combination with each other and therapeutic agents (e.g., a chemotherapeutic agent). Also encompassed in the present invention are compositions comprising combinations of antibodies with or without therapeutic agents.

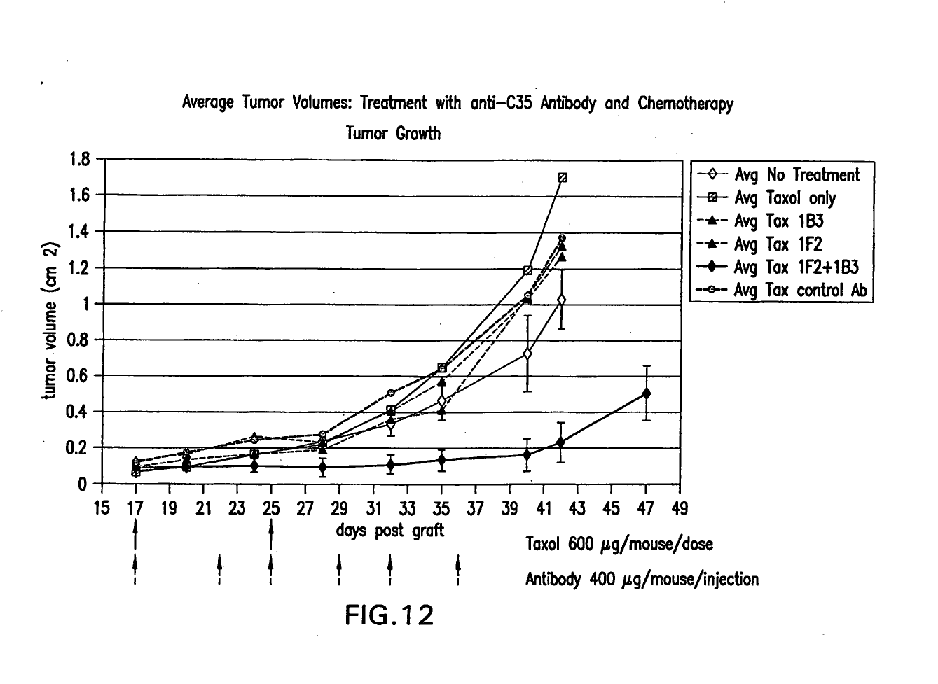

In some preferred embodiments, MAb 163 can be administered in combination with MAb 165 and a chemotherapeutic agent. Similarly, FIG. 12 illustrates that the murine C35 antibodies 1F2 and 1B3 in combination with paclitaxel are effective in reducing tumor volume in mice.

In embodiments where the subject with cancer is a human, the antibodies administered are preferably fully human or humanized. These humanized antibodies can include, but are not limited to MAb 165, or a humanized form of any murine C35 antibody disclosed herein, for example, humanized versions of 1F2 and/or 1B3. Also encompassed within the present invention are affinity optimized versions of the antibodies, including, but not limited to MAb 163, MAb 165, 1B3, and 1F2.

The methods and compositions of the invention can be used to treat hyperproliferative diseases, disorders, and/or conditions, including neoplasms. Examples of hyperproliferative diseases, disorders, and/or conditions that can be treated by the method of the invention include, but are not limited to neoplasms located in the: prostate, colon, abdomen, bone, breast, digestive system, liver, pancreas, peritoneum, endocrine glands (adrenal, parathyroid, pituitary, testicles, ovary, thymus, thyroid), eye, head and neck, nervous (central and peripheral), lymphatic system, pelvic, skin, soft tissue, spleen, thoracic, and urogenital.

Other examples of such hyperproliferative disorders include, but are not limited to: Acute Childhood Lymphoblastic Leukemia, Acute Lymphoblastic Leukemia, Acute Lymphocytic Leukemia, Acute Myeloid Leukemia, Adrenocortical Carcinoma, Adult (Primary) Hepatocellular Cancer, Adult (Primary) Liver Cancer, Adult Acute Lymphocytic Leukemia, Adult Acute Myeloid Leukemia, Adult Hodgkin's Disease, Adult Hodgkin's Lymphoma, Adult Lymphocytic Leukemia, Adult Non-Hodgkin's Lymphoma, Adult Primary Liver Cancer, Adult Soft Tissue Sarcoma, AIDS-Related Lymphoma, AIDS-Related Malignancies, Anal Cancer, Astrocytoma, Bile Duct Cancer, Bladder Cancer, Bone Cancer, Brain Stem Glioma, Brain Tumors, Breast Cancer, Cancer of the Renal Pelvis and Ureter, Central Nervous System (Primary) Lymphoma, Central Nervous System Lymphoma, Cerebellar Astrocytoma, Cerebral Astrocytoma, Cervical Cancer, Childhood (Primary) Hepatocellular Cancer, Childhood (Primary) Liver Cancer, Childhood Acute Lymphoblastic Leukemia, Childhood Acute Myeloid Leukemia, Childhood Brain Stem Glioma, Childhood Cerebellar Astrocytoma, Childhood Cerebral Astrocytoma, Childhood Extracranial Germ Cell Tumors, Childhood Hodgkin's Disease, Childhood Hodgkin's Lymphoma, Childhood Hypothalamic and Visual Pathway Glioma, Childhood Lymphoblastic Leukemia, Childhood Medulloblastoma, Childhood Non-Hodgkin's Lymphoma, Childhood Pineal and Supratentorial Primitive Neuroectodermal Tumors, Childhood Primary Liver Cancer, Childhood Rhabdomyosarcoma, Childhood Soft Tissue Sarcoma, Childhood Visual Pathway and Hypothalamic Glioma, Chronic Lymphocytic Leukemia, Chronic Myelogenous Leukemia, Colon Cancer, Cutaneous T-Cell Lymphoma, Endocrine Pancreas Islet Cell Carcinoma, Endometrial Cancer, Ependymoma, Epithelial Cancer, Esophageal Cancer, Ewing's Sarcoma and Related Tumors, Exocrine Pancreatic Cancer, Extracranial Germ Cell Tumor, Extragonadal Germ Cell Tumor, Extrahepatic Bile Duct Cancer, Eye Cancer, Female Breast Cancer, Gaucher's Disease, Gallbladder Cancer, Gastric Cancer, Gastrointestinal Carcinoid Tumor, Gastrointestinal Tumors, Germ Cell Tumors, Gestational Trophoblastic Tumor, Hairy Cell Leukemia, Head and Neck Cancer, Hepatocellular Cancer, Hodgkin's Disease, Hodgkin's Lymphoma, Hypergammaglobulinemia, Hypopharyngeal Cancer, Intestinal Cancers, Intraocular Melanoma, Islet Cell Carcinoma, Islet Cell Pancreatic Cancer, Kaposi's Sarcoma, Kidney Cancer, Laryngeal Cancer, Lip and Oral Cavity Cancer, Liver Cancer, Lung Cancer, Lymphoproliferative Disorders, Macroglobulinemia, Male Breast Cancer, Malignant Mesothelioma, Malignant Thymoma, Medulloblastoma, Melanoma, Mesothelioma, Metastatic Occult Primary Squamous Neck Cancer, Metastatic Primary Squamous Neck Cancer, Metastatic Squamous Neck Cancer, Multiple Myeloma, Multiple Myeloma/Plasma Cell Neoplasm, Myelodysplastic Syndrome, Myelogenous Leukemia, Myeloid Leukemia, Myeloproliferative Disorders, Nasal Cavity and Paranasal Sinus Cancer, Nasopharyngeal Cancer, Neuroblastoma, Non-Hodgkin's Lymphoma During Pregnancy, Nonmelanoma Skin Cancer, Non-Small Cell Lung Cancer, Occult Primary Metastatic Squamous Neck Cancer, Oropharyngeal Cancer, Osteo-/Malignant Fibrous Sarcoma, Osteosarcoma/Malignant Fibrous Histiocytoma, Osteosarcoma/Malignant Fibrous Histiocytoma of Bone, Ovarian Epithelial Cancer, Ovarian Germ Cell Tumor, Ovarian Low Malignant Potential Tumor, Pancreatic Cancer, Paraproteinemias, Purpura, Parathyroid Cancer, Penile Cancer, Pheochromocytoma, Pituitary Tumor, Plasma Cell Neoplasm/Multiple Myeloma, Primary Central Nervous System Lymphoma, Primary Liver Cancer, Prostate Cancer, Rectal Cancer, Renal Cell Cancer, Renal Pelvis and Ureter Cancer, Retinoblastoma, Rhabdomyosarcoma, Salivary Gland Cancer, Sarcoidosis Sarcomas, Sezary Syndrome, Skin Cancer, Small Cell Lung Cancer, Small Intestine Cancer, Soft Tissue Sarcoma, Squamous Neck Cancer, Stomach Cancer, Supratentorial Primitive Neuroectodermal and Pineal Tumors, T-Cell Lymphoma, Testicular Cancer, Thymoma, Thyroid Cancer, Transitional Cell Cancer of the Renal Pelvis and Ureter, Transitional Renal Pelvis and Ureter Cancer, Trophoblastic Tumors, Ureter and Renal Pelvis Cell Cancer, Urethral Cancer, Uterine Cancer, Uterine Sarcoma, Vaginal Cancer, Visual Pathway and Hypothalamic Glioma, Vulvar Cancer, Waldenstrom's Macroglobulinemia, Wilms' Tumor, and any other hyperproliferative disease, besides neoplasia, located in an organ system listed above.

In some particular embodiments, the hyperproliferative disorder is a cancer of a tissue or organ selected from the group consisting of breast, bladder, liver, colon, ovary and skin.

The methods and compositions of the present invention can be used to treat premalignant conditions and to prevent progression to a neoplastic or malignant state, including but not limited to those disorders described above. Such uses are indicated in conditions known or suspected of preceding progression to neoplasia or cancer, in particular, where non-neoplastic cell growth consisting of hyperplasia, metaplasia, or most particularly, dysplasia has occurred (for review of such abnormal growth conditions, see Robbins and Angell, 1976, Basic Pathology, 2d Ed., W. B. Saunders Co., Philadelphia, pp. 68-79.)

Hyperplasia is a form of controlled cell proliferation, involving an increase in cell number in a tissue or organ, without significant alteration in structure or function. Hyperplastic disorders which can be treated by the method of the invention include, but are not limited to, angiofollicular mediastinal lymph node hyperplasia, angiolymphoid hyperplasia with eosinophilia, atypical melanocytic hyperplasia, basal cell hyperplasia, benign giant lymph node hyperplasia, cementum hyperplasia, congenital adrenal hyperplasia, congenital sebaceous hyperplasia, cystic hyperplasia, cystic hyperplasia of the breast, denture hyperplasia, ductal hyperplasia, endometrial hyperplasia, fibromuscular hyperplasia, focal epithelial hyperplasia, gingival hyperplasia, inflammatory fibrous hyperplasia, inflammatory papillary hyperplasia, intravascular papillary endothelial hyperplasia, nodular hyperplasia of prostate, nodular regenerative hyperplasia, pseudoepitheliomatous hyperplasia, senile sebaceous hyperplasia, and verrucous hyperplasia.

Metaplasia is a form of controlled cell growth in which one type of adult or fully differentiated cell substitutes for another type of adult cell. Metaplastic disorders which can be treated by the method of the invention include, but are not limited to, agnogenic myeloid metaplasia, apocrine metaplasia, atypical metaplasia, autoparenchymatous metaplasia, connective tissue metaplasia, epithelial metaplasia, intestinal metaplasia, metaplastic anemia, metaplastic ossification, metaplastic polyps, myeloid metaplasia, primary myeloid metaplasia, secondary myeloid metaplasia, squamous metaplasia, squamous metaplasia of amnion, and symptomatic myeloid metaplasia.

Dysplasia is frequently a forerunner of cancer, and is found mainly in the epithelia; it is the most disorderly form of non-neoplastic cell growth, involving a loss in individual cell uniformity and in the architectural orientation of cells. Dysplastic cells often have abnormally large, deeply stained nuclei, and exhibit pleomorphism. Dysplasia characteristically occurs where there exists chronic irritation or inflammation. Dysplastic disorders which can be treated by the method of the invention include, but are not limited to, anhidrotic ectodermal dysplasia, anterofacial dysplasia, asphyxiating thoracic dysplasia, atriodigital dysplasia, bronchopulmonary dysplasia, cerebral dysplasia, cervical dysplasia, chondroectodermal dysplasia, cleidocranial dysplasia, congenital ectodermal dysplasia, craniodiaphysial dysplasia, craniocarpotarsal dysplasia, craniometaphysial dysplasia, dentin dysplasia, diaphysial dysplasia, ectodermal dysplasia, enamel dysplasia, encephalo-ophthalmic dysplasia, dysplasia epiphysialis hemimelia, dysplasia epiphysialis multiplex, dysplasia epiphysialis punctata, epithelial dysplasia, faciodigitogenital dysplasia, familial fibrous dysplasia of jaws, familial white folded dysplasia, fibromuscular dysplasia, fibrous dysplasia of bone, florid osseous dysplasia, hereditary renal-retinal dysplasia, hidrotic ectodermal dysplasia, hypohidrotic ectodermal dysplasia, lymphopenic thymic dysplasia, mammary dysplasia, mandibulofacial dysplasia, metaphysial dysplasia, Mondini dysplasia, monostotic fibrous dysplasia, mucoepithelial dysplasia, multiple epiphysial dysplasia, oculoauriculovertebral dysplasia, oculodentodigital dysplasia, oculovertebral dysplasia, odontogenic dysplasia, opthalmomandibulomelic dysplasia, periapical cemental dysplasia, polyostotic fibrous dysplasia, pseudoachondroplastic spondyloepiphysial dysplasia, retinal dysplasia, septo-optic dysplasia, spondyloepiphysial dysplasia, and ventriculoradial dysplasia.

Additional pre-neoplastic disorders which can be treated by the methods and compositions of the invention include, but are not limited to, benign dysproliferative disorders (e.g., benign tumors, fibrocystic conditions, tissue hypertrophy, intestinal polyps, colon polyps, and esophageal dysplasia), leukoplakia, keratoses, Bowen's disease, Farmer's Skin, solar cheilitis, and solar keratosis.

In preferred embodiments, the methods and compositions of the invention are used to inhibit growth, progression, and/or metastasis of cancers, in particular those listed above.

In preferred embodiments, the methods and compositions of the present invention can be used to treat, inhibit growth, progression, and/or metastasis of cancers, in particular a cancer selected from the group consisting of breast cancer, ovarian cancer, bladder cancer, prostate cancer, pancreatic cancer, colon cancer, and melanoma.

The antibody or antibodies administered to treat a hyperproliferative disease may optionally be administered with an agent capable of inducing apoptosis. Apoptosis-inducing therapies include chemotherapeutic agents (also known as antineoplastic agents), radiation therapy, and combination radiotherapy and chemotherapy.

In some preferred embodiments, the C35 antibody or antibodies administer to treat the hyperproliferative disease, for example cancer, is/are administered with a chemotherapeutic agent. For example, the present invention includes a method of treating cancer comprising administering at least two C35 antibodies with a therapeutic agent.

Exemplary therapeutic agents are vinca alkaloids, epipodophyllotoxins, anthracycline antibiotics, actinomycin D, plicamycin, puromycin, gramicidin D, paclitaxel (Taxol™., Bristol Myers Squibb), coichicine, cytochalasin B, emetine, maytansine, and amsacrine (or “mAMSA”). The vinca alkaloid class is described in Goodman and Gilman's The Pharmacological Basis of Therapeutics (7th ed.), (1985), pp. 1277-1280. Exemplary of vinca alkaloids are vincristine, vinblastine, and vindesine. The epipodophyllotoxin class is described in Goodman and Gilman's The Pharmacological Basis of Therapeutics (7th ed.), (1985), pp. 1280-1281. Exemplary of epipodophyllotoxins are etoposide, etoposide orthoquinone, and teniposide. The anthracycline antibiotic class is described in Goodman and Gilman's The Pharmacological Basis of Therapeutics (7th ed.), (1985), pp. 1283-1285. Exemplary of anthracycline antibiotics are daunorubicin, doxorubicin, mitoxantraone, and bisanthrene. Actinomycin D, also called Dactinomycin, is described in Goodmand and Gilman's The Pharmacological Basis of Therapeutics (7th ed.), (1985), pp. 1281-1283. Plicamycin, also called mithramycin, is described in Goodmand and Gilman's The Pharmacological Basis of Therapeutics (7th ed), (1985), pp. 1287-1288. Additional chemotherapeutic agents include cisplatin (Platinol™., Bristol Myers Squibb), carboplatin (Paraplatin™., Bristol Myers Squibb), mitomycin (Mutamycin™., Bristol Myers Squibb), altretamine (Hexylen™, U.S. Bioscience, Inc.), cyclophosphamide (Cytoxan™, Bristol Myers Squibb), lomustine (CCNU) (CeeNU™ Bristol Myers Squibb), carmustine (BCNU) (BiCNU™, Bristol Myers Squibb).

Exemplary chemotherapeutic agents also include aclacinomycin A, aclarubicin, acronine, acronycine, adriamycin, aldesleukin (interleukin-2), altretamine (hexamethylmelamine), aminoglutethimide, aminoglutethimide (cytadren), aminoimidazole carboxamide, amsacrine (m-AMSA; amsidine), anastrazole (arimidex), ancitabine, anthracyline, anthramycin, asparaginase (elspar), azacitdine, azacitidine (ladakamycin), azaguanine, azaserine, azauridine, 1,1′,1″-phosphinothioylidynetris aziridine, azirino(2′, 3′:3,4)pyrrolo[1,2-a]indole-4,7-dione, BCG (theracys), BCNU, BCNU chloroethyl nitrosoureas, benzamide, 4-(bis(2-chloroethyl)amino)benzenebutanoic acid, bicalutamide, bischloroethyl nitrosourea, bleomycin, bleomycin (blenozane), bleomycins, bromodeoxyuridine, broxuridine, busulfan (myleran), carbamic acid ethyl ester, carboplatin, carboplatin (paraplatin), carmustine, carmustine (BCNU; BiCNU), chlorambucil (leukeran), chloroethyl nitrosoureas, chorozotocin (DCNU), chromomycin A3, cis-retinoic acid, cisplatin (cis-ddpl; platinol), cladribine (2-chlorodeoxyadenosine; 2cda; leustatin), coformycin, cycloleucine, cyclophosphamide, cyclophosphamide anhydrous, chlorambucil, cytarabine, cytarabine, cytarabine HCl (cytosar-u), 2-deoxy-2-(((methylnitrosoamino)carbonyl)amino)-D-glucose, dacarbazine, dactinomycin (cosmegen), daunorubicin, Daunorubincin HCl (cerubidine), decarbazine, decarbazine (DTIC-dome), demecolcine, dexamethasone, dianhydrogalactitol, diazooxonorleucine, diethylstilbestrol, docetaxel (taxotere), doxorubicin HCl (adriamycin), doxorubicin hydrochloride, eflomithine, estramustine, estramustine phosphate sodium (emcyt), ethiodized oil, ethoglucid, ethyl carbamate, ethyl methanesulfonate, etoposide (VP16-213), fenretinide, floxuridine, floxuridine (fudr), fludarabine (fludara), fluorouracil (5-FU), fluoxymesterone (halotestin), flutamide, flutamide (eulexin), fluxuridine, gallium nitrate (granite), gemcitabine (gemzar), genistein, 2-deoxy-2-(3-methyl-3-nitrosoureido)-D-glucopyranose, goserelin (zoladex), hexestrol, hydroxyurea (hydra), idarubicin (idamycin), ifosfagemcitabine, ifosfamide (iflex), ifosfamide with mesna (MAID), interferon, interferon alfa, interferon alfa-2a, alfa-2b, alfa-n3, interleukin-2, iobenguane, iobenguane iobenguane, irinotecan (camptosar), isotretinoin (accutane), ketoconazole, 4-(bis(2-chloroethyl)amino)-L-phenylalanine, L-serine diazoacetate, lentinan, leucovorin, leuprolide acetate (LHRH-analog), levamisole (ergamisol), lomustine (CCNU; cee-NU), mannomustine, maytansine, mechlorethamine, mechlorethamine HCl (nitrogen mustard), medroxyprogesterone acetate (provera, depo provera), megestrol acetate (menace), melengestrol acetate, melphalan (alkeran), menogaril, mercaptopurin, mercaptopurine (purinethol), mercaptopurine anhydrous, MESNA, mesna (mesne), methanesulfonic acid, ethyl ester, methotrexate (mtx; methotrexate), methyl-ccnu, mimosine, misonidazole, mithramycin, mitoantrone, mitobronitol, mitoguazone, mitolactol, mitomycin (mutamycin), mitomycin C, mitotane (o,p′-DDD; lysodren), mitoxantrone, mitoxantrone HCl (novantrone), mopidamol, N,N-bis(2-chloroethyl)tetrahydro-2H-1,3,2-oxazaphosphorin-2-amine-2-oxide, N-(1-methylethyl)-4-((2-methylhydrazino)methyl)benzamide, N-methyl-bis(2-chloroethyl)amine, nicardipine, nilutamide (nilandron), nimustine, nitracrine, nitrogen mustard, nocodazole, nogalamycin, octreotide (sandostatin), pacilataxel (taxol), paclitaxel, pactamycin, pegaspargase (PEGx-1), pentostatin (2′-deoxycoformycin), peplomycin, peptichemio, photophoresis, picamycin (mithracin), picibanil, pipobroman, plicamycin, podofilox, podophyllotoxin, porfiromycin, prednisone, procarbazine, procarbazine HCl (matulane), prospidium, puromycin, puromycin aminonucleoside, PUVA (psoralen+ultraviolet a), pyran copolymer, rapamycin, s-azacytidine, 2,4,6-tris(1-aziridinyl)-s-triazine, semustine, showdomycin, sirolimus, streptozocin (zanosar), suramin, tamoxifen citrate (nolvadex), taxon, tegafur, teniposide (VM-26; vumon), tenuazonic acid, TEPA, testolactone, thio-tepa, thioguanine, thiotepa (thioplex), tilorone, topotecan, tretinoin (vesanoid), triaziquone, trichodermin, triethylene glycol diglycidyl ether, triethylenemelamine, triethylenephosphoramide, triethylenethiophosphoramide, trimetrexate (neutrexin), tris(1-aziridinyl)phosphine oxide, tris(1-aziridinyl)phosphine sulfide, tris(aziridinyl)-p-benzoquinone, tris(aziridinyl)phosphine sulfide, uracil mustard, vidarabine, vidarabine phosphate, vinblastine, vinblastine sulfate (velban), vincristine sulfate (oncovin), vindesine, vinorelbine, vinorelbine tartrate (navelbine), (I)-mimosine, 1-(2-chloroethyl)-3-(4-methylcyclohexyl)-1-nitrosourea, (8S-cis)-10-((3-amino-2,3,6-trideoxy-alpha-L-lyxo-hexopyranosyl)oxy)-7,8,9,1,0-tetrahydro-6,8,11-trihydroxy-8-(hydroxyacetyl)-1-methoxy-5, 12-naphthacenedione, 131-meta-iodobenzyl guanidine (1-131 MIBG), 5-(3,3-dimethyl-1-triazenyl)-1H-imidazole4-carboxamide, 5-(bis(2-chloroethyl)amino)-2,4(1H,3H)-pyrimidinedione, 2,4,6-tris(1-aziridinyl)-s-thiazine, 2,3,5-tris(1-aziridinyl)-2,5-cyclohexadiene-1,4-dione, 2-chloro-N-(2-chloroethyl)-N-methylethanamine, N,N-bis(2-chloroethyl)tetrahydro-2H-1,3,2-oxazaphosphorin-2-amine-2-oxide, 3-deazauridine, 3-iodobenzylguanidine, 5,12-naphthacenedione, 5-azacytidine, 5-fluorouracil, (1 aS,8S,8aR,8bS)-6-amino-8-(((aminocarbonyl)oxy)methyl)-1,1a, 2,8,8a,8b-hexahydro-8a-methoxy-5-methylazirino(2′,3′:3,4)pyrrolo[1,2-a]indole-4,7-dione, 6-azauridine, 6-mercaptopurine, 8-azaguanine, and combinations thereof.

In a particular embodiment, the chemotherapeutic agent used in the methods of the present invention is paclitaxel. In another particular embodiment, the chemotherapeutic agent used in the methods of the present invention is adriamycin.

Preferred therapeutic agents and combinations thereof may be administered as an apoptosis-inducing therapy include Doxorubicin and Doxetaxel, Topotecan, Paclitaxel (Taxol), Carboplatin and Taxol, Cisplatin and radiation, 5-fluorouracil (5-FU), 5-FU and radiation, Toxotere, Fludarabine, Ara C, Etoposide, Vincristine, and Vinblastin.

Chemotherapeutic agents that may be administered in the method of the invention include, but are not limited to, antibiotic derivatives (e.g., doxorubicin, bleomycin, daunorubicin, and dactinomycin); antiestrogens (e.g., tamoxifen); antimetabolites (e.g., fluorouracil, 5-FU, methotrexate, floxuridine, interferon alpha-2b, glutamic acid, plicamycin, mercaptopurine, and 6-thioguanine); cytotoxic agents (e.g., carmustine, BCNU, lomustine, CCNU, cytosine arabinoside, cyclophosphamide, estramustine, hydroxyurea, procarbazine, mitomycin, busulfan, cis-platin, and vincristine sulfate); hormones (e.g., medroxyprogesterone, estramustine phosphate sodium, ethinyl estradiol, estradiol, megestrol acetate, methyltestosterone, diethylstilbestrol diphosphate, chlorotrianisene, and testolactone); nitrogen mustard derivatives (e.g., mephalen, chorambucil, mechlorethamine (nitrogen mustard) and thiotepa); steroids and combinations (e.g., bethamethasone sodium phosphate); and others (e.g., dicarbazine, asparaginase, mitotane, vincristine sulfate, vinblastine sulfate, and etoposide).

In a specific embodiment, antibodies of the invention are administered in combination with CHOP (cyclophosphamide, doxorubicin, vincristine, and prednisone) or any combination of the components of CHOP.

TABLE 5
COMMONLY USED CHEMOTHERAPY DRUGS
FOR MAJOR CANCER INDICATIONS

1. Breast cancer: Adjuvant therapy (systemic therapy as an adjunct
to or in addition to surgery). Doxorubicin (Adriamycin), cyclo-
phosphamide, and taxanes [paclitaxel (Taxol) and docetaxel
(Taxotere)]. These three drugs are also active in metastatic
breast cancer but if the patient has already received them as
adjuvant therapy the commonly used drugs are capecitabine (Xeloda),
gemcitabine (Gemzar), vinorelbine (Navelbine). Commonly prescribed
hormonal agents for bone metastases of hormone receptor positive
tumors are: tamoxifen and aromatase inhibitors (Arimidex, Femara,
Aromasin).
2. Colon cancer: 5-FU plus leucovorin, irinotecan (camptosar),
oxaliplatin, and capecitabine.
3. Lung cancer: Cisplatin, carboplatin, paclitaxel, docetaxel,
gemcitabine, vinorelbine.
4. Prostate cancer: Docetaxel, estramustine, mitoxantrone
(Novantrone), and prednisone.
5. Non-Hodgkin's Lymphoma: Cyclophosphamide, doxorubicin,
vincristine (Oncovin), and prednisone.

The present invention is also directed to the use of at least two C35 antibodies in the preparation of a medicament for treating cancer. In one embodiment, the use further comprises administering a chemotherapeutic agent. In specific embodiments, the antibodies are administered concurrently. In other embodiments, the antibodies are administered sequentially. In other embodiments, the antibodies are administered at varying intervals. In some embodiments, the chemotherapeutic agent is administered concurrently with one or more of the antibodies. In other embodiments, the chemotherapeutic agent is administered on a different time course than the antibodies, as described elsewhere herein.

In some embodiments, the methods of the present invention are directed to administering C35 antibodies with therapeutic radiation. Optionally, these methods can also administration of a chemotherapeutic agent. For example, in some embodiments, the present invention can include administering at least one C35 antibody with a chemotherapeutic agent and therapeutic radiation.

Therapeutic radiation includes, for example, fractionated radiotherapy, nonfractionated radiotherapy and hyperfractionated radiotherapy, and combination radiation and chemotherapy. Types of radiation also include ionizing (gamma) radiation, particle radiation, low energy transmission (LET), high energy transmission (HET), ultraviolet radiation, infrared radiation, visible light, and photosensitizing radiation. As used herein, chemotherapy includes treatment with a single chemotherapeutic agent or with a combination of agents. In a subject in need of treatment, chemotherapy may be combined with surgical treatment or radiation therapy, or with other antineoplastic treatment modalities.

In further embodiments, the antibodies of the invention or combinations thereof are administered in combination with an antiviral agent. Antiviral agents that may be administered with the antibodies of the invention include, but are not limited to, acyclovir, ribavirin, amantadine, and remantidine.

Antibodies of the invention or combinations thereof may also be administered with antiemetics such as 2-(ethylthio)-10-(3-(4-methyl-1-piperazinyl)propyl)-10H-phenothiazine (ethylthioperazine), 1-(p-chloro-alpha-phenylbenzyl)-4-(m-methylbenzyl)-piperazine (meclozine, meclizine), etc., and combinations thereof. Polynucleotides and polypeptides of the invention may also be administered with other therapeutic agents, and combinations thereof, disclosed herein or known in the art.

Conventional nonspecific immunosuppressive agents, that may be administered in combination with the antibodies of the invention or combinations thereof include, but are not limited to, steroids, cyclosporine, cyclosporine analogs, cyclophosphamide methylprednisone, prednisone, azathioprine, FK-506, 15-deoxyspergualin, and other immunosuppressive agents that act by suppressing the function of responding T cells.

In specific embodiments, antibodies of the invention or combinations thereof are administered in combination with immunosuppressants. Immunosuppressants preparations that may be administered with the antibodies of the invention include, but are not limited to, ORTHOCLONE™ (OKT3), SANDIMMUNE™/NEORAL™/SANGDYA™ (cyclosporin), PROGRAF™ (tacrolimus), CELLCEP™ (mycophenolate), Azathioprine, glucorticosteroids, and RAPAMUNE™ (sirolimus). In a specific embodiment, immunosuppressants may be used to prevent rejection of organ or bone marrow transplantation.

In an additional embodiment, antibodies of the invention are administered alone or in combination with one or more intravenous immune globulin preparations. Intravenous immune globulin preparations that may be administered with the antibodies of the invention include, but not limited to, GAMMAR™, IVEEGAM™, SANDOGLOBULIN™, GAMMAGARD S/D™, and GAMIMUNE™. In a specific embodiment, antibodies of the invention are administered in combination with intravenous immune globulin preparations in transplantation therapy (e.g., bone marrow transplant).

In an additional embodiment, the antibodies of the invention are administered alone or in combination with an anti-inflammatory agent. Anti-inflammatory agents that may be administered with the antibodies of the invention include, but are not limited to, glucocorticoids and the nonsteroidal anti-inflammatories, aminoarylcarboxylic acid derivatives, arylacetic acid derivatives, arylbutyric acid derivatives, arylcarboxylic acids, arylpropionic acid derivatives, pyrazoles, pyrazolones, salicylic acid derivatives, thiazinecarboxamides, e-acetamidocaproic acid, S-adenosylmethionine, 3-amino-4-hydroxybutyric acid, amixetrine, bendazac, benzydamine, bucolome, difenpiramide, ditazol, emorfazone, guaiazulene, nabumetone, nimesulide, orgotein, oxaceprol, paranyline, perisoxal, pifoxime, proquazone, proxazole, and tenidap.

In an additional embodiment, the antibodies of the invention are administered in combination with cytokines. Cytokines that may be administered with the antibodies of the invention include, but are not limited to, IL2, IL3, IL4, IL5, IL6, IL7, IL10, IL12, IL13, IL15, anti-CD40, CD40L, IFN-gamma and TNF-alpha. In another embodiment, antibodies of the invention may be administered with any interleukin, including, but not limited to, IL-1alpha, IL-1beta, IL-2, IL-3, IL4, IL-5, IL-6, IL-7, IL-8, IL-9, IL-10, IL-11, IL-12, IL-13, IL-14, IL-15, IL-16, IL-17, IL-18, IL-19, IL-20, and IL-21.

In an additional embodiment, the antibodies of the invention are administered in combination with angiogenic proteins. Angiogenic proteins that may be administered with the antibodies of the invention include, but are not limited to, Glioma Derived Growth Factor (GDGF), as disclosed in European Patent Number EP-399816; Platelet Derived Growth Factor-A (PDGF-A), as disclosed in European Patent Number EP-682110; Platelet Derived Growth Factor-B (PDGF-B), as disclosed in European Patent Number EP-282317; Placental Growth Factor (P1GF), as disclosed in International Publication Number WO 92/06194; Placental Growth Factor-2 (P1GF-2), as disclosed in Hauser et al., Growth Factors, 4:259-268 (1993); Vascular Endothelial Growth Factor (VEGF), as disclosed in International Publication Number WO 90/13649; Vascular Endothelial Growth Factor-A (VEGF-A), as disclosed in European Patent Number EP-506477; Vascular Endothelial Growth Factor-2 (VEGF-2), as disclosed in International Publication Number WO 96/39515; Vascular Endothelial Growth Factor B (VEGF-3); Vascular Endothelial Growth Factor B-186 (VEGF-B186), as disclosed in International Publication Number WO 96/26736; Vascular Endothelial Growth Factor-D (VEGF-D), as disclosed in International Publication Number WO 98/02543; Vascular Endothelial Growth Factor-D (VEGF-D), as disclosed in International Publication Number WO 98/07832; and Vascular Endothelial Growth Factor-E (VEGF-E), as disclosed in German Patent Number DE19639601. The above mentioned references are incorporated herein by reference herein.

In an additional embodiment, the antibodies of the invention are administered in combination with hematopoietic growth factors. Hematopoietic growth factors that may be administered with the antibodies of the invention include, but are not limited to, LEUKINE™ (SARGRAMOSTIM™) and NEUPOGEN™ (FILGRASTIM™).

In an additional embodiment, the antibodies of the invention are administered in combination with Fibroblast Growth Factors. Fibroblast Growth Factors that may be administered with the antibodies of the invention include, but are not limited to, FGF-1, FGF-2, FGF-3, FGF-4, FGF-5, FGF-6, FGF-7, FGF-8, FGF-9, FGF-10, FGF-11, FGF-12, FGF-13, FGF-14, and FGF-15.

### Timing of Administration

Any of the apoptosis inducing therapies described herein may be administered concurrently with one or more of the C35 antibodies of the present invention. In some embodiments, two or more C35 antibodies are administered concurrently. In other embodiments, the C35 antibodies are administered separately. For example, the first C35 antibody could be administered at one time and then the second C35 antibody could be administered later the same day or one or more days after the day the first C35 antibody is administered. Administration of multiple C35 antibodies may occur before, after, or concurrently with administration of a chemotherapeutic agent, for example, paclitaxel (Taxol™), adriamycin, or any other agent described herein. For example, one or two or more of the C35 antibodies could be administered at the same time or on the same day as the paclitaxel, adriamycin or other agent. Alternatively, the paclitaxel, adriamycin or other agent could be administered on a day where no C35 antibodies are administered, for example, on a day before administering at least one C35 antibody or an a day following the administration of at least one C35 antibody.

In some embodiments, the apoptosis inducing agent can be administered following the administration of at least one C35 antibody. For example, the apoptosis inducing agent can be administered about 1, 2, 3, 4, 5, 6, 7, 8, 9, 10, 11, 12, 13, 14, 15, 16, 17, 18, 19, 20, 21, 22, 23 or 24 hours after administering at least one C35 antibody to the subject in need of treatment. In some embodiments, the apoptosis inducing agent can be administered about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or 31 days after administering at least one C35 antibody to the subject in need of treatment. In preferred embodiments, the apoptosis inducing therapy is a chemotherapeutic agent, for example, paclitaxel.

In some embodiments, the apoptosis inducing agent can be administered prior to the administration of at least one C35 antibody. For example, the apoptosis inducing agent can be administered about 1, 2, 3, 4, 5, 6, 7, 8, 9, 10, 11, 12, 13, 14, 15, 16, 17, 18, 19, 20, 21, 22, 23 or 24 hours before administering at least one C35 antibody to the subject in need of treatment. In some embodiments, the apoptosis inducing agent can be administered about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or 31 days before administering at least one C35 antibody to the subject in need of treatment. In preferred embodiments, the apoptosis inducing therapy is a chemotherapeutic agent, for example, paclitaxel or adriamycin.

In one embodiment, the chemotherapeutic agent is administered at weekly intervals during the course of treatment. In a specific embodiment, the chemotherapeutic agent is administered once per week for two weeks during the course of treatment. In a more specific embodiment, the chemotherapeutic agent is administered once per week during the first two weeks of the treatment course. In some embodiments, the at least two C35 antibodies are administered once, twice, or three times per week during a course of treatment. In a specific embodiment, the C35 antibodies are administered twice per week during a course of treatment.

In one embodiment, the at least two C35 antibodies are administered twice weekly and the apoptosis-inducing agent is administered once per week. In one embodiment, the apoptosis-inducing agent is administered on the first day of treatment and a second dose of apoptosis-inducing agent is administered one week later, while the C35 antibodies are administered twice weekly.

In particular embodiments, a course of treatment can last one week, two weeks, three weeks, four weeks, five weeks, six weeks, seven weeks, eight weeks, one month, two months, three months four months, five months, six months, seven months, eight months, nine months, ten months, eleven months, or one year. The duration of the course of treatment will depend on the type of cancer, the antibodies used, the chemotherapeutic agent, age of patient, etc. These parameters can be determined by one of skill in the art.

### Demonstration of Therapeutic Activity

The methods and antibodies of the invention are preferably tested in vitro, and then in vivo for the desired therapeutic or prophylactic activity, prior to use in humans. For example, in vitro assays to demonstrate the therapeutic or prophylactic utility of a compound or pharmaceutical composition include the effect of a compound on a cell line or a patient tissue sample. The effect of the compound or composition on the cell line and/or tissue sample can be determined utilizing techniques known to those of skill in the art including, but not limited to, rosette formation assays and cell lysis assays. In accordance with the invention, in vitro assays which can be used to determine whether administration of a specific compound is indicated, include in vitro cell culture assays in which a patient tissue sample is grown in culture, and exposed to or otherwise administered a compound, and the effect of such compound upon the tissue sample is observed.

### Kits

The present invention provides kits that can be used in the above methods. In one embodiment, a kit comprises one or more antibodies of the invention, preferably one or more purified antibodies, in one or more containers. In a specific embodiment, the kits of the present invention contain a substantially isolated polypeptide comprising an epitope which is specifically immunoreactive with an antibody included in the kit. Preferably, the kits of the present invention further comprise a control antibody which does not react with the polypeptide of interest. In another specific embodiment, the kits of the present invention contain a means for detecting the binding of an antibody to a polypeptide of interest (e.g., the antibody may be conjugated to a detectable substrate such as a fluorescent compound, an enzymatic substrate, a radioactive compound or a luminescent compound, or a second antibody which recognizes the first antibody may be conjugated to a detectable substrate).

In another specific embodiment of the present invention, the kit is a diagnostic kit for use in screening serum containing antibodies specific against proliferative and/or cancerous polynucleotides and polypeptides. Such a kit may include a control antibody that does not react with the polypeptide of interest. Such a kit may include a substantially isolated polypeptide antigen comprising an epitope which is specifically immunoreactive with at least one anti-polypeptide antigen antibody. Further, such a kit includes means for detecting the binding of said antibody to the antigen (e.g., the antibody may be conjugated to a fluorescent compound such as fluorescein or rhodamine which can be detected by flow cytometry). In specific embodiments, the kit may include a recombinantly produced or chemically synthesized polypeptide antigen. The polypeptide antigen of the kit may also be attached to a solid support.

In a more specific embodiment the detecting means of the above-described kit includes a solid support to which said polypeptide antigen is attached. Such a kit may also include a non-attached reporter-labeled anti-human antibody. In this embodiment, binding of the antibody to the polypeptide antigen can be detected by binding of the said reporter-labeled antibody.

In an additional embodiment, the invention includes a diagnostic kit for use in screening samples containing antigens of the polypeptide of the invention. The diagnostic kit includes a substantially isolated antibody specifically immunoreactive with polypeptide or polynucleotide antigens, and means for detecting the binding of the polynucleotide or polypeptide antigen to the antibody. In one embodiment, the antibody is attached to a solid support. In a specific embodiment, the antibody may be a monoclonal antibody. The detecting means of the kit may include a second, labeled monoclonal antibody. Alternatively, or in addition, the detecting means may include a labeled, competing antigen.

In one diagnostic configuration, test sample is reacted with a solid phase reagent having a surface-bound antigen obtained by the methods of the present invention. After binding with specific antigen antibody to the reagent and removing unbound sample components by washing, the reagent is reacted with reporter-labeled anti-human antibody to bind reporter to the reagent in proportion to the amount of bound anti-antigen antibody on the solid support. The reagent is again washed to remove unbound labeled antibody, and the amount of reporter associated with the reagent is determined. Typically, the reporter is an enzyme which is detected by incubating the solid phase in the presence of a suitable fluorometric, luminescent or calorimetric substrate (Sigma, St. Louis, Mo.).

The solid surface reagent in the above assay is prepared by known techniques for attaching protein material to solid support material, such as polymeric beads, dip sticks, 96-well plate or filter material. These attachment methods generally include non-specific adsorption of the protein to the support or covalent attachment of the protein, typically through a free amine group, to a chemically reactive group on the solid support, such as an activated carboxyl, hydroxyl, or aldehyde group. Alternatively, streptavidin coated plates can be used in conjunction with biotinylated antigen(s).

Thus, the invention provides an assay system or kit for carrying out this diagnostic method. The kit generally includes a support with surface-bound recombinant antigens, and a reporter-labeled anti-human antibody for detecting surface-bound anti-antigen antibody.

### Gene Therapy

In a specific embodiment, nucleic acids comprising sequences encoding antibodies such as C35 antibodies, or functional derivatives thereof, are administered to treat, inhibit or prevent a disease or disorder associated with aberrant expression and/or activity of C35, by way of gene therapy. Gene therapy refers to therapy performed by the administration to a subject of an expressed or expressible nucleic acid. In this embodiment of the invention, the nucleic acids produce their encoded protein that mediates a therapeutic effect.

Any of the methods for gene therapy available in the art can be used according to the present invention. Exemplary methods are described below.

For general reviews of the methods of gene therapy, see Goldspiel et al., Clinical Pharmacy 12:488-505 (1993); Wu and Wu, Biotherapy 3:87-95 (1991); Tolstoshev, Ann. Rev. Pharmacol. Toxicol. 32:573-596 (1993); Mulligan, Science 260:926-932 (1993); and Morgan and Anderson, Ann. Rev. Biochem. 62:191-217 (1993); May, TIBTECH 11(5):155-215 (1993). Methods commonly known in the art of recombinant DNA technology which can be used are described in Ausubel et al. (eds.), Current Protocols in Molecular Biology, John Wiley & Sons, NY (1993); and Kriegler, Gene Transfer and Expression, A Laboratory Manual, Stockton Press, NY (1990).

In a preferred aspect, the compound comprises nucleic acid sequences encoding an antibody, said nucleic acid sequences being part of expression vectors that express the antibody or fragments or chimeric proteins or heavy or light chains thereof in a suitable host. In particular, such nucleic acid sequences have promoters operably linked to the antibody coding region, said promoter being inducible or constitutive, and, optionally, tissue-specific. In another particular embodiment, nucleic acid molecules are used in which the antibody coding sequences and any other desired sequences are flanked by regions that promote homologous recombination at a desired site in the genome, thus providing for intrachromosomal expression of the antibody encoding nucleic acids (Koller and Smithies, Proc. Natl. Acad. Sci. USA 86:8932-8935 (1989); Zijlstra et al., Nature 342:435-438 (1989). In specific embodiments, the expressed antibody molecule is a single chain antibody; alternatively, the nucleic acid sequences include sequences encoding both the heavy and light chains, or fragments thereof, of the antibody.

Delivery of the nucleic acids into a patient may be either direct, in which case the patient is directly exposed to the nucleic acid or nucleic acid-carrying vectors, or indirect, in which case, cells are first transformed with the nucleic acids in vitro, then transplanted into the patient. These two approaches are known, respectively, as in vivo or ex vivo gene therapy.

In a specific embodiment, the nucleic acid sequences are directly administered in vivo, where it is expressed to produce the encoded product. This can be accomplished by any of numerous methods known in the art, e.g., by constructing them as part of an appropriate nucleic acid expression vector and administering it so that they become intracellular, e.g., by infection using defective or attenuated retrovirals or other viral vectors (see U.S. Pat. No. 4,980,286), or by direct injection of naked DNA, or by use of microparticle bombardment (e.g., a gene gun; Biolistic, Dupont), or coating with lipids or cell-surface receptors or transfecting agents, encapsulation in liposomes, microparticles, or microcapsules, or by administering them in linkage to a peptide which is known to enter the nucleus, by administering it in linkage to a ligand subject to receptor-mediated endocytosis (see, e.g., Wu and Wu, J. Biol. Chem. 262:4429-4432 (1987)) (which can be used to target cell types specifically expressing the receptors), etc. In another embodiment, nucleic acid-ligand complexes can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., PCT Publications WO 92/06180; WO 92/22635; WO92/20316; WO93/14188, WO 93/20221). Alternatively, the nucleic acid can be introduced intracellularly and incorporated within host cell DNA for expression, by homologous recombination (Koller and Smithies, Proc. Natl. Acad. Sci. USA 86:8932-8935 (1989); Zijlstra et al., Nature 342:435-438 (1989)).

In a specific embodiment, viral vectors that contains nucleic acid sequences encoding an antibody of the invention are used. For example, a retroviral vector can be used (see Miller et al., Meth. Enzymol. 217:581-599 (1993)). These retroviral vectors contain the components necessary for the correct packaging of the viral genome and integration into the host cell DNA. The nucleic acid sequences encoding the antibody to be used in gene therapy are cloned into one or more vectors, which facilitates delivery of the gene into a patient. More detail about retroviral vectors can be found in Boesen et al., Biotherapy 6:291-302 (1994), which describes the use of a retroviral vector to deliver the mdr1 gene to hematopoietic stem cells in order to make the stem cells more resistant to chemotherapy. Other references illustrating the use of retroviral vectors in gene therapy are: Clowes et al., J. Clin. Invest. 93:644-651 (1994); Kiem et al., Blood 83:1467-1473 (1994); Salmons and Gunzberg, Human Gene Therapy 4:129-141 (1993); and Grossman and Wilson, Curr. Opin. in Genetics and Devel. 3:110-114 (1993).

Adenoviruses are other viral vectors that can be used in gene therapy. Adenoviruses are especially attractive vehicles for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Kozarsky and Wilson, Current Opinion in Genetics and Development 3:499-503 (1993) present a review of adenovirus-based gene therapy. Bout et al., Human Gene Therapy 5:3-10 (1994) demonstrated the use of adenovirus vectors to transfer genes to the respiratory epithelia of rhesus monkeys. Other instances of the use of adenoviruses in gene therapy can be found in Rosenfeld et al., Science 252:431-434 (1991); Rosenfeld et al., Cell 68:143-155 (1992); Mastrangeli et al., J. Clin. Invest. 91:225-234 (1993); PCT Publication WO94/12649; and Wang, et al., Gene Therapy 2:775-783 (1995). In a preferred embodiment, adenovirus vectors are used.

Adeno-associated virus (AAV) has also been proposed for use in gene therapy (Walsh et al., Proc. Soc. Exp. Biol. Med. 204:289-300 (1993); U.S. Pat. No. 5,436,146).

Another approach to gene therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a patient.

In this embodiment, the nucleic acid is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcell-mediated gene transfer, spheroplast fusion, etc. Numerous techniques are known in the art for the introduction of foreign genes into cells (see, e.g., Loeffler and Behr, Meth. Enzymol. 217:599-618 (1993); Cohen et al., Meth. Enzymol. 217:618-644 (1993); Cline, Pharmac. Ther. 29:69-92m (1985) and may be used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny.

The resulting recombinant cells can be delivered to a patient by various methods known in the art. Recombinant blood cells (e.g., hematopoietic stem or progenitor cells) are preferably administered intravenously. The amount of cells envisioned for use depends on the desired effect, patient state, etc., and can be determined by one skilled in the art.

Cells into which a nucleic acid can be introduced for purposes of gene therapy encompass any desired, available cell type, and include but are not limited to epithelial cells, endothelial cells, keratinocytes, fibroblasts, muscle cells, hepatocytes; blood cells such as Tlymphocytes, Blymphocytes, monocytes, macrophages, neutrophils, eosinophils, megakaryocytes, granulocytes; various stem or progenitor cells, in particular hematopoietic stem or progenitor cells, e.g., as obtained from bone marrow, umbilical cord blood, peripheral blood, fetal liver, etc.

In a preferred embodiment, the cell used for gene therapy is autologous to the patient.

In an embodiment in which recombinant cells are used in gene therapy, nucleic acid sequences encoding an antibody are introduced into the cells such that they are expressible by the cells or their progeny, and the recombinant cells are then administered in vivo for therapeutic effect. In a specific embodiment, stem or progenitor cells are used. Any stem and/or progenitor cells which can be isolated and maintained in vitro can potentially be used in accordance with this embodiment of the present invention (see e.g. PCT Publication WO 94/08598; Stemple and Anderson, Cell 71:973-985 (1992); Rheinwald, Meth. Cell Bio. 21A:229 (1980); and Pittelkow and Scott, Mayo Clinic Proc. 61:771 (1986)).

In a specific embodiment, the nucleic acid to be introduced for purposes of gene therapy comprises an inducible promoter operably linked to the coding region, such that expression of the nucleic acid is controllable by controlling the presence or absence of the appropriate inducer of transcription.

### IX. PHARMACEUTICAL COMPOSITIONS. AND ADMINISTRATION METHODS

Methods of preparing and administering one or more C35 antibodies, or antigen-binding fragments, variants, or derivatives thereof of the invention to a subject in need thereof are well known to or are readily determined by those skilled in the art. The route of administration of one or more C35 antibodies, or antigen-binding fragments, variants, or derivatives thereof may be, for example, oral, parenteral, by inhalation or topical. The term parenteral as used herein includes, e.g., intravenous, intraarterial, intraperitoneal, intramuscular, subcutaneous, rectal or vaginal administration. While all these forms of administration are clearly contemplated as being within the scope of the invention, a form for administration would be a solution for injection, in particular for intravenous or intraarterial injection or drip. Usually, a suitable pharmaceutical composition for injection may comprise a buffer (e.g. acetate, phosphate or citrate buffer), a surfactant (e.g. polysorbate), optionally a stabilizer agent (e.g. human albumin), etc. However, in other methods compatible with the teachings herein, C35 antibodies, or antigen-binding fragments, variants, or derivatives thereof of the invention can be delivered directly to the site of the adverse cellular population thereby increasing the exposure of the diseased tissue to the therapeutic agent.

As previously discussed, at least one C35 antibody, or more preferably at least two or more C35 antibodies, or antigen-binding fragments, variants, or derivatives thereof of the invention may be administered in a pharmaceutically effective amount for the in vivo treatment of cancer. In a preferred embodiment, two C35 antibodies or antigen-binding fragments, variants, or derivatives thereof of the invention may be administered in a pharmaceutically effective amount for the in vivo treatment of cancer.

In this regard, it will be appreciated that the disclosed antibodies will be formulated so as to facilitate administration and promote stability of the active agent. Preferably, pharmaceutical compositions in accordance with the present invention comprise a pharmaceutically acceptable, non-toxic, sterile carrier such as physiological saline, non-toxic buffers, preservatives and the like. For the purposes of the instant application, a pharmaceutically effective amount of a C35 antibody, or antigen-binding fragment, variant, or derivative thereof, conjugated or unconjugated, shall be held to mean an amount sufficient to achieve effective binding to a target and to achieve a benefit, e.g., to ameliorate symptoms of a disease or disorder or to detect a substance or a cell.

In one embodiment, the entire antibody dose is provided in a single bolus. Alternatively, the dose can be provided by multiple administrations, such as an extended infusion method or by repeated injections administered over a span of hours or days, for example, a span of about 2 to about 4 days. Also see Examples 5, 910, and 14 and Tables 7-10.

In some embodiments, the two or more C35 antibodies are administered together in the same pharmaceutical preparation. In other embodiments the antibodies are administered as separate pharmaceutical preparations, either concurrently or sequentially.

Formulations and methods of administration that can be employed when the compound comprises a nucleic acid or an immunoglobulin are described above; additional appropriate formulations and routes of administration can be selected from among those described herein below.

Various delivery systems are known and can be used to administer a compound of the invention, e.g., encapsulation in liposomes, microparticles, microcapsules, recombinant cells capable of expressing the compound, receptor-mediated endocytosis (see, e.g., Wu and Wu, J. Biol. Chem. 262:4429-4432 (1987)), construction of a nucleic acid as part of a retroviral or other vector, etc. Methods of introduction include but are not limited to intradermal, intramuscular, intraperitoneal, intravenous, subcutaneous, intranasal, epidural, and oral routes. The compounds or compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compounds or compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the pharmaceutical compounds or compositions of the invention locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. Preferably, when administering a protein, including an antibody, of the invention, care must be taken to use materials to which the protein does not absorb.

In another embodiment, the compound or composition can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.)

In yet another embodiment, the compound or composition can be delivered in a controlled release system. In one embodiment, a pump may be used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989)). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, J., Macromol. Sci. Rev. Macromol. Chem. 23:61 (1983); see also Levy et al., Science 228:190 (1985); During et al., Ann. Neurol. 25:351 (1989); Howard et al., J. Neurosurg. 7.1:105 (1989)). In yet another embodiment, a controlled release system can be placed in proximity of the therapeutic target, i.e., the brain, thus requiring only a fraction of the systemic dose (see, e.g., Goodson, in Medical Applications of Controlled Release, supra, vol. 2, pp. 115-138 (1984)).

Other controlled release systems are discussed in the review by Langer (Science 249:1527-1533 (1990)).

In a specific embodiment where the compound of the invention is a nucleic acid encoding a protein, the nucleic acid can be administered in vivo to promote expression of its encoded protein, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see e.g., Joliot et al., Proc. Natl. Acad. Sci. USA 88:1864-1868 (1991)), etc. Alternatively, a nucleic acid can be introduced intracellularly and incorporated within host cell DNA for expression, by homologous recombination.

The present invention also provides pharmaceutical compositions. Such compositions comprise a therapeutically effective amount of a compound, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the compound, preferably in purified form, together with a suitable amount of carrier so as to provide the form for proper administration to the patient. The formulation should suit the mode of administration.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compounds of the invention can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

The amount of the compound of the invention which will be effective in the treatment, inhibition and prevention of a disease or disorder associated with aberrant expression and/or activity of a polypeptide of the invention can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances. Effective doses may be extrapolated from dose-response curves derived from in vitro or animal model test systems.

For antibodies, the dosage administered to a patient is typically about 0.1 mg/kg to about 100 mg/kg of the patient's body weight. Preferably, the dosage administered to a patient is between about 0.1 mg/kg and about 20 mg/kg of the patient's body weight, more preferably about 1 mg/kg to about 10 mg/kg of the patient's body weight. In some embodiments the two or more C35 antibodies are administered at a total dose of about 10 mg/kg to about 50 mg/kg of the patient's body weight. In another embodiment the antibodies are administered at a total dose of about 20 mg/kg to about 40 mg/kg. Generally, human antibodies have a longer half-life within the human body than antibodies from other species due to the immune response to the foreign polypeptides. Thus, lower dosages of human antibodies and less frequent administration is often possible. Further, the dosage and frequency of administration of antibodies of the invention may be reduced by enhancing uptake and tissue penetration of the antibodies by modifications such as, for example, lipidation. Also see Example 5.

As discussed above, the invention also provides a pharmaceutical pack or kit comprising one or more containers filled with one or more of the ingredients of the pharmaceutical compositions of the invention. For example, the pharmaceutical pack or kit may contain the antibody preparation comprising two or more C35 antibodies and the chemotherapeutic agent, such as paclitaxel or adriamycin. In some embodiments, the antibodies are in the same container. In other embodiments, the antibodies are in separate containers. In some embodiments, the chemotherapeutic agent is in the same container as the antibody preparation. In other embodiments, the chemotherapeutic agent is in a separate container. Optionally associated with such container(s) can be a notice in the form prescribed by a governmental agency regulating the manufacture, use or sale of pharmaceuticals or biological products, which notice reflects approval by the agency of manufacture, use or sale for human administration.

Antibodies can be used to assay levels of polypeptides encoded by polynucleotides of the invention in a biological sample using classical immunohistological methods known to those of skill in the art (e.g., see Jalkanen, et al., J. Cell. Biol. 101:976-985 (1985); Jalkanen, et al., J. Cell. Biol. 105:3087-3096 (1987)). Other antibody-based methods useful for detecting protein gene expression include immunoassays, such as the enzyme linked immunosorbent assay (ELISA) and the radioimmunoassay (RIA). Suitable antibody assay labels are known in the art and include enzyme labels, such as, glucose oxidase; radioisotopes, such as iodine (¹³¹I, ¹²⁵I, ¹²³I, ¹²¹I, carbon (¹⁴C), sulfur (³⁵S), tritium (³H), indium (¹¹⁵mIn, ¹¹³ in, ¹¹²In, ¹¹¹In), and technetium (⁹⁹Tc, ⁹⁹mTc), thallium (201Ti), gallium (⁶⁸Ga, ⁶⁷Ga), palladium (¹⁰³Pd), molybdenum (⁹⁹Mo), xenon (¹³³Xe), fluorine (¹⁸F), ¹⁵³Sm, ¹⁷⁷Lu, ¹⁵⁹Gd, ¹⁴⁹Pm, ¹⁴⁰La, ¹⁷⁵Yb, ¹⁶⁶Ho, ⁹⁰Y, ⁴⁷Sc, ¹⁸⁶Re, ⁸⁸Re, ¹⁴²Pr, ¹⁰⁵Rh, ⁹⁷Ru; luminescent labels, such as luminol; and fluorescent labels, such as fluorescein and rhodamine, and biotin.

In addition to assaying levels of polypeptide of the present invention in a biological sample, proteins can also be detected in vivo by imaging. Antibody labels or markers for in vivo imaging of protein include those detectable by X-radiography, NMR or ESR. For X-radiography, suitable labels include radioisotopes such as barium or cesium, which emit detectable radiation but are not overtly harmful to the subject. Suitable markers for NMR and ESR include those with a detectable characteristic spin, such as deuterium, which may be incorporated into the antibody by labeling of nutrients for the relevant hybridoma.

A protein-specific antibody or antibody fragment which has been labeled with an appropriate detectable imaging moiety, such as a radioisotope (for example, ¹³¹I, 1¹²In, ⁹⁹mTc, (¹³¹I, ¹²⁵I, ¹²³I, ¹²¹I), carbon (¹⁴C), sulfur (35S), tritium (³H), indium (¹¹⁵mIn, ¹¹³mIn, ¹¹²In, ¹¹¹In), and technetium (⁹⁹Tc, ⁹⁹mTc), thallium (²⁰¹Ti), gallium (⁶⁸Ga, ⁶⁷Ga), palladium (¹⁰³Pd), molybdenum (⁹⁹Mo), xenon (¹³³Xe), fluorine (¹⁸F, ¹⁵³Sm, ¹⁷⁷Lu, ⁵⁹Gd, ¹⁴⁹ Pm, ¹⁴⁰La, ¹⁷⁵Yb, ¹⁶⁶Ho, ⁹⁰Y, ⁴⁷Sc, ¹⁸⁶Re, ¹⁸⁸Re, ¹⁴²Pr, ¹⁰⁵Rh, ⁹⁷Ru), a radio-opaque substance, or a material detectable by nuclear magnetic resonance, is introduced (for example, parenterally, subcutaneously or intraperitoneally) into the mammal to be examined for immune system disorder. It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of ⁹⁹mTc. The labeled antibody or antibody fragment will then preferentially accumulate at the location of cells which express the polypeptide encoded by a polynucleotide of the invention. In vivo tumor imaging is described in S. W. Burchiel et al., “Immunopharmacokinetics of Radiolabeled Antibodies and Their Fragments” (Chapter 13 in Tumor Imaging The Radiochemical Detection of Cancer, S. W. Burchiel and B. A. Rhodes, eds., Masson Publishing Inc. (1982)).

In one embodiment, the invention provides a method for the specific delivery of compositions of the invention to cells by administering polypeptides of the invention (e.g., polypeptides encoded by polynucleotides of the invention and/or antibodies) that are associated with heterologous polypeptides or nucleic acids. In one example, the invention provides a method for delivering a therapeutic protein into the targeted cell. In another example, the invention provides a method for delivering a single stranded nucleic acid (e.g., antisense or ribozymes) or double stranded nucleic acid (e.g., DNA that can integrate into the cell's genome or replicate episomally and that can be transcribed) into the targeted cell.

Techniques known in the art may be applied to label polypeptides of the invention (including antibodies). Such techniques include, but are not limited to, the use of bifunctional conjugating agents (see e.g., U.S. Pat. Nos. 5,756,065; 5,714,631; 5,696,239; 5,652,361; 5,505,931; 5,489,425; 5,435,990; 5,428,139; 5,342,604; 5,274,119; 4,994,560; and 5,808,003; the contents of each of which are hereby incorporated by reference in its entirety).

### X. DIAGNOSTICS

The invention further provides a diagnostic method useful during diagnosis of cancer, which involves measuring the expression level of C35 protein or transcript in tissue or other cells or body fluid from an individual and comparing the measured expression level with standard C35 expression levels in normal tissue or body fluid, whereby an increase in the expression level compared to the standard is indicative of a disorder.

C35-specific antibodies can be used to assay protein levels in a biological sample using classical immunohistological methods known to those of skill in the art (e.g., see Jalkanen, et al., J. Cell. Biol. 101:976-985 (1985); Jalkanen, et al., J. Cell Biol. 105:3087-3096 (1987)). Other antibody-based methods useful for detecting protein expression include immunoassays, such as the enzyme linked immunosorbent assay (ELISA), immunoprecipitation, or western blotting. Suitable assays are described in more detail elsewhere herein.

By “assaying the expression level of C35 polypeptide” is intended qualitatively or quantitatively measuring or estimating the level of C35 polypeptide in a first biological sample either directly (e.g., by determining or estimating absolute protein level) or relatively (e.g., by comparing to the disease associated polypeptide level in a second biological sample). Preferably, C35 polypeptide expression level in the first biological sample is measured or estimated and compared to a standard C35 polypeptide level, the standard being taken from a second biological sample obtained from an individual not having the disorder or being determined by averaging levels from a population of individuals not having the disorder. As will be appreciated in the art, once the “standard” C35 polypeptide level is known, it can be used repeatedly as a standard for comparison.

By “biological sample” is intended any biological sample obtained from an individual, cell line, tissue culture, or other source of cells potentially expressing C35. Methods for obtaining tissue biopsies and body fluids from mammals are well known in the art.

C35 antibodies for use in the diagnostic methods described above include any C35 antibody which specifically binds to a C35 gene product, as described elsewhere herein.

### XI. IMMUNOASSAYS

C35 antibodies, or antigen-binding fragments, variants, or derivatives thereof of the invention may be assayed for immunospecific binding by any method known in the art. The immunoassays which can be used include but are not limited to competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few. Such assays are routine and well known in the art (see, e.g., Ausubel et al., eds, Current Protocols in Molecular Biology, John Wiley & Sons, Inc., New York, Vol. 1 (1994), which is incorporated by reference herein in its entirety). Exemplary immunoassays are described briefly below (but are not intended by way of limitation).

Immunoprecipitation protocols generally comprise lysing a population of cells in a lysis buffer such as RIPA buffer (1% NP-40 or Triton X-100, 1% sodium deoxycholate, 0.1% SDS, 0.15 M NaCl, 0.01 M sodium phosphate at pH 7.2, 1% Trasylol) supplemented with protein phosphatase and/or protease inhibitors (e.g., EDTA, PMSF, aprotinin, sodium vanadate), adding the antibody of interest to the cell lysate, incubating for a period of time (e.g., 1-4 hours) at 4° C., adding protein A and/or protein G sepharose beads to the cell lysate, incubating for about an hour or more at 4° C., washing the beads in lysis buffer and resuspending the beads in SDS/sample buffer. The ability of the antibody of interest to immunoprecipitate a particular antigen can be assessed by, e.g., western blot analysis. One of skill in the art would be knowledgeable as to the parameters that can be modified to increase the binding of the antibody to an antigen and decrease the background (e.g., pre-clearing the cell lysate with sepharose beads). For further discussion regarding immunoprecipitation protocols see, e.g., Ausubel et al., eds, Current Protocols in Molecular Biology, John Wiley & Sons, Inc., New York, Vol. 1 (1994) at 10.16.1.

Western blot analysis generally comprises preparing protein samples, electrophoresis of the protein samples in a polyacrylamide gel (e.g., 8%-20% SDS-PAGE depending on the molecular weight of the antigen), transferring the protein sample from the polyacrylamide gel to a membrane such as nitrocellulose, PVDF or nylon, blocking the membrane in blocking solution (e.g., PBS with 3% BSA or non-fat milk), washing the membrane in washing buffer (e.g., PBS-Tween 20), blocking the membrane with primary antibody (the antibody of interest) diluted in blocking buffer, washing the membrane in washing buffer, blocking the membrane with a secondary antibody (which recognizes the primary antibody, e.g., an anti-human antibody) conjugated to an enzymatic substrate (e.g., horseradish peroxidase or alkaline phosphatase) or radioactive molecule (e.g., ³²p or 125l) diluted in blocking buffer, washing the membrane in wash buffer, and detecting the presence of the antigen. One of skill in the art would be knowledgeable as to the parameters that can be modified to increase the signal detected and to reduce the background noise. For further discussion regarding western blot protocols see, e.g., Ausubel et al., eds, Current Protocols in Molecular Biology, John Wiley & Sons, Inc., New York Vol. 1 (1994) at 10.8.1.

ELISAs comprise preparing antigen, coating the well of a 96 well microtiter plate with the antigen, adding the antibody of interest conjugated to a detectable compound such as an enzymatic substrate (e.g., horseradish peroxidase or alkaline phosphatase) to the well and incubating for a period of time, and detecting the presence of the antigen. In ELISAs the antibody of interest does not have to be conjugated to a detectable compound; instead, a second antibody (which recognizes the antibody of interest) conjugated to a detectable compound may be added to the well. Further, instead of coating the well with the antigen, the antibody may be coated to the well. In this case, a second antibody conjugated to a detectable compound may be added following the addition of the antigen of interest to the coated well. One of skill in the art would be knowledgeable as to the parameters that can be modified to increase the signal detected as well as other variations of ELISAs known in the art. For further discussion regarding ELISAs see, e.g., Ausubel et al., eds, Current Protocols in Molecular Biology, John Wiley & Sons, Inc., New York, Vol. 1 (1994) at 11.2.1.

The binding affinity of an antibody to an antigen and the off-rate of an antibody-antigen interaction can be determined by competitive binding assays. One example of a competitive binding assay is a radioimmunoassay comprising the incubation of labeled antigen (e.g., ³H or ¹²⁵I with the antibody of interest in the presence of increasing amounts of unlabeled antigen, and the detection of the antibody bound to the labeled antigen. The affinity of the antibody of interest for a particular antigen and the binding off-rates can be determined from the data by scatchard plot analysis. Competition with a second antibody can also be determined using radioimmunoassays. In this case, the antigen is incubated with antibody of interest is conjugated to a labeled compound (e.g., ³H or ¹²⁵I) in the presence of increasing amounts of an unlabeled second antibody.

C35 antibodies, or antigen-binding fragments, variants, or derivatives thereof of the invention, additionally, be employed histologically, as in immunofluorescence, immunoelectron microscopy or non-immunological assays, for in situ detection of cancer antigen gene products or conserved variants or peptide fragments thereof. In situ detection may be accomplished by removing a histological specimen from a patient, and applying thereto a labeled C35 antibody, or antigen-binding fragment, variant, or derivative thereof, preferably applied by overlaying the labeled antibody (or fragment) onto a biological sample. Through the use of such a procedure, it is possible to determine not only the presence of C35 protein, or conserved variants or peptide fragments, but also its distribution in the examined tissue. Using the present invention, those of ordinary skill will readily perceive that any of a wide variety of histological methods (such as staining procedures) can be modified in order to achieve such in situ detection.

Immunoassays and non-immunoassays for C35 gene products or conserved variants or peptide fragments thereof will typically comprise incubating a sample, such as a biological fluid, a tissue extract, freshly harvested cells, or lysates of cells which have been incubated in cell culture, in the presence of a detectably labeled antibody capable of binding to C35 or conserved variants or peptide fragments thereof, and detecting the bound antibody by any of a number of techniques well-known in the art.

The biological sample may be brought in contact with and immobilized onto a solid phase support or carrier such as nitrocellulose, or other solid support which is capable of immobilizing cells, cell particles or soluble proteins. The support may then be washed with suitable buffers followed by treatment with the detectably labeled C35 antibody, or antigen-binding fragment, variant, or derivative thereof. The solid phase support may then be washed with the buffer a second time to remove unbound antibody. Optionally the antibody is subsequently labeled. The amount of bound label on solid support may then be detected by conventional means.

By “solid phase support or carrier” is intended any support capable of binding an antigen or an antibody. Well-known supports or carriers include glass, polystyrene, polypropylene, polyethylene, dextran, nylon, amylases, natural and modified celluloses, polyacrylamides, gabbros, and magnetite. The nature of the carrier can be either soluble to some extent or insoluble for the purposes of the present invention. The support material may have virtually any possible structural configuration so long as the coupled molecule is capable of binding to an antigen or antibody. Thus, the support configuration may be spherical, as in a bead, or cylindrical, as in the inside surface of a test tube, or the external surface of a rod. Alternatively, the surface may be flat such as a sheet, test strip, etc. Preferred supports include polystyrene beads. Those skilled in the art will know many other suitable carriers for binding antibody or antigen, or will be able to ascertain the same by use of routine experimentation.

The binding activity of a given lot of C35 antibody, or antigen-binding fragment, variant, or derivative thereof may be determined according to well known methods. Those skilled in the art will be able to determine operative and optimal assay conditions for each determination by employing routine experimentation.

There are a variety of methods available for measuring the affinity of an antibody-antigen interaction, but relatively few for determining rate constants. Most of the methods rely on either labeling antibody or antigen, which inevitably complicates routine measurements and introduces uncertainties in the measured quantities.

Surface plasmon reasonance (SPR) as performed on BIAcore offers a number of advantages over conventional methods of measuring the affinity of antibody-antigen interactions: (i) no requirement to label either antibody or antigen; (ii) antibodies do not need to be purified in advance, cell culture supernatant can be used directly; (iii) real-time measurements, allowing rapid semi-quantitative comparison of different monoclonal antibody interactions, are enabled and are sufficient for many evaluation purposes; (iv) biospecific surface can be regenerated so that a series of different monoclonal antibodies can easily be compared under identical conditions; (v) analytical procedures are fully automated, and extensive series of measurements can be performed without user intervention. BIAapplications Handbook, version AB (reprinted 1998), BIACORE code No. BR-1001-86; BIAtechnology Handbook, version AB (reprinted 1998), BIACORE code No. BR-001-84.

SPR based binding studies require that one member of a binding pair be immobilized on a sensor surface. The binding partner immobilized is referred to as the ligand. The binding partner in solution is referred to as the analyte. In some cases, the ligand is attached indirectly to the surface through binding to another immobilized molecule, which is referred as the capturing molecule. SPR response reflects a change in mass concentration at the detector surface as analytes bind or dissociate.

Based on SPR, real-time BIAcore measurements monitor interactions directly as they happen. The technique is well suited to determination of kinetic parameters. Comparative affinity ranking is extremely simple to perform, and both kinetic and affinity constants can be derived from the sensorgram data.

When analyte is injected in a discrete pulse across a ligand surface, the resulting sensorgram can be divided into three essential phases: (i) Association of analyte with ligand during sample injection; (ii) Equilibrium or steady state during sample injection, where the rate of analyte binding is balanced by dissociation from the complex; (iii) Dissociation of analyte from the surface during buffer flow.

The association and dissociation phases provide information on the kinetics of analyte-ligand interaction (kₐ and k_{d}, the rates of complex formation and dissociation, k_{d}/kₐ=K_{D}). The equilibrium phase provides information on the affinity of the analyte-ligand interaction (K_{D}).

BIAevaluation software provides comprehensive facilities for curve fitting using both numerical integration and global fitting algorithms. With suitable analysis of the data, separate rate and affinity constants for interaction can be obtained from simple BIAcore investigations. The range of affinities measurable by this technique is very broad ranging from mM to pM.

Epitope specificity is an important characteristic of a monoclonal antibody. Epitope mapping with BIAcore, in contrast to conventional techniques using radioimmunoassay, ELISA or other surface adsorption methods, does not require labeling or purified antibodies, and allows multi-site specificity tests using a sequence of several monoclonal antibodies. Additionally, large numbers of analyses can be processed automatically.

Pair-wise binding experiments test the ability of two MAbs to bind simultaneously to the same antigen. MAbs directed against separate epitopes will bind independently, whereas MAbs directed against identical or closely related epitopes will interfere with each other's binding. These binding experiments with BIAcore are straightforward to carry out.

For example, one can use a capture molecule to bind the first MAb, followed by addition of antigen and second MAb sequentially. The sensorgrams will reveal: 1. how much of the antigen binds to first MAb, 2. to what extent the second MAb binds to the surface-attached antigen, 3. if the second MAb does not bind, whether reversing the order of the pair-wise test alters the results.

Peptide inhibition is another technique used for epitope mapping. This method can complement pair-wise antibody binding studies, and can relate functional epitopes to structural features when the primary sequence of the antigen is known. Peptides or antigen fragments are tested for inhibition of binding of different MAbs to immobilized antigen. Peptides which interfere with binding of a given MAb are assumed to be structurally related to the epitope defined by that MAb.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2nd Ed., Sambrook et al., ed., Cold Spring Harbor Laboratory Press: (1989); Molecular Cloning: A Laboratory Manual, Sambrook et al., ed., Cold Springs Harbor Laboratory, New York (1992), DNA Cloning, D. N. Glover ed., Volumes I and II (1985); Oligonucleotide Synthesis, M. J. Gait ed., (1984); Mullis et al. U.S. Pat. No. 4,683,195; Nucleic Acid Hybridization, B. D. Hames & S. J. Higgins eds. (1984); Transcription And Translation, B. D. Hames & S. J. Higgins eds. (1984); Culture Of Animal Cells, R. I. Freshney, Alan R. Liss, Inc., (1987); Immobilized Cells And Enzymes, IRL Press, (1986); B. Perbal, A Practical Guide To Molecular Cloning (1984); the treatise, Methods In Enzymology, Academic Press, Inc., N.Y.; Gene Transfer Vectors For Mammalian Cells, J. H. Miller and M. P. Calos eds., Cold Spring Harbor Laboratory (1987); Methods In Enzymology, Vols. 154 and 155 (Wu et al. eds.); Immunochemical Methods In Cell And Molecular Biology, Mayer and Walker, eds., Academic Press, London (1987); Handbook Of Experimental Immunology, Volumes I-IV, D. M. Weir and C. C. Blackwell, eds., (1986); Manipulating the Mouse Embryo, Cold Spring Harbor Laboratory Press, Cold Spring Harbor, N.Y., (1986); and in Ausubel et al., Current Protocols in Molecular Biology, John Wiley and Sons, Baltimore, Md. (1989).

General principles of antibody engineering are set forth in Antibody Engineering, 2nd edition, C. A. K. Borrebaeck, Ed., Oxford Univ. Press (1995). General principles of protein engineering are set forth in Protein Engineering, A Practical Approach, Rickwood, D., et al., Eds., IRL Press at Oxford Univ. Press, Oxford, Eng. (1995). General principles of antibodies and antibody-hapten binding are set forth in: Nisonoff, A., Molecular Immunology, 2nd ed., Sinauer Associates, Sunderland, Mass. (1984); and Steward, M. W., Antibodies, Their Structure and Function, Chapman and Hall, New York, N.Y. (1984). Additionally, standard methods in immunology known in the art and not specifically described are generally followed as in Current Protocols in Immunology, John Wiley & Sons, New York; Stites et al. (eds), Basic and Clinical—Immunology (8th ed.), Appleton & Lange, Norwalk, Conn. (1994) and Mishell and Shiugi (eds), Selected Methods in Cellular Immunology, W.H. Freeman and Co., New York (1980).

Standard reference works setting forth general principles of immunology include Current Protocols in Immunology, John Wiley & Sons, New York; Klein, J., Immunology: The Science of Self-Nonself Discrimination, John Wiley & Sons, New York (1982); Kennett, R., et al., eds., Monoclonal Antibodies, Hybridoma: A New Dimension in Biological Analyses, Plenum Press, New York (1980); Campbell, A., “Monoclonal Antibody Technology” in Burden, R., et al., eds., Laboratory Techniques in Biochemistry and Molecular Biology, Vol. 13, Elsevere, Amsterdam (1984), Kuby Immunnology 4^{th} ed. Ed. Richard A. Goldsby, Thomas J. Kindt and Barbara A. Osborne, H. Freemand & Co. (2000); Roitt, I., Brostoff, J. and Male D., Immunology 6^{th} ed. London: Mosby (2001); Abbas A., Abul, A. and Lichtman, A., Cellular and Molecular Immunology Ed. 5, Elsevier Health Sciences Division (2005); Kontermann and Dubel, Antibody Engineering, Springer Verlan (2001); Sambrook and Russell, Molecular Cloning: A Laboratory Manual. Cold Spring Harbor Press (2001); Lewin, Genes VIII, Prentice Hall (2003); Harlow and Lane, Antibodies: A Laboratory Manual, Cold Spring Harbor Press (1988); Dieffenbach and Dveksler, PCR Primer Cold Spring Harbor Press (2003).

All of the references cited above, as well as all references cited herein and herein below, are incorporated herein by reference in their entireties.

### EXAMPLES

### Example 1

### C35 Exposed on Surface Membrane of Breast Tumor Cells Following Radiation Induced Apoptosis

A line of continuously growing breast tumor cells that express the C35 tumor antigen was either irradiated with 300 Gy or left untreated. After continued in vitro culture for several days to allow apoptosis to develop, cells were harvested, washed and stained with 50 ng of 1F2 monoclonal anti-C35 antibody or a mouse IgG antibody control each conjugated to the fluorescent dye Alexa 647. Following 50 minutes incubation at 25° C., cells were stained with Annexin V and propidium iodide (PI) using a standard commercial kit (Pharmingen). Cells were analyzed for staining with Annexin V, propidium iodide and Alexa 647 by flow cytometry employing standard protocols.

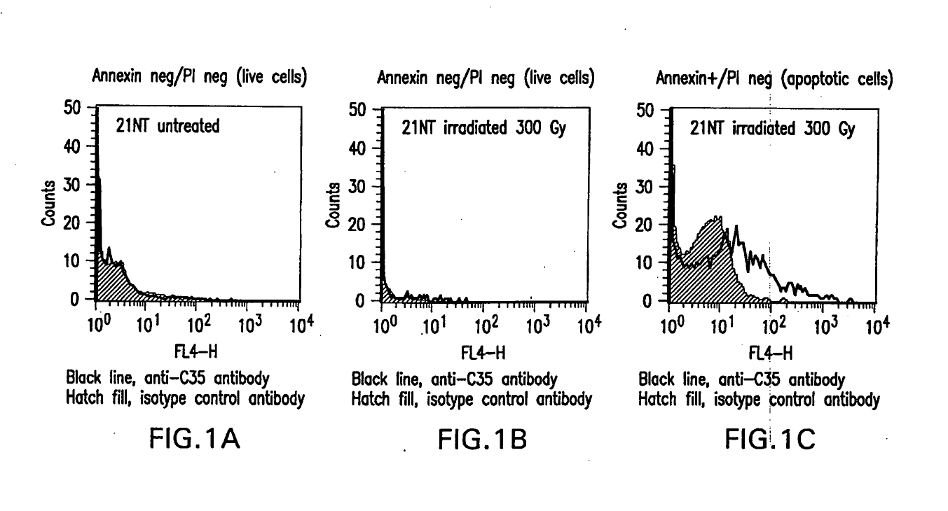

The results in FIG. 1 show that untreated live cells (PI negative), that are not undergoing apoptosis (Annexin V negative), do not express C35 on the surface membrane as evidenced by absence of differential staining with anti-C35 antibody and the isotype control antibody (FIG. 1A). Similarly, irradiated tumor cells that remain viable (PI negative) and have not been induced to undergo apoptosis (Annexin V negative) also do not express C35 on the tumor cell surface membrane (FIG. 1B). In striking contrast, irradiated tumor cells that are viable (PI negative), but undergoing apoptosis (Annexin V positive), are clearly differentially stained with anti-C35 antibodies as compared to isotype control antibody (FIG. 1C).

### Example 2

### C35 Exposed on Surface Membrane of Breast Tumor Cells Following Drug Induced Apoptosis

A line of continuously growing breast tumor cells that express the C35 tumor antigen was either treated with 6 ug/ml mitomycin C or left untreated. After continued in vitro culture for 48 hours to allow apoptosis to develop, cells were harvested, washed and stained with 50 ng of 1F2 monoclonal anti-C35 antibody or a mouse IgG antibody control each conjugated to the fluorescent dye Alexa 647. Following 50 minutes incubation at 25° C., cells were stained with Annexin V and propidium iodide (PI) using a standard commercial kit (Pharmingen). Cells were analyzed for staining with Annexin V, propidium iodide and Alexa 647 by flow cytometry employing standard protocols.

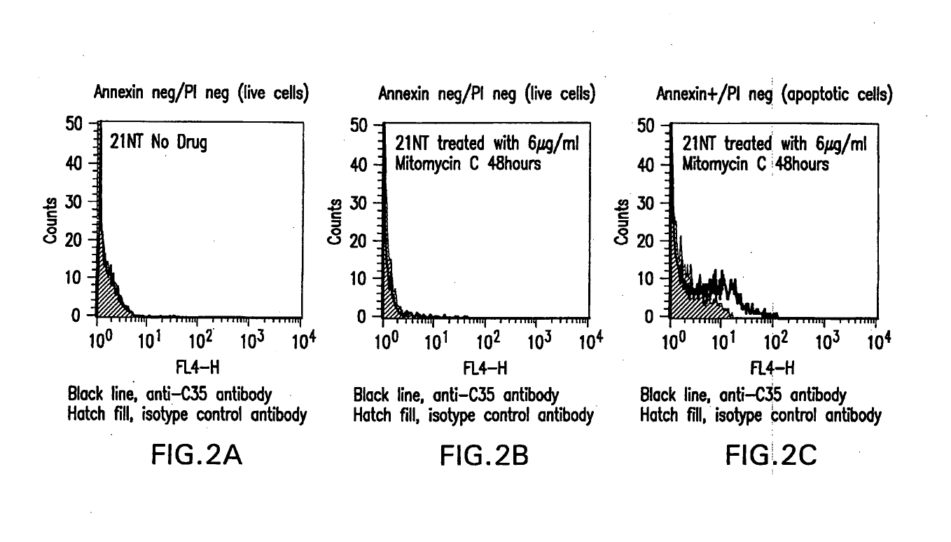

The results in FIG. 2 show that untreated live cells (PI negative), that are not undergoing apoptosis (Annexin V negative), do not express C35 on the surface membrane as evidenced by absence of differential staining with anti-C35 antibody and the isotype control antibody (FIG. 2A). Similarly, mitomycin C treated tumor cells that remain viable (PI negative) and have not been induced to undergo apoptosis (Annexin V negative) also do not express C35 on the tumor cell surface membrane (FIG. 2B). In striking contrast, mitomycin C treated tumor cells that are viable (PI negative), but undergoing apoptosis (Annexin V positive), are clearly differentially stained with anti-C35 antibodies as compared to isotype control antibody (FIG. 2C).

### Example 3

### Expression of an Antibody in Mammalian Cells

The polypeptide of the present invention can be expressed in a mammalian cell. A typical mammalian expression vector contains a promoter element, which mediates the initiation of transcription of mRNA, a protein coding sequence, and signals required for the termination of transcription and polyadenylation of the transcript. Additional elements include enhancers, Kozak sequences and intervening sequences flanked by donor and acceptor sites for RNA splicing. Highly efficient transcription is achieved with the early and late promoters from SV40, the long terminal repeats (LTRs) from Retroviruses, e.g., RSV, HTLVI, HIVI and the early promoter of the cytomegalovirus (CMV). However, cellular elements can also be used (e.g., the human actin promoter).

Suitable expression vectors for use in practicing the present invention include, for example, vectors such as pSVL and pMSG (Pharmacia, Uppsala, Sweden), pRSVcat (ATCC 37152), pSV2dhfr (ATCC 37146), pBC12MI (ATCC 67109), pCMVSport 2.0, and pCMVSport 3.0. Mammalian host cells that could be used include, human Hela, 293, H9 and Jurkat cells, mouse NIH3T3 and C127 cells, Cos 1, Cos 7 and CV1, quail QC1-3 cells, mouse L cells and Chinese hamster ovary (CHO) cells.

Alternatively, the polypeptide can be expressed in stable cell lines containing the polynucleotide integrated into a chromosome. The co-transfection with a selectable marker such as DHFR, gpt, neomycin, hygromycin allows the identification and isolation of the transfected cells.

The transfected gene can also be amplified to express large amounts of the encoded protein. The DHFR (dihydrofolate reductase) marker is useful in developing cell lines that carry several hundred or even several thousand copies of the gene of interest. (See, e.g., Alt, F. W., et al., J. Biol. Chem. 253:1357-1370 (1978); Hamlin, J. L. and Ma, C., Biochem. et Biophys. Acta, 1097:107-143 (1990); Page, M. J. and Sydenham, M. A., Biotechnology 9:64-68 (1991).) Another useful selection marker is the enzyme glutamine synthase (GS) (Murphy et al., Biochem J. 227:277-279 (1991); Bebbington et al., Bio/Technology 10:169-175 (1992). Using these markers, the mammalian cells are grown in selective medium and the cells with the highest resistance are selected. These cell lines contain the amplified gene(s) integrated into a chromosome. Chinese hamster ovary (CHO) and NSO cells are often used for the production of proteins.

Derivatives of the plasmid pSV2-dhfr (ATCC Accession No. 37146), the expression vectors pC4 (ATCC Accession No. 209646) and pC6 (ATCC Accession No. 209647) contain the strong promoter (LTR) of the Rous Sarcoma Virus (Cullen et al., Molecular and Cellular Biology, 438-447 (March, 1985)) plus a fragment of the CMV-enhancer (Boshart et al., Cell 41:521-530 (1985).) Multiple cloning sites, e.g., with the restriction enzyme cleavage sites BamHI, XbaI and Asp718, facilitate the cloning of the gene of interest. The vectors also contain the 3′ intron, the polyadenylation and termination signal of the rat preproinsulin gene, and the mouse DHFR gene under control of the SV40 early promoter.

Specifically, the plasmid pC6, for example, is digested with appropriate restriction enzymes and then dephosphorylated using calf intestinal phosphates by procedures known in the art. The vector is then isolated from a 1% agarose gel.

A polynucleotide of the present invention is amplified according to protocols known in the art. If a naturally occurring signal sequence is used to produce the polypeptide of the present invention, the vector does not need a second signal peptide. Alternatively, if a naturally occurring signal sequence is not used, the vector can be modified to include a heterologous signal sequence. (See, e.g., WO 96/34891.)

The amplified fragment is isolated from a 1% agarose gel using a commercially available kit (“Geneclean,” BIO 101 Inc., La Jolla, Calif.). The fragment then is digested with appropriate restriction enzymes and again purified on a 1% agarose gel.

The amplified fragment is then digested with the same restriction enzyme and purified on a 1% agarose gel. The isolated fragment and the dephosphorylated vector are then ligated with T4 DNA ligase. E. coli HB101 or XL-1 Blue cells are then transformed and bacteria are identified that contain the fragment inserted into plasmid pC6 using, for instance, restriction enzyme analysis.

Chinese hamster ovary cells lacking an active DHFR gene are used for transfection. Five μg of the expression plasmid pC6 or pC4 is cotransfected with 0.5 μg of the plasmid pSVneo using lipofectin (Felgner et al., supra). The plasmid pSV2-neo contains a dominant selectable marker, the neo gene from Tn5 encoding an enzyme that confers resistance to a group of antibiotics including G418. The cells are seeded in alpha minus MEM supplemented with 1 mg/ml G418. After 2 days, the cells are trypsinized and seeded in hybridoma cloning plates (Greiner, Germany) in alpha minus MEM supplemented with 10, 25, or 50 ng/ml of metothrexate plus 1 mg/ml G418. After about 10-14 days single clones are trypsinized and then seeded in 6-well petri dishes or 10 ml flasks using different concentrations of methotrexate (50 nM, 100 nM, 200 nM, 400 nM, 800 nM). Clones growing at the highest concentrations of methotrexate are then transferred to new 6-well plates containing even higher concentrations of methotrexate (1 μM, 2 μM, 5 μM, 10 mM, 20 mM). The same procedure is repeated until clones are obtained which grow at a concentration of 100-200 μM. Expression of the desired gene product is analyzed, for instance, by SDS-PAGE and Western blot or by reversed phase HPLC analysis.

### Example 4

### Radiolabeled C35-Specific Antibodies Concentrate in Necrotic Regions of Viable Tumors Expressing C35

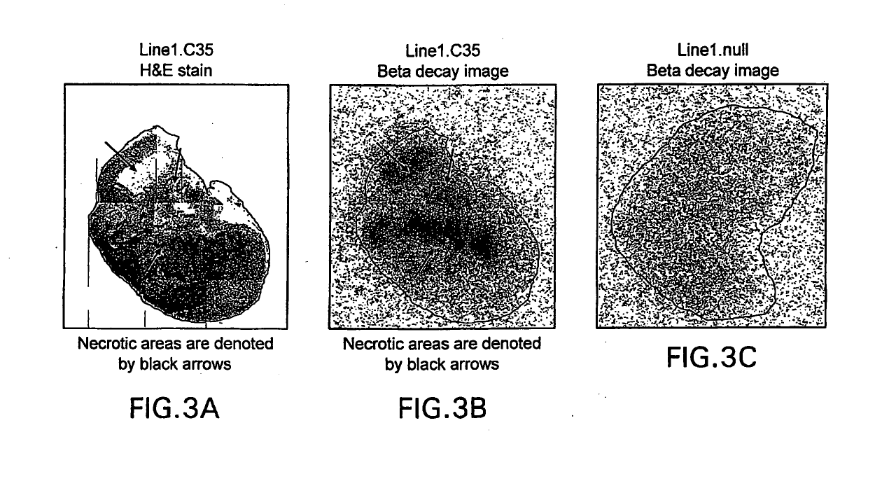

BALB/c mice were engrafted on opposite flanks with syngeneic non-small cell lung cancer derived Line 1 tumor cells that either had or had not been transfected with human C35. C35 protein expression was confirmed by immunohistochemical staining with anti-C35 antibodies. After 14 days in vivo growth, animals received intravenous injection of ¹²⁵I-labeled anti-C35 antibody. Animals were sacrificed 120 hrs after injection of radiolabeled antibodies and the concentration of anti-C35 antibodies in C35-positive and C35-negative tumors was determined by exposure of a tumor section to film. As shown in FIG. 3, radiolabeled anti-C35 antibodies concentrated only in the C35-positive and not the C35-negative tumors. Comparison of the distribution of label and an H&E stain for intact cells within the tumors, confirmed that under these conditions the labeled anti-C35 antibodies concentrated specifically in the necrotic regions of the C35-positive tumor.

### Example 5

### Protocol for Administration of Dosimetric and Therapeutic Radiolabeled Antibody

The radiolabeled antibody (or antibody fragment) compositions, which include both the dosimetric radiolabeled antibody and the therapeutic radiolabeled antibody, are administered intravenously or intraarterially in the form of an injection. The injectable radiolabeled antibody compositions will be infused into a vein or artery over the course of 5 minutes to about 60 minutes, preferably from 15 minutes to 30 minutes. Where the tumor is supplied by a known artery, intraarterial administration is preferred for the therapeutic radiolabeled antibody compositions. Both the dosimetric radiolabeled antibody and the therapeutic radiolabeled antibody will be administered as sterile aqueous solutions typically in physiologic phosphate-buffered saline or other vehicle suitable for parenteral injection. The initial dosimetric radiolabeled antibody dose will be approximately 5-100 mg of antibody which will deliver approximately 5-50mCi radiation. Approximately 5-10 days following the dosimetric dose, the therapeutic radiolabeled antibody will be administered at a dose of approximately 10-500 mg which will deliver as much as 300 mCi radiation for each therapeutic dose. This dosimetric/therapeutic regimen may be repeated. See also, U.S. Pat. No. 5,057,313 and U.S. Pat. No. 5,120,525.

### Example 6

### Cloning of Anti-C35 Mouse and Human Antibody Variable Region Genes into Deposited TOPO Clones

The immunoglobulin heavy and light chain variable regions were cloned into the TOPO vector (Invitrogen) by PCR amplification of the V region and TA cloning into the TOPO vector. This ligation system does not require restriction enzyme digestion (although the TOPO vector does incorporate EcoRI sites to allow subsequent excision of inserts). TA cloning takes advantage of naturally added 3′ A overhangs in the PCR amplification product of Taq polymerase which can then pair with 5′ T overhangs in the linearized vector provided in the TOPO cloning kit (Invitrogen).

To PCR amplify variable region genes for insertion into TOPO, we employed a downstream primer complementary to the 5′ end of the constant region sequence (different for heavy and light chains and for mouse and human) and a known fixed primer sequence added at the 5′ end of the variable region by 5′ RACE using the Invitrogen GeneRacer kit. These methods are well known to those skilled in the art.

### Example 7

### Cloning Variable Genes from Deposited Topo Clones into PCMV Expression Constructs Generation of Pcmv Expression Constructs

The construction of vaccinia transfer plasmids—pVHE, pVKE and pVLE—has been described in a previous patent application (US 2002 0123057 A1, “In vitro Methods of Producing and Identifying Immunoglobulin Molecules in Eukaryotic Cells”, published Sep. 5, 2002). To generate the mammalian expression vectors to express the immunoglobulin heavy and light chains, the expression cassettes, from NotI to SalI, were excised from these vaccinia transfer plasmids and cloned into the pCMV-Script vector (whose XhoI site in the vector multiple cloning site was destroyed by fill-in and blunt end ligation), resulting in the generation of pCMV-VH, pCMV-VK and pCMV-VL vectors. These expression cassettes contain the signal peptide, cloning sites for the V genes and the constant regions from the membrane-bound μ heavy chain and the κ light chain genes.

In pCMV-VH, the cassette contains the signal peptide from amino acid position −19 relative to the start codon [aa(−19)] to aa(−3), followed by aa(109 to 113) of the VH genes and the whole heavy chain constant region. The selected VH genes, from aa(−4) to aa(110) can be cloned into pCMV-VH at BssHII [aa(−4 to −3)] and BstEII [aa(109-110)] sites.

In pCMV-VK (kappa), the cassette contains the signal peptide from aa(−19) to aa(−2), followed by aa(104 to 107) of the VK genes and the whole kappa chain constant region. The selected VK genes, from aa(−3) to aa(105) can be cloned into pCMV-VK at ApaLI [aa(−3 to −2)] and XhoI [aa(104-105)] sites.

In pCMV-VL (lambda), the cassette contains the signal peptide from aa(−19) to aa(−2), followed by aa(103 to 107) of the VL genes and the whole kappa chain constant region. The selected VL genes, from aa(−3) to aa(104) can be cloned into pCMV-VL at ApaLI [aa(−3 to −2)] and HindIII [aa(103-104)] sites. The resulting lambda light chain will exhibit the VλCκ chimeric structure.

To express the selected antibodies as secreted human IgG1, the constant region of IgG1 was cloned from B cells or bone marrow cells by RT-PCR. The primer set used was:

5′ forward primer:

(SEQ ID NO: 15)

5′-ATTAGGATCCGGTCACCGTCTCCTCAGCC-3′
3′ reverse primer:

(SEQ ID NO: 16)

5′-ATTAGTCGACTCATTTACCCGGAGACAGGGA-3′

The resulting PCR product exhibits the following structure: BamHI-BstEII(aa109-110)-(aa111-113)-Cγ₁-TGA-SalI. The PCR product was subcloned into pBluescriptII/KS at BamHI and SalI sites to carry out site directed mutagenesis employing standard protocols to remove the internal BstEII located at the CHI region via silent mutation. The resulting Cy, was then subcloned into pCMV-VH at BstEII and SalI to generate pCMV-Cy, to direct the expression of secreted IgG1 heavy chain, once a VH gene is subcloned into this vector at BssHII/BstEII.

The sequence of IgG1-secreted, human gamma1 heavy chain leader and constant region cassette for insertion of V genes follows.

Underline=restriction sites
Bold=Constant region
Bold/italics=Signal peptide

(SEQ ID NO:17)
Not 1 NcoI
gcggccgcaaaccatgggatggagctgtatcatcctcttcttggtagcaa
cagctacag
BssHII BsteII
gcgcgcatatggtcaccgtctcctc

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

Sal1

[symbol]

The sequence of human light chain leader and kappa constant region cassette for insertion of Vκ genes follows.

```
(SEQ ID NO: 18)
  Not 1 NcoI
gcggccgcaaaccatgggatggagctgtatcatcctcttcttggtagcaa
cagctacag
    ApaL1 XhoI
gcgtgcacttgactcgagatcaaa

[symbol]

[symbol]

[symbol]

[symbol]

[symbol]

[symbol]

[symbol]

[symbol]

[symbol]

[symbol]

[symbol]

[symbol]




                                                                                                                Sal1

[symbol]

[symbol]

[symbol]

```

The sequence of human light chain leader and kappa constant region cassette for insertion of Vλ genes follows.

```
(SEQ ID NO:19)
    Not 1 Ncol
gcggccgcaaaccatgggatggagctgtatcatcctcttcttggtagcaacagctacag
                      ApaL1 HindIII
gcgtgcacttgactcgagaagcttaccgtcct

[symbol]

[symbol]

[symbol]

[symbol]

[symbol]

[symbol]

[symbol]

[symbol]

[symbol]

[symbol]

[symbol]

[symbol]

[symbol]

[symbol]




      SAL 1

[symbol]

```

To construct vectors that express secreted human antibodies of other isotypes, including secreted forms of IgG2, IgG3, IgG4, IgA, IgD, IgE and IgM, the same approach can be taken to clone the respective constant regions, to mutagenize any internal BstEII site, and to substitute the Cγ₁ with the constant regions of other isotypes between the BstEII and SalI sites in the pCMV-Cγ₁ vector.

To clone the constant regions of other isotypes, the following primer pairs were used:

(SEQ ID NO:20)

| | |
|---|---|
| IgG2-F: | 5′-ATTAGGATCCGGTCACCGTCTCCTCAGCC-3′ |

(SEQ ID NO:21)

| | |
|---|---|
| IgG2-R: | 5′-ATTAGTCGACTCATTTACCCGGAGACAGGGA-3′ |

(SEQ ID NO:22)

| | |
|---|---|
| IgG3-F: | 5′-ATTAGGATCCGGTCACCGTCTCCTCAGCT-3′ |

(SEQ ID NO:23)

| | |
|---|---|
| IgG3-R: | 5′-ATTAGTCGACTCATTTACCCGGAGACAGGGA-3′ |

(SEQ ID NO:24)

| | |
|---|---|
| IgG4-F: | 5′-ATTAGGATCCGGTCACCGTCTCCTCAGCT-3′ |

(SEQ ID NO:25)

| | |
|---|---|
| IgG4-R: | 5′-ATTAGTCGACTCATTTACCCAGAGACAGGGA-3′ |

(SEQ ID NO:26)

| | |
|---|---|
| IgA1-F: | 5′-ATTAGGATCCGGTCACCGTCTCCTCAGCAT-3′ |

(SEQ ID NO:27)

| | |
|---|---|
| IgA1-R: | 5′-ATTAGTCGACTCAGTAGCAGGTGCCGTCCAC-3′ |

(SEQ ID NO:28)

| | |
|---|---|
| IgA2-F: | 5′-ATTAGGATGCGGTCACCGTCTCCTGAGCAT-3′ |

(SEQ ID NO:29)

| | |
|---|---|
| IgA2-R: | 5′-ATTAGTCGACTCAGTAGCAGGTGCCGTCGAC-3′ |

(SEQ ID NO:30)

| | |
|---|---|
| IgD-F: | 5′-ATTAGGATCCGGTCACCGTCTCCTCAGCAC-3′ |

(SEQ ID NO:31)

| | |
|---|---|
| IgD-R: | 5′-ATTAGTCGACTCATTTCATGGGGCCATGGTC-3′ |

(SEQ ID NO:32)

| | |
|---|---|
| IgE-F: | 5′-ATTAGGATCCGGTCACCGTCTCCTCAGCC-3′ |

(SEQ ID NO:33)

| | |
|---|---|
| IgE-R: | 5′-ATTAGTCGACTCATTTACCGGGATTTACAGA-3′ |

(SEQ ID NO:34)

| | |
|---|---|
| IgM-F: | 5′-ATTAGGATCCGGTCACCGTCTCCTCAGGG-3′ |

(SEQ ID NO:35)

| | |
|---|---|
| IgM-R: | 5′-ATTAGTCGACTCAGTAGCAGGTGCCAGCTGT-3′ |

Note that, because of the high degree of sequence conservation, primers used are the same between IgG1 and IgG2, between IgG3 and IgG4, and between IgA1 and IgA2.

Cloning Variable genes from Topo clones into pCMV expression constructs
Step 1: Generation of V-gene fragments.
A. Human v-Genes

MMH1

1. Digest MMH1 plasmid DNA (clone H0009) with BssHII (GCGCGC(SEQ ID NO:36)) and BsteII (GGTCACC (SEQ ID NO:37)) using standard protocols.

2. Resolve DNA on agarose gel using standard protocols.

3. Excise 357 bp fragment from gel and isolate DNA using standard protocols.

MMK1

1. Digest MMK1 plasmid DNA (clone L0010) with ApaL1 (GTGCAC(SEQ ID NO:38)) and Xho1 (CTCGAG (SEQ ID NO:39)) using standard protocols.

2. Resolve DNA on agarose gel using standard protocols.

3. Excise 343 bp fragment from gel and isolate DNA using standard protocols.

B. Mouse Hybridoma v-Genes:

1F2 VK

1. The mouse hybridoma v-gene must be PCR amplified from the ATCC deposited clone 1F2K using the following primers. This is necessary to create a chimeric antibody of the mouse v-gene with human constant region in the human kappa light chain constant region expression cassette.

1F2VK forward primer:

(SEQ ID NO:40)

5′-tatccgtgcactccCAAATTGTTCTCACCCAGTCTCCAG-3′
1F2VK reverse primer:

(SEQ ID NO:41)

5′-atattctcgAGCTTGGTCCCCCCTCCGAA-3′

(Lowercase=non-homologous to mouse 1F2 VK sequence, includes restriction site. CAPITALS=homologous to mouse 1F2 VK sequence)

2. Digest 331 bp PCR product with ApaL1 (GTGCAC (SEQ ID NO:42)) and XhoI (CTCGAG (SEQ ID NO:43)) using standard protocols.

3. Resolve DNA on agarose gel using standard protocols.

4. Excise 315 bp digested fragment from gel and isolate DNA using standard protocols.

1F2 VH

1. The mouse hybridoma v-gene must be PCR amplified from the ATCC deposited clone 1F2G using the following primers. This is necessary to create a chimeric antibody of the mouse v-gene with human constant region in the human heavy chain constant region expression cassette.

1F2VH forward primer:

(SEQ ID NO:44)

5′-tataagcgcgcactccGATGTACAGCTTCAGGAGTCAGGAC
1F2VH reverse primer:

(SEQ ID NO:45)

5′-atattgGTGACCAGAGTCCCTTGGCCCC-3′

(Lowercase=Non-Homologous, Contains Restriction Sites. Capitals=Homologous)

2. Digest 360 bp PCR product with BssHII (GCGCGC (SEQ ID NO:36)) and BsteII (GGTCACC (SEQ ID NO:37)) using standard protocols.

3. Resolve DNA on agarose gel using standard protocols.

4. Excise gel slice containing 343 bp digested DNA fragment and isolate DNA using standard protocols.

1B3VK

1. The mouse hybridoma v-gene must be pcr amplified from the deposited clone 1B3K using the following primers. This is necessary to create a chimeric antibody of the mouse v-gene with human constant region in the human kappa light chain constant region expression cassette.

1B3VK forward primer:

(SEQ ID NO:46)

5′-tatccgtgcactccGATGTCCAGATAACCCAGTCTCCATC-3′
1B3VK reverse primer:

(SEQ ID NO:47)

5′-atattctcgAGCTTGGTCCCAGCACCGAA-3′

(Lowercase=Non-Homologous, Contains Restriction Sites. Capitals=Homologous)

2. Digest 334 bp PCR product with ApaL1 (GTGCAC (SEQ ID NO:42)) and Xho1 (CTCGAG (SEQ ID NO:43)) using standard protocols.

3. Resolve DNA on agarose gel using standard protocols.

4. Excise gel slice containing digested 322 bp DNA fragment and isolate DNA using standard protocols

1B3VH

1. The mouse hybridoma v-gene must be pcr amplified from the deposited clone I B3G using the following primers. This is necessary to create a chimeric antibody of the mouse v-gene with human constant region in the human heavy chain constant region expression cassette.

1B3VH forward primer:

(SEQ ID NO:48)

5′-tataagcgcgcactccGAGGTGCAGCTTCAGGAGTCAGGAC-3′
1B3VH reverse primer:

(SEQ ID NO:49)

5′-atattGGTGACCGTGGTCCCAGCG-3′

(Lowercase=non-homologous, contains restriction sites. CAPITALS=homologous 2. Digest 378 bp PCR product with BssHII (GCGCGC (SEQ ID NO:36)) and BsteII (GGTCACC (SEQ ID NO:37)) using standard protocols.

3. Resolve DNA on agarose gel using standard protocols.

4. Excise gel slice containing 366 bp digested DNA fragment and isolate DNA using standard protocols.

Step 2: Assembly of Expression Constructs

1. Digest pCMV-VH and pCMV-VK expression vectors with the appropriate enzymes using standard protocols:
a. pCMV-VH—BsteII and BssHII
b. pCMV-VK-ApaL1 and Xho I

2. Resolve DNA on agarose gel and excise linearized vector using standard protocols.

3. Isolate DNA from gel slice using standard protocols.

4. Ligate light chain v-genes into pCMV-VK and heavy chain v-genes into pCMV-VH using standard protocols.

5. Transform ligated DNA into competent cells and isolate plasmid DNA using standard protocols.

### Example 8

### Sequences of Immunoglobulin Constant Regions

The following genes and encoded amino acids sequences may be used to prepare humanized antibodies, human variant antibodies, chimeric antibodies, and fragments thereof.

Homo sapiens G2 gene for immunoglobulin constant region (IgG2 (n-) allotype)

| (GenBank No. Z49802) | |
|---|---|
| (SEQ ID NO:50) | |
| 1 | tcttctctct gcagagcgca aatgttgtgt cgagtgccca ccgtgcccag gtaagccagc |
| 61 | ccaggcctcg ccctccagct caaggcggga caggtgccct agagtagcct gcatccaggg |
| 121 | acaggcccca gctgggtgct gacacgtcca cctccatctc ttcctcagca ccacctgtgg |
| 181 | caggaccgtc agtcttcctc ttccccccaa aacccaagga caccctcatg atctcccgga |
| 241 | cccctgaggt cacgtgcgtg gtggtggacg tgagccacga agaccccgag gtccagttca |
| 301 | actggtacgt ggacggcgtg gaggtgcata atgccaagac aaagccacgg gaggagcagt |
| 361 | tcaacagcac gttccgtgtg gtcagcgtcc tcaccgttgt gcaccaggac tggctgaacg |
| 421 | gcaaggagta caagtgcaag gtctccaaca aaggcctccc agcccccatc gagaaaacca |
| 481 | tctccaaaac caaaggtggg acccgcgggg tatgagggcc acatggacag acggcggctt |
| 541 | cggcccaccc tctgccctgg gagtgaccgc tgtgccaacc tctgtcccta cagggcagcc |
| 601 | ccgagaacca caggtgtaca ccctgccccc atcccgggag gagatgacca agaaccaggt |
| 661 | cagcctgacc tgcctggtca aaggcttcta ccccagcgac atcgccgtgg agtgggagag |
| 721 | caatgggcag ccggagaaca actacaagac cacacctccc atgctggact ccgacggctc |
| 781 | cttcttcctc tacagcaagc tcaccgtgga caagagcagg tggcagcagg ggaacgtctt |
| 841 | ctcatgctcc gtgatgcatg aggctctgca caaccactac acgcagaaga gcctctccct |
| 901 | gtctccgggt aaatgagtgc cacggccggc aagcc |

H. sapiens G2 gene for immunoglobulin constant region (IgG2 (n+) allotype) (GenBank

| (GenBank No. Z49801) | |
|---|---|
| (SEQ ID NO:51) | |
| 1 | tcttctctct gcagagcgca aatgttgtgt cgagtgccca ccgtgcccag gtaagccagc |
| 61 | ccaggcctcg ccctccagct caaggcggga caggtgccct agagtagcct gcatccaggg |
| 121 | acaggcccca gctgggtgct gacacgtcca cctccatctc ttcctcagca ccacctgtgg |
| 181 | caggaccgtc agtcttcctc ttccccccaa aacccaagga caccctcatg atctcccgga |
| 241 | cccctgaggt cacgtgcgtg gtggtggacg tgagccacga agaccccgag gtccagttca |
| 301 | actggtacgt ggacggcgtg gaggtgcata atgccaagac aaagccacgg gaggagcagt |
| 361 | tcaacagcac gttccgtgtg gtcagcgtcc tcaccgttgt gcaccaggac tggctgaacg |
| 421 | gcaaggagta caagtgcaag gtctccaaca aaggcctccc agcccccatc gagaaaacca |
| 481 | tctccaaaac caaaggtggg acccgcgggg tatgagggcc acatggacag acggcggctt |
| 541 | cggcccaccc tctgccctgg gagtgaccgc tgtgccaacc tctgtcccta cagggcagcc |
| 601 | ccgagaacca caggtgtaca ccctgccccc atcccgggag gagatgacca agaaccaggt |
| 661 | cagcctgacc tgcctggtca aaggcttcta ccccagcgac atcgccgtgg agtgggagag |
| 721 | caatgggcag ccggagaaca actacaagac cacacctccc atgctggact ccgacggctc |
| 781 | cttcttcctc tacagcaagc tcaccgtgga caagagcagg tggcagcagg ggaacgtctt |
| 841 | ctcatgctcc gtgatgcatg aggctctgca caaccactac acgcagaaga gcctctccct |
| 901 | gtctccgggt aaatgagtgc cacggccggc aagcc |

Homo sapiens CH gene encoding immunoglobulin, constant region, heavy chain, alpha-2 subunit (GenBank No. AJ012264) (SEQ ID NO:52)

| | |
|---|---|
| 1 | ctcgaggacc tgctcttagg ttcagaagcg aacctcacgt gcacactgac cggcctgaga |
| 61 | gatgcctctg gtgccacctt cacctggacg ccctcaagtg ggaagagcgc tgttcaagga |
| 121 | ccacctgagc gtgacctctg tggctgctac agcgtgtcca gtgtcctgcc tggctgtgcc |
| 181 | cagccatgga accatgggga gaccttcacc tgcactgctg cccaccccga gttgaagacc |
| 241 | ccactaaccg ccaacatcac aaaatccggt gggtccagac cctgctcggg gccctgctca |
| 301 | gtgctctggt ttgcaaagca tattcctggc ctgcctcctc cctcccaatc ctgggctcca |
| 361 | gtgctcatgc caagtacaca gggaaactga ggcaggctga ggggccagga cacagcccag |
| 421 | ggtgcccacc agagcagagg ggctctctca tcccctgccc agccccctga cctggctctc |
| 481 | taccctccag gaaacacatt ccggcccgag gtccacctgc tgccgccgcc gtcggaggag |
| 541 | ctggccctga acgagctggt gacgctgacg tgcctggcac gtggcttcag ccccaaggat |
| 601 | gtgctggttc gctggctgca ggggtcacag gagctgcccc gcgagaagta cctgacttgg |
| 661 | gcatcccggc aggagcccag ccagggcacc accacctacg ctgtaaccag catactgcgc |
| 721 | gtggcagctg aggactggaa gaagggggag accttctcct gcatggtggg ccacgaggcc |
| 781 | ctgccgctgg ccttcacaca gaagaccatc gaccgcatgg cgggtaaacc cacccacatc |
| 841 | aatgtgtctg ttgtcatggc ggaggcggat ggcacctgct actgagccgc ccgcctgtcc |
| 901 | ccacccctga ataaactcca tgctccccca agcagcccca cgcttccatc cggcgcctgt |
| 961 | ctgtccatcc tcagggtctc agcacttggg aaagggccag ggcatggaca gggaagaata |
| 1021 | ccccctgccc tgagcctcgg ggggcccctg gcacccccat gagactttcc accctggtgt |
| 1081 | gagtgtgagt tgtgagtgtg agagtgtgtg gtgcaggagg cctcgctggt gtgagatctt |
| 1141 | aggtctgcca aggcaggcac agcccaggat gggttctgag agacgcacat gccccggaca |
| 1201 | gttctgagtg agcagtggca tggccgtttg tccctgagag agccgcctct ggctgtagct |
| 1261 | gggagggaat agggagggta aaaggagcag gctagccaag aaaggcgcag gtagtggcag |
| 1321 | gagtggcgag ggagtgaggg gctggactcc agggccccac tgggaggaca agctccagga |
| 1381 | gggccccacc accctagtgg gtgggcctca ggacgtccca ctgacgcatg caggaagggg |
| 1441 | cacctcccct taaccacact gctctgtacg gggcacgtgg gcacacatgc acactcacac |
| 1501 | tcacatatac gcctgagccc tgcaggagtg gaacgttcac agcccagacc cagttccaga |
| 1561 | aaagccaggg gagtcccctc ccaagccccc aagctcagcc tgctccccca ggcccctctg |
| 1621 | gcttccctgt gtttccactg tgcacagctc agggaccaac tccacagacc cctcccaggc |
| 1681 | agcccctgct ccctgcctgg ccaagtctcc catcccttcc taagcccaac taggacccaa |
| 1741 | agcatagaca gggaggggcc gcgtggggtg gcatcagaag |

Homo sapiens constant region, heavy chain, alpha-2 subunit (GenBank No. CAA09968.1)

(SEQ ID NO:53)

LEDLLLGSEANLTCTLTGLRDASGATFTWTPSSGKSAVQGPPERDLCGCY
SVSSVLPGCAQPWNHGETFTCTAAHPELKTPLTANITKSGNTFRPEVHLL
PPPSEELALNELVTLTCLARGFSPKDVLVRWLQGSQELPREKYLTWASRQ
EPSQGTTTYAVTSILRVAAEDWKKGETFSCMVGHEALPLAFTQKTIDRMA
GKPTHINVSVVMAEADGTCY

Homo sapiens partial mRNA for immunoglobulin heavy chain constant region alpha 1 (IGHA1 gene) (GenBank No. AJ294729) (SEQ ID NO:54)

| | |
|---|---|
| 1 | gcaagcttga ccagccccaa ggtcttcccg ctgagcctct gcagcaccca gccagatggg |
| 61 | aacgtggtca tcgcctgcct ggtccagggc ttcttccccc aggagccact cagtgtgacc |
| 121 | tggagcgaaa gcggacaggg cgtgaccgcc agaaacttcc cacccagcca ggatgcctcc |
| 181 | ggggacctgt acaccacgag cagccagctg accctgccgg ccacacagtg cctagccggc |
| 241 | aagtccgtga catgccacgt gaagcactac acgaatccca gccaggatgt gactgtgccc |
| 301 | tgcccagttc cctcaactcc acctacccca tctccctcaa ctccacctac cccatctccc |
| 361 | tcatgctgcc acccccgact gtcactgcac cgaccggccc tcgaggacct gctcttaggt |
| 421 | tcagaagcga acctcacgtg cacactgacc ggcctgagag atgcctcagg tgtcaccttc |
| 481 | acctggacgc cctcaagtgg gaagagcgct gttcaaggac cacctgaccg tgacctctgt |
| 541 | ggctgctaca gcgtgtccag tgtcctgtcg ggctgtgccg agccatggaa ccatgggaag |
| 601 | accttcactt gcactgctgc ctaccccgag tccaagaccc cgctaaccgc caccctctca |
| 661 | aaatccggaa acacattccg gcccgaggtc cacctgctgc cgccgccgtc ggaggagctg |
| 721 | gccctgaacg agctggtgac gctgacgtgc ctggcacgtg gcttcagccc caaggatgtg |
| 781 | ctggttcgct ggctgcaggg gtcacaggag ctgccccgcg agaagtacct gacttgggca |
| 841 | tcccggcagg agcccagcca gggcaccacc accttcgctg tgaccagcat actgcgcgtg |
| 901 | gcagccgagg actggaagaa gggggacacc ttctcctgca tggtgggcca cgaggccctg |
| 961 | ccgctggcct tcacacagaa gaccatcgac cgcttggcgg gtaaacccac ccatgtcaat |
| 1021 | gtgtctgttg tcatggcgga ggtggacggc acctgctac |

Immunoglobulin heavy chain constant region alpha 1 (GenBank No. CAC20453.1) (SEQ ID NO:55)

ASLTSPKVFPLSLCSTQPDGNVVIACLVQGFFPQEPLSVTWSESGQGVTA
RNFPPSQDASGDLYTTSSQLTLPATQCLAGKSVTCHVKHYTNPSQDVTVP
CPVPSTPPTPSPSTPPTPSPSCCHPRLSLHRPALEDLLLGSEANLTCTLT
GLRDASGVTFTWTPSSGKSAVQGPPDRDLCGCYSVSSVLSGCAEPWNHGK
TFTCTAAYPESKTPLTATLSKSGNTFRPEVHLLPPPSEELALNELVTLTC
LARGFSPKDVLVRWLQGSQELPREKYLTWASRQEPSQGTTTFAVTSILRV
AAEDWKKGDTFSCMVGHEALPLAFTQKTIDRLAGKPTHVNVSVVMAEVDG
TCY

Additional constant region sequences (human IgM1, IgM2, IgD1, IgA1, IgG1, IgG3, IgE1, IgE2, kappa, and lambda; mouse IgM1, kappa, and lambda; rabbit IgM1, kappa, and lambda; and dog IgM1) may be found at pages 296-300 of FUNDAMENTAL IMMUNOLOGY (3d ed.), William E. Paul (ed.), Raven Press, New York, N.Y. (1993). Many other constant region sequences (polynucleotide and amino acid) are know in the art and may be used in the present invention.

### Example 9

### Combination Radioimmunotherapy and Chemotherapy

The combination of chemotherapy and anti-C35 radioimmunotherapy was shown to be more effective at reducing tumor volume than either therapy alone. In a first experiment, the effect of combination radioimmunotherapy with ¹³¹I labeled 1B3 anti-C35 monoclonal antibody and chemotherapy with 5-FluoroUracil (FU) at 150 mg/kg, together with Leucovorin (LV) at 100 mg/kg was tested in Swiss nude mice grafted with Colau.C35 tumor cells. Colau.C35 are a C35 antigen positive clone of Colau cells that were tissue culture adapted from a human colon carcinoma and transduced with a C35 retroviral recombinant.

Chemotherapy was initiated on day 11 following tumor graft and 300 μCi of ¹³¹I-labeled 1B3 anti-C35 monoclonal antibody was administered on day 14. Tumor growth was followed for up to 8 weeks.

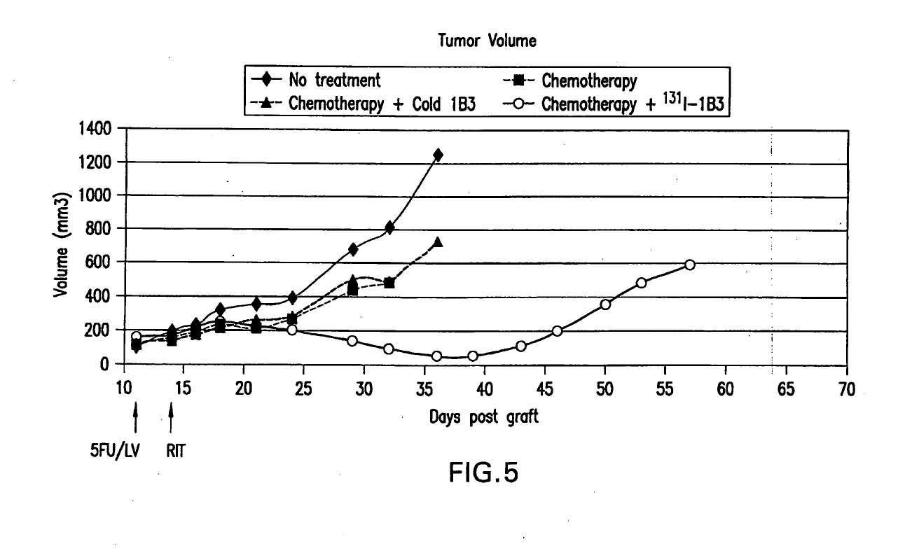

The results in FIG. 5 show inhibition of tumor growth in the group that received combination radioimmunotherapy and chemotherapy in comparison to the group that received chemotherapy alone or chemotherapy and non-radiolabeled (“cold”) 1B3 anti-C35 antibody. Standard parameters of growth inhibition were calculated for the group receiving combination radioimmunotherapy and chemotherapy in comparison to the untreated control group.

As shown in FIG. 5, Tumor Doubling Delay (TDD) equals 3.8 (at 400 mm³ tumor volume) where TDD equals (Treated—Control{in days to the specified volume})/TVDT and TVDT=Tumor Volume Doubling Time of Control {during exponential growth phase}. Log Cell Kill (LCK) is defined as TDD/3.3=1.15, which meets the accepted standard for an effective tumor therapy (See, e.g., Skipper H E et al., Cancer Chemotherapy Rep. 35:1-111 (1964); Coldman A J and Goldie J H, Mathematical Biosciences 65:291-307 (1983); and Norton L and Simon R, Cancer Treat. Rep. 61:1307-1317 (1977)).

In a second experiment, the effect of combination radioimmunotherapy with ¹³¹I labeled 1B3 anti-C35 monoclonal antibody and chemotherapy with cisplatin at 2 mg/kg on day 15 and 18 was tested in Swiss nude mice grafted with Colau.C35 tumor cells. Cispaltin was administered on days 15 and 18 following tumor graft. 300 μCi of ¹³¹I-labeled 1B3 anti-C35 monoclonal antibody was administered on day 21. Tumor growth was followed for up to 10 weeks.

In the same experiment, separate groups of Swiss nude mice grafted with Colau.C35 tumor cells were treated with either 5-FluoroUracil (5FU) at 180 mg/kg, together with Leucovorin (LV) at 120 mg/kg or this same chemotherapy regimen administered on day 18 followed by 300 μCi of ¹³¹I-labeled 1B3 anti-C35 monoclonal antibody administered on day 21.

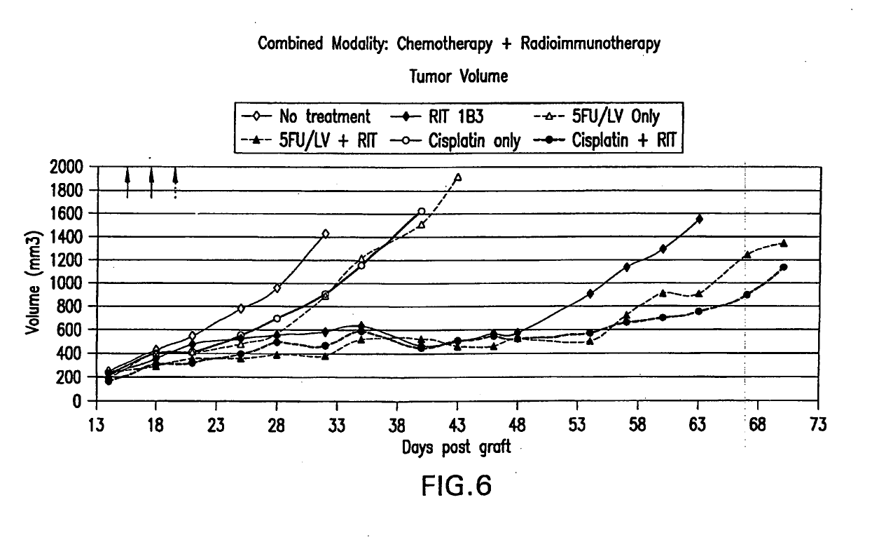

The results in FIG. 6 show some inhibition of Colau.C35 tumor growth in the group that received chemotherapy alone (either cisplatin or 5FU/LV), greater inhibition in the group that received ¹³¹I-labeled 1B3 anti-C35 monoclonal antibody, and even greater inhibition of tumor growth in the group that received combination chemotherapy and ¹³¹I-labeled 1B3 anti-C35 monoclonal antibody. See Table 6, below.

TABLE 6
COMPARISON OF EFFECTS OF THERAPEUTIC
MODALITIES ON TUMOR VOLUME IN FIG. 6

| | 5FU/LV | Cisplatin | RIT: | 5FU/LV + | Cisplatin + |
|---|---|---|---|---|---|
| | alone | alone | ¹³¹I-1B3 | RIT | RIT |
| T − C | 5 | 5 | 29 | 37 | 40 |
| (days) | | | | | |
| TDD | 0.66 | 0.66 | 3.84 | 4.89 | 5.29 |
| LCK | 0.20 | 0.20 | 1.16 | 1.48 | 1.60 |

RIT = radioimmunotherapy
T − C = difference in time for treated (T) and control (C) tumors to reach a given volume (1200 mm³)
TDD = Tumor doubling delay = T − C/tumor volume doubling time of untreated
LCK = Log Cell Kill = TDD/3.32

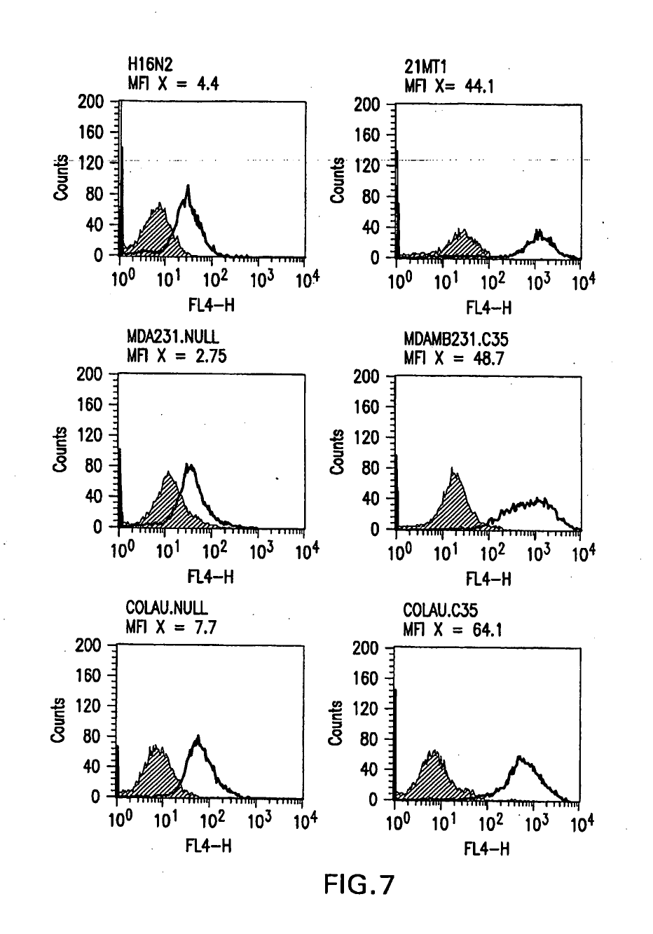

For convenience of use in these experimental models, a tumor xenograft was selected that grew relatively rapidly and had to be transduced with recombinant C35. However, the success of combination therapy was not due to abnormally high levels of C35 expression in the transduced tumor. FIG. 7 shows that C35 expression was very similar in tumors such as 21MT1 that naturally express C35, and in C₃₋₅-transduced tumors such as Colau.C35 and MDA231.C35. Cells were stained with Alexa-647 conjugated anti-C35 MAb 1F2 or isotype control. “MFI X” is the ratio of the mean fluorescence intensity of 1F2/mean fluorescence intensity of isotype control. H16N2, derived from normal breast epithelium, and MDAMB231, a breast tumor, and Colau, a colon tumor, express low basal levels of C35. 21MT1, derived from breast carcinoma, naturally expresses high levels of C35. Colau and MDA231 were transduced with empty vector (null) or human C35 recombinant vector. All tumors were grown in vivo, tumors were excised, dissociated and stained.

### Example 10

### Determination of Maximum Tolerated Dose (MTD) of a Chemotherapeutic Agent

Because of concerns regarding cumulative dose-limiting bone marrow toxicity when combining chemotherapy and radioimmunotherapy, it is necessary to determine the Maximum Tolerated Dose (MTD) of combination therapy and, where toxicity of the two therapeutic agents is additive, adopt strategies that will permit administration of both toxic agents. MTD is established by regulatory criteria related to the time required for platelet and white cell recovery in peripheral circulation. Such standards are familiar to those skilled in the art. In the case of murine models, an often employed surrogate definition of MTD is the maximum dose that results in an average of less than 20% weight loss or less than 10% mortality. Currently established MTD for the most common chemotherapeutic agents employed in standard clinical protocols or in animal models are shown in Tables 7 to 10, below.

TABLE 7
REPRESENTATIVE CHEMOTHERAPY PROTOCOLS
IN XENOGRAFT MODELS (NUDE MICE)

| Cytotoxic | Maximum | | | | |
|---|---|---|---|---|---|
| Drug | Tolerated Dose | | Dose + RIT | | Schedule |
| Fluorouracil/ | 180/120 | mg/kg ¹ | 150/100 | mg/kg | bolus, i.v. |
| leucovorin | | | | | |
| Oxaliplatin | 5 | mg/kg ³ | TBD | | i.p. every |
| | | | | | day for 5 |
| | | | | | cycles |
| Cisplatin | 4 | mg/kg ^{4, 5} | 2 | mg/kg | i.v. every 3 |
| | | | | | days for 2 |
| | | | | | cycles |
| Irinotecan | 15 | mg/kg ³ | TBD | | i.p. every |
| | | | | | day for 5 |
| | | | | | cycles |
| Taxol | No toxicity ² | | 30 | mg/kg ^{4, 6} | i.v. every 7 |
| | | | | | days for 2- |
| | | | | | 3 cycles |
| Cyclo- | No toxicity ² | | 175 | mg/kg ^{4, 7} | i.v. every 7 |
| phosphamide | | | | | days for 2 |
| | | | | | cycles |
| Adriamycin | 10 | mg/kg ¹ | 8 | mg/kg | i.v. every 4 |
| | | | | | days for 3 |
| | | | | | cycles |

| | | | | |
|---|---|---|---|---|
| Gemcitabine | No toxicity ⁵ | 120 | mg/kg ⁵ | i.p. every 3 |
| | | | | days for 4 |
| | | | | cycles |

| | | | | |
|---|---|---|---|---|
| Vinorelbine | 20 | mg/kg ⁴ | TBD | bolus, i.v. |

| | | | | |
|---|---|---|---|---|
| External Beam | No toxicity ² | 20 | Gy | delivered |
| Irradiation | | | | locally |

Notes
TBD: To be determined
¹ MTD confirmed by present inventors. Maximum Tolerated Dose is defined as ≧20% average weight loss and/or >10% lethality.
² Non-toxic dose at indicated schedule.
³ Fichtner I et al. Anticancer drug response and expression of molecular markers in early-passage xenotransplanted colon carcinomas. Eur J Can 2004. 40: 298-307.
⁴ Villena-Heinsen C et al. Human ovarian cancer xenografts in nude mice: chemotherapy trials with paclitaxel, cisplatin, vinorelbine, and titanocene dichloride. Anticancer Drugs 1998. 9: 557-563.
⁵ Higgins B. et al. Antitumor activity of erlotinib (OSI-774, Tarceva) alone or in combination in human non-small cell lung cancer tumor xenograft models. Anti-Cancer Drugs 2004. 15: 503-512.
⁶ Kraeber-Bodere F. et al. Enhanced Antitumor Activity of Combined pretargeted radioimmunotherapy and Paclitaxel in Medullary Thyroid Cancer Xenograft. Mol Can Ther 2002. 1: 267-274
⁷ Kraus-Berthier, L et al. Histology and sensitivity to anticancer Drugs of two human non-small cell lung carcinomas implanted in the pleural cavity of nude mice. 2000. Clin Can Res 6: 297-304.

TABLE 8
REPRESENTATIVE BREAST CANCER
CHEMOTHERAPY PROTOCOLS

| Regimen | Cytotoxic Drug | Dosage | Schedule |
|---|---|---|---|
| AC | doxorubicin | 60 mg/m2 IV | Repeat every |
| | cyclophosphamide | | 3 weeks for |
| | | | 4 cycles |
| CAF | cyclophosphamide | 600 mg/m2 IV | Repeat every |
| | | 100 mg/m2/day PO | 28 days for |
| | | on days 1-14 | 6 cycles |
| | doxorubicin | 30 mg/m2 IV on | |
| | | days 1 and 8 | |
| | fluorouracil | 500 mg/m2 IV on | |
| | | days 1 and 8 | |
| CMF | cyclophosphamide | 100 mg/m2/day PO | Repeat every |
| | | on days 1-14 | 28 days for |
| | | | 6 cycles |
| | methotrexate | 40 mg/m2 IV on | |
| | | days 1 and 8 | |
| | fluorouracil | 600 mg/m2 IV on | |
| | | days 1 and 8 | |
| | vinorelbine | 30 mg/m2 IV on | Repeat every |
| | | days 1 and 8 | 21 days for |
| | | | 6-8 cycles |
| | paclitaxel | 175 mg/m2 IV | Repeat every |
| | | | 21 days for |
| | | | 6 cycles |

Sources: DataMonitor Pipeline Insight: Breast Cancer June 2004; http://www.bccancer.bc.ca

TABLE 9
REPRESENTATIVE COLON CANCER
THERAPY PROTOCOLS

| Regimen | Drug | Dosage | Schedule |
|---|---|---|---|
| | Leucovorin | 20 mg/m2/day × | Every 28 days × 6 |
| | | 5 days (d 1-5) IV | cycles |
| | | prior to fluorouracil | |
| | Fluorouracil | 425 mg/m2/day × | |
| | | 5 days (d 1-5) IV | |
| | Irinotecan | 350 20 mg/m2 IV | Repeat every 21 |
| | | | days for 2-6 |
| | | | cycles depending |
| | | | on clinical |
| | | | benefit and |
| | | | toxicity |
| FOLFOX | Oxaliplatin | 100 mg/m2 IV | Repeat every 14 |
| | Leucovorin | 400 mg/m2 IV | days for a |
| | | | maximum of 12 |
| | | | cycles |
| | Fluorouracil | 400 mg/m2 IV | |
| | | bolus after the | |
| | | Leucovorin, THEN | |
| | Fluorouracil | 2400 mg/m2 IV | |
| | | over 46 hours | |
| FOLFIRI | Irinotecan | 180 mg/m2 IV | Repeat every 14 |
| | Leucovorin | 400 mg/m2 IV | days for a |
| | | | maximum of 12 |
| | | | cycles |
| | Fluorouracil | 400 mg/m2 IV | |
| | | bolus after the | |
| | | Leucovorin, THEN | |
| | Fluorouracil | 2400 mg/m2 IV | |
| | | over 46 hours | |

Source: http://www.bccancer.bc.ca

TABLE 10
REPRESENTATIVE LUNG CANCER
CHEMOTHERAPY PROTOCOLS

| Drug | Dose | Schedule |
|---|---|---|
| Docetaxel | 75 mg/m2 IV | Repeat every 21 days × 6 |
| | | cycles |
| Docetaxel | 75 mg/m2 IV | Repeat every 21 days × 4 |
| | | cycles |
| Cisplatin | 75 mg/m2 IV | |
| Cisplatin | 75 mg/m2 IV on Day 1 | Repeat every 21 days × 6 |
| | | cycles |
| Gemcitabine | 1250 mg/m2 IV on Day 1 | |
| | and Day 8 | |

Source: http://www.bccancer.bc.ca

Effective strategies to reduce the combined MTD of treatment with chemotherapy and radioimmunotherapy include: reducing the dose of chemotherapeutic agent administered to a level which does not result in additive toxicity when administered in conjunction with radioimmunotherapy at its MTD. (see Example 10A, below); selecting a chemotherapeutic agent that does not contribute additive toxicity when employed in combination with radioimmunotherapy. (see Example 10B, below); and reducing the bone marrow toxicity of the radioimmunotherapeutic agent (see Example 10C, below).

### A. Reducing the Dose of Chemotherapeutic Agent to Reduce Toxicity.

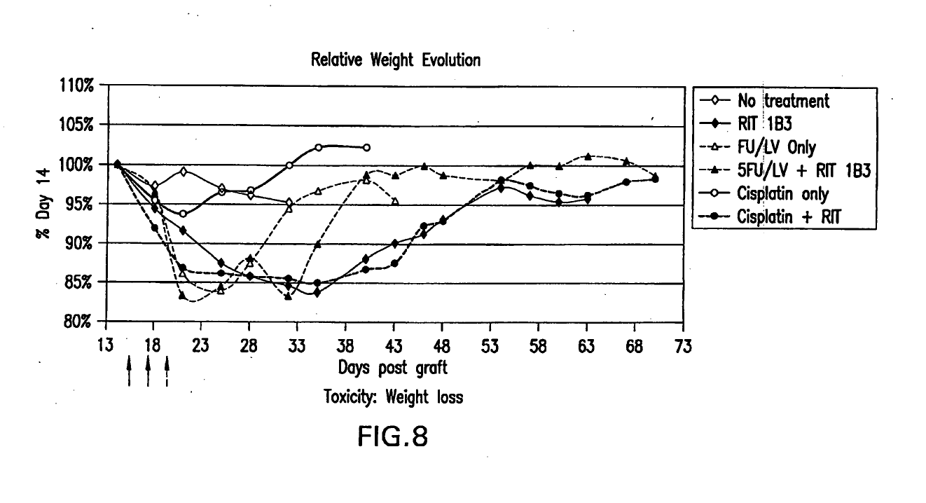

2 mg/kg of cisplatin (approximately 50% of MTD) was administered to Swiss nude mice on days 15 and 18 followed 72 hours later by ¹³¹I-labeled 1B3 anti-C35 monoclonal antibody administered at its MTD (300 μCi). As shown in FIG. 8, the combined toxicity of cisplatin and radioimmunotherapeutic as determined by weight loss was not significantly different from toxicity of the radioimmunotherapeutic alone administered at its MTD.

B. Chemotherapeutic Agent does not Contribute to Additive Toxicity.

5-FluoroUracil at the MTD of 180 mg/kg together with Leucovorin at 120 mg/kg were administered on day 18 followed by ¹³¹I-labeled 1B3 anti-C35 monoclonal antibody administered at its MTD (300 μCi) on day 21. As shown, in FIG. 8, the MTD of the combination of the two toxic agents was not exceeded, even though each agent was administered at its individual MTD.

### C. Reduced Bone Marrow Toxicity of the Radioimmunotherapeutic Agent.

An alternative strategy is to reduce the bone marrow toxicity of the radioimmunotherapeutic by biochemical modifications that result in accelerated clearance of radiolabeled antibody from peripheral circulation. Appropriate modifications include use of different antibody isotypes such as IgG3, IgA, IgD or IgE as indicated in Table 10 or deletion of the CH2 domain of IgG, which is responsible for its extended serum half life (See Mueller B M, R A Reisfeld, and S D Gillies, Proc. Natl. Acad. Sci. USA 87:5702-5705 (1990); Slavin-Chiorini D C, et al., Int. J. Cancer 53:97-103 (1993)).

TABLE 11
SERUM HALF-LIFE OF HUMAN
IMMUNOGLOBULIN ISOTYPES

| Immunoglobulin Isotype | Serum Half-Life | |
|---|---|---|
| IgG1 | 21 | days |
| IgG2 | 20 | days |
| IgG3 | 7 | days |
| IgG4 | 21 | days |
| IgM | 10 | days |
| IgA | 6 | days |
| IgD | 3 | days |
| IgE | 2 | days |

Other strategies to engineer antibodies and/or antibody fragments, or otherwise modify antibody structure so as to reduce serum half-life are also applicable. Examples of such engineered antibodies and/or antibody fragments include, but are not limited to, domain-deleted antibodies, Fab, F(ab′)2, scFv, minibodies, diabodies, triabodies, tetrabodies, etc.

### Example 111

### C35 Peptide Epitopes of 1B3 and 1F2 Antibodies

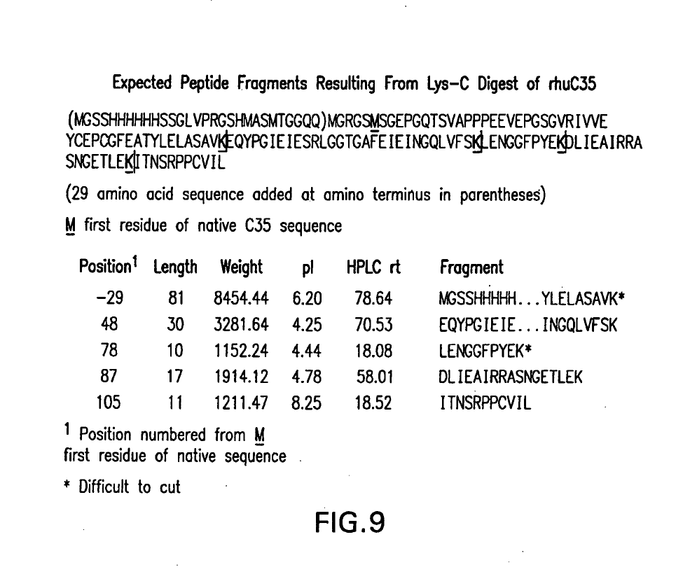

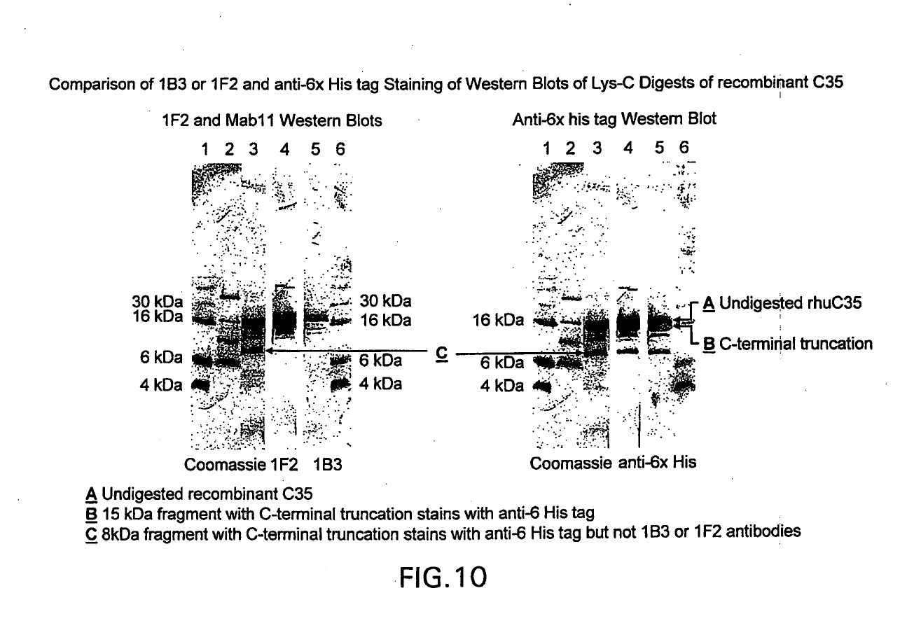

To localize the epitope specificity of 1B3 and 1F2 antibodies, recombinant human C35 (rhC35) synthesized with a 6×His tag in E. coli was digested with Lys-C endoproteinase. This enzyme cuts after lysine (K) residues in the protein sequence. FIG. 9 shows the expected peptide fragments following complete digestion of rhC35 with Lys-C. The full sequence of rhC35, including the amino terminal 6×His tag addition is shown. Amino acid positions are numbered relative to the amino terminal methionine (M) of the native human sequence. Note that digestion at the first and third lysine followed by negatively charged residues is inefficient and some longer combination fragments may be generated. Lys-C endoproteinase was added to purified rhC35 at a 50:1 weight ratio and incubated for 18 hours at 37° C. in 25 mM Tris, pH 8.0. The digest was ethanol precipitated and resolubilized in reducing Tricine sample buffer. After heating 5 minutes at 100° C., samples were separated by electrophoresis on a 16% Tricine gel (Invitrogen). Peptides were transferred to PVDF membranes and the blots depicted in FIG. 10 were either stained with Coomassie blue (lanes 1-3 of both left and right panels) to detect all peptides or processed with one of the following: 1 μg/ml of the murine 1F2 anti-C35 antibody followed by alkaline phosphatase conjugated goat anti-mouse antibody (lane 4 of left panel); 1 μg/ml of the 1B3 anti-C35 immunoglobulin variable regions linked to human constant regions (MAb11) followed by alkaline phosphatase conjugated goat anti-human antibody (lane 5 of left panel); or anti-6×His tag mouse antibody (Amersham) followed by alkaline phosphatase conjugated goat anti-mouse antibody (lane 4 and 5 of right panel). BCIP/NBT substrate was added and developed to detect the secondary reagents. Molecular weight markers are indicated in the flanking lanes of both panels.

In FIG. 10, the indicated band A migrates at the position of undigested rhC35. Note that band B stains with 1F2 but not MAb11 (1B3) anti-C35 antibody while band C stains with neither 1F2 nor MAb11 (1B3) antibody. Since bands B and C both do stain with anti-6×His tag antibody to the 6×His tag at the amino terminus of rhC35, it may be concluded that both fragments lack C-terminal peptide fragments. In the case of the approximately 15 kDa band, the missing epitope required for staining with MAb11 (1B3) is the 11 amino acid C-terminal C35 peptide ITNSRPPCVIL representing residues 105-115 of the native C35 sequence. This epitope is not required for staining with 1F2. In contrast, the 1F2 antibody does not react with the approximately 8 kDa band C which is, in addition, lacking residues 48-104 of the native C35 sequence. The results demonstrate that 1B3 antibody is specific for an epitope within C35 residues 105-115 (ITNSRPPCVIL), whereas 1F2 antibody is specific for an epitope within the C35 residues 48-104.

### Example 12

### Human Antibodies Related to 1B3 anti-C35 Monoclonal Antibody

Two antibodies which are human-derived except for having the same immunoglobulin heavy chain CDR3 region as the mouse 1B3 anti-C35 monoclonal antibody were generated by the method disclosed in US 2002 0123057 A1, published 5 Sep. 2002.

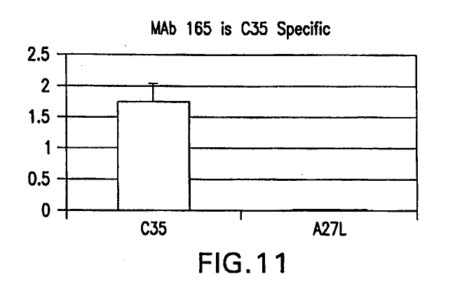

MAb 165 comprises the 141D10 VH H732 heavy chain variable region (SEQ ID NOS: 56 and 57), and the UH8 VK L120 kappa light chain variable region (SEQ ID NOS: 58 and 59). As shown in FIG. 11, MAb 165 is C35-specific. 141D10 recombinant vaccinia virus was co-infected into HeLa cells with UH8 recombinant vaccinia virus. The resulting secreted antibody was tested for binding to C35 or control protein A27L (vaccinia virus protein) by ELISA.

MAb 171 comprises the MSH3 VH H835 heavy chain variable region (SEQ ID NOS:60 and 61) and the UH8 VK L120 kappa light chain variable region (SEQ ID NOS: 58 and 59).

### Example 13

### Identification of C35 Peptide Epitopes Recognized by Anti-C35 Antibodies

Rabbit polyclonal antibodies were raised to recombinant C35 employing standard immunization methods well known in the art. Overlapping peptides 15 amino acids in length were synthesized corresponding to the 115 amino acid long C35 protein sequence beginning at each amino acid residue from 1 to 101. Peptides are named based on the C35 position of the amino terminus residue in each 15 amino acid peptide. In each peptide that overlapped the cysteine residues at positions 30, 33 and 112 of the natural C35 sequence, alanine was substituted for cysteine to avoid formation of disulfide crosslinked peptides.

Wells of 96 well Maxisorp microtiter plates were coated with either 2 μg C35 protein, or 14 μg or 40 μg of the indicated 15 amino acid peptide derived from the C35 protein sequence and binding of antibodies in the rabbit C35 immune serum was determined as described in detail below. Data in Table 12, below, is shown for the positive and negative controls and for those C35 derived peptides for which positive binding was detected. Variations in the level of peptide binding can be due to either differences in the concentration of specific antibody species, differences in antibody affinity or a combination thereof.

### A. Peptide Sample Preparation

C35 peptide 15-mers in 100% DMSO, 10 mg/ml, were aliquotted into 1.5 ml tubes, 40 μg/tube, under sterile conditions, then speed vacuumed to remove DMSO. The peptides were resuspended in PBS, pH7.2, 1 ml/tube, mixed well and spun down. Each peptide concentration (“peptide solution”) was 40 μg/ml. Peptide solution should be stored at −20° C. until use. Once thawed, it should be kept at 4° C. for no more than 2 weeks.

### B. Coating Samples on the Maxisorp Plates

For 14 μg/ml peptide coated plates, 65 μl PBS, pH7.2 per well was added, followed by 35 μl peptide solution per well (for a total volume of 100 μl/well), and mixed well. For 40 μg/ml peptide coated plates, 100 μl of peptide solution was placed directly on the plate, 100 μl/well. Control C35 protein was diluted into PBS, pH7.2 to 2 μg/ml and added to the plates, 100 μl/well. Coated plates were incubated at room temperature for 2 hours, then 4° C. overnight.

### C. ELISA Conditions

Each plate was washed 3 times on a plate washer. Plates were blocked with “blocking buffer” (PBS, pH7.2 and 10% FBS) at room temperature for 2 hours, followed by washing each plate 3 times on plate washer. The primary antibody, rabbit anti-human C35 polyclonal antibody (made by Bethyl, 62902 batch) was diluted into “assay diluent” (PBS, pH7.2 plus 0.05% Tween 20 and 10% FBS) and added to the plates, 100 μl/well. Plates were incubated at room temperature for 2 hours. For 14 μg/ml peptide coated plates, 100 ng/ml of primary antibody was added. For 40 μg/ml peptide coated plates, 1 μg/ml of primary antibody was added. Plates were washed 5 times on a plate washer. The secondary antibody, HRP (horseradish peroxidase conjugated goat anti-rabbit Fc polyclonal antibody from Zymed, was diluted at a 1:20,000 dilution into assay diluent and added to the plates, 100 μl/well. The plates were incubated at room temperature for 2 hours. Plates were washed 7 times on a plate washer. Substrate was added as per the kit manufacturers instructions and incubated at room temperature in the dark for 15 minutes. The reaction was stopped by adding 2NH₂SO₄, 100 μl/well. Absorbance at 450-570 nm was read immediately.

TABLE 12

ANTIBODY BINDING TO C35 EPITOPES
Absorbance @ 450-570 nm

| | | | |
|---|---|---|---|
| Peptide | Sequence | 14 μg/ml | 40 μg/ml |

| | | | |
|---|---|---|---|
| None | | 0.009 | 0.013 |
| without | | | |
| primary rabbit | G35 protein | 0.009 | 0.009 |
| | or peptides | | |
| antibody | | | |
| C35 protein | C35 protein | 3.765 | 3.5225 |
| P1 | MSGEPGQTSVAPPPE | 0.257 | |
| P2 | SGEPGQTSVAPPPEE | 0.108 | 1.227 |
| P3 | GEPGQTSVAPPPEEV | 1.458 | 3.349 |
| P4 | EPGQTSVAPPPEEVE | 1.254 | 3.282 |
| P5 | PGQTSVAPPPEEVEP | 2.719 | 3.383 |
| P6 | GQTSVAPPPEEVEPG | 2.483 | 3.381 |
| P7 | QTSVAPPPEEVEPGS | 2.635 | 3.388 |
| P8 | TSVAPPPEEVEPGSG | 0.059 | 0.394 |
| P9 | SVAPPPEEVEPGSGV | 2.31 | 3.367 |
| P10 | VAPPPEEVEPGSGVR | 1.736 | 3.407 |
| P11 | APPPEEVEPGSGVRI | 1.526 | 3.317 |
| P12 | PPPEEVEPGSGVRIV | 0.836 | |
| P13 | PPEEVEPGSGVRIVV | 0.385 | 2.522 |
| P14 | PEEVEPGSGVRIVVE | 0.127 | 0.972 |
| P15 | EEVEPGSGVRIVVEY | 0.039 | 0.334 |
| P16 | EVEPGSGVRIVVEYA | 0.027 | 0.172 |
| P62 | TGAFEIEINGQLVFS | 0.023 | 0.107 |
| P63 | GAFEIEINGQLVFSK | 0.079 | 0.557 |
| P64 | AFEIEINGQLVFSKL | 0.055 | 0.423 |
| P65 | FEIEINGQLVFSKLE | 0.043 | 0.269 |
| P66 | EIEINGQLVFSKLEN | 0.028 | 0.182 |
| P80 | NGGFPYEKDLIEAIR | 0.018 | 0.1 |
| P81 | GGFPYEKDLIEAIRR | 0.032 | 0.204 |
| P82 | GFPYEKDLIEAIRRA | 0.02 | 0.18 |
| P83 | FPYEKDLIEAIRRAS | 0.025 | 0.19 |
| P84 | PYEKDLIEAIRRASN | 0.037 | 0.391 |
| P85 | YEKDLIEAIRRASNG | 0.024 | 0.179 |
| P86 | EKDLIEAIRRASNGE | 0.023 | 0.166 |
| P88 | DLIEAIRRASNGETL | 0.025 | 0.135 |
| P89 | LIEAIRRASNGETLE | 0.015 | 0.053 |
| P90 | IEAIRRASNGETLEK | 0.04 | 0.403 |
| P91 | EAIRRASNGETLEKI | 0.023 | 0.144 |
| P92 | AIRRASNGETLEKIT | 0.04 | 0.267 |
| P93 | IRRASNGETLEKITN | 0.051 | 0.389 |
| P94 | RRASNGETLEKITNS | 0.061 | 0.526 |
| P95 | RASNGETLEKITNSR | 0.062 | 0.462 |
| P97 | SNGETLEKITNSRPP | 0.046 | 0.373 |
| P98 | NGETLEKITNSRPPA | 0.035 | 0.213 |
| P99 | GETLEKITNSRPPAV | 0.026 | 0.18 |
| P100 | ETLEKITNSRPPAVI | 0.017 | 0.124 |
| P101 | TLEKITNSRPPAVIL | 0.472 | 2.519 |

### Example 14

### Combination of Two C35 Antibodies and Chemotherapy

As illustrated in FIG. 12 and described in this example, methods of treating cancer directed to the administration of two C35 antibodies in combination with a chemotherapeutic agent were tested. Five million C35-positive MDA231 tumor cells were implanted subcutaneously in the mammary fat pads of Swiss nude mice. Groups of at least five mice each received the following treatments, beginning on day 17 post-graft:
1. No Treatment (Control Group)
2. 30 mg/kg i.p. injection of paclitaxel on days 17 and 24.
3. 30 mg/kg i.p. injection of paclitaxel on days 17 and 24, together with 400 μg per i.v. injection (20 mg/kg) of 1F2 murine monoclonal antibody starting on day 17 and continuing twice weekly for three weeks.
4. 30 mg/kg i.p. injection of paclitaxel on days 17 and 24, together with 400 μg per i.v. injection (20 mg/kg) of 1B2 murine monoclonal antibody starting on day 17 and continuing twice weekly for three weeks.
5. 30 mg/kg i.p. injection of paclitaxel on days 17 and 24, together with a combination of 400 μg per i.v. injection (20 mg/kg) of 1F2 and 400 μg per i.v. injection (20 mg/kg) of 1B3 murine monoclonal antibodies (40 mg/kg total), starting on day 17 and continuing twice weekly for three weeks.
6. 30 mg/kg i.p. injection of paclitaxel on days 17 and 24, together with 400 μg per i.v. injection of monoclonal isotype control antibody starting on day 17 and continuing twice weekly for three weeks.

Average mouse tumor volume was measured at various time points post-graft. Two measurements were taken with vernier calipers on each tumor; tumor volume was calculated using the formula (length×width²)/2. The results are illustrated in FIG. 12 and demonstrate that the combination of two murine anti-C35 antibodies, 1F2 and 1B3, together with chemotherapy (paclitaxel) inhibited growth of a C35-positive tumor in vivo. As shown in FIG. 12, neither the 1F2 nor 1B3 administered individually with the chemotherapeutic agent paclitaxel was effective in inhibiting tumor growth in mice. Tumor growth in the mice treated with the combination of 1F2 and 1B3 with paclitaxel resumed approximately one week following the last antibody treatment, correlating with the expected half-life of the antibodies in vivo.

### Example 15

### MAB 163 Demonstrates Tumor Specific Binding to C35

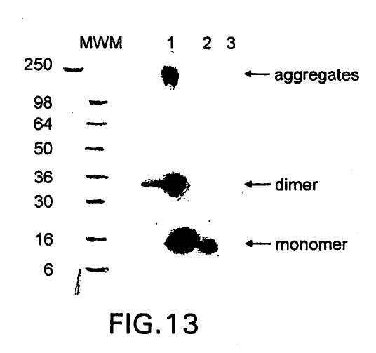

As illustrated in FIG. 13, MAb 163 was shown to demonstrate tumor specific binding using Western Blot detection. Samples were resuspended in Laemli buffer, reduced with β-ME and heat, run on 4-20% SDS-PAGE, and transferred to PVDF membrane. The membrane was incubated with MAb163 (4.4 ug/ml), followed by detection with goat anti-human IgG-HRP and developed by chemiluminescence.

The following lanes are shown in FIG. 13: Lane 1: recombinant human C35 protein (rC35), purified from E. coli (100 ng/lane); Lane 2: 21MT1-D human breast tumor cell lysate (100,000 cell equivalents/lane); and Lane 3: H16N2 normal immortalized human breast cell line lysate (100,000 cell equivalents/lane). The molecular weight markers are indicated in kiloDaltons, on left of the figure.

This experiment demonstrated that MAb 163 binds native C35 monomer in tumor cell lysate; however, binding is absent in the normal cell line. MAb 163 also binds the rC35 monomer (16 kD), dimer (˜32 kD), and larger aggregates. Recombinant C35 (lane 1) is slightly larger than native C35 (lane 2) due to the presence of a 6×his tag in rC35.

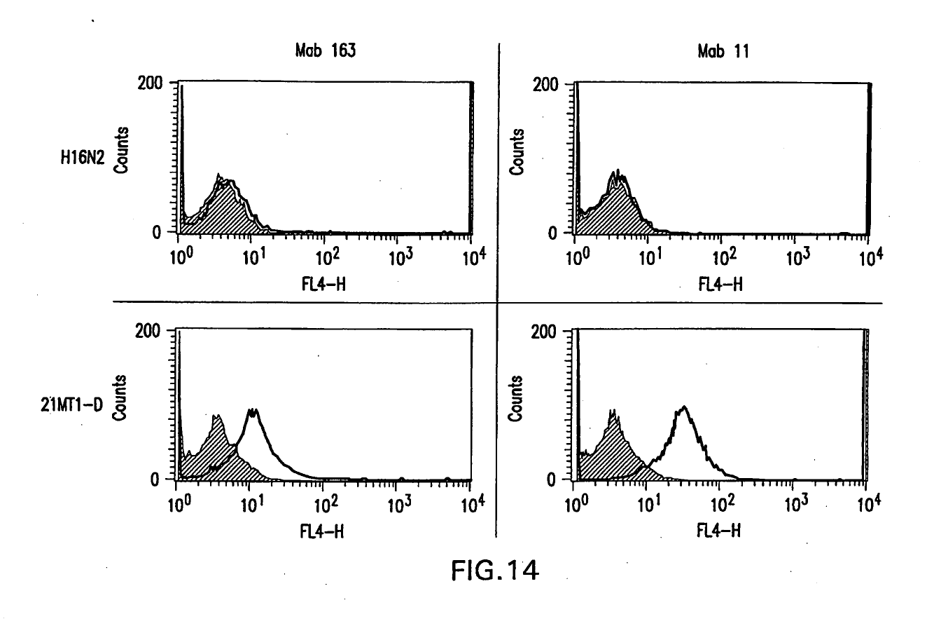

FIG. 14 illustrates an analysis of MAb 163 specificity by flow cytometry. Intracellular FACS was performed as follows: H16N₂ (C35-negative normal immortalized breast cell line) and 21MT1 (C35-positive breast tumor cell line) cells were fixed and permeabilized, followed by a 45 minute incubation of 0.5 million cells with 1 μg of anti-C35 antibodies (either MAb 163 or Mab 11) or isotype matched control antibody conjugated to Alexa-647, using Xenon-labeling kit (Molecular Probes). Washed cells were then analyzed on FACS Calibur (BD Biosciences). Staining with the isotype control Mab is shown as the filled area in FIG. 14; staining with anti-C35 Mab is shown as the open line. The shift of the open line representing MAb163 or Mab11 in the 21MT specificity of the C35 antibodies.

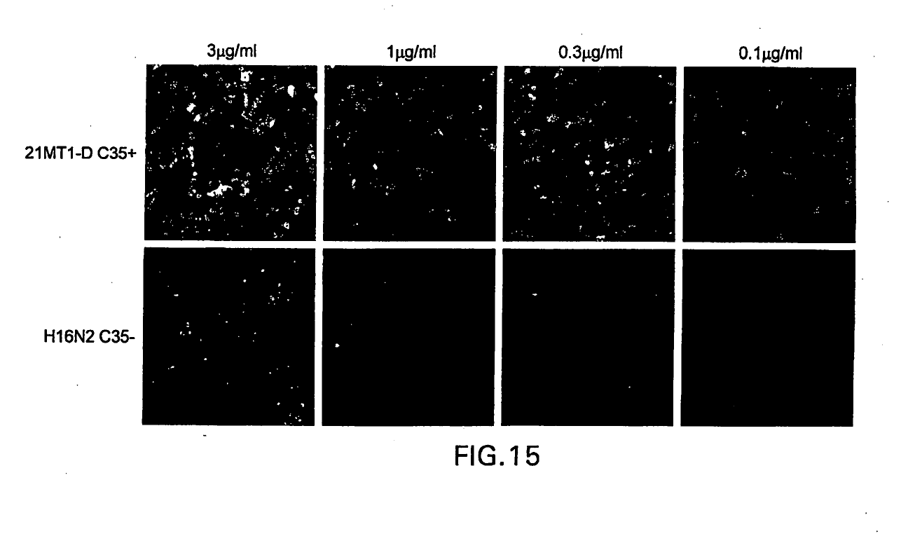

Immunofluorescence testing with MAb 163 in human mammary cell lines also confirmed that MAb 163 specifically binds C35. The images in FIG. 15 were generated using the following protocol. Paraformaldehyde-fixed cells (either C35+ or C35−) were incubated with various concentrations of MAb163 (3 μg/ml, 1 μg/ml, 0.3 μg/ml, or 0.1 μg/ml), followed by detection with secondary antibody, anti-human IgG conjugated to APC, and visualized on FMAT. The results of this testing is shown in FIG. 15, where the C35-negative H16N₂ cells do not show any immunofluorescence staining and C35-positive 21MT1-D cells show dose-dependent immunofluorescence.

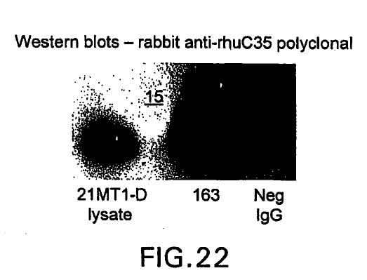

As illustrated in FIG. 22, Mab163 was used to immunoprecipitate C35 from cell lysates of the 21MT1-D C35-expressing cell line. Briefly, 10 ml of MAb163 in CHO cell supernatant was buffer-exchanged into PBS and concentrated to 1 ml. Concentrated MAb 163 was added to protein A beads for greater than one hour. After washing, protein A/MAb163 was added to C35-positive 21MT1-D cell lysates for 2 hours. After washing the beads, the protein was eluted with reducing sample buffer at 100° C.

Immunoprecipitated samples were analyzed by Western blot using rabbit anti-C35 polyclonal sera. As shown in the left lane of FIG. 22, lysate from 100,000 21MT1-D cells was included on the Western blot as a control. The number “15” in FIG. 22 indicates a molecular weight marker at 15 kDalton. FIG. 22 shows that MAb163 (identified as “163” in the center) immunoprecipitated C35 protein, whereas an IgG negative control antibody (identified as “Neg IgG” on the right side) did not.

### Example 16

### Mab 163 Affinity Testing

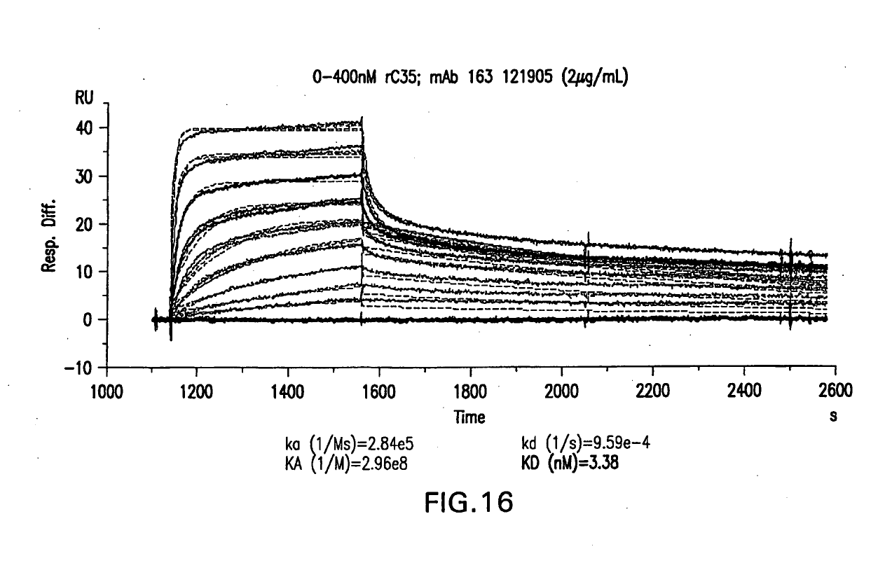

As illustrated in FIG. 16, experiments were conducted to determine the KA and KD for MAb 163 using a 1:1 kinetic model. To measure affinity using Biacore, a CM5 chip surface was prepared by immobilizing Goat Anti-Human IgG Fc through amine coupling. The monoclonal human antibody MAb 163 was then captured by flow over the chip containing the Goat Anti-Human IgG. The rC35 was then serial diluted into two separate series to cover a wide range of concentration points and binding efficiencies, and then the C35 was allowed to flow over the chip containing bound MAb 163. The binding was recorded in a series of sensograms. The binding was evaluated using BIAevaluation software, where the Ka and Kd were calculated by fitting the curves and correcting for Mass Transfer. These experiments yielded the following results for MAb 163: (a) ka (1/Ms)=2.84e5; (b) kd (1/s)=9.59e4; (c) KA (1/M)=2.96e8; and (d) KD (nM)=3.38.

### Example 17

### Mab 163 Epitope Mapping

To localize the epitope specificity of MAb 163, recombinant human C35 (rhC35), synthesized with a 6×His tag in E. coli, was digested with Lys-C endoproteinase. This enzyme cuts after lysine (K) residues in the protein sequence. Samples were separated by electrophoresis on a 10 well, 16% Tricine gel (Invitrogen). Detection was performed by Western blotting using incubation with human antibodies, followed by goat anti-human secondary antibody conjugated to horseradish peroxidase, and detection with TMB (3,3′,5,5′-tetramethylbenzidine) as chromogen. Coomassie blue staining was used to detect all peptides. See Example 11, supra, for additional details of the protocol.

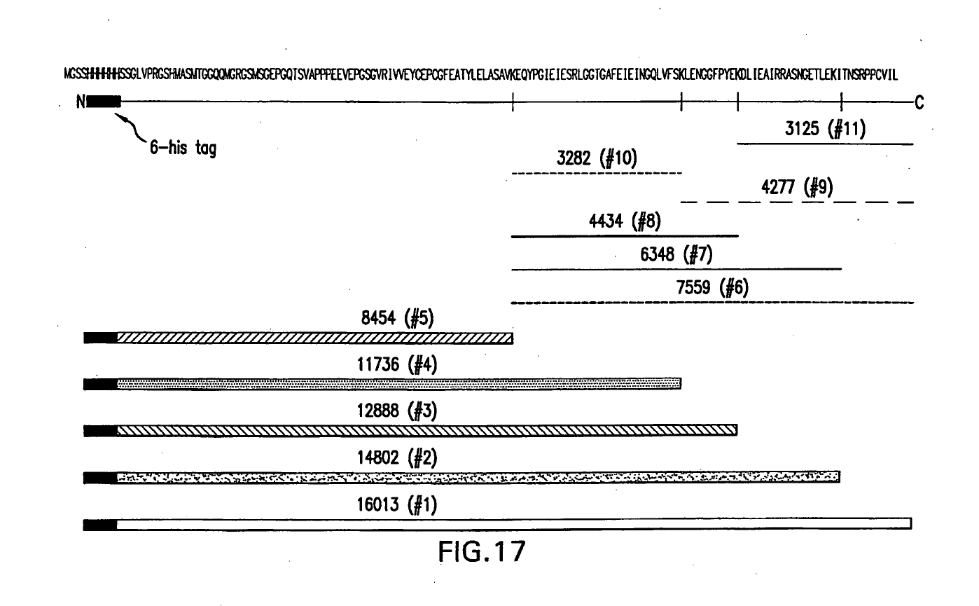

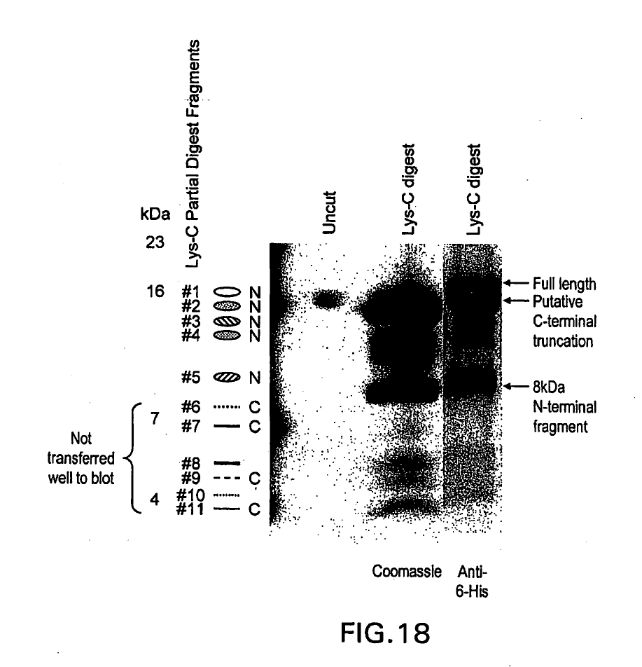

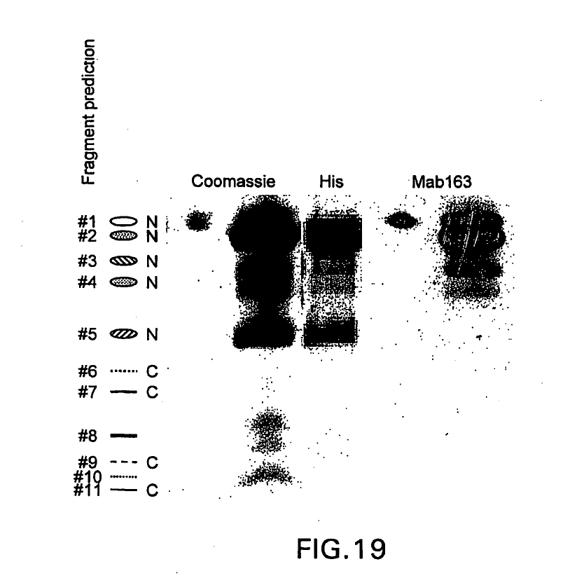

FIG. 17 shows the expected peptide fragments following partial digestion of 6-His-tagged recombinant human C35 (rhC35) with Lys-C endoprotease. Each of the 11 predicted digestion fragments is represented by a different style of line, which corresponds to the same predicted fragments as shown for comparison to the left of the Western blots in FIGS. 18 and 19.

FIG. 18 shows the observed peptide fragments following a Lys-C digestion of rhC35 using Coomassie blue staining and anti-6-His staining. The predicted partial digest fragments are shown to the left of the blots for comparison. As noted, the smaller predicted fragments (i.e., 6-11) did not transfer well to the blot.

FIG. 19 shows a Western blot comparison of MAb 163 staining to the Coomassie blue and anti-6-His blots, identifying the fragment containing the C435 epitope to which MAb 163 binds. The predicted fragments are shown to the left of the blots for comparison. MAb 163 binding to C35 fragments corresponding to predicted fragments 14 can be seen in FIG. 19, but there is no binding to predicted fragments 5-11.

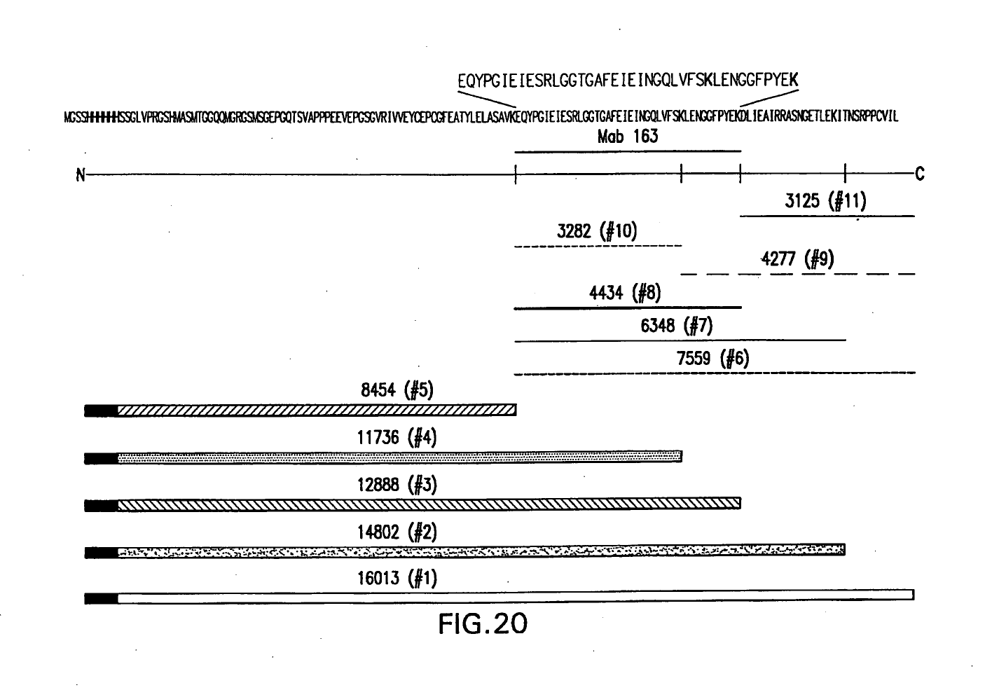

The results, as depicted in FIG. 20, demonstrate that MAb 163 is specific for an epitope within amino acid residues 48 to 87 of C35, with the amino acid positions numbered relative to the amino terminal methionine of the native human sequence (see FIG. 9). This region has the following amino acid sequence: EQYPGIEIESRLGGTGAFEIEINGQLVFSKLENGGFPYEK.

### Example 18

### Human Anti-C35 Mabs or Herceptin Inhibit In Vitro Proliferation of C35+/Her2+Tumor Cell Line

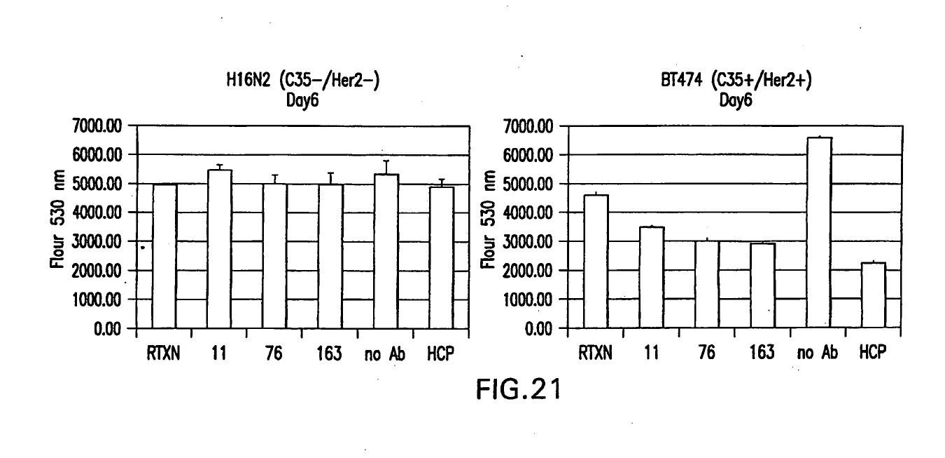

FIG. 21 shows the results of cell proliferation assays performed using BT474 cells, a C35-positive/Her2-negative breast tumor cell line, and H16N₂ cells, a C35-negative/Her2-negative normal breast cell line. The cells were seeded in triplicate and allowed to adhere overnight. The cells were synchronized in G0 for 24 hours with 90 μM quinidine. Following a wash to release the cells from G0, antibodies were added at 150 μg/ml, or approximately 1M. Rituxan (human anti-CD20) was as a negative antibody control because all breast lines tested are CD20-negative. Herceptin (anti-Her2/neu) was used as positive antibody control for Her2-positive tumor cell lines. The anti-C35 antibodies used in the experiment were MAb 163, MAb 11 (chimeric 1B3), and MAb 76 (Chimeric 1F2).

Alamar blue was added at various time points to analyze proliferation. Alamar blue reduces and changes color in presence of several metabolic enzymes. Alamar blue can be reduced by NADPH, FADH, FMNH, NADH, similar to MTT, but can also be reduced by cytochromes, unlike MTT. Following a 90 minute incubation with alamar blue, fluorescence at 530 nm was detected on fluorescent microplate reader. The data presented in FIG. 21 is from day 6 of in vitro culture with or without antibody addition.

The results of the proliferation assays indicate that human anti-C35 antibodies or Herceptin inhibit proliferation of BT474, a C35-positive/Her2-positive breast tumor cell line, but not of H16N2, a C35-negative/Her2-negative normal breast cell line. There is a low level of spontaneous induction of apoptosis in vitro that creates targets for anti-C35 antibody and appears to contribute to the reduced cell proliferation in the presence of anti-C35 antibody. Sensitivity to this inhibitory effect in vitro appears to be greater than in vivo as it is mediated by individual antibodies and does not require a combination of specificities or a chemotherapy (which induces widespread cell death in vitro).

### Example 19

### Prevention of Tumor Growth by Combination of Two C35 Antibodies and Adriamycin

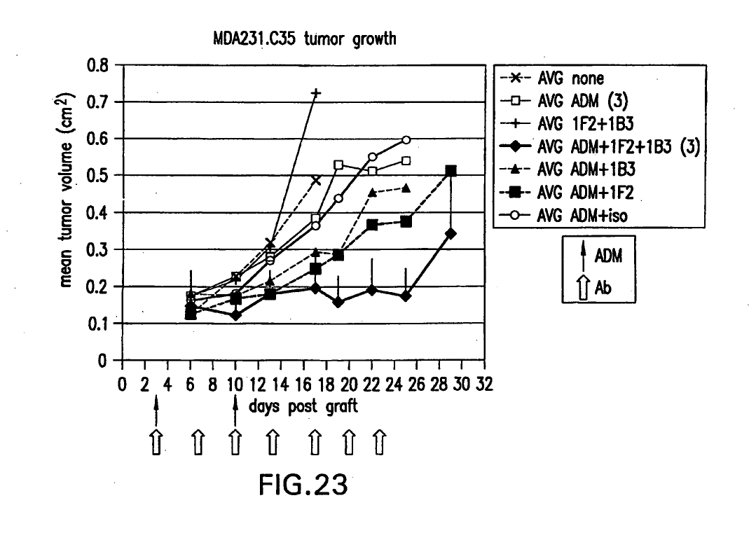

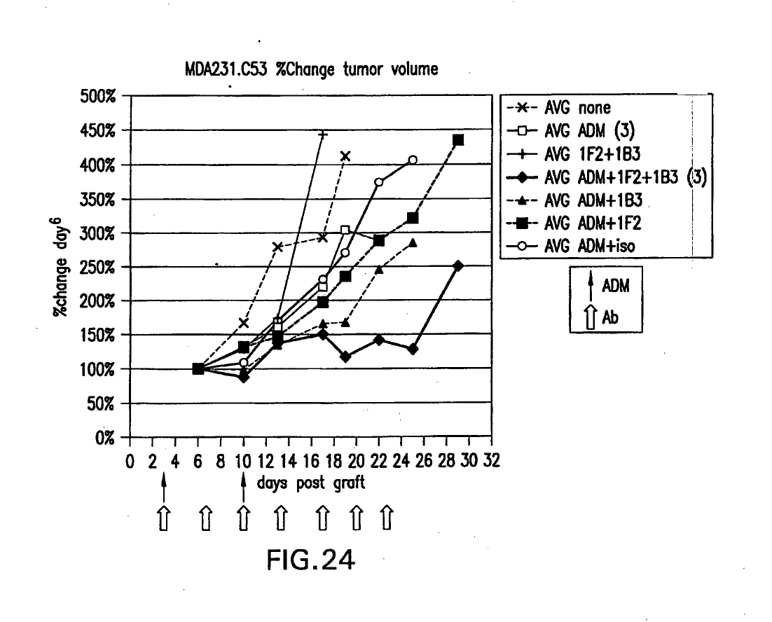

As illustrated in FIGS. 23 and 24, and as described in this example, a combination of two C35 antibodies with a chemotherapeutic agent were tested for the effect on tumor growth in vivo. As in Example 14, above, five million C35-positive MDA231 tumor cells were implanted subcutaneously in the mammary fat pads of Swiss nude mice. Groups of six mice each received the following treatments, beginning on day 3 post-graft:
1. No treatment (control group);
2. 8 mg/kg i.v. administration of adriamycin on days 3 and 10 post-graft;
3. 20 mg/kg i.v. administration of 1B3 murine monoclonal antibody and 20 mg/kg i.v. administration of 1F2 on days 3, 7, 10, 13, 17, 20, and 23 post-graft;
4. 8 mg/kg i.v. administration of adriamycin on days 3 and 10, together with 40 mg/kg i.v. administration of 1F2 murine monoclonal antibody on days 3, 7, 10, 13, 17, 20, and 23 post-graft.
5. 8 mg/kg i.v. administration of adriamycin on days 3 and 10, together with 40 mg/kg i.v. administration of 1B2 murine monoclonal antibody on days 3, 7, 10, 13, 17, 20, and 23 post-graft.
6. 8 mg/kg i.v. administration of adriamycin on days 3 and 10, together with a combination of 20 mg/kg i.v. administration of 1F2 and 20 mg/kg i.v. administration of 1B3 (40 mg/kg total antibodies) on days 3, 7, 10, 13, 17, 20, and 23 post-graft.
7. 8 mg/kg i.v. administration of adriamycin on days 3 and 10, together with 40 mg/kg i.v. administration of monoclonal isotype control antibody on days 3, 7, 10, 13, 17, 20, and 23 post-graft.

As in Example 14, above, average mouse tumor volume was measured at various time points post-graft. The results, illustrated in FIGS. 23 and 24, demonstrate that a 40 mg/kg total dose of murine C35 antibodies 1B3 (20 mg/kg dose) and 1F2 (20 mg/kg dose), in combination with an 8 mg/kg dose of adriamycin (doxorubicin), was more effective in preventing tumor growth in mice grafted with MDA231.C35 tumors than a 40 mg/kg total dose of 1B3 and 1F2, a 40 mg/kg total dose of 1B3 with adriamycin, a 40 mg/kg total dose of 1F2 with adriamycin, adriamycin alone, an IgG isotype antibody control, or no treatment. By day 18, post-graft, only the combination of 1B3 and 1F2 with adriamycin was effective at preventing tumor growth (FIGS. 23 and 24). Tumor growth in the mice treated with the combination of 1F2 and 1B3 with adriamycin resumed within a week following the last antibody treatment on day 23 post-graft (FIGS. 23 and 24).

1. A method of killing cancer cells that express C35, the method comprising administering to said cells (a) a first C35 antibody or antigen binding fragment thereof that specifically binds C35; (b) a second C35 antibody or antigen binding fragment thereof that specifically binds C35; and (c) a therapeutic agent.

2. The method of claim 1, wherein said method is performed in vivo.

3. The method of claim 2, wherein said method is performed in a mammal.

4. The method of claim 3, wherein said mammal is a human.

5. The method of claim 1, wherein said first and second C35 antibodies or fragments each bind to a different C35 epitope.

6. The method of claim 1 wherein at least one of said first or second C35 antibodies or fragments binds a C35 epitope selected from the group consisting of a C35 epitope located within amino acid residues 105-115 of SEQ ID NO:2, a C35 epitope located within amino acid residues 48-87 of SEQ ID NO:2, and a C35 epitope located within amino acid residues 48-104 of SEQ ID NO:2.

7. The method of claim 1, wherein said therapeutic agent is a chemotherapeutic agent.

8. The method of claim 7, wherein said chemotherapeutic agent is selected from the group consisting of cisplatin, carboplatin, paclitaxel, adriamycin, docetaxel, taxotere, gemcitabine, and vinorelbine.

9. The method of claim 8, wherein said chemotherapeutic agent is paclitaxel.

10. The method of claim 8, wherein said chemotherapeutic agent is adriamycin.

11. The method of claim 1, wherein said therapeutic agent is administered prior to administering at least one of said first or second C35 antibodies.

12. The method of claim 1, wherein said therapeutic agent is administered after administering at least one of said first or second C35 antibodies.

13. The method of claim 1, wherein said therapeutic agent is administered concurrently with at least one of said first or second C35 antibodies.

14. The method of claim 1, wherein said first and second C35 antibodies are administered concurrently.

15. The method of claim 1, wherein said first and second C35 antibodies are administered sequentially.

16. The method of claim 1, wherein each of said C35 antibodies or fragments is administered at a dose of about 0.1 mg/kg to about 100 mg/kg of a patient's body weight.

17. The method of claim 1, wherein at least one of said first or second C35 antibodies or fragments is selected from the group consisting of 1F2, 1B3, MAbc0009, MAb 163, MAb 165, MAb 171, and variants or derivatives thereof.

18. The method of claim 17, wherein one of said first or second C35 antibodies or fragments is MAb 163 or a variant or derivative thereof.

19. The method of claim 17, wherein one of said first or second C35 antibodies or fragments is 1B3 or a variant or derivative thereof.

20. The method of claim 17, wherein one of said first or second C35 antibodies or fragments is 1F2 or a variant or derivative thereof.

21. The method of claim 17, wherein said first and second C35 antibodies are selected from the group consisting of 1F2, 1B3, MAbc0009, MAb 163, MAb 165, MAb 171, and variants or derivatives thereof.

22. The method of claim 21, wherein said first and second C35 antibodies are 1B3 and 1F2 or variants or derivatives thereof.

23. The method of claim 1, wherein the cancer cells are selected from the group consisting of breast cancer, liver cancer, ovarian cancer, bladder cancer, lung cancer, prostate cancer, pancreatic cancer, colon cancer, and melanoma.

24. The method of claim 23, wherein the cancer cells are breast cancer cells.

25. The method of claim 23, wherein the cancer cells are liver cancer cells.

26. The method of claim 1, wherein said method comprises administering more than two C35 antibodies or fragments thereof.

27. An isolated antibody or antigen-binding fragment thereof which specifically binds to the same C35 epitope as the reference antibody MAb 163.

28-48. (canceled)

49. A composition comprising the antibody or fragment thereof of claim 27, and a carrier.

50-111. (canceled)

112. An isolated polynucleotide comprising a nucleic acid sequence encoding at least one complementarity determining region (CDR) or a variant thereof of the MAb 163 monoclonal antibody, wherein said polynucleotide encodes a polypeptide that specifically binds to C35.

113-117. (canceled)

118. A vector comprising the polynucleotide of claim 112.

119-125. (canceled)

126. A composition comprising the polynucleotide of claim 112.

127-149. (canceled)

150. A host cell comprising the polynucleotide of claim 112.

151. (canceled)

152. A method of producing an anti-C35 antibody or antigen-binding fragment thereof, comprising culturing the host cell of claim 150, and recovering said antibody or fragment.

153. An anti-C35 antibody, or antigen-binding fragment thereof, produced by the method of claim 152.

154. An isolated polypeptide comprising an immunoglobulin heavy chain variable region (VH), wherein the CDR1, CDR2, and CDR3 regions of said VH are identical respectively, except for fewer than 10 amino acid substitutions, to reference heavy chain CDR1, CDR2, and CDR3 sequences consisting of SEQ ID NO:63, SEQ ID NO:64, and SEQ ID NO:65, and wherein an antibody or antigen-binding fragment thereof comprising said VH specifically binds to C35.

155-180. (canceled)

181. A composition comprising the polypeptide of claim 154, wherein an antibody or antigen-binding fragment thereof comprising said VH specifically binds to C35.

182-198. (canceled)

199. An isolated antibody or antigen binding fragment thereof comprising the polypeptide of claim 154.

200. A method for treating cancer comprising administering to an animal suffering from cancer an effective amount of the composition of claim 49.

201-202. (canceled)

203. A composition comprising: (a) a first C35 antibody that specifically binds to C35; (b) a second C35 antibody that specifically binds to C35; and (c) a therapeutic agent.

204-209. (canceled)

210. A method of detecting the presence of C35, the method comprising: (a) contacting a sample or cell with an antibody or antigen binding fragment thereof according to claim 27; and (b) detecting the binding of said antibody or antigen binding fragment thereof to C35.

211-213. (canceled)

214. An isolated antibody or antigen binding fragment thereof comprising at least one, two, three, four, five or six CDRs of the MAb 163 monoclonal antibody, wherein said antibody or fragment specifically binds C35.

215-220. (canceled)


SEQUENCE LISTING (77 sequences)

SEQ ID NO: 1 - DNA, 354 bp, Homo sapiens
  CDS (7)..(354)
gccgcgatgagcggggagccggggcagacgtccgtagcgccccctcccgaggaggtcgagccgggcagtggggtccgcatcgtggtggagtactgtgaaccctgcggcttcgaggcgacctacctggagctggccagtgctgtgaaggagcagtatccgggcatcgagatcgagtcgcgcctcgggggcacaggtgcctttgagatagagataaatggacagctggtgttctccaagctggagaatgggggctttccctatgagaaagatctcattgaggccatccgaagagccagtaatggagaaaccctagaaaagatcaccaacagccgtcctccctgcgtcatcctgtga

SEQ ID NO: 2 - protein, 115 aa, Homo sapiens
MSGEPGQTSVAPPPEEVEPGSGVRIVVEYCEPCGFEATYLELASAVKEQYPGIEIESRLGGTGAFEIEINGQLVFSKLENGGFPYEKDLIEAIRRASNGETLEKITNSRPPCVIL

SEQ ID NO: 3 - DNA, 627 bp, Mus sp.
gaatttagcggccgcgaattcgcccttcgactggagcacgggacactgacatggactgaaggagtagaaaacatctctctcattagaggttgatctttgaggaaaacagggtgttgcctaaaggatgaaagtgttgagtctgttgtacctgttgacagccattcctggtatcctgtctgatgtacagcttcaggagtcaggacctggcctcgtgaaaccttctcagtctctgtctctcacctgctctgtcactggctactccatcaccagtggttatttctggaactggatccggcagtttccagggaacaaactggaatggatgggctacataagctacgacggtagcaataactccaacccatctctcaaaaatcgaatctccttcactcgtgacacatctaagaaccagtttttcctgaagtttaattctgtgactactgacgactcagctgcatattactgtacaagaggaactacggggtttgcttactggggccaagggactctggtcactgtctctgcagccaaaacgacacccccatctgtctatccactggcccctggatctgctgcccaaactaactccaagggcgaattcgtttaaacctgcaggactagtccctt

SEQ ID NO: 4 - protein, 116 aa, Mus sp.
DVQLQESGPGLVKPSQSLSLTCSVTGYSITSGYFWNWIRQFPGNKLEWMGYISYDGSNNSNPSLKNRISFTRDTSKNQFFLKFNSVTTDDSAAYYCTRGTTGFAYWGQGTLVTVSA

SEQ ID NO: 5 - DNA, 540 bp, Mus sp.
cgcgaattcgcccttcgactggagcacgaggacactgacatggactgaaggagtagaaaaattagctagggaccaaaattcaaagacagaatggattttcaggtgcagattttcagcttcctgctaatcagtgcctcagtcagaatgtccagaggacaaattgttctcacccagtctccagcaatcatgtctgcatctccaggggagaaggtcaccatatcctgcagtgccagctcaagtgtaagttacatgaactggtaccagcagaagccaggatcctcccccaaaccctggatttatcacacatccaacctggcttctggagtccctgctcgcttcagtggcagtgggtctgggacctcttactctctcacaatcagcagcatggaggctgaagatgctgccacttattactgccaacagtatcatagttacccacccacgttcggaggggggaccaagctggaaataaaacgggctgatgctgcaccaactgtatccatcttcccaccatccagtgagcaaagggcgaattcgttt

SEQ ID NO: 6 - protein, 106 aa, Mus sp.
QIVLTQSPAIMSASPGEKVTISCSASSSVSYMNWYQQKPGSSPKPWIYHTSNLASGVPARFSGSGSGTSYSLTISSMEAEDAATYYCQQYHSYPPTFGGGTKLEIK

SEQ ID NO: 7 - DNA, 618 bp, Mus sp.
cgcgaattcgcccttcgactggagcacgaggacactggacatggactgaaggagtagaaaatctctctcactggaggctgatttttgaagaaaggggttgtagcctaaaagatgatggtgttaagtcttctgtacctgttgacagcccttccgggtatcctgtcagaggtgcagcttcaggagtcaggacctagcctcgtgaaaccttctcagactctgtccctcacctgttctgtcactggcgactccatcaccagtggttactggaactggatccggaaattcccaggaaataaacttgaatacgtggggtacataagctacagtggtggcacttactacaatccatctctcaaaagtcgaatctccatcactcgagacacatccaagaaccactactacctgcagttgaattctgtgactactgaggacacagccacatattactgtgcaagaggtgcttactacgggggggccttttttccttacttcgatgtctggggcgctgggaccacggtcaccgtctcctcagccaaaacgacacccccatctgtctatccactggcccctggatctgctgcccaaactaactccaagggcgaattcgtttaaacctgc

SEQ ID NO: 8 - protein, 122 aa, Mus sp.
EVQLQESGPSLVKPSQTLSLTCSVTGDSITSGYWNWIRKFPGNKLEYVGYISYSGGTYYNPSLKSRISITRDTSKNHYYLQLNSVTTEDTATYYCARGAYYGGAFFPYFDVWGAGTTVTVSS

SEQ ID NO: 9 - DNA, 528 bp, Mus sp.
gaattcgcccttcccctggagcacgaggacactgacatggactgaaggagtagaaaatcagttcctgccaggacacagtttagatatgaggttccaggttcaggttctggggctccttctgctctggatatcaggtgcccactgtgatgtccagataacccagtctccatcttttcttgctgcatctcctggagaaaccattactattaattgcagggcaagtaagtacattagcaaacatttagtctggtatcaggagaaacctggagaaactaaaaagcttcttatctactctggatccactttgcaatctggacttccatcaaggttcagtggcagtggatctggtacagatttcactctcaccatcagtagcctggagcctgaagattttgcaatgtattactgtcaacagcataatgaatacccgctcacgttcggtgctgggaccaagctggagctgaaacgggctgatgctgcaccaactgtatccatcttcccaccatccagtgagcaaagggcgaattc

SEQ ID NO: 10 - protein, 107 aa, Mus sp.
DVQITQSPSFLAASPGETITINCRASKYISKHLVWYQEKPGETKKLLIYSGSTLQSGLPSRFSGSGSGTDFTLTISSLEPEDFAMYYCQQHNEYPLTFGAGTKLELK

SEQ ID NO: 11 - DNA, 553 bp, Homo sapiens
gaattcgcccttaattgcggccgcaaaccatgggatggagctgtatcatcctcttcttggtagcaacagctacaggcgcgcactccgaggtgcagctggtggagtctgggggaggcgtggtccagcctgggaggtccctgagactctcctgtgcagcgtctggattcaacttcggtacctatgccatgcactgggtccgccaggctcaaggcaaggggctggagtgggtggcactcatatggtatgatggaactaagaaatactatgcagactccgtgaagggccgatacaccatctccagagacaattcccagaacacgctgtatctgcaaatgaacaccctgagagccgacgacacggctgtgtattactgtgcgaaatcaaaactccaggggcgcgttatagactactggggccagggaaccctggtcaccgtctcctcagcctccaccaagggcccatcggtcttccccctggcaccctcctccaagagcacctctgggggcacagcggccctgggctgcctggtcaaggactacttaagggcgaattc

SEQ ID NO: 12 - protein, 119 aa, Homo sapiens
EVQLVESGGGVVQPGRSLRLSCAASGFNFGTYAMHWVRQAQGKGLEWVALIWYDGTKKYYADSVKGRYTISRDNSQNTLYLQMNTLRADDTAVYYCAKSKLQGRVIDYWGQGTLVTVSS

SEQ ID NO: 13 - DNA, 528 bp, Homo sapiens
gaattcgcccttaattgcggccgcaaacatgggatggagctgtatcatcctcttcttggtagcaacagctacaggcgtgcactccgacatccagatgacccagtctccagactccctggctgtgtctctgggcgagagggccaccatcaactgcaagtccagccagagtgttttatacagctccaacaataagaactacttagcttggtaccagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcaatattatagtactcctctgtggacgttcggccaagggaccaagctcgagatcaaacgaactgtggctgcaccatctgtcttcatcttcccgccatctgatgagcagttgaaatctggaactgcctctgttgtgtgcctgctgaaaagggcgaattc

SEQ ID NO: 14 - protein, 113 aa, Homo sapiens
IQMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTPLWTFGQGTKLEIK

SEQ ID NO: 15 - DNA, 29 bp, Artificial
  Primer used in expression of selected antibodies as secreted human IgG1
attaggatccggtcaccgtctcctcagcc

SEQ ID NO: 16 - DNA, 31 bp, Artificial
  Primer used in expression of selected antibodies as secreted human IgG1
attagtcgactcatttacccggagacaggga

SEQ ID NO: 17 - DNA, 1084 bp, Homo sapiens
gcggccgcaaaccatgggatggagctgtatcatcctcttcttggtagcaacagctacaggcgcgcatatggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtcgtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgagtcgac

SEQ ID NO: 18 - DNA, 413 bp, Homo sapiens
gcggccgcaaaccatgggatggagctgtatcatcctcttcttggtagcaacagctacaggcgtgcacttgactcgagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaggtcgac

SEQ ID NO: 19 - DNA, 422 bp, Homo sapiens
gcggccgcaaaccatgggatggagctgtatcatcctcttcttggtagcaacagctacaggcgtgcacttgactcgagaagcttaccgtcctacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaggtcgac

SEQ ID NO: 20 - DNA, 29 bp, Artificial
  IgG2-F primer
attaggatccggtcaccgtctcctcagcc

SEQ ID NO: 21 - DNA, 31 bp, Artificial; identical to SEQ ID NO: 16
  IgI2-R  primer

SEQ ID NO: 22 - DNA, 29 bp, Artificial
  IgG3-F  primer
attaggatccggtcaccgtctcctcagct

SEQ ID NO: 23 - DNA, 31 bp, Artificial; identical to SEQ ID NO: 16
  IgG3-R  primer

SEQ ID NO: 24 - DNA, 29 bp, Artificial
  IgG4-F  primer
attaggatccggtcaccgtctcctcagct

SEQ ID NO: 25 - DNA, 31 bp, Artificial
  IgG4-R  primer
attagtcgactcatttacccagagacaggga

SEQ ID NO: 26 - DNA, 30 bp, Artificial
  IgA1-F  primer
attaggatccggtcaccgtctcctcagcat

SEQ ID NO: 27 - DNA, 31 bp, Artificial
  IgA1-R  primer
attagtcgactcagtagcaggtgccgtccac

SEQ ID NO: 28 - DNA, 30 bp, Artificial; identical to SEQ ID NO: 26
  IgA2-F  primer

SEQ ID NO: 29 - DNA, 31 bp, Artificial; identical to SEQ ID NO: 27
  IgA2-R  primer

SEQ ID NO: 30 - DNA, 30 bp, Artificial
  IgD-F  primer
attaggatccggtcaccgtctcctcagcac

SEQ ID NO: 31 - DNA, 31 bp, Artificial
  IgD-R  primer
attagtcgactcatttcatggggccatggtc

SEQ ID NO: 32 - DNA, 29 bp, Artificial
  IgE-F primer
attaggatccggtcaccgtctcctcagcc

SEQ ID NO: 33 - DNA, 31 bp, Artificial
  IgE-R
attagtcgactcatttaccgggatttacaga

SEQ ID NO: 34 - DNA, 29 bp, Artificial
  IgM-F
attaggatccggtcaccgtctcctcaggg

SEQ ID NO: 35 - DNA, 31 bp, Artificial
  IgM-R
attagtcgactcagtagcaggtgccagctgt

SEQ ID NO: 36 - DNA, 6 bp, Artificial
  BssHII site
gcgcgc

SEQ ID NO: 37 - DNA, 7 bp, Artificial
  BsteII site
ggtcacc

SEQ ID NO: 38 - DNA, 6 bp, Artificial
  ApaL1 site
gtgcac

SEQ ID NO: 39 - DNA, 6 bp, Artificial
  Xho1 site
ctcgag

SEQ ID NO: 40 - DNA, 39 bp, Artificial
  1F2VK forward primer
tatccgtgcactcccaaattgttctcacccagtctccag

SEQ ID NO: 41 - DNA, 29 bp, Artificial
  1F2VK reverse primer
atattctcgagcttggtcccccctccgaa

SEQ ID NO: 42 - DNA, 6 bp, Artificial
  ApaL1 site
gtgcac

SEQ ID NO: 43 - DNA, 6 bp, Artificial
  Xho1 site
ctcgag

SEQ ID NO: 44 - DNA, 41 bp, Artificial
  1F2VH forward primer
tataagcgcgcactccgatgtacagcttcaggagtcaggac

SEQ ID NO: 45 - DNA, 28 bp, Artificial
  1F2VH reverse primer
atattggtgaccagagtcccttggcccc

SEQ ID NO: 46 - DNA, 40 bp, Artificial
  1B3VK forward primer
tatccgtgcactccgatgtccagataacccagtctccatc

SEQ ID NO: 47 - DNA, 29 bp, Artificial
  1B3VK reverse primer
atattctcgagcttggtcccagcaccgaa

SEQ ID NO: 48 - DNA, 41 bp, Artificial
  1B3VH forward primer
tataagcgcgcactccgaggtgcagcttcaggagtcaggac

SEQ ID NO: 49 - DNA, 24 bp, Artificial
  1B3VH reverse primer
atattggtgaccgtggtcccagcg

SEQ ID NO: 50 - DNA, 935 bp, Homo sapiens
tcttctctctgcagagcgcaaatgttgtgtcgagtgcccaccgtgcccaggtaagccagcccaggcctcgccctccagctcaaggcgggacaggtgccctagagtagcctgcatccagggacaggccccagctgggtgctgacacgtccacctccatctcttcctcagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaaggtgggacccgcggggtatgagggccacatggacagacggcggcttcggcccaccctctgccctgggagtgaccgctgtgccaacctctgtccctacagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgagtgccacggccggcaagcc

SEQ ID NO: 51 - DNA, 935 bp, Homo sapiens
tcttctctctgcagagcgcaaatgttgtgtcgagtgcccaccgtgcccaggtaagccagcccaggcctcgccctccagctcaaggcgggacaggtgccctagagtagcctgcatccagggacaggccccagctgggtgctgacacgtccacctccatctcttcctcagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcatggaggtgcataatgccaagacaaagccacgggaggagcagttcaacagcacgttccgtgtggtcagcgtcctcaccgtcgtgcaccaggactggctgaacggcaaggagtacaagtgcaaggtctccaacaaaggcctcccagcccccatcgagaaaaccatctccaaaaccaaaggtgggacccgcggggtatgagggccacatggacagacggcggcttcggcccaccctctgccctgggagtgaccgctgtgccaacctctgtccctacagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaatgagtgccacggccggcaagcc

SEQ ID NO: 52 - DNA, 1780 bp, Homo sapiens
ctcgaggacctgctcttaggttcagaagcgaacctcacgtgcacactgaccggcctgagagatgcctctggtgccaccttcacctggacgccctcaagtgggaagagcgctgttcaaggaccacctgagcgtgacctctgtggctgctacagcgtgtccagtgtcctgcctggctgtgcccagccatggaaccatggggagaccttcacctgcactgctgcccaccccgagttgaagaccccactaaccgccaacatcacaaaatccggtgggtccagaccctgctcggggccctgctcagtgctctggtttgcaaagcatattcctggcctgcctcctccctcccaatcctgggctccagtgctcatgccaagtacacagggaaactgaggcaggctgaggggccaggacacagcccagggtgcccaccagagcagaggggctctctcatcccctgcccagccccctgacctggctctctaccctccaggaaacacattccggcccgaggtccacctgctgccgccgccgtcggaggagctggccctgaacgagctggtgacgctgacgtgcctggcacgtggcttcagccccaaggatgtgctggttcgctggctgcaggggtcacaggagctgccccgcgagaagtacctgacttgggcatcccggcaggagcccagccagggcaccaccacctacgctgtaaccagcatactgcgcgtggcagctgaggactggaagaagggggagaccttctcctgcatggtgggccacgaggccctgccgctggccttcacacagaagaccatcgaccgcatggcgggtaaacccacccacatcaatgtgtctgttgtcatggcggaggcggatggcacctgctactgagccgcccgcctgtccccacccctgaataaactccatgctcccccaagcagccccacgcttccatccggcgcctgtctgtccatcctcagggtctcagcacttgggaaagggccagggcatggacagggaagaataccccctgccctgagcctcggggggcccctggcacccccatgagactttccaccctggtgtgagtgtgagttgtgagtgtgagagtgtgtggtgcaggaggcctcgctggtgtgagatcttaggtctgccaaggcaggcacagcccaggatgggttctgagagacgcacatgccccggacagttctgagtgagcagtggcatggccgtttgtccctgagagagccgcctctggctgtagctgggagggaatagggagggtaaaaggagcaggctagccaagaaaggcgcaggtagtggcaggagtggcgagggagtgaggggctggactccagggccccactgggaggacaagctccaggagggccccaccaccctagtgggtgggcctcaggacgtcccactgacgcatgcaggaaggggcacctccccttaaccacactgctctgtacggggcacgtgggcacacatgcacactcacactcacatatacgcctgagccctgcaggagtggaacgttcacagcccagacccagttccagaaaagccaggggagtcccctcccaagcccccaagctcagcctgctcccccaggcccctctggcttccctgtgtttccactgtgcacagctcagggaccaactccacagacccctcccaggcagcccctgctccctgcctggccaagtctcccatcccttcctaagcccaactaggacccaaagcatagacagggaggggccgcgtggggtggcatcagaag

SEQ ID NO: 53 - protein, 220 aa, Homo sapiens
LEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTYAVTSILRVAAEDWKKGETFSCMVGHEALPLAFTQKTIDRMAGKPTHINVSVVMAEADGTCY

SEQ ID NO: 54 - DNA, 1059 bp, Homo sapiens
gcaagcttgaccagccccaaggtcttcccgctgagcctctgcagcacccagccagatgggaacgtggtcatcgcctgcctggtccagggcttcttcccccaggagccactcagtgtgacctggagcgaaagcggacagggcgtgaccgccagaaacttcccacccagccaggatgcctccggggacctgtacaccacgagcagccagctgaccctgccggccacacagtgcctagccggcaagtccgtgacatgccacgtgaagcactacacgaatcccagccaggatgtgactgtgccctgcccagttccctcaactccacctaccccatctccctcaactccacctaccccatctccctcatgctgccacccccgactgtcactgcaccgaccggccctcgaggacctgctcttaggttcagaagcgaacctcacgtgcacactgaccggcctgagagatgcctcaggtgtcaccttcacctggacgccctcaagtgggaagagcgctgttcaaggaccacctgaccgtgacctctgtggctgctacagcgtgtccagtgtcctgtcgggctgtgccgagccatggaaccatgggaagaccttcacttgcactgctgcctaccccgagtccaagaccccgctaaccgccaccctctcaaaatccggaaacacattccggcccgaggtccacctgctgccgccgccgtcggaggagctggccctgaacgagctggtgacgctgacgtgcctggcacgtggcttcagccccaaggatgtgctggttcgctggctgcaggggtcacaggagctgccccgcgagaagtacctgacttgggcatcccggcaggagcccagccagggcaccaccaccttcgctgtgaccagcatactgcgcgtggcagccgaggactggaagaagggggacaccttctcctgcatggtgggccacgaggccctgccgctggccttcacacagaagaccatcgaccgcttggcgggtaaacccacccatgtcaatgtgtctgttgtcatggcggaggtggacggcacctgctac

SEQ ID NO: 55 - protein, 353 aa, Homo sapiens
ASLTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPDRDLCGCYSVSSVLSGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 56 - DNA, 366 bp, Artificial
  Chimeric mouse/human 141D10 VH H732
caggtgcagctgcaggagtcgggcccaggactggtgaagcctccggagaccctgtccctcacctgcaatgtctctggtggctctatcggtagatactattggaactggatccgacagtccccagggaaggggctggagtggattggccatatccattacagtgggagcaccatctaccatccctccctcaagagtcgagtcagcatatcgctggacacgtccaagaaccaggtctccctgaagttgagttctgtgaccgctgcggacacggccgtgtattactgtgcacgaggtgcttactacgggggggccttttttccttacttcgatgtctggggccaagggaccacggtcaccgtctcctca

SEQ ID NO: 57 - protein, 122 aa, Artificial
  Chimeric mouse/human 141D10 VH H732
QVQLQESGPGLVKPPETLSLTCNVSGGSIGRYYWNWIRQSPGKGLEWIGHIHYSGSTIYHPSLKSRVSISLDTSKNQVSLKLSSVTAADTAVYYCARGAYYGGAFFPYFDVWGQGTTVTVSS

SEQ ID NO: 58 - DNA, 321 bp, Artificial
  Chimeric mouse/human UH8 VK L120
gacatccagatgacccagtctccatcctccctgtctgcatctatgggagacagagtcaccatcacttgccgggcgagtcagggcattaggaatcatttagcctggtatcagcagaaaccagggaaagctcctaatctcctgatctctgctgcatccactttgcaatcaggggtcccaactcgattcagtggcagtggatctggaacagatttcactctcaccatcagcagcctgcagcctgaagactctgcaacttattactgccaacagtataatcggtaccccctcactttcggccaagggaccaagctcgagatcaaa

SEQ ID NO: 59 - protein, 107 aa, Artificial
  Chimeric mouse/human UH8 VK L120
DIQMTQSPSSLSASMGDRVTITCRASQGIRNHLAWYQQKPGKAPNLLISAASTLQSGVPTRFSGSGSGTDFTLTISSLQPEDSATYYCQQYNRYPLTFGQGTKLEIK

SEQ ID NO: 60 - DNA, 369 bp, Artificial
  Chimeric mouse/human MSH3 VH (H835)
caggtgcagctgcaggagtcgggaggaggcttagttcagcctggggggtccctgagactctcttgtgcaggctctggattcaccttcagtagttactggatgcactgggtccgccaagctccagggaaggggctggtgtgggtctcacgtattgacactgatgggagtaccacaacctacgcggactccgtgaagggccgattcaccatctccagagacaacgccaagaacacactgtatctgcaaatgaacagcctgagagtcgaggacacggccgtgtattactgtgcacgaggtgcttactacgggggggccttttttccttacttcgatgtctggggccaagggaccacggtcaccgtctcctca

SEQ ID NO: 61 - protein, 123 aa, Artificial
  Chimeric mouse/human MSH3 VH (H835)
QVQLQESGGGLVQPGGSLRLSCAGSGFTFSSYWMHWVRQAPGKGLVWVSRIDTDGSTTTYADSVKGRFTISRDNAKNTLYLQMNSLRVEDTAVYYCARGAYYGGAFFPYFDVWGQGTTVTVSS

SEQ ID NO: 62 - protein, 125 aa, Artificial sequence
  VH of MAb163 (H730)
EVQLVESGGGLVKPGGSLRLSCEVSGITFSNAWMSWVRQAPGKGLEWVGRIKSKTDGGTTDYAAPVKGRFTISRDDSKNTLYLQMNSLKTEDTAVYYCSIGYYYDSSFKYGMDVWGQGTTVTVSS

SEQ ID NO: 63 - protein, 10 aa, Artificial sequence
  VH CDR 1 of MAb163 (H730)
GITFSNAWMS

SEQ ID NO: 64 - protein, 19 aa, Artificial sequence
  VH CDR 2 of MAb163 (H730)
RIKSKTDGGTTDYAAPVKG

SEQ ID NO: 65 - protein, 14 aa, Artificial sequence
  VH CDR 3 of MAb163 (H730)
GYYYDSSFKYGMDV

SEQ ID NO: 66 - protein, 107 aa, Artificial sequence
  VL of MAb163 (L74)
DIQMTQSPATLSASVGDRVTITCRASQSISRWLAWYQQKPGQAPKVLIYKASTLQSGVPSRFSGSGSGTEFSLTINSLQPDDFATYYCQQYYSYLRTFGQGTKLEIK

SEQ ID NO: 67 - protein, 11 aa, Artificial sequence
  VL CDR 1 of MAb163 (L74)
RASQSISRWLA

SEQ ID NO: 68 - protein, 7 aa, Artificial sequence
  VL CDR 2 of MAb163 (L74)
KASTLQS

SEQ ID NO: 69 - protein, 9 aa, Artificial sequence
  VL CDR 3 of MAb163 (L74)
QQYYSYLRT

SEQ ID NO: 70 - DNA, 375 bp, Artificial sequence
  VH of MAb163 (H730)
gaggtgcagctggtggagtctgggggaggcttggtaaagccgggggggtcccttagactctcctgtgaagtctctggaatcactttcagtaatgcctggatgagctgggtccgccaggctccagggaaggggctggagtgggttggccgtattaaaagcaaaactgatggtgggacaacagactacgctgcacccgtgaaaggcagattcaccatctcaagagatgattcaaaaaacacgctgtatctgcaaatgaacagcctgaaaaccgaggacacagccgtgtattattgtagcatagggtattactatgatagtagtttcaaatacggtatggacgtctggggccaagggaccacggtcaccgtctcctca

SEQ ID NO: 71 - DNA, 321 bp, Artificial sequence
  VL of MAb163 (L74)
gacatccagatgacccagtctcctgccaccctgtctgcatctgtaggagacagagtcaccatcacttgccgggccagtcagagtattagtcggtggttggcctggtatcagcagaagccaggacaagcccctaaagtcttgatctataaggcgtctactttacaaagtggggtcccatcaaggttcagcggcagtgggtctgggacagaattcagtctcaccatcaacagcctgcagcctgatgattttgcaacttattattgccaacagtattatagttatcttcggacgttcggccaagggaccaagctcgagatcaaa

SEQ ID NO: 72 - DNA, 30 bp, Artificial sequence
  CDR 1 nucleotide (H730)
ggaatcactttcagtaatgcctggatgagc

SEQ ID NO: 73 - DNA, 57 bp, Artificial sequence
  CDR 2 nucleotide (H730)
cgtattaaaagcaaaactgatggtgggacaacagactacgctgcacccgtgaaaggc

SEQ ID NO: 74 - DNA, 42 bp, Artificial sequence
  CDR 3 nucleotide (H730)
gggtattactatgatagtagtttcaaatacggtatggacgtc

SEQ ID NO: 75 - DNA, 33 bp, Artificial sequence
  CDR 1 nucleotide (L74)
cgggccagtcagagtattagtcggtggttggcc

SEQ ID NO: 76 - DNA, 21 bp, Artificial sequence
  CDR 2 nucleotide (L74)
aaggcgtctactttacaaagt

SEQ ID NO: 77 - DNA, 27 bp, Artificial sequence
  CDR 3 nucleotide (L74)
caacagtattatagttatcttcggacg